(19) United States
(12) Patent Application Publication
(10) Pub. No.: US 2007/0032640 A1
(43) Pub. Date: Feb. 8, 2007

(54) Cytokine receptor

(76) Inventors: Joseph Noozhumutry Varghese, Victoria (AU); Richard J. Simpson, Richmond (AU); Robert Lorenz Moritz, Victoria (AU); Meizhen Lou, Victoria (AU); Hong Ji, Victoria (AU); Kim Matthew Branson, Victoria (AU); Brian John Smith, Victoria (AU)

Correspondence Address: FOLEY AND LARDNER;SUITE 500, 3000 K STREET NW, WASHINGTON, DC, 20007, US

(21) Appl. No.: 10/489,705

(22) PCT Filed: Sep. 16, 2002
(86) PCT No.: PCT/AU02/01255

§ 371 Date: May 20, 2004

(30) Foreign Application Priority Data
Sep. 14, 2001 (AU) 7695

(51) Int. Cl. C07K 14/54; G06F 19/00; G01N 33/48; G01N 33/50; A61K 38/20

(52) U.S. Cl. 530/351; 702/19; 424/85.2

(57) ABSTRACT

A crystalline composition comprising a crystal of the IL-6 receptor I chain is provided. Also provided are methods of using the crystal and related structural information to screen for and design compounds that interact with IL-6R, or variants thereof. Also provided arc methods of modulating an IL-6 receptor comprising contacting the IL-6 receptor with a compound identified by the screening method of the invention.

### FIELD OF THE INVENTION

The present invention relates generally to structural studies of the interleukin-6 (IL-6) receptor. More particularly, the present invention relates to the crystal structure of the IL-6 receptor α chain (IL-6R). Even more particularly, the instant invention relates to the crystal structure of an extracellular portion of IL-6R and to methods of using the crystal and related structural information to screen for and design compounds that interact with IL-6R, or variants of thereof.

### BACKGROUND OF THE INVENTION

Interleukin-6 (IL-6) is a multifunctional cytokine that plays a central role in host defense due to its wide range of immune and haematopoietic activities and its potent ability to induce acute phase response (for review see Simpson et al. 1997, Protein Sci 6, 929-55). It appears to represent an important frontline component of the body's armory against infection or tissue damage (IL-6 knockout mice have impaired immune and acute phase responses. IL-6 was originally referred to by such diverse names as interferon-β2, 26K factor, B-cell stimulating factor 2, hybridoma growth factor, plasmacytoma growth factor, hepatocyte-stimulatory factor, a haematopoietic factor and cytotoxic T-cell differentiation factor—each name reflecting a different biological activity controlled by the same protein. Over the last ten years it has become clear that the functional pleiotropy of Interleukin-6 has implicated this cytokine in the pathology of many human diseases such as multiple myeloma, rheumatoid arthritis, Castleman's disease, AIDS, mesangial proliferative glomerulonephritis, Kaposi's sarcoma, sepsis, osteoporosis and psoriasis. Given the association of abnormal IL-6 production and clinical disorders there is great interest in the development of functional agonists and antagonists as potential therapeutic agents in the treatment of IL-6 associated diseases.

The biological activities of IL-6 are mediated by the IL-6 receptor complex, which consists of two membrane proteins, the ligand binding α-chain receptor (IL-6R, gp80) and the signal transducing β-chain, gp130, which also forms part of the receptor complexes of LIF, OSM, CNTF, IL-11 as well as CT-1. Co-expression of IL-6R and gp130 results in both low- and high-affinity binding sites for IL-6, the relative amounts of the two chains dictating the ratio between the two affinity states. High affinity binding is likely due to the ability of IL-6 to interact simultaneously with sites on the IL6-R and gp130. Typically the difference in affinity between low- and high-affinity binding is ˜100-fold; e.g., on human myeloma U266 cells, IL-6 first binds IL-6R with an affinity of ˜1 nM, and the IL-6/IL-6R complex then binds gp130 with a resulting affinity of ˜10 pM. Binding of IL-6 has been demonstrated on a variety of human cells. Although human IL-6R shows broad distribution (e.g., activated B-cells, resting T-cells, B lymphoblastoid cell types, hepatoma lines, myeloma and monocyte cell lines), some cells lack this type of receptor. Normal cells express between 10² and 10³ receptors, while human myeloma U266 and EB virus-transformed CESS cells have up to 10⁴ receptors.

The cDNA of the human IL-6R encodes a protein of 468 amino acids, including a signal peptide of 19 amino acids, an extracellular region of 339 amino acids, a transmembrane domain of 28 amino acids, and a short cytoplasmic domain of 82 amino acids (Yamasaki et al., 1988, Science 241: 825-828). There are six potential N-linked glycosylation sites on the extracellular domain, the mature 80 kDa IL-6R is a glycosylated form of the predicted 50 kDa precursor. Secondary structural predictions indicate that the extracellular region is highly modular, consisting of three domains (D1, D2 and D3) of approximately 100 residues. These domains consist of an N-terminal domain D1 characteristic of the immunoglobulin (Ig) superfamily and a cytokine binding domain (CBD), which consists of two fibronectin type III-like (FN III) domains (a subclass of the Ig-fold). The CBD of IL-6R is characteristic of the class I cytokine receptors. In common with other members of this receptor family, the N-terminal FIII domain (D2) of the CBD has four conserved cysteines, while the C-terminus FIII domain (D3) has a conserved sequence motif, the “WSXWS” motif. The Ig domain D1 has been deleted without major effect on the binding of IL-6 or signal transduction, suggesting that the CBD mediates binding to IL-6 and gp130. The transmembrane and cytoplasmic domains of the IL-6R are not necessary for signal transduction, as shown by the fact that the complex of IL-6 and extracellular “soluble” domain of IL-6R induces signal transduction on cells expressing gp130.

Notwithstanding the known biology of the IL-6R complex, the design of IL-6R complex agonists or antagonists is impeded greatly by the lack of three-dimensional structural information available for this complex. Accordingly, knowledge of the three-dimensional structure coordinates of the IL-6R complex would be useful in facilitating the design of potential selective agonists/antagonists which, in turn, are expected to have therapeutic utility.

### SUMMARY OF THE INVENTION

The present inventors have now obtained three-dimensional structural information concerning IL-6 receptor. The information presented in the present application can be used to develop compounds which interact with the IL-6 receptor for use in therapeutic applications.

Accordingly, in a first aspect the present invention provides a method of selecting or designing a compound that interacts with the IL-6 receptor and modulates an activity mediated by the receptor, the method comprising the step of assessing the stereochemical complementarity between the compound and a topographic region of the receptor, wherein the receptor is characterised by

(i) amino acids 1-299 of the IL-6 receptor positioned at atomic coordinates as shown in Appendix I, or structural coordinates having a root mean square deviation from the backbone atoms of said amino acids of not more than 1.5 Å; or

(ii) one or more subsets of said amino acids related to the coordinates shown in Appendix I by whole body translations and/or rotations.

In a preferred embodiment of the first aspect, the structural coordinates have a root mean square deviation from the backbone atoms of said amino acids of not more than 1.0 Å and more preferably not more than 0.7 Å.

By “stereochemical complementarity” we mean that the compound or a portion thereof makes a sufficient number of energetically favourable contacts with the receptor, or topographical region thereof, as to have a net reduction of free energy on binding to the receptor, or topographical region thereof.

In a second aspect the present invention provides computer-assisted method for identifying potential compounds able to interact with the IL-6 receptor and thereby modulate an activity mediated by the receptor, using a programmed computer comprising a processor, an input device, and an output device, comprising the steps of:
(a) inputting into the programmed computer, through the input device, data comprising the atomic coordinates of amino acids 1-299 of the IL-6 receptor as shown in Appendix I, or structural coordinates having a root mean square deviation from the backbone atoms of said amino acids of not more than 1.5 Å, or one or more subsets of said amino acids, or one or more subsets of said amino acids related to the coordinates shown in Appendix I by whole body translations and/or rotations;
(b) generating, using computer methods, a set of atomic coordinates of a structure that possesses stereochemical complementarity to the atomic coordinates of amino acids 1-299 of the IL-6 receptor as shown in Appendix I, or structural coordinates having a root mean square deviation from the backbone atoms of said amino acids of not more than 1.5 Å, or one or more subsets of said amino acids, or one or more subsets of said amino acids related to the coordinates shown in Appendix I by whole body translations and/or rotations, thereby generating a criteria data set;
(c) comparing, using the processor, the criteria data set to a computer database of chemical structures;
(d) selecting from the database, using computer methods, chemical structures which are similar to a portion of said criteria data set; and
(e) outputting, to the output device, the selected chemical structures which are complementary to or similar to a portion of the criteria data set.

In a third aspect the present invention provides a computer for producing a three-dimensional representation of a molecule or molecular complex, wherein the computer comprises:
(a) a machine-readable data storage medium comprising a data storage material encoded with machine-readable data, wherein the machine readable data comprise the atomic coordinates of amino acids 1-299 of the IL-6 receptor as shown in Appendix I, or structural coordinates having a root mean square deviation from the backbone atoms of said amino acids of not more than 1.5 Å, or one or more subsets of said amino acids, or one or more subsets of said amino acids related to the coordinates shown in Appendix I by whole body translations and/or rotations;
(b) a working memory for storing instructions for processing the machine-readable data;
(c) a central-processing unit coupled to the working memory and to the machine-readable data storage medium, for processing the machine-readable data into the three dimensional representation; and
(d) an output hardware coupled to the central processing unit, for receiving the three-dimensional representation.

In a fourth aspect the present invention provides a compound able to bind to the IL-6 receptor and to modulate an activity mediated by the receptor, the compound being obtained by a method according to the present invention.

In a fifth aspect the present invention provides a compound which possesses stereochemical complementarity to a topographic region of the IL-6 receptor and which modulates an activity mediated by the receptor, wherein the receptor is characterised by
(i) amino acids 1-299 of the IL-6 receptor positioned at atomic coordinates as shown in Appendix I, or structural coordinates having a root mean square deviation from the backbone atoms of said amino acids of not more than 1.5 Å; or
(ii) one or more subsets of said amino acids related to the coordinates shown in Appendix I by whole body translations and/or rotations;

with the proviso that the compound is not a naturally occurring ligand of a molecule of the IL-6 receptor or a mutant thereof.

By “mutant” we mean a ligand which has been modified by one or more point mutations, insertions of amino acids or deletions of amino acids.

In a sixth aspect, the present invention provides a pharmaceutical composition comprising a compound according to the present invention together with a pharmaceutically acceptable carrier or diluent. The present invention also provides the use of a compound or pharmaceutical composition of the invention in a method of preventing or treating a disease associated with signalling by the IL-6 receptor.

In a related aspect the present invention provides a method of preventing or treating a disease associated with signalling by the IL-6 receptor which method comprises administering to a subject in need thereof a compound according to the present invention.

In yet another aspect, the present invention provides a method for evaluating the ability of a chemical entity to interact with the IL-6 receptor, said method comprising the steps of:
(a) creating a computer model of at least one region of the IL-6 receptor using structure coordinates wherein the root mean square deviation between said structure coordinates and the structure coordinates of amino acids 1-299 of IL-6 receptor as set forth in Appendix I is not more than about 1.5 Å;
(b) employing computational means to perform a fitting operation between the chemical entity and said computer model of the binding surface; and
(c) analysing the results of said fitting operation to quantify the association between the chemical entity and the binding surface model.

In a further aspect the present invention provides a method of selecting or designing a compound that interferes with the formation of the IL-6, IL-6R, gp130 hexameric complex, the method comprising the step of assessing the stereochemical complementarity between the compound and a topographic region of the complex, wherein the complex is characterised by

(i) the amino acids of IL-6, IL-6 receptor and gp130 positioned at atomic coordinates as shown in Appendix II, or structural coordinates having a root mean square deviation from the backbone atoms of said amino acids of not more than 1.5 Å; or

(ii) one or more subsets of said amino acids related to the coordinates shown in Appendix II by whole body translations and/or rotations.

In another aspect the present invention provides computer-assisted method for identifying compounds that interfere with the formation of the IL-6, IL-6R, gp130 hexameric complex, using a programmed computer comprising a processor, an input device, and an output device, comprising the steps of:
(a) inputting into the programmed computer, through the input device, data comprising the atomic coordinates of amino acids IL-6, IL-6R and gp130 as shown in Appendix II, or structural coordinates having a root mean square deviation from the backbone atoms of said amino acids of not more than 1.5 Å, or one or more subsets of said amino acids, or one or more subsets of said amino acids related to the coordinates shown in Appendix II by whole body translations and/or rotations;
(b) generating, using computer methods, a set of atomic coordinates of a structure that possesses stereochemical complementarity to the atomic coordinates the IL-6, IL-6R, gp130 hexameric complex as shown in Appendix II, or structural coordinates having a root mean square deviation from the backbone atoms of said amino acids of not more than 1.5 Å, or one or more subsets of said amino acids, or one or more subsets of said amino acids related to the coordinates shown in Appendix II by whole body translations and/or rotations, thereby generating a criteria data set;
(c) comparing, using the processor, the criteria data set to a computer database of chemical structures;
(d) selecting from the database, using computer methods, chemical structures which are similar to a portion of said criteria data set; and
(e) outputting, to the output device, the selected chemical structures which are complementary to or similar to a portion of the criteria data set.

In another aspect the present invention provides a computer for producing a three-dimensional representation of a molecule or molecular complex, wherein the computer comprises:
(a) a machine-readable data storage medium comprising a data storage material encoded with machine-readable data, wherein the machine readable data comprise the atomic coordinates of the IL-6, IL-6R, gp130 hexameric complex as shown in Appendix II, or structural coordinates having a root mean square deviation from the backbone atoms of said amino acids of not more than 1.5 Å, or one or more subsets of said amino acids, or one or more subsets of the coordinates shown in Appendix II by whole body translations and/or rotations;
(b) a working memory for storing instructions for processing the machine-readable data;
(c) a central-processing unit coupled to the working memory and to the machine-readable data storage medium, for processing the machine-readable data into the three dimensional representation; and
(d) an output hardware coupled to the central processing unit, for receiving the three-dimensional representation.

In another aspect the present invention provides a compound which possesses stereochemical complementarity to a topographic region of the IL-6, IL-6R, gp130 hexameric complex and which modulates an activity mediated by the complex, wherein the complex is characterised by
(i) the amino acids of IL-6, IL-6R and gp130 positioned at atomic coordinates as shown in Appendix II, or structural coordinates having a root mean square deviation from the backbone atoms of said amino acids of not more than 1.5 Å; or
(ii) one or more subsets of said amino acids related to the coordinates shown in Appendix II by whole body translations and/or rotations.

In yet another aspect, the present invention provides a method for evaluating the ability of a chemical entity to interact with the IL-6, IL-6R, gp130 hexameric complex, said method comprising the steps of:
(a) creating a computer model of at least one region of the IL-6, IL-6R, gp130 hexameric complex using structure coordinates wherein the root mean square deviation between said structure coordinates and the structure coordinates set forth in Appendix I is not more than about 1.5 Å;
(b) employing computational means to perform a fitting operation between the chemical entity and said computer model; and
(c) analysing the results of said fitting operation to quantify the association between the chemical entity and the model.

In a still further aspect the present invention consists in a crystalline composition comprising an IL-6 receptor or portion thereof or variant of these.

As will be readily understood by persons skilled in this field the methods of the present invention provide a rational method for designing and selecting compounds which interact with the IL-6 receptor. In many cases these compounds can be developed further to increase activity. Such further development is routine in this field and will be assisted by the structural information provided in this application. It is intended that in particular embodiments the methods of the present invention includes such further developmental steps.

In another aspect the present invention consists in a method of assessing the interaction between a compound and the IL-6 receptor, the method comprising exposing a crystalline composition comprising IL-6 receptor or portion thereof or variant of these to the compound and measuring the leveling of binding of the compound to the crystal.

In yet a further aspect, the invention provides a method of using molecular replacement to obtain structural information about a molecule or a molecular complex of unknown structure, comprising the steps of:
(i) crystallising said molecule or molecular complex;
(ii) generating an X-ray diffraction pattern from said crystallized molecule or molecular complex;
(iii) applying at least a portion of the structure coordinates set forth in Appendix I to the X-ray diffraction pattern to generate a three-dimensional electron density map of at least a portion of the molecule or molecular complex whose structure is unknown.

The term “molecular replacement” refers to a method that involves generating a preliminary model of an IL-6 extracellular domain crystal whose structure coordinates are unknown, by orienting and positioning a molecule whose structure coordinates are known (e.g., IL-6 receptor extracellular coordinates from Appendix I) within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal (Lattman, 1985, Methods in Enzymology 115: 55-77; M. G. Rossmann, ed., “The Molecular Replacement Method”, Int. Sci. Rev. Ser., No. 13, Gordon & Breach, New York, 1972).

In yet a further aspect the present invention provides a method for preventing or treating a disease associated with signaling by the IL-6 receptor which method comprises administering to a subject in need thereof a compound identified by a method comprising the step of assessing the stereochemical complementarity between the compound and a topographic region of the receptor, wherein the receptor comprises:
(i) amino acids 1-299 of the IL-6 receptor positioned at atomic coordinates as shown in Appendix I, or structural coordinates having a root mean square deviation from the backbone atoms of said amino acids of not more than 1.5 Å; or
(ii) one or more subsets of said amino acids related to the coordinates shown in Appendix I by whole body translations and/or rotations.

In yet a further aspect the present invention provides a method for preventing or treating a disease associated with signaling by the IL-6 receptor which method comprises administering to a subject in need thereof a compound identified by a method comprising the step of assessing the stereochemical complementarity between the compound and a topographic region of an IL-6, IL-6R, gp130 hexameric complex, wherein the hexameric complex comprises:
(i) the amino acids of IL-6, IL-6 receptor and gp130 positioned at atomic coordinates as shown in Appendix II, or structural coordinates having a root mean square deviation from the backbone atoms of said amino acids of not more than 1.5 Å; or
(ii) one or more subsets of said amino acids related to the coordinates shown in Appendix II by whole body translations and/or rotations.

Throughout this specification, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### DETAILED DESCRIPTION

The present inventors have determined the three-dimensional structure of the extracellular region of the IL-6R. The X-ray structure of IL-6R reveals a structure consisting of the N-terminal Ig domain (D1) linked to a classical CBD (D2, D3). The first 5 residues of the N-terminus are poorly defined and amino acids past residue 299 are not visible in the X-ray structure. The D2 and D3 are connected at about 90° to each other and D1 is connected at about 45° to D2. The three domains lie on a similar plane making the receptor a long ‘flat’ structure. Carbohydrate is observed at 4 sites (five hexoses on the Asn226 site), all only on one of the ‘flat’ faces of the molecule, indicating that this face would have very restricted involvement in the signalling complex. This is consistent with evidence of expression of functional sIL-6R in Escherichia coli. The N-terminus Cys6 is disulphide bonded to Cys174 in D2, and this anchoring results in an unusual β-sheet arrangement of D1. D1 has an s-type Ig topology (0.7 Å rmsd with the ligand binding domain of fibroblast growth factor (Stauber et al., 2000, PNAS 97: 49-54), but the first ‘a’ strand of the 3-stranded β-sheet has moved to form a fifth ‘h’ strand of the 4-stranded β-sheet. This could be a result of crystal packing and represent a degree of flexibility of this domain, acting as a conformational switch, allowing D1 to rotate about 20° by β-strand shifting. This could also explain the higher disorder in this domain resulting in lack of isomorphism between different crystals of IL-6R. As in other cytokine FnIII domains, the domains are separated by a double proline motif (Pro94-Pro95, Pro199-Pro200 and Pro302-Pro303 at the end of D1, D2 and D3 respectively).

The other characteristic of the CBD of IL-6R is the long tryptophane-arginine ladder (from the C-terminus end of D3 is Arg239, Phe246, Arg237, Trp287, Arg274, Trp284, Gln276) found in other class 1 CBD structures (Carr et al., 2001). This ladder incorporates the conserved WSXWS motif (Residues 284 to 287 in IL-6R) located in the COOH-terminal of CBDs. The polypeptide backbone of this motif has an unusual left-handed 3₁₀ helix similar to a poly-proline helix, stabilised by the large tryptophane and arginine stacking and not by main chain H-bonds. This results in a long surface stripe of positive charge (from the guanidinium and tryptophane nitrogens) running along the side of the inside elbow portion of D3, and a groove formed by the 3₁₀ helix running parallel to it. The function of this structure is unknown, but the similarities with other CBDs would suggest that it could be involved in a general receptor transport system and not in IL-6 binding.

Following the fibroblast growth factor paradigm (Stauber et al., 2000), it would be expected that IL-6 would bind in the region of the outer elbow formed at the junction of D2 and D3, characterised by 4 loops (L1 to L4) from D2 and 3 loops (L5 to L7) from D3. IL-6 would engage residues in these loops, and mutation in these residues would affect IL-6 binding. Mutational analyses indicate that most of the mutations, when mapped onto the crystal structure, have altered binding due to alterations in the structural integrity of the molecule, in particular mutations in the tryptophane/arginine ladder. Discarding these ‘structural’ mutations, there are clusters of mutations in loops L3, L5 and L7 that reduce binding to IL-6. This site is at the juncture of D2 and D3 domains, and can be inferred to be primarily responsible for IL-6 binding.

In the crystal lattice two molecules of IL-6R related by a crystallographic 2-fold axis are closely associated along the length of each molecule. The association is primarily a hydrophobic contact around the 2-fold axis involving Phe134 and Phe168 of domain D2, the salt-link Glu97-Arg274, and H-bonding of Glu283 with the main chain nitrogens of Thr186. The buried accessible surface area of each molecule is about 1230 Å² (shape and electrostatic complementarity of 0.722 and 0.728), and is what could be expected for a protein-protein interaction in solution. Furthermore the buried surface of a protein complex does not have to be as extensive for membrane bound receptors to interact, compared to complexes formed where one or both components are in solution. So it is consistent with IL6-R forming dimers on the cell surface of the type observed in the crystal.

The orientation of the dimer is such that the interaction is along the carbohydrate-free ‘flat’ face of the molecule. In this model, domain D3 comes up away from the membrane at 45° and D2 comes down towards the membrane at 30°, with finally D1 running parallel to the membrane. The dimer thus appears as a bridge structure like the common β-receptor of IL-3, with a 50 Å tunnel of triangular cross-section (base ˜40 Å; height ˜22 Å). Non-structural mutations in IL-6R that affect gp130 signalling are found on the loops L1, L3, L5 and L6, and also in a patch of residues at the dimer interface consisting of residues 140 to 142, 167 to 171 (reduce signalling (and 182 to 186 (increase signalling) of the N-terminal side of the d, f and g strands of D2 respectively. These is also mutations in a patch of residues (209 to 213 and 261 to 263) on the membrane distal side of domain D2 that reduce binding.

The recent structure of the Ig and CBD domain gp130 and viral IL-6 complex (Chow et al., 2001, Science 291: 2150-5) reveals a possible dimeric relationship between the IL-6 binding domains of gp130. Thus incorporating the dimeric relationship between all the IL-6 binding receptor domains it is possible to construct a model for the signalling complex that is substantially different from current proposed models and consistent with the available mutational data. Here two IL-6 molecules bind to the IL-6R dimer, followed by two gp130 each binding to both IL-6 molecules in a similar way to the viral IL-6/gp130 fragment complex. gp130 would bind with its second CBD pointing away from the membrane. The remaining three FnIII domains of gp130 would head down towards the membrane, and the signalling activated by dimerisation of the membrane proximal FnIII domains under the bridge of the IL-6R dimer likely through disulphide crosslinking.

Clearly the information provided in this application will enable rational design/selection of compounds which will interact with IL-6R.

Accordingly, in a first aspect the present invention provides a method of selecting or designing a compound that interacts with an IL-6 receptor and modulates an activity mediated by the receptor, the method comprising

(a) assessing the stereochemical complementarity between a compound and a topographic region of the receptor, wherein the receptor comprises:
(i) amino acids 1-299 of the IL-6 receptor positioned at atomic coordinates as shown in Appendix I, or structural coordinates having a root mean square deviation from the backbone atoms of said amino acids of not more than 1.5 Å; or
(ii) one or more subsets of said amino acids related to the coordinates shown in Appendix I by whole body translations and/or rotations;

(b) obtaining a compound which possesses stereochemical complementarity to a topographic region of the receptor; and

(c) testing the compound for its ability to modulate an activity associated with the receptor.

In a preferred embodiment of the first aspect, the subset of amino acids is selected from the group consisting of the subset of amino acids representing the D1 domain (residues 1 to 93), the subset of amino acids representing the D2 domain (residues 94 to 194) and the subset of amino acids representing the D3 domain (residues 195 to 299).

As discussed above, following the fibroblast growth factor receptor paradigm it is expected that IL-6 will bind to IL-6R in the region of the outer elbow formed at the junction of D2 and D3, characterised by 4 loops (L1 to L4) from D2 and 3 loops (L5 to L7) from D3. The ligand binding surfaces of IL-6R are therefore defined by residues 106-110, 133-138, 160-168, 190-193, 227-233, 250-256 and 276-281.

Accordingly, in a preferred embodiment the compound is selected or designed to interact with the IL-6 receptor in a manner such as to interfere with the binding of natural ligand to one or more of the residues of IL-6R selected from the group consisting of 106-110, 133-138, 160-168, 190-193, 227-233, 250-256, 276-281 and combinations thereof.

The present inventors have determined that the IL-6R homodimer involves interaction along the carbohydrate free flat faces of the two IL-6 receptors. This dimer interface is defined by residues 1-5, 19-23, 65-69, 93-99, 118, 119, 132-141, 166-172, 179-196, 241-250, 261, 262, 272-276 and 282-290.

Accordingly, in a preferred embodiment the compound is selected or designed to interact with the IL-6 receptor in a manner such as to interfere with IL-6R homodimer formation.

The compound may interfere with ligand binding or dimer formation in a number of ways. For example the compound may bind or interact with the receptor at or near one or more of the specified residues or corresponding regions and by steric overlap and/or electrostatic repulsion prevent natural ligand binding or dimer formation. Alternatively the compound may bind to the receptor so as to interfere allosterically with natural ligand binding or dimer or formation.

It is presently preferred, however, that the compound binds or interacts with the receptor at or near one or more of the specified residues.

The present inventors have also identified two other regions of the IL-6 receptor that are of particular interest in the development of compounds which affect IL-6 receptor activity. The first is an apparent allosteric switch in D1. It was observed that the first ‘a’ strand of the 3-stranded β-sheet of D1 has moved to form a fifth ‘h’ strand of the 4-stranded β-sheet. This could be a result of crystal packing and represent a degree of flexibility of this domain, acting as a conformational switch, allowing D1 to rotate about 20° by b-strand shifting. This proposed allosteric movement can be blocked by designing a molecule to bind to this switch region and block the putative movement. This region is defined by the residues 11, 45, 46, 55, 62-66, 69-72, 75, 81, 88, 90-93, 122-124 and 178.

The second is the long tryptophane-arginine ladder which incorporates the conserved WSXWS motif. The polypeptide backbone of this motif has an unusual left-handed 3₁₀ helix similar to a poly-proline helix, stabilised by the large tryptophane and arginine stacking and not by main chain H-bonds. This results in a long surface stripe of positive charge (from the guanidinium and tryptophane nitrogens) running along the side of the inside elbow portion of D3, and a groove formed by the 3/10 helix running parallel to it. The function of this structure is unknown, but the similarities with other CBDs would suggest that it could be involved in a general receptor transport system and not in IL-6 binding. Blocking this region could be useful to regulate transport of receptor to the cell surface. This region is defined by the following residues 233-239, 244-248 and 270-290. The WSXWS motif is residues 284-287.

Accordingly in a preferred embodiment of the first aspect of the present invention, the method comprises selecting or designing a compound which has portions that match residues positioned on;
(i) the ligand binding surface of IL-6R defined by amino acids 106-110, 133-138, 160-168, 190-193, 227-233, 250-256 and 276-281; or
(ii) the IL-6R dimer interface defined by amino acids 1-5, 19-23, 65-69, 93-99, 118, 119, 132-141, 166-172, 179-196, 241-250, 261, 262, 272-276 and 282-290; or
(iii) amino acids 11, 45, 46, 55, 62-66, 69-72, 75, 81, 88, 90-93, 122-124 and 178 of IL-6R; or
(iv) amino acids residues 233-239, 244-248 and 270-290 of IL-6R.

By “match” we mean that the identified portions interact with the surface residues, for example, via hydrogen bonding or by enthalpy-reducing Van der Waals interactions which promote desolvation of the biologically active compound with the receptor, in such a way that retention of the compound by the receptor is favoured energetically.

In a further preferred embodiment of the present invention, the stereochemical complementarity is such that the compound has a K_{d} for the receptor site of less than 10⁻⁶M. More preferably, the K_{d} value is less than 10⁻⁸M and more preferably less than 10⁻⁹M.

In preferred embodiments of the first aspect of the present invention, the compound is selected or modified from a known compound identified from a data base.

For example this specification provides information regarding the portions of IL-6 which are involved in receptor binding. With this information IL-6 variants may be designed in which specific residues are modified or altered such that the variant is able to bind to the receptor but not initiate signalling. It would be expected that such a variant would compete with the natural ligand for binding to the receptor. Such a variant would therefore be an antagonist. In a similar manner variants which would act as agonists could be designed. In this case the modifications or alterations would be selected such as to increase the strength of interaction between the receptor and the variant so as to lead to increased signalling.

As will be understood by those skilled in this field knowledge of the structure of a protein complex is of assistance in the development of mutants of one of the proteins with enhanced affinity for its protein partner. Structural information can be used to select residues on one or more of the protein interfaces in the complex for alteration by methods such as site-directed mutagenesis or phage display. For example, amino acid positions in growth hormone which were allowed to vary were chosen in part from the crystal structure of the complex of growth hormone bound to two molecules of the human growth hormone extracellular region (Lowman and Wells, 1993, J. Mol. Biol. 234: 564-578.). Using a model of the granulocyte colony-stimulating factor (G-CSF) receptor ligand binding domain, residues of the receptor were chosen for mutagenesis by analogy with the structure of human growth hormone bound to its receptors (Layton et al., 1997, J Biol. Chem. 272: 29735-29741.). Some of the mutant G-CSF receptors were found to bind G-CSF with slightly enhanced affinity. The structure of the complex could also be used to design mutations which would potentially increase the binding affinity, for example by increasing the amount of hydrogen bonds and/or van der Waals interactions across the interface.

The modification of protein residues to enhance protein binding affinity is not restricted to those residues in the relevant protein-protein interfaces. Modification of residues outside of an interface may lead to alterations due to changes in the long-range electrostatic interactions between the two interacting proteins which changes the rate of association and subsequently the equilibrium binding constant (Seizer and Schreiber, 1999, J Mol Biol. 287: 409-419.; Seizer et al, 2000, Nat Struct Biol. 7: 537-541.). The contribution of mutations to the association rate can be calculated and has been used to increase the association rate (without greatly changing the dissociation rate) and the affinity of beta-lactamase inhibitory protein to TEM1 beta-lactamase by a factor of 250 (Seizer et al., 2000).

By “variant” we mean that the natural sequence of IL-6 has been modified by one or more point mutations, insertions of amino acids, deletions of amino acids or replacement of amino acids, in particular using non-natural amino acids such as D-isomers of natural amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, beta-alanine, fluoro-amino acids, designer amino acids such as β-methyl amino acids, Cα-methyl amino acids, Nα-methyl amino acids, β-naphthalimo amino acids and amino acid analogues in general.

In a second aspect the present invention provides computer-assisted method for identifying potential compounds able to interact with the IL-6 receptor and thereby modulate an activity mediated by the receptor, using a programmed computer comprising a processor, an input device, and an output device, comprising the steps of:
(a) inputting into the programmed computer, through the input device, data comprising the atomic coordinates of amino acids 1-299 of the IL-6 receptor as shown in Appendix I, or structural coordinates having a root mean square deviation from the backbone atoms of said amino acids of not more than 1.5 Å, or one or more subsets of said amino acids, or one or more subsets of said amino acids related to the coordinates shown in Appendix I by whole body translations and/or rotations;
(b) generating, using computer methods, a set of atomic coordinates of a structure that possesses stereochemical complementarity to the atomic coordinates of amino acids 1-299 of the IL-6 receptor as shown in Appendix I, or structural coordinates having a root mean square deviation from the backbone atoms of said amino acids of not more than 1.5 Å, or one or more subsets of said amino acids, or one or more subsets of said amino acids related to the coordinates shown in Appendix I by whole body translations and/or rotations, thereby generating a criteria data set;
(c) comparing, using the processor, the criteria data set to a computer database of chemical structures;
(d) selecting from the database, using computer methods, chemical structures which are similar to a portion of said criteria data set; and
(e) outputting, to the output device, the selected chemical structures which are complementary to or similar to a portion of the criteria data set.

In a preferred embodiment the subset of amino acids are the amino acids defining the ligand binding surface(s) or dimer interface or allosteric switch or the tryptophane-arginine ladder.

In a preferred embodiment of the second aspect, the method is used to identify potential compounds which have the ability to decrease an activity mediated by the receptor.

In a further preferred embodiment of the second aspect, the method further comprises the step of selecting one or more chemical structures from step (e) which interact with the IL-6 receptor in a manner such as to:
(i) interfere with the binding of natural ligand to one or more of the residues of IL-6R selected from the group consisting of 106-110, 133-138, 160-168, 190-193, 227-233, 250-256, 276-281 and combinations thereof; or
(ii) interfere with IL-6R homodimer formation.

In a still further preferred embodiment of the second aspect, the method further comprises the step of selecting one or more chemical structures from step (e) which interact with:—
(i) one or more of the residues of IL-6R selected from the group consisting of 106-110, 133-138, 160-168, 190-193, 227-233, 250-256, 276-281 and combinations thereof; or
(ii) the IL-6R dimer interface defined by amino acids 1-5, 19-23, 65-69, 93-99, 118, 119, 132-141, 166-172, 179-196, 241-250, 261, 262, 272-276 and 282-290; or
(iii) one or more of the residues of IL-6R selected from the group consisting of 11, 45, 46, 55, 62-66, 69-72, 75, 81, 88, 90-93, 122-124, 178 and combinations thereof; or
(iv) one or more of the residues of IL-6R selected from the group consisting of 233-239, 244-248, 270-290 and combinations thereof.

In a further preferred embodiment of the second aspect, the method further comprises the step of obtaining a compound with a chemical structure selected in steps (d) and (e), and testing the compound for the ability to decrease an activity mediated by the receptor.

In a further preferred embodiment of the second aspect, the method further comprises the step of obtaining a molecule with a chemical structure selected in steps (d) and (e), and testing the compound for the ability to increase an activity mediated by the receptor molecule.

The present invention also provides a method of screening of a putative compound having the ability to modulate the activity of the IL-6 receptor, comprising the steps of identifying a putative compound by a method according to the first or second aspects, and testing the compound for the ability to increase or decrease an activity mediated by the molecule. In one embodiment, the test is carried out in vitro. Preferably, the in vitro test is a high throughput assay. In another embodiment, the test is carried out in vivo.

In a third aspect the present invention provides a computer for producing a three-dimensional representation of a molecule or molecular complex, wherein the computer comprises:
(a) a machine-readable data storage medium comprising a data storage material encoded with machine-readable data, wherein the machine readable data comprise the atomic coordinates of amino acids 1-299 of the IL-6 receptor as shown in Appendix I, or structural coordinates having a root mean square deviation from the backbone atoms of said amino acids of not more than 1.5 Å, or one or more subsets of said amino acids, or one or more subsets of said amino acids related to the coordinates shown in Appendix I by whole body translations and/or rotations;
(b) a working memory for storing instructions for processing the machine-readable data;
(c) a central-processing unit coupled to the working memory and to the machine-readable data storage medium, for processing the machine-readable data into the three dimensional representation; and
(d) an output hardware coupled to the central processing unit, for receiving the three-dimensional representation.

In a preferred embodiment the subset of amino acids are the amino acids defining at least one ligand binding surface(s) or dimer or allosteric switch or the tryptophane-arginine ladder.

In a fourth aspect the present invention provides a compound able to bind to the IL-6 receptor and to modulate an activity mediated by the molecule, the compound being obtained by a method according to the present invention.

In a preferred embodiment of the fourth aspect, the compound is a mutant of the natural ligand of the IL-6 receptor, where at least one mutation occurs in the region of the natural ligand which interacts with the receptor.

In a fifth aspect the present invention provides a compound which possesses stereochemical complementarity to a topographic region of the IL-6 receptor and which modulates an activity mediated by the receptor, wherein the receptor is characterised by
(i) amino acids 1-299 of the IL-6 receptor positioned at atomic coordinates as shown in Appendix I, or structural coordinates having a root mean square deviation from the backbone atoms of said amino acids of not more than 1.5 Å; or
(ii) one or more subsets of said amino acids related to the coordinates shown in Appendix I by whole body translations and/or rotations.

with the proviso that the compound is not a naturally occurring ligand of the IL-6 receptor or a mutant thereof.

By “mutant” we mean a ligand which has been modified by one or more point mutations, insertions of amino acids or deletions of amino acids.

In a preferred embodiment of the fifth aspect, the topographic region of the receptor is defined by amino acids 106-110, 133-138, 160-168, 190-193, 227-233, 250-256 and 276-281, or the IL-6R dimer interface defined by amino acids 1-5, 19-23, 65-69, 93-99, 118, 119, 132-141, 166-172, 179-196, 241-250, 261, 262, 272-276 and 282-290, or amino acids 11, 45, 46, 55, 62-66, 69-72, 75, 81, 88, 90-93, 122-124 and 178, or amino acids 233-239, 244-248 and 270-290 and combinations thereof.

In preferred embodiments of the fourth and fifth aspects, the stereochemical complementarity between the compound and the receptor site is such that the compound has a Kd for the receptor site of less than 10⁻⁶M, more preferably less than 10⁻⁸M.

In other embodiments of the fourth and fifth aspects, the compound decreases an activity mediated by the IL-6 receptor.

In a sixth aspect; the present invention provides a pharmaceutical composition for preventing or treating a disease associated with signalling by the IL-6 receptor which comprises a compound according to the fourth or fifth aspects of the present invention and a pharmaceutically acceptable carrier or diluent.

In another aspect the present invention provides a method of preventing or treating a disease associated with signalling by the IL-6 receptor which method comprises administering to a subject in need thereof a compound according to the fourth or fifth aspects of the present invention. Preferably, the disease is selected from multiple myeloma, lymphoma, inflammation, rheumatoid arthritis, prostate cancer, Castleman's disease, AIDS, mesangial proliferative glomerulonephritis, Kaposi's sarcoma, sepsis, osteoporosis and psoriasis.

In yet another aspect, the present invention provides a method for evaluating the ability of a chemical entity to interact with the IL-6 receptor, said method comprising the steps of:
(a) creating a computer model of at least one region of the IL-6 receptor using structure coordinates wherein the root mean square deviation between said structure coordinates and the structure coordinates of amino acids 1-299 of IL-6 receptor as set forth in Appendix I is not more than about 1.5 Å;
(b) employing computational means to perform a fitting operation between the chemical entity and said computer model of the binding surface; and
(c) analysing the results of said fitting operation to quantify the association between the chemical entity and the binding surface model.

In a preferred embodiment of this aspect of the invention the region of IL-6R is selected from the group consisting of the ligand binding surface defined by amino acids 106-110, 133-138, 160-168, 190-193, 227-233, 250-256, 276-281 and combinations thereof, or the IL-6R dimer interface defined by amino acids 1-5, 19-23, 65-69, 93-99, 118, 119, 132-141, 166-172, 179-196, 241-250, 261, 262, 272-276 and 282-290, or amino acids 11, 45, 46, 55, 62-66, 69-72, 75, 81, 88, 90-93, 122-124 and 178, or amino acids 233-239, 244-248 and 270-290 and combinations thereof 97, 134, 140-142, 167-171, 209-213, 261-263 and 274.

As discussed above the present inventors have developed a model of the IL-6, IL-6R, gp130 hexameric complex. The coordinates for this complex are set out in Appendix II where the six molecules are identified as follows:—

| | |
|---|---|
| AAAA | gp130 |
| BBBB | gp130 |
| CCCC | IL-6R |
| DDDD | IL-6R |
| EEEE | IL-6 |
| FFFF | IL-6 |

The interfaces in this complex are as follows:—

IL-6R/gp130 Interface:
on IL-6R: residues 135-138; 161-169
on gp130: residues 140-149

IL-6/IL-6R Interface (Site I)
on IL-6: residues 41; 45; 51; 52; 56; 60; 62; 69-71; 75-79; 166; 169; 172-173; 176-177; 179-180; 183-184
on IL-6R: residues 1; 6; 107-108; 137-139; 162-168; 190; 193; 226-231; 277-281

IL-6/gp130 Interface (Site II)
on IL-6: residues 20; 24-25; 27; 29; 31-32; 35-36; 39-40; 42; 110-115; 117-119; 121-123; 125-126; 129; 179; 183;
on gp130: residues 114; 116; 142; 144-145; 147-148; 163-172; 193-196; 226-227; 229; 231-232; 281-283; 285;

IL-6/gp130 Interface (Site III)
on IL-6: residues 63-64; 93-94; 97-98; 128; 130-147; 151-152; 155; 159; 162-163;
on gp130: residues 1-5; 8-15; 49; 75-78; 132-137; 148-154; 163; 177-184.

This information enables the selection or design of compounds that interfere with this hexamer formation.

Accordingly, in a further aspect the present invention provides a method of selecting or designing a compound that interferes with the formation of the IL-6, IL-6R, gp130 hexameric complex, the method comprising the step of assessing the stereochemical complementarity between the compound and a topographic region of the complex, wherein the complex is characterised by

(i) the amino acids of IL-6, IL-6 receptor and gp130 positioned at atomic coordinates as shown in Appendix II, or structural coordinates having a root mean square deviation from the backbone atoms of said amino acids of not more than 1.5 Å; or

(ii) one or more subsets of said amino acids related to the coordinates shown in Appendix II by whole body translations and/or rotations.

In another aspect the present invention provides computer-assisted method for identifying compounds that interfere with the formation of the IL-6, IL-6R, gp130 hexameric complex, using a programmed computer comprising a processor, an input device, and an output device, comprising the steps of:
(a) inputting into the programmed computer, through the input device, data comprising the atomic coordinates of amino acids IL-6, IL-6R and gp130 as shown in Appendix II, or structural coordinates having a root mean square deviation from the backbone atoms of said amino acids of not more than 1.5 Å, or one or more subsets of said amino acids, or one or more subsets of said amino acids related to the coordinates shown in Appendix II by whole body translations and/or rotations;
(b) generating, using computer methods, a set of atomic coordinates of a structure that possesses stereochemical complementarity to the atomic coordinates the IL-6, IL-6R, gp130 hexameric complex as shown in Appendix II, or structural coordinates having a root mean square deviation from the backbone atoms of said amino acids of not more than 1.5 Å, or one or more subsets of said amino acids, or one or more subsets of said amino acids related to the coordinates shown in Appendix II by whole body translations and/or rotations, thereby generating a criteria data set;
(c) comparing, using the processor, the criteria data set to a computer database of chemical structures;
(d) selecting from the database, using computer methods, chemical structures which are similar to a portion of said criteria data set; and
(e) outputting, to the output device, the selected chemical structures which are complementary to or similar to a portion of the criteria data set.

In another aspect the present invention provides a computer for producing a three-dimensional representation of a molecule or molecular complex, wherein the computer comprises:
(a) a machine-readable data storage medium comprising a data storage material encoded with machine-readable data, wherein the machine readable data comprise the atomic coordinates of the IL-6, IL-6R, gp130 hexameric complex as shown in Appendix II, or structural coordinates having a root mean square deviation from the backbone atoms of said amino acids of not more than 1.5A, or one or more subsets of said amino acids, or one or more subsets of the coordinates shown in Appendix II by whole body translations and/or rotations;
(b) a working memory for storing instructions for processing the machine-readable data;
(c) a central-processing unit coupled to the working memory and to the machine-readable data storage medium, for processing the machine-readable data into the three dimensional representation; and
(d) an output hardware coupled to the central processing unit, for receiving the three-dimensional representation.

In another aspect the present invention provides a compound which possesses stereochemical complementarity to a topographic region of the IL-6, IL-6R, gp130 hexameric complex and which modulates an activity mediated by the complex, wherein the complex is characterised by
(i) the amino acids of IL-6, IL-6R and gp130 positioned at atomic coordinates as shown in Appendix II, or structural coordinates having a root mean square deviation from the backbone atoms of said amino acids of not more than 1.5 Å; or
(ii) one or more subsets of said amino acids related to the coordinates shown in Appendix II by whole body translations and/or rotations.

In yet another aspect, the present invention provides a method for evaluating the ability of a chemical entity to interact with the IL-6, IL-6R, gp130 hexameric complex, said method comprising the steps of:
(a) creating a computer model of at least one region of the IL-6, IL-6R, gp130 hexameric complex using structure coordinates wherein the root mean square deviation between said structure coordinates and the structure coordinates set forth in Appendix I is not more than about 1.5 Å;
(b) employing computational means to perform a fitting operation between the chemical entity and said computer model; and
(c) analysing the results of said fitting operation to quantify the association between the chemical entity and the model.

The compound may interfere with hexamer formation in a number of ways. For example the compound may bind or interact with one of the molecules at or near one or more of the specified residues or corresponding regions and by steric overlap and/or electrostatic repulsion prevent natural ligand binding or dimer formation. Alternatively the compound may bind to one of the molecules so as to interfere allosterically with hexamer formation.

It is presently preferred, however, that the compound binds or interacts with at least one of the molecules positioned at the interfaces.

Accordingly in a preferred embodiment of the present invention, the method comprises selecting or designing a compound which has portions that match at least one region of the molecules that make up the hexamer, in which the region is selected from the group consisting of;
(i) amino acids 20, 24, 25, 27, 29, 31, 32, 35, 36, 39, 40-42, 45, 51, 52, 56, 60, 62-64, 69-71, 75-79, 93, 94, 110-115, 117-119, 121-123, 125, 126, 128-147, 151, 152, 155, 159, 161-169, 172, 173, 176, 177, 179, 180, 183 and 184 of IL-6 and combinations thereof; and
(ii) amino acids 1-5, 8-15, 49, 75-78, 114, 116, 132-137, 140-154, 163-172, 177-184, 193-196, 226, 227, 229, 231, 232, 281-283 and 285 of gp130 and combinations thereof; and
(iii) amino acids 1, 6, 107, 108, 135-139, 161-169, 190, 193, 226-231 and 277-281 of IL-6R and combinations thereof.

In a still further aspect the present invention consists in a crystalline composition comprising IL-6 receptor or portion thereof or variant of these.

As will be readily understood by persons skilled in this field the methods of the present invention provide a rational method for designing and selecting compounds which interact with the IL-6 receptor. In the majority of cases these compounds will require further development in order to increase activity. Such further development is routine in this field and will be assisted by the structural information provided in this application. It is intended that in particular embodiments the methods of the present invention includes such further developmental steps.

In another aspect the present invention consists in a method of assessing the interaction between a compound and the IL-6 receptor, the method comprising exposing a crystalline composition comprising IL-6 receptor or portion thereof or variant of these to the compound and measuring the leveling of binding of the compound to the crystal.

Accordingly, in another aspect the present invention consists in a method of designing or selecting a compound which modulates IL-6R signalling, the method comprising subjecting a compound obtained by a method according to any one of the previous aspects of the present invention to biological screens and assessing the ability of the compound to modulate IL-6R signalling.

In yet a further aspect, the invention provides a method of utilizing molecular replacement to obtain structural information about a molecule or a molecular complex of unknown structure, comprising the steps of:
(i) crystallising said molecule or molecular complex;
(ii) generating an X-ray diffraction pattern from said crystallized molecule or molecular complex;
(iii) applying at least a portion of the structure coordinates set forth in Appendix I to the X-ray diffraction pattern to generate a three-dimensional electron density map of at least a portion of the molecule or molecular complex whose structure is unknown.

The structure of the IL-6 receptor can be used to design mutant IL-6 receptor molecules or fragments thereof for use as therapeutics.

Accordingly, the present invention also provides a compound comprising an extracellular fragment of IL-6R, wherein the extracellular fragment is modified at one or more amino acids of IL-6R selected from the group consisting of:
(i) amino acids 106-110, 133-138, 160-168, 190-193, 227-233, 250-256 and 276-281; or
(ii) amino acids 1-5, 19-23, 65-69, 93-99, 118, 119, 132-141, 166-172, 179-196, 241-250, 261, 262, 272-276 and 282-290; or
(iii) amino acids 11, 45, 46, 55, 62-6, 69-72, 75, 81, 55, 90-93, 122-124 and 178; or
(iv) amino acids 233-239, 244-248 and 270-290; or
(v) amino acids 1, 6, 107, 108, 135-139, 161-169, 190, 193, 226-231 and 277-281.

In a preferred embodiment of this aspect of the invention, the modification is made to an amino acid which is not listed in Table 2.

In a further preferred embodiment, the IL-6R fragment comprises residues 1 to 325 of SEQ ID NO:1.

In one embodiment of this aspect, the modification increases the affinity of the IL-6R fragment for one or more of its natural ligands when compared to the unmodified fragment.

It will be appreciated by those skilled in the art that these compounds may be formulated into pharmaceutical compositions and used to treat diseases associated with signaling of the IL-6R.

The present inventors have now obtained three dimensional structural information about the IL-6 receptor which enables a more accurate understanding of how the binding of ligand leads to signal transduction. Such information provides a rational basis for the development of ligands for specific therapeutic applications, something that heretofore could not have been predicted de nova from available sequence data.

Those of skill in the art will understand that a set of structural coordinates determined by X-ray crystallography is not without standard error. For the purposes of this invention, any set of structure coordinates for IL-6R or an IL-6R complex that have a root mean square derivation of protein backbone atoms of less than 1.5 Å when superimposed (using backbone atoms) on the structure coordinates listed in Appendix I or II shall be considered identical.

The precise mechanisms underlying the binding of agonists and antagonists to the IL-6 receptor are not fully clarified. However, the binding of ligands to the receptor site, preferably with an affinity in the order of 10⁻⁸M or higher, is understood to arise from enhanced stereochemical complementarity relative to naturally occurring IL-6 receptor ligands.

Such stereochemical complementarity, pursuant to the present invention, is characteristic of a molecule that matches intra-site surface residues lining the groove of the receptor site as enumerated by the coordinates set out in Appendix I. By “match” we mean that the identified portions interact with the surface residues, for example, via hydrogen bonding or by non-covalent Van der Waals and Coulomb interactions which promote desolvation of the biologically active compound within the site, in such a way that retention of the biologically active compound within the groove is favoured energetically.

Substances which are complementary to the shape and electrostatics or chemistry of the receptor site characterised by amino acids positioned at atomic coordinates set out in Appendix I will be able to bind to the receptor, and when the binding is sufficiently strong, substantially prohibit binding of the naturally occurring ligands to the site.

It will be appreciated that it is not necessary that the complementarity between ligands and the receptor site extend over all residues lining the groove in order to inhibit binding of the natural ligand.

In general, the design of a molecule possessing stereochemical complementarity can be accomplished by means of techniques that optimize, chemically and/or geometrically, the “fit” between a molecule and a target receptor. Known techniques of this sort are reviewed by Sheridan and Venkataraghavan, 1987, Acc. Chem Res. 20: 322; Goodford, 1984, J. Med. Chem. 27: 557; Beddell, 1985, Chem. Soc. Reviews 279; Hol, 1986, Angew. Chem. 25: 767, Verlinde and Hol, 1984, Structure 2: 577, Walters et al., 1998, Drug Discovery Today 3: 160; Langer and Hoffmann, 2001, Current Pharmaceutical Design 7: 509; Good, 2001, Current Opinion in Drug Disc. Devel. 5, 301; Gane and Dean, 2000, Curr. Opinion Struct. Biol. 10: 401. the respective contents of which are hereby incorporated by reference. See also Blundell et al., 1987, Nature 326: 347 (drug development based on information regarding receptor structure) and Loughney et al., 1999, Med. Chem. Res. 9: 579 (database mining application on the growth hormone receptor).

There are two preferred approaches to designing a molecule, according to the present invention, that complements the stereochemistry of the IL-6 receptor. The first approach is to in silico directly dock molecules from a three-dimensional structural database, to the receptor site, using mostly, but not exclusively, geometric criteria to assess the goodness-of-fit of a particular molecule to the site. In this approach, the number of internal degrees of freedom (and the corresponding local minima in the molecular conformation space) is reduced by considering only the geometric (hard-sphere) interactions of two rigid bodies, where one body (the active site) contains “pockets” or “grooves” that form binding sites for the second body (the complementing molecule, as ligand).

This approach is illustrated by Kuntz et al., 1982, J. Mol. Biol. 161: 269, and Ewing et al., 2001, J. Comput-Aid. Mol. Design 15: 411, the contents of which are hereby incorporated by reference, whose algorithm for ligand design is implemented in a commercial software package, DOCK version 4.0, distributed by the Regents of the University of California and further described in a document, provided by the distributor, which is entitled “Overview of the DOCK program suite” the contents of which are hereby incorporated by reference. Pursuant to the Kuntz algorithm, the shape of the cavity represented by the IL-6 receptor site is defined as a series of overlapping spheres of different radii. One or more extant databases of crystallographic data, such as the Cambridge Structural Database System maintained by Cambridge University (University Chemical Laboratory, Lensfield Road, Cambridge CB2 1EW, U.K.), the Protein Data Bank maintained by the Research Collaboratory for Structural Bioinformatics (Rutgers University, N.J., U.S.A.), LeadQuest (Tripos Associates, Inc., St. Louis, Mo.), Available Chemicals Directory (Molecular Design Ltd., San Leandro, Calif.), and the NCI database (National Cancer Institute, U.S.A) is then searched for molecules which approximate the shape thus defined.

Molecules identified in this way, on the basis of geometric parameters, can then be modified to satisfy criteria associated with chemical complementarity, such as hydrogen bonding, ionic interactions and Van der Waals interactions. Different scoring functions can be employed to rank and select the best molecule from a database. See for example Bohm and Stahl, 1999, M. Med. Chem. Res. 9: 445. The software package FlexX, marketed by Tripos Associates, Inc. (St. Louis, Mo.) is another program that can be used in this direct docking approach (see Rarey, M. et al., J. Mol. Biol. 1996, 261: 470).

The second preferred approach entails an assessment of the interaction of respective chemical groups (“probes”) with the active site at sample positions within and around the site, resulting in an array of energy values from which three-dimensional contour surfaces at selected energy levels can be generated. The chemical-probe approach to ligand design is described, for example, by Goodford, 1985, J. Med. Chem. 28:849, the contents of which are hereby incorporated by reference, and is implemented in several commercial software packages, such as GRID (product of Molecular Discovery Ltd., West Way House, Elms Parade, Oxford OX2 9LL, U.K.). Pursuant to this approach, the chemical prerequisites for a site-complementing molecule are identified at the outset, by probing the active site with different chemical probes, e.g., water, a methyl group, an amine nitrogen, a carboxyl oxygen, and a hydroxyl. Favoured sites for interaction between the active site and each probe are thus determined, and from the resulting three-dimensional pattern of such sites a putative complementary molecule can be generated. This may be done either by programs that can search three-dimensional databases to identify molecules incorporating desired pharmacophore patterns or by programs which using the favoured sites and probes as input perform de novo design.

Programs suitable for searching three-dimensional databases to identify molecules bearing a desired pharmacophore include: MACCS-3D and ISIS/3D (Molecular Design Ltd., San Leandro, Calif.), ChemDBS-3D (Chemical Design Ltd., Oxford, U.K.), and Sybyl/3 DB Unity (Tripos Associates, Inc., St. Louis, Mo.).

Programs suitable for pharmacophore selection and design include: DISCO (Abbott Laboratories, Abbott Park, Ill.), Catalyst (Accelrys, San. Diego, Calif.), and ChemDBS-3D (Chemical Design Ltd., Oxford, U.K.).

Databases of chemical structures are available from a number of sources including Cambridge Crystallographic Data Centre (Cambridge, U.K.), Molecular Design, Ltd., (San Leandro, Calif.), Tripos Associates, Inc. (St. Louis, Mo.), and Chemical Abstracts Service (Columbus, Ohio).

De novo design programs include Ludi (Biosym Technologies Inc., San Diego, Calif.), Leapfrog (Tripos Associates, Inc.), Aladdin (Daylight Chemical Information Systems, Irvine, Calif.), and LigBuilder (Peking University, China).

Those skilled in the art will recognize that the design of a mimetic may require slight structural alteration or adjustment of a chemical structure designed or identified using the methods of the invention.

The invention may be implemented in hardware or software, or a combination of both. However, preferably, the invention is implemented in computer programs executing on programmable computers each comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program is preferably implemented in a high level procedural or object-oriented programming language to communicate with a computer system. However, the programs can be implemented in assembly or machine language, if desired. In any case, the language may be compiled or interpreted language.

Each such computer program is preferably stored on a storage medium or device (e.g., ROM or magnetic diskette) readable by a general or special purpose programmable computer, for configuring and operating the computer when the storage media or device is read by the computer to perform the procedures described herein. The inventive system may also be considered to be implemented as a computer-readable storage medium, configured with a computer program, where the storage medium so configured causes a computer to operate in a specific and predefined manner to perform the functions described herein.

Modulators of II-6 Receptors

The present invention provides the atomic coordinates of the crystal structure of the soluble extracellular portion of an IL-6 receptor alpha subunit and the atomic coordinates of the crystal structure of a hexameric complex consisting of both IL-6 receptor subunits (sIL-6α and gp130) together with the ligand IL-6. As described above, these coordinates can be used in methods of the invention to screen in silico, for potential modulators of IL-6R activity.

Accordingly, the present invention provides a compound which modulates the activity of an IL-6R, said compound identified/identifiable by a method of the invention. Preferably said compound is specific for IL-6R, for example said compounds exhibits at least 10 times, preferably at least 50 times more activity towards IL-6R than other cytokine receptors, such as an IGR receptor.

In one embodiment, said method comprises the step of assessing the stereochemical complementarity between the compound and a topographic region of the receptor, wherein the receptor comprises:
(i) amino acids 1-299 of the IL-6 receptor positioned at atomic coordinates as shown in Appendix I, or structural coordinates having a root mean square deviation from the backbone atoms of said amino acids of not more than 1.5 Å; or
(ii) one or more subsets of said amino acids related to the coordinates shown in Appendix I by whole body translations and/or rotations.

Preferably the topographic region of the IL-6 receptor is selected from:
(i) a ligand binding surface defined by residues 106-110, 133-138, 160-168, 190-193, 227-233, 250-256 or 276-281 and combinations thereof;
(ii) the homodimer interface defined by resides 1-5, 19-23, 65-69, 93-99, 118, 119, 132-141, 166-172, 179-196, 241-250, 261, 262, 272-276 or 282-290 and combinations thereof;
(iii) residues 11, 45, 46, 55, 62-66, 69-72, 75, 81, 88, 90-93, 122-124 and 178; and
(iv) residues 233-239, 244-248 and 270-290.

Compounds designed according to the methods of the present invention may be assessed by a number of in vitro and in vivo assays of hormone function. For example, the identification of IL-6 receptor antagonists of may be undertaken using a solid-phase receptor binding assay. Potential antagonists may be screened for their ability to inhibit the binding of europium-labelled IL-6 receptor ligands to soluble, recombinant IL-6 receptor in a microplate-based format. Europium is a lanthanide fluorophore, the presence of which can be measured using time-resolved fluorometry. The sensitivity of this assay matches that achieved by radioisotopes, measurement is rapid and is performed in a microplate format to allow high-sample throughput, and the approach is gaining wide acceptance as the method of choice in the development of screens for receptor agonists/antagonists (see Apell et al., J. Biomolec. Screening 3:19-27, 1998: Inglese et al., Biochemistry 37:2372-2377, 1998).

Binding affinity and inhibitor potency may be measured for candidate inhibitors using biosensor technology.

Biological assays to measure the activity of IL-6R are well known in the field. In particular various assay systems are described in Hammacher et al., 1998, J. Biol. Chem. 273, 22701-22707; and Hammacher et al, 1994, Protein Science, 3, 2280-2293, the disclosures of which are incorporated by reference.

The methods of the invention have been used to identify a number of compounds that potentially are agonist or antagonists of IL-6 activity. These compounds have subsequently been tested in vitro to determine biological activity and specificity. Compounds showing specific IL-6R agonist or antagonist activity have been modeled using the crystal structure of the invention to determine the site of binding and the amino acid residues of IL-6Rα that are contacted by the compounds. These results have enabled the generation of general structural criteria for both a class of antagonists and a class of agonists that occupy all or part of the IL-6 ligand binding surface defined by loops L1 to L7 as described above.

Antagonists

In a preferred embodiment, an antagonist of an IL-6 receptor is a compound of formula A-B-C, wherein

A consists of three fused 5-, 6- or 7-membered, saturated, unsaturated or aryl rings, optionally containing one or more heteroatoms and optionally substituted;

or two non-fused 5-, 6- or 7-membered saturated, unsaturated or aryl rings, optionally containing one or more heteroatoms and optionally substituted;

C consists of three fused 5-, 6- or 7-membered, saturated, unsaturated or aryl rings, optionally containing one or more heteroatoms and optionally substituted; or two non-fused 5-, 6- or 7-membered saturated, unsaturated or aryl rings, optionally containing one or more heteroatoms and optionally substituted; and

B is an aliphatic linker having a length substantially equivalent to an ethylene moiety, for example a linker of similar length and geometry to a peptide bond i.e. with a length of about 3.5 Å;

wherein said compound has stereocomplementarity to a ligand binding topographic region of:

(i) amino acids 1-299 of the IL-6 receptor positioned at atomic coordinates as shown in Appendix I, or structural coordinates having a root mean square deviation from the backbone atoms of said amino acids of not more than 1.5 Å; or

(ii) one or more subsets of said amino acids related to the coordinates shown in Appendix I by whole body translations and/or rotations;

said ligand binding topographic region being defined by residues 106-110, 133-138, 160-168, 190-193, 227-233, 250-256 and 276-281 of said IL-6 receptor, or combinations thereof.

Ring substituents should be selected so as not to disrupt the stereocomplementarity with the IL-6 ligand binding surface. Consequently, substituents are generally selected from C₁-C₄ alkyl, halogen, OR²⁴ or NR²⁵R²⁶, where R²⁴, R²⁵ and R²⁶ are each independently hydrogen or C₁-C₄ alkyl. Moreover, the rings of A are preferably joined such that A is in a substantially “straight” configuration in relation to the linker B. Equally, the rings of A are preferably joined such that C is in a substantially “straight” configuration in relation to the linker B.

Preferably A has the following formula: 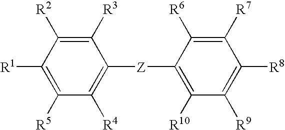
wherein
Z is a bond; or Z, R⁴ and R¹⁰ taken together form an optionally substituted, saturated, unsaturated or aryl ring having 5, 6 or 7 members, preferably 5 or 6 members, optionally containing one or more heteroatoms selected from O, N and S; or Z, R³ and R⁶ taken together form an aryl ring or a heteroaryl, cycloalkyl, cycloalkenyl or heterocyclyl ring having 5, 6 or 7 members, preferably 5 or 6 members, wherein said aryl ring or said heteroaryl, cycloalkyl, cycloalkenyl, heterocyclyl ring are optionally substituted;
R⁷, R⁸ or R⁹ are bonded to linker B;
R¹, R² and R⁵ are each independently, hydrogen, C₁-C₄ alkyl, halogen, OR²⁴ or NR²⁵R²⁶, where R²⁴, R²⁵ and R²⁶ are each independently hydrogen or C₁-C₄ alkyl;
R³ and R⁶ unless bonded together with Z are each independently, hydrogen, C₁-C₄ alkyl, halogen, O, OR²⁴ or NR²⁵R²⁶, where R²⁴, R²⁵ and R²⁶ are each independently hydrogen or C₁-C₄ alkyl;
R⁴ and R¹⁰ unless bonded together with Z, are each independently, hydrogen, C₁-C₄ alkyl, halogen, O, OR²⁴ or NR²⁵R²⁶, where R²⁴, R²⁵ and R²⁶ are each independently hydrogen or C₁-C₄ alkyl;
R⁷, R⁸ and R⁹ unless bonded to linker B, are each independently, hydrogen, C₁-C₄ alkyl, halogen, O, OR²⁴ or NR²⁵R²⁶, where R²⁴, R²⁵ and R²⁶ are each independently hydrogen or C₁-C₄ alkyl;

Preferably the linker B has the following formula: 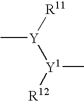
wherein Y and Y¹ are each independently C, O, S or N, provided that Y and Y¹ are not both O, N or S;
R¹¹ and R¹² are each independently hydrogen, C₁-C₄ alkyl, halogen, O, OR²⁴ or NR²⁵R²⁶, where R²⁴, R²⁵ and R²⁶ are each independently hydrogen or C₁-C₄ alkyl;
Preferably at least Y and R¹¹ together, or Y¹ and R¹² together are CO. Preferably Y or Y¹ are NR²⁵R²⁶, where R²⁴, R²⁵ and R²⁶ are each independently hydrogen or C₁-C₄ alkyl, preferably hydrogen.

Preferably C has the following formula: 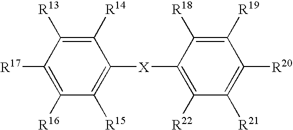
wherein
X is a bond; or X, R¹⁴ and R¹⁸ taken together form an optionally substituted, saturated, unsaturated or aryl ring having 5, 6 or 7 members, preferably 5 or 6 members, optionally containing one or more heteroatoms selected from O, N and S; or X, R¹⁵ and R²² taken together form an aryl ring or a heteroaryl, cycloalkyl, cycloalkenyl or heterocyclyl ring having 5, 6 or 7 members, preferably 5 or 6 members, wherein said aryl ring or said heteroaryl, cycloalkyl, cycloalkenyl, heterocyclyl ring are optionally substituted;
R¹³, R¹⁶ or R¹⁷ are bonded to linker B;
R¹⁹, R²⁰ and R²¹ are each independently, hydrogen, C₁-C₄ alkyl, halogen, O, OR²⁷ or NR²⁸R²⁹, where R²⁷, R²⁸ and R²⁹ are each independently hydrogen or C₁-C₄ alkyl;
R¹⁴ and R¹⁸ unless bonded together with Z are each independently, hydrogen, C₁-C₄ alkyl, halogen, O, OR²⁷ or NR²⁸R²⁹, where R²⁷, R²⁸ and R²⁹ are each independently hydrogen or C₁-C₄ alkyl;
R¹⁵ and R²² unless bonded together with Z, are each independently, hydrogen, C₁-C₄ alkyl, halogen, O, OR²⁷ or NR²⁸R²⁹, where R²⁷, R²⁸ and R²⁹ are each independently hydrogen or C₁-C₄ alkyl;
R¹³, R¹⁶ and R¹⁷ unless bonded to linker B, are each independently, hydrogen, C₁-C₄ alkyl, halogen, O, OR²⁷ or NR²⁸R²⁹, where R²⁷, R²⁸ and R²⁹ are each independently hydrogen or C₁-C₄ alkyl;

In a highly preferred embodiment, said antagonist has the following formula I: 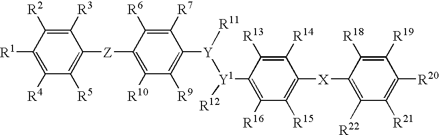
Wherein X, Y, Y¹, Z and R¹ to R²⁹ are as defined above.

More preferably, the antagonist is a compound of formula II: 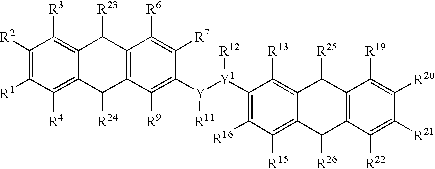
wherein X, Y, Y¹, Z and R¹ to R²⁹ are as defined above; and
R²³, R²⁴, R²⁵ and R²⁶ are each independently hydrogen, C₁-C₄ alkyl, halogen, O, OR²⁷ or NR²⁸R²⁹, where R²⁷, R²⁸ and R²⁹ are each independently hydrogen or C₁-C₄ alkyl, preferably O.
Agonists

In a preferred embodiment, an agonist of an IL-6 receptor is a compound of formula A-B-D, wherein

A is as defined above for antagonists;

D consists of one, or two fused, 5-, 6- or 7-membered, saturated, unsaturated or aryl rings, optionally containing one or more heteroatoms and optionally substituted; and

B is an aliphatic linker as defined above for antagonists;

wherein said compound has stereocomplementarity to a ligand binding topographic region of:

(i) amino acids 1-299 of the IL-6 receptor positioned at atomic coordinates as shown in Appendix I, or structural coordinates having a root mean square deviation from the backbone atoms of said amino acids of not more than 1.5 Å; or

(ii) one or more subsets of said amino acids related to the coordinates shown in Appendix I by whole body translations and/or rotations;

said ligand binding topographic region being defined by residues 106-110, 133-138, 160-168, 190-193, 227-233, 250-256 and 276-281 of said IL-6 receptor, or combinations thereof.

Ring substituents should be selected so as not to disrupt the stereocomplementarity with the IL-6 ligand binding surface. Consequently, substituents are generally selected from C₁-C₄ alkyl, halogen, OR²⁴ or NR²⁵R²⁶, where R²⁴, R²⁵ and R²⁶ are each independently hydrogen or C₁-C₄ alkyl. The length of the D moiety is less than that of the C moiety of the antagonist compounds. Consequently, substituents should be selected so as avoid the length of the D moiety exceeding the equivalent of two aryl rings. Moreover, where D consists of two fused rings, the rings are preferably joined such that D is in a substantially “straight” configuration in relation to the linker B.
Preferably D has the following formula: 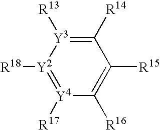
wherein
Y², Y³ or Y⁴ are each independently C, O, N or S, provided that at least two of Y², Y³ and Y⁴ are C;
R¹³, R¹⁷ or R¹⁸ are bonded to linker B;
R¹⁴, R¹⁵ and R¹⁶ are each independently, hydrogen, C₁-C₄ alkyl, halogen, O, OR²⁷ or NR²⁸R²⁹, where R²⁷, R²⁸ and R²⁹ are each independently hydrogen or C₁-C₄ alkyl; or
R¹⁶ is hydrogen, C₁-C₄ alkyl, halogen, O, OR²⁷ or NR²⁸R²⁹, where R²⁷, R²⁸ and R²⁹ are each independently hydrogen or C₁-C₄ alkyl, and R¹⁴ and R¹⁵ together form an optionally substituted, saturated, unsaturated or aryl ring having 5, 6 or 7 members, optionally containing one or more heteroatoms selected from O, N and S; or
R¹⁴ is hydrogen, C₁-C₄ alkyl, halogen, O, OR²⁷ or NR²⁸R²⁹, where R²⁷, R²⁸ and R²⁹ are each independently hydrogen or C₁-C₄ alkyl R¹⁵ and R¹⁶ together form an optionally substituted, saturated, unsaturated or aryl ring having 5, 6 or 7 members, optionally containing one or more heteroatoms selected from O, N and S;
R¹³, R¹⁷ and R¹⁸ unless bonded to linker B, are each independently, hydrogen, C₁-C₄ alkyl, halogen, O, OR²⁷ or NR²⁸R²⁹, where R²⁷, R²⁸ and R²⁹ are each independently hydrogen or C₁-C₄ alkyl;
In a preferred embodiment, said agonist has the following formula III: 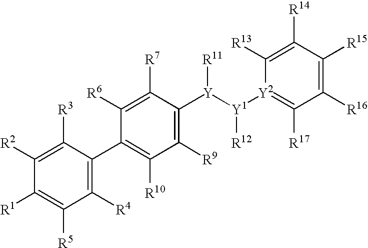
Wherein Y, Y¹, Y², Z and R¹ to R²⁹ are as defined above. Preferably Y² is N.
In an alternative preferred embodiment, said agonist has the following formula IV: 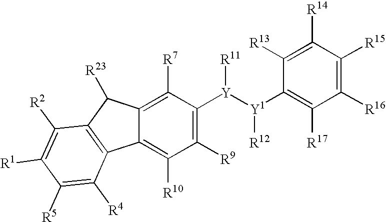
wherein X, Y, Y¹, R¹ to R¹³ and R¹⁷ are as defined above;
R¹⁴, R¹⁵ and R¹⁶ are each independently, hydrogen, C₁-C₄ alkyl, halogen, O, OR²⁷ or NR²⁸R²⁹, where R²⁷, R²⁸ and R²⁹ are each independently hydrogen or C₁-C₄ alkyl; preferably R¹⁴, R¹⁵ and R¹⁶ are each independently, hydrogen, C₁-C₂ alkyl, NH₂, OH or halogen.
R²³ is hydrogen, C₁-C₄ alkyl, halogen, O, OR²⁷ or NR²⁸R²⁹, where R²⁷, R²⁸ and R²⁹ are each independently hydrogen or C₁-C₄ alkyl, preferably O.

Reference above to halogen includes I, Br, Cl and F. Preferably the halogen is Br or Cl.

Where reference is made to C₁ to C₄ alkyl above, C₁ to C₃ alkyl is preferred, with C₁ to C₂ alkyl being especially preferred. Where rings include heteroatoms, selected from N, O and S, it is preferred that any particular ring has less than three heteroatoms, preferably less than two heteroatoms. Where reference is made to rings having 5, 6 or 7 members, it is preferred that rings have 5 or 6 members.

Uses of Modulators of IL-6R

The compounds described above may be used to modulate IL-6R activity in cells, i.e. activate or inhibit IL-6R activity. Given that aberrant IL-6R/IL-6 activity is implicated in a range of disorders, the compounds described above may also be used to treat, ameliorate or prevent disorders characterised by abnormal IL-6 signalling. Examples of such disorders include multiple myeloma, rheumatoid arthritis, Castleman's disease, AIDS, mesangial proliferative glomerulonephritis, Kaposi's sarcoma, sepsis, osteoporosis and psoriasis.

Administration

Compounds of the invention, i.e. modulators of IL-6R identified or identifiable by the screening methods of the invention, may preferably be combined with various components to produce compositions of the invention. Preferably the compositions are combined with a pharmaceutically acceptable carrier or diluent to produce a pharmaceutical composition (which may be for human or animal use).

The formulation will depend upon the nature of the compound and the route of administration but typically they can be formulated for topical, parenteral, intramuscular, oral, intravenous, intra-peritoneal, intranasal inhalation, lung inhalation, intradermal or intra-articular administration. The compound may be used in an injectable form. It may therefore be mixed with any vehicle which is pharmaceutically acceptable for an injectable formulation, preferably for a direct injection at the site to be treated, although it may be administered systemically.

The pharmaceutically acceptable carrier or diluent may be, for example, sterile isotonic saline solutions, or other isotonic solutions such as phosphate-buffered saline. The compounds of the present invention may be admixed with any suitable binder(s), lubricant(s), suspending agents), coating agent(s), solubilising agent(s). It is also preferred to formulate the compound in an orally active form.

In general, a therapeutically effective daily oral or intravenous dose of the compounds of the invention, including compounds of the invention and their salts, is likely to range from 0.01 to 50 mg/kg body weight of the subject to be treated, preferably 0.1 to 20 mg/kg. The compounds of the invention and their salts may also be administered by intravenous infusion, at a dose which is likely to range from 0.001-10 mg/kg/hr.

Tablets or capsules of the compounds may be administered singly or two or more at a time, as appropriate. It is also possible to administer the compounds in sustained release formulations.

Typically, the physician will determine the actual dosage which will be most suitable for an individual patient and it will vary with the age, weight and response of the particular patient. The above dosages are exemplary of the average case. There can, of course, be individual instances where higher or lower dosage ranges are merited, and such are within the scope of this invention.

For some applications, preferably the compositions are administered orally in the form of tablets containing excipients such as starch or lactose, or in capsules or ovules either alone or in admixture with excipients, or in the form of elixirs, solutions or suspensions containing flavouring or colouring agents.

The compositions (as well as the compounds alone) can also be injected parenterally, for example intracavernosally, intravenously, intramuscularly or subcutaneously. In this case, the compositions will comprise a suitable carrier or diluent.

For parenteral administration, the compositions are best used in the form of a sterile aqueous solution which may contain other substances, for example enough salts or monosaccharides to make the solution isotonic with blood.

For buccal or sublingual administration the compositions may be administered in the form of tablets or lozenges which can be formulated in a conventional manner.

For oral, parenteral, buccal and sublingual administration to subjects (such as patients), the daily dosage level of the compounds of the present invention and their pharmaceutically acceptable salts and solvates may typically be from 10 to 500 mg (in single or divided doses). Thus, and by way of example, tablets or capsules may contain from 5 to 100 mg of active compound for administration singly, or two or more at a time, as appropriate. As indicated above, the physician will determine the actual dosage which will be most suitable for an individual patient and it will vary with the age, weight and response of the particular patient.

The routes of administration and dosages described are intended only as a guide since a skilled practitioner will be able to determine readily the optimum route of administration and dosage for any particular patient depending on, for example, the age, weight and condition of the patient.

The present invention will now be described further with reference to the following examples, which are illustrative only and non-limiting. The examples refer to the figures:

### DETAILED DESCRIPTION OF THE FIGURES

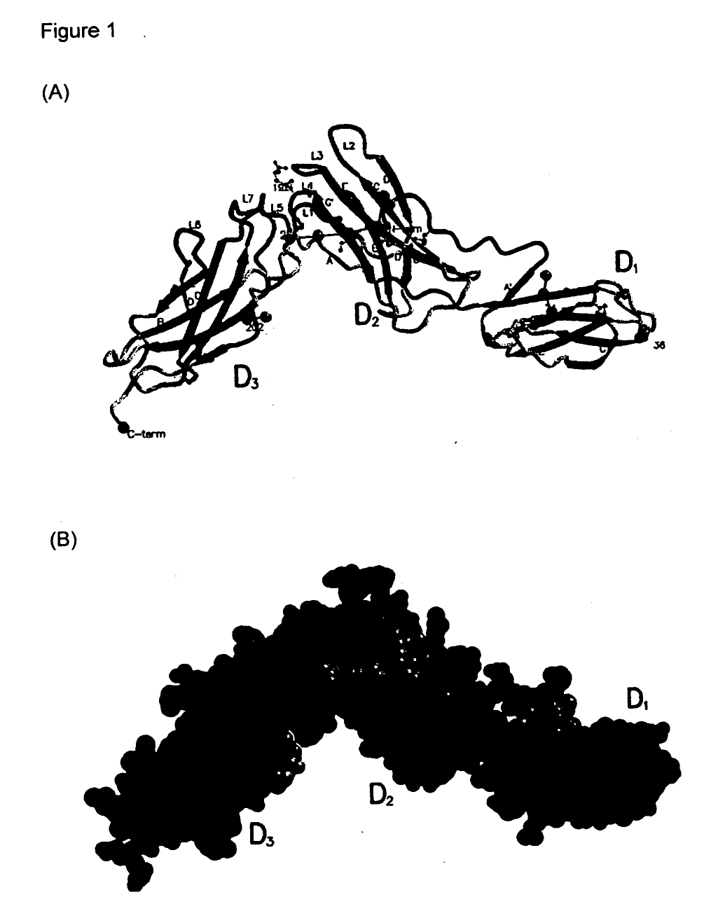

FIG. 1

(a) A MOLSCRIPT diagram of sIL-6R indicating the β-sheet arrangement (shades of green, orange, blue for domains D₁, D₂ and D₃ and respectively). The different β-strand shades represent the separate β-sheets in the structure. Four asparagine (blue spheres) linked sites are shown with their associated carbohydrate moieties represented by yellow spheres linked by yellow bonds. The N-terminal residues 1 to 15 (gray tube), is tethered to strand F of D₂ by a disulfide bond. The 3₁₀ helices (purple), the loops (red) L1 to L7 (L1 to L7 consist of the polypeptides S106 to N110 (L1), K133 to P138 (L2), A160 to F168 (L3), Q190 to G193 (L4), S227 to R233 (L5), M250 to H256 (L6) and Q276 to Q281 (L7)) that could interact with IL-6 around the juncture of D₂ and D₃ are also shown. The single cysteine residue disulfide linked to C192 is also shown, as are the disulfide links.

(b) The domain structure (CPK model) of sIL-6R showing domain D₁ in green, D₂ in red and D₃ in blue, with carbohydrate in yellow spheres.

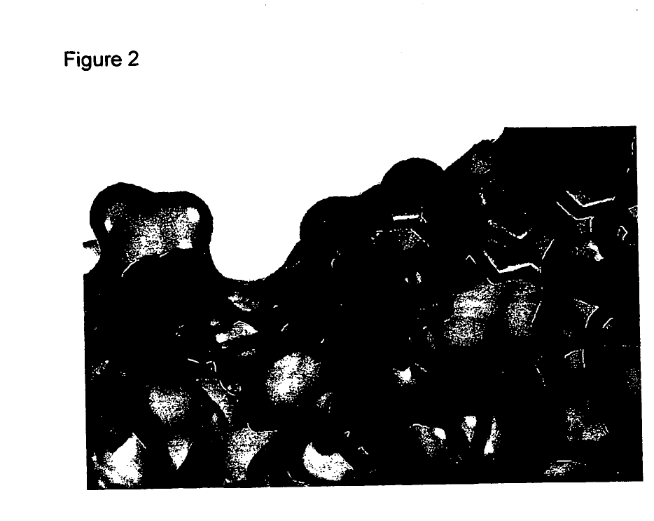

FIG. 2

A view of the surface associated with the WSXWS (green bonds) sequence motif in IL-6R and its association with the other residues of the tryptophane/arginine ladder (yellow bonds) in D₃. The backbone of the WSXWS motif is shown as part of the 3₁₀ helix found in cytokine binding domains, and a groove on the surface lies above this helix. A stripe of positively charged residues (arginines) lie parallel to this grove. Nitrogen and oxygen atoms of the side chains are coloured blue and red spheres respectively as are the surfaces in proximity to these atoms.

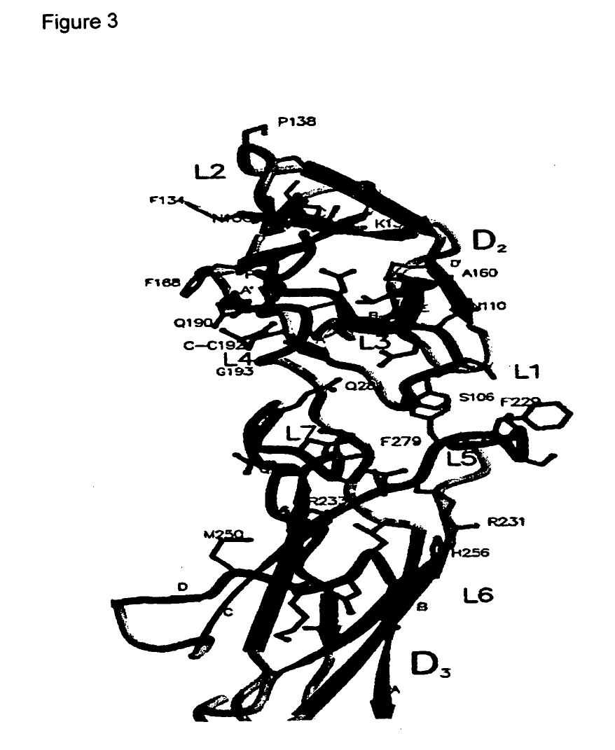

FIG. 3

A MOLSCRIPT diagram of the CBD of sIL-6R viewed from the top of FIG. 1a showing the IL-6 binding loops L1 to L7. The side-chains of the loops are shown as ball and stick representations, and the remaining structure as in FIG. 1a. The IL-6 binding loops are represented by a backbone worm representation coloured red (L1), yellow (L2), green (L3), cyan (L4), pink (L5), dark green (L6) and orange (L7) respectively. The side-chain bonds are colour matched to the respective loops. Oxygen, nitrogen and sulphur atoms in the loop side-chains are coloured as red, blue and yellow spheres respectively (57). The cysteinyl-cysteine C192 is also shown on loop L4.

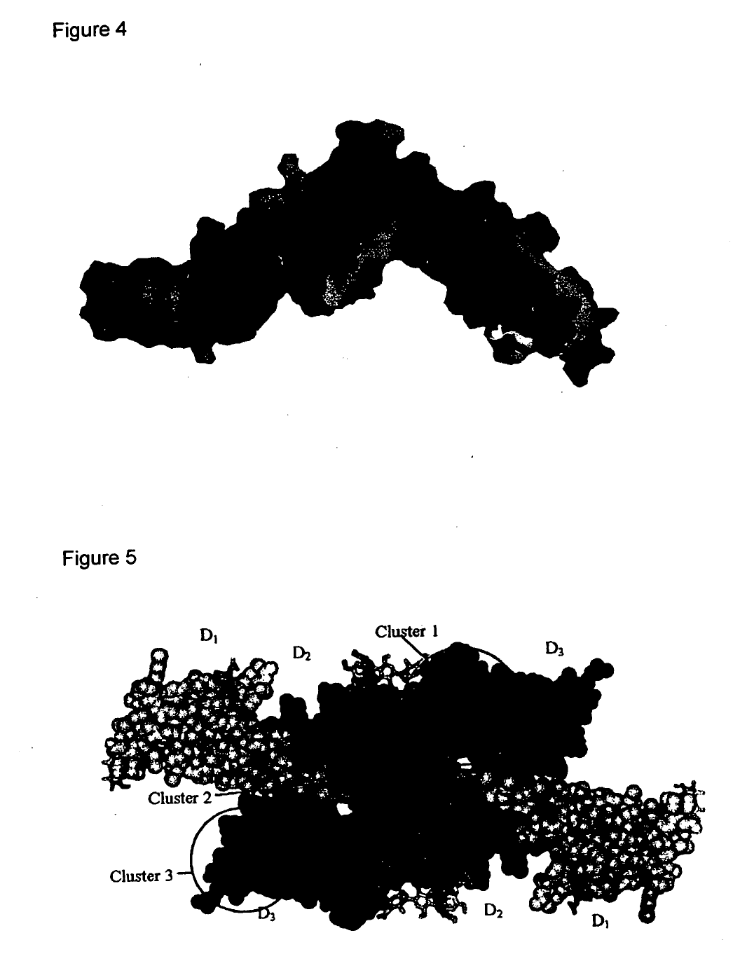

FIG. 4

The dimer interface of IL-6R showing the region of interaction by red, green, blue, purple and cyan areas representing interdimeric distances of less than 5, 4, 3, 2, and 1 Å respectively. The crystal 2-fold axis is vertical and the membrane is horizontal below the molecule.

FIG. 5

A MOLSCRIPT CPK representation of a crystallographic dimer of IL-6R, viewed towards the membrane anchors, showing the three mutational clusters found on the IL-6R structure. The crystallographic 2-fold axis lies at the centre of the figure and perpendicular to the paper and the dimer interface lies east to west through the two-fold axis. Residues in the upper molecule in the figure are coloured to represent mutations that effect IL-6 binding to IL-6R (red, pink and green spheres representing the atoms of residues which have <25%, 25% to 75% and >75% of the binding activity of the wild type respectively). Residues in the lower molecule are coloured to represent mutations that effect gp130 signalling (red, pink and green spheres representing the atoms of residues which have <25%, 25% to 75% and >75% of the signalling of wild type respectively). Residues in domains D₁, D₂ and D₃ that there is no mutational data or alter the structure are coloured white, light grey and grey respectively. The cysteinyl-cysteine C192 is coloured dark grey, and carbohydrate is represented by yellow bonds.

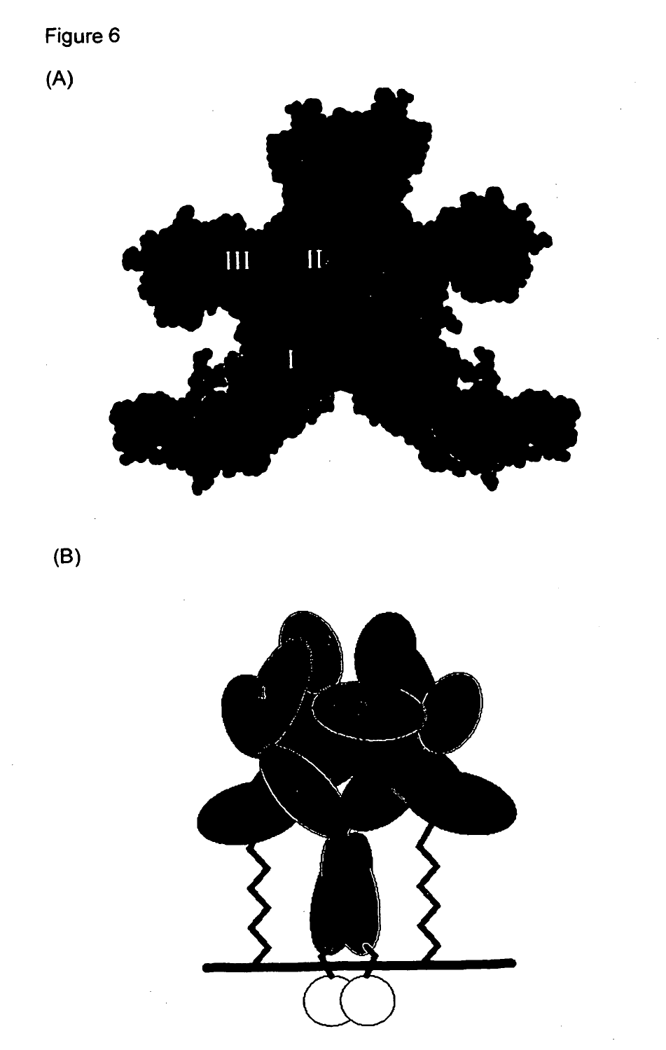

FIG. 6

(a) Spacefilling model of the proposed hexameric receptor complex, consisting of two molecules each of IL-6, IL-6R and gp130 chains (shown in purple and blue, light and dark green, orange and red respectively). IL-6 is labeled with the binding sites I, II and III.

(b) A modular representation of the hexameric complex (D₁ to D₆ are labeled 1 to 6), coloured as in (a), but with the additional three fibronectin domains (D₄ to D₆) of gp130, that have not been modeled, placed to indicate how the association of the two D_{5′} modules from both gp130 molecules, under the ‘tunnel’ of the IL-6R dimer, can activate signaling by dimerisation of the cytoplasmic domains (white circles).

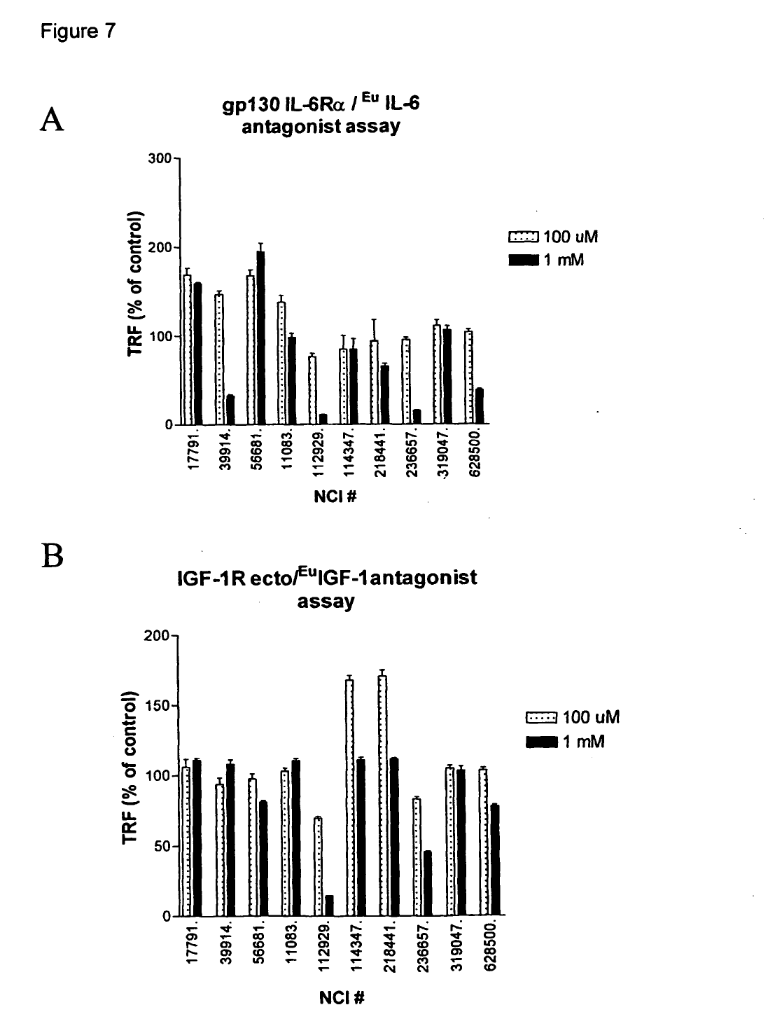

FIG. 7

Solid phase receptor binding assays. Ten compounds selected from Table 3 (NCI compounds) were tested for their ability to modulate the binding of europium-labelled IL-6 to soluble, recombinant IL-6 receptor (A) or the binding of europium-labelled IGF-1 to soluble, recombinant IGF-1 receptor (B) in a microplate-based format. Binding to the receptor is measured by time resolved fluorescence (TRF) as a % of control.

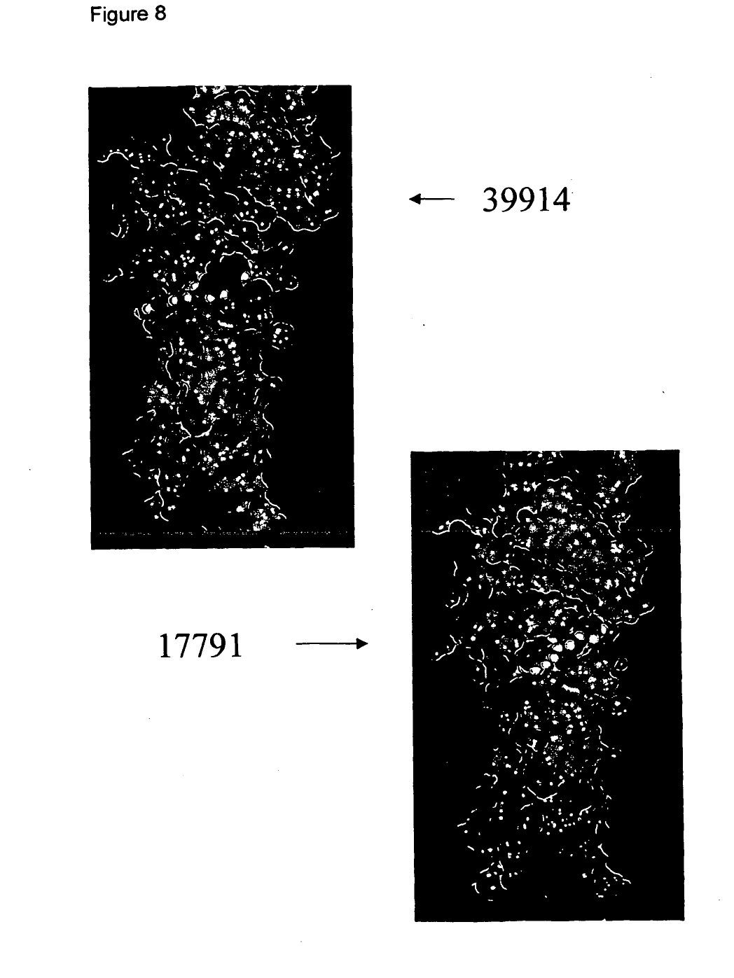

FIG. 8

Model of the IL-6 receptor showing the site of binding of an antagonist (NCI compound 39914) (A) and an agonist (NCI compound 17791) (B).

### EXAMPLES

### Example 1

### Crystallisation and Structural Analysis of sIL-6

Methods

Expression, Purification and Crystallization of sIL-6R

Monomeric sIL-6R was purified from the conditioned medium of glycosylation defective mutant Chinese hamster ovary (CHO) cell line Lec3.2.8.1 (Stanley, 1989, Mol. Cell Biol. 9: 377-383), transfected with the construct pEE14sIL6RL325, which encodes sIL-6R. Briefly, sIL-6R transformed Lec3.2.8.1 cells were grown in fermentation apparatus with a working volume of 1.25 L (New Brunswick Celligen Plus fermenter, New Brunswick, USA). Conditioned media was concentrated 20-fold by ultrafiltration. sIL-6R was purified from concentrated Lec3.2.8.1 cell media by binding to a 5 mL column of human IL-6-Sepharose. SIL-6R was further purified by preparative size-exclusion chromatography and concentrated to 10 mg/mL using a 10000 MWCO centrifugal concentrator (Centricon, Millipore, USA).

The protein (8 mg/ml in 5 mM Tris-HCl pH 8.0) was crystallised in hanging drops by vapour diffusion against a reservoir containing 50 mM ammonium sulphate, 18% PEG 3350, 2.5 mM Sodium citrate and at pH 5.6. Crystals grew in the space group P4₃2₁2 (a=51.4 Å, c=305.4 Å) with 51% solvent. To ensure isomorphism for heavy metal derivatives 0.1% gluteraldehyde was added to the well solution for 16 hrs. Crystals were then transferred to a stabilising solution of 0.1M lithium sulphate with 2.5 mM Tris-HCl at pH 6.5, and finally to a freezing solution of 20% PEG 3350, 16% glycerol, 0.1 M lithium sulphate, 2.5 mM sodium citrate and 2.5 mM Tris-HCl at pH 6.5.

Diffraction Data

X-ray data were collected from crystals flash-frozen to 100K in a nitrogen stream. Three data sets were collected for each of the native, and two platinum derivatives (PIP: di-u-iodo-bis(ethylene-diamine)-di-platinum(II)nitrate; PTN: K2Pt(NO₂)₄). Three data sets were collected for each native and two derivatives. A data set with the c* axis parallel to the spindle, one perpendicular to the spindle axis and one low resolution data set, using R-AXIS II and IV image-plate detectors fitted with both mirror and monocapillary optics (Balaic et al., 1996, J. Synchrotron Rad. 3: 289-295) respectively. A 2.4 Å native data set was collected at the Advance Photon Source (Argonne, U.S.A.) on beam line BM14C using an ADSC Quantum-4 CCD-detector. All data sets were collected at 0.5° or 1.0° oscillations and processed and scaled using HKL (Otwinowski and Minor, 1997, Macromolec. Crystallog. Pt A. 276: 307-326) (Table 1).

TABLE 1
Data collection and MIR phasing statistics

| | Native | PIP | PTN |
|---|---|---|---|
| Data Collection | | | |
| Resolution (Å) | 2.4 | 3.0 | 3.0 |
| Completeness* (%) | 99.4(100) | 91.6(63.4) | 95.7(80.2) |
| Multiplicity | 18.6 | 6.5 | 5.6 |
| <I/σ(I)>* | 39.0(3.7) | 15.4(2.2) | 10.5(1.0) |
| R_{merge}* | 0.11(0.57) | 0.05(0.38) | 0.13(0.58) |
| Wilson B (Å²) | 85.3 | 67.7 | 79.2 |
| MIR Phasing | | | |
| Resolution (Å) | | 50-4.0 | 50-4.0 |
| Sites per molecule | | 3^{†} | 4 |
| Total occupation | | 0.77 | 0.95 |
| Phasing Power^{‡?} | | 1.4(1.7)[1.1] | 1.1(1.3)[1.1] |
| R_{Cullis}^{‡} | | 0.71(0.76)[0.93] | 0.82(0.82)[0.92] |

*Numbers in parentheses are values in the high resolution shell, where R_{merge} = Σ(Iᵢ − <I>)/ΣIᵢ summed over all independent reflections.
^{†}Each site was split into pairs.
^{‡}The three numbers represent summation over: centric(acentric)[acentric anomalous] reflections.
^{?}Root mean square (rms) fₕ/residual = √(Σ(F_{deri} − F_{PH})²), where fₕ and F_{PH} are the calculated heavy-atom and derivative structure factors, respectively.
^{¶}R_{cullis} = Σ||fₕ| − (|F_{deri}| − |Fₙₐₜᵢ|)/|Σ||F_{deri}| − |Fₙₐₜᵢ||, where F_{deri} and Fₙₐₜᵢ are the observed derivative and native structure factors, respectively

Structure Solution.

The heavy atom sites of the PIP and PTN derivatives were determined by Patterson methods (CCP4, 1994, Acta Crystallogr. D. Biol. Crystallogr. 50: 760-763) and SOLVE (Terwilliger and Berendzen, 1999, Acta Crystallogr. D. Biol. Crystallogr. 55: 849-861). The space group and hand of the heavy atom sites was determined using the heavy atom anomalous data from both derivatives. The positions of sites were refined to 4 Å using SHARP (de la Fortelle and Bricogne, 1997, Macromolec. Crystallog. Pt A. 276: 472-494) and phasing statistics are shown on Table 1. The 4 Å solvent-flattened (51% solvent) MIRAS-phased electron density map was skeletonised and used to trace the C_{□}chain of the protein. This map clearly showed the CBD module, and the Ig domain. A partial atomic model of the FnIII domains and part of the Ig domain was built using Lego fragments in “O” (Jones et al., 1991, Acta Crystallogr. A. 47: 110-119). N-linked carbohydrate, were included, at asparagines N36, N74, N₂O₂ and N226 and 80 solvent molecules. The disulfide bonds were consistent with the earlier mass spectrometric data. The model was then refined using CNS (Brunger et al., 1998, Acta Crystallogr. D. Biol Crystallogr. 54: 905-921), extending the resolution to 2.4 Å by an iterative process involving model rebuilding and gradual identification and refinement of the residues on the longer loops linking the strands. The quality of the electron density and the thermal parameters of the Ig domain were poorer than the CBD indicating a higher degree of disorder. Residues 1 to 5 and 82 to 84 were set to zero occupancy as the density was poor and residues beyond 299 (C-terminus) were not observable. The side chains of loops 49 to 52 and 136 to 138 appeared highly mobile or disordered. Additional solvent (including two sulphate ions) was added during the course of the refinement. A previously reported cysteine disulfide bonded to the free C192 was observed and built into the model. The final R-factor was 0.22 (R-free of 0.29 over 5% of the data) with r.m.s.d. in bond lengths and angles being 0.015 Å and 1.96° respectively with overall anisotropic temperature factors of B₁₁=B₂₂=−6.4 Å₂ and B₃₃=12.7 Å₂ added to the individual atomic isotropic B-values.

The coordinates of the IL-6R structure are provided herein as Appendix I.

Modeling the Complex

The model of the hexameric interleukin-6 signal transduction complex (see FIG. 6a.) was assembled using a parallel version of the protein-protein docking algorithm FTDOCK 2.0 (Gabb et al., 1997, J. Mol. Biol. 272: 106-120). The protein chains were taken from the NMR structure (PDB code 1IL6) for IL-6 (Xu et al., 1997, J. Mol. Biol. 268: 468-481), the X-ray structure of gp130 from the viral IL-6 gp130 complex (Chow et al., 2001, Science 291: 2150-2155) (PDB code 1I1R) and the IL-6R dimer. Using a grid spacing of 0.5 Å and incorporating electrostatic treatment the following protein/protein docking calculations were undertaken. IL-6 was docked with IL-6R, IL-6 with IL-6R dimer, IL-6 with gp130 and gp130 to IL-6R. Complexes were re-scored (Moont et al., 1999, Proteins 35: 364-373) and the top ranking complexes satisfying the available mutational data (Table 2, Salvati et al., 1995, J. Biol. Chem. 270: 12242-12249) was subject to rigid body refinement and side-chain rotamer optimization using the program MULTIDOCK (Jackson et al., 1998, J. Mol. Biol. 276: 265-285). There was no significant difference in the docking solutions produced for IL-6 to IL-6R and IL-6 to the IL-6R dimer so the solutions of IL-6 to IL-6R dimer were used, with each alpha chain in the dimer binding its cognate IL-6 for assembly of the complex. The final complex was built by superimposing the chains in common from the best solutions using INSIGHTII (Accelrys Inc, San Diego, Calif., USA). The resulting complex was then minimized using CNS (Brunger et al., 1998), with a harmonic restraint applied to the α-carbon backbone.

The coordinates of the IL-6R/IL-6/gp130 hexamer are provided herein as Appendix II.

Results and Discussion

The IL-6R Extracellular Structure

The complete extracellular region of human IL-6R was expressed and secreted from the Chinese hamster ovary (CHO) cell line, Lec3.2.8.1, which produces carbohydrate-deficient glycoprotein (Stanley, 1989). This was necessary because normal CHO cell lines produce heterogeneously glycosylated protein, which did not crystallise. The Lec3.2.8.1 cell secretes a form of the extracellular domain of IL-6R (residues 1 to 325) which was purified and crystallized to diffract to 2.4 Å. The IL-6R 3-dimensional structure was determined by multiple isomorphous replacement with anomalous scattering (MIRAS) from two heavy atom derivatives.

Our X-ray structure of the extracellular domain of the IL-6R (FIG. 1) consists of the N-terminal Ig domain (D₁, residues 1 to 93) linked to a classical is CBD (D₂, residues 94 to 194; D₃, residues 195 to 299). The first 5 residues of the N-terminus are poorly defined and amino acids past residue 299 are not visible in the X-ray structure. The three domains lie on a similar plane making the receptor a long ‘flat’ structure in which the domains D₂ and D₃ are connected at about 90° to each other and D₁ is connected at about 135° to D₂. Carbohydrate is attached at 4 sites on one of the faces of the molecule (FIG. 1), indicating that this face is unlikely to be involved in either the binding of IL-6 or the formation of the signalling complex with gp130. This conclusion is supported by the expression of functional sIL-6R in Escherichia coli which lacks carbohydrate.

The N-terminal cysteine residue C6 is disulfide bonded to residue C174 in D₂, and there is an unusual β-sheet arrangement of D₁ (FIG. 1a). D₁ has an S-type Ig topology very similar to the ligand-binding domain of fibroblast growth factor (LBD-FGF)), but the first ‘A’ strand of the 3-stranded β-sheet of the LBD-FGF Ig domain is not observed in IL-6R. It appears that these residues have ‘peeled away’ from the sheet when compared to this strand in LBD-FGF (0.7 Å r.m.s.d. between the α-carbons of the two domain structures excluding the ‘A’ strand atoms). The C-terminus of this domain forms a fifth ‘A′’ strand of what would be a 4-stranded β-sheet in an S-type Ig topology. This arrangement of the ‘A’ strand of the β-sheet could be a result of crystal packing and we suggest there is a degree of flexibility of this domain, which acts as a conformational switch, to allow D₁ to rotate up to 20° by β-strand shifting. This flexibility could explain what appears to be a higher degree of disorder in this domain when compared to the other domains as indicated by the higher temperature factors of residues in domain D₁ compared to D₂ or D₃ (mean B values for domains D₁, D₂ and D₃ are 68 Å², 49 Å² and 44 Å² respectively). The molecules of IL-6R in the crystal are packed head to tail in a double helix arrangement with the helical axis along the 4-fold crystal screw axis in direction of the long cell edge (c-axis) and the pitch and diameter of the helix will vary if the angle between domains D₁ and D₂ changes. Such flexibility of domains would explain the lack of isomorphism between different crystals of IL-6R that were observed in this study.

A characteristic of the CBD of IL-6R is the long tryptophan-arginine ladder (from the N-terminus end of D₃ is R239, F246, R237, W287, R274, W284, Q276) which is also found in other class 1 CBD structures. This ladder incorporates the conserved WSXWS motif (residues 284 to 287 in IL-6R) located in the carboxy terminus region of domain D₃ of the CBD (FIG. 2). The polypeptide backbone of the WSXWS sequence has an unusual motif: a left-handed 3₁₀ helix similar to a polyproline helix. This helix is stabilised by the stacking of the indole side chains of the two tryptophan residues of the WSXWS sequence with arginine side-chains (R237 and R274), and H-bonding of serine side chain hydroxyls but not by main chain H-bonds. The ladder produces a long surface stripe of positive charge (from the guanidinium and tryptophan indol nitrogens) running along D₃ near the inside elbow of D₂-D₃, and a groove formed by the 3₁₀ helix running parallel to the stripe (FIG. 2). Although this structure is conserved in other known structures of CBDs its functional role has not as yet been clarified. It could be implicated in the folding of the CBD, complex formation or involved in a general receptor transport system.

Following the paradigm of growth hormone (de Vos et al., 1992), and the available mutational data, it would be expected that IL-6 would bind in the region of the outer elbow formed at the junction of D₂ and D₃ (FIG. 3), characterised by 4 loops (S106-N110 (L1), K133-P138 (L2), A160-F168 (L3), Q190-G193 (L4)) from D₂ and 3 loops (S227-R233 (L5), M250-H256 (L6) and Q276-Q281 (L7)) from D₃ (FIG. 3). These loops present a long and narrow (43 Å by 9.5 Å) potential binding area held in place by the rigid D₂ and D₃ framework of the cytokine binding domains. IL-6 would engage residues in some of these loops.

A Dimer in the Crystal

In the crystal lattice, two molecules of IL-6R are related by a crystallographic 2-fold axis and are closely associated along the length of each molecule (FIG. 4). These two molecules (with a total buried accessible surface area of about 2460 Å²) represent a potential physiological dimer of IL-6R. The association is primarily a hydrophobic contact around the 2-fold axis involving F134 and F168 of domain D₂, the salt-link E97-R274, and hydrogen-bonding of the side chain carboxylate of E283 with the main chain amide of T186. The buried accessible surface area (FIG. 4) of each molecule (surface- and electrostatic-complementarity of 0.722 and 0.728 respectively) is much larger than that of a protein-protein interaction surface in solution (typically ˜800 Å²). Furthermore the buried surface of membrane-associated receptor complexes do not have to be as extensive as their counterparts in solution, since their degrees of freedom are restricted to the two-dimensional surface of the membrane. Hence the dimeric association observed here in the crystal may well represent a complex formed on the cell surface.

The orientation of the monomers with the dimer is such that the interaction is along the carbohydrate-free ‘flat’ face of the molecule (FIG. 5). In this crystal structure, if the 2-fold axis of the dimer is perpendicular to the membrane surface, then domain D₃ projects away from the membrane at 45° and D2 points down towards the membrane at 30ₒ. D₁ would then run parallel to the membrane (FIG. 4). The dimer appears as a bridge structure (FIG. 6) similar to the common β-receptor of IL-3, with a 50 Å long tunnel of triangular cross-section (base ˜40 Å; height ˜22 Å).

Structural Implications of Mutations

Mapping of the mutational data onto the crystal structure of IL-6R provide some insights into regions on the surface of IL-6R that are critical to binding of IL-6 and implicated in signalling through gp130. Analysis of these data indicate that most of the mutations, when mapped onto the crystal structure, cause altered binding due to compromised structural integrity of the molecule, in particular mutations in the tryptophan/arginine ladder, cysteines forming disulfide bonds, and buried hydrophobic residues forming the hydrophobic core. The non-structural mutations occur on three clusters on the surface of IL-6R (Table 2, FIG. 5).

There is clearly a cluster of residues (see FIG. 5, Cluster 1) that form the binding site to IL-6. The mutations of residues having the greatest effect on IL-6 binding are those at P162 and E163 from L3; S228, F229, Y230 and R231 from L5 and E278 and F279 from L7 (Table 1). Two of these loops, L5 and L7, are both situated on D₃ and this is consistent with the report that D₃ by itself is able to bind IL-6 (18). This site at the juncture of D₂ and D₃ domains, and can be inferred to be primarily responsible for IL-6 binding.

Non-structural mutations in IL-6R that affect gp130 signalling (FIG. 5) can be classified onto two main clusters. The first occurs at a hydrophobic patch (residues F134, F168 and Y169) at the dimer interface (FIG. 5, Cluster 2) located around the crystallographic two fold axis relating the two molecules of the dimer. Mutations here reduce signalling but have no effect on binding. There are also other residues, not included in Cluster 2, on domain D₃ in this interface that reduce signalling but not binding (see FIG. 5). These data indicate that mutations that would be expected to interfere with the formation of the IL-6R dimer have a significant effect on IL-6 signalling but not on IL-6 binding. 14 The other cluster of mutations effecting gp130 signalling occurs on a patch of residues (FIG. 5, Cluster 3) centred around residue H261 on domain D₃. Mutational studies in this region of IL-6R were based on the growth hormone paradigm, which demonstrated a contact region between the second CBD domains of the growth hormone complex (de Vos et al., 1992, Science 255: 306-312). This patch is clearly not involved in IL-6 binding or IL-6R dimerisation, but when mutated this region effects signalling. This is most likely a region that is involved in the formation of the IL-6/IL-6R complex with gp130.

It has been predicted previously that two SSFY sequence repeats in IL-6R are involved in binding sites to IL-6. The first repeat S165-Y169 is located in L3 of D₂. Mutations in S167 and Y169 reduce signalling, but this is likely due to the structural importance of these buried residues. F168 is solvent exposed and forms the central part of Cluster 2 that is involved in IL-6R dimer formation, not IL-6 binding. A second aromatic residue, F134, is the other major residue in Cluster 2. The second SSFY repeat (S227-Y230) is located in L5 on D₃ of IL-6R (FIG. 3). These residues (apart from S227) are critical to IL-6 binding and are all surface exposed residues.

It has been reported previously that N211, H261 and D262 together form a group of residues that affect signalling but not binding of IL-6 to IL-6R. Surprisingly, these residues cluster with W214 and V259, that when mutated (to N and Q respectively) increase the signalling of IL-6 over wild type. The role of the cysteinyl-cysteine C192 remains unsolved. The residue is on the periphery of the IL-6 binding site. When C192 is mutated to alanine, the binding of IL-6 to IL-6R and signalling are slightly increased. The human IL-6R sequence is the only vertebrate sequence in the database to date that has a cysteine in this position: pig and cow having a tyrosine and mouse, and rat having a leucine.

TABLE 2
Structural Analysis of Selected Mutational Data

| | | | | |
|---|---|---|---|---|
| Loop | Binding | Signalling | Comment | R |

Cluster1 IL-6/IL-6R binding interface

| | | | | | |
|---|---|---|---|---|---|
| P162 | L3 | <10% | N.T. | PE-LQ double mutant. Glu may | Y |
| E163 | L3 | | | interact with IL-6, Pro unlikely to | |
| S228 | L3 | 100% | <25% | a range of mutants. possible minor | K |
| | | | | IL-6 cluster I residue | |
| F229 | L5 | <5% | 0% | IL-6 binding site residue | K |
| Y230 | L5 | <5% | 0% | a range of mutants. IL-6 binding site | K |
| | | | | residue | |
| R231 | L5 | 0% | N.T. | RL-SI double mutant. R231 is likely to | Y |
| L232 | L5 | | | be a IL-6 binding site residue. L232 is | |
| | | | | a buried hydrophobic residue | |
| E278 | L7 | 0% | N.T. | EF-AI double mutant. IL-6 binding site | Y |
| | | | | residue | |
| F279 | L7 | | | | |

Cluster2 IL-6R dimer interface

| | | | | | |
|---|---|---|---|---|---|
| F134 | L2 | >100% | 70% | F-L mutation, one of two Fs in IL-6R | Y |
| | | | | dimer | |
| S167 | L3 | 50-100% | 5-25% | buried in loop & forms H bonds - | K |
| | | | | many mutants | |
| F168 | L3 | 100-130-% | 50%-110% | A range of mutants. Central aromatic | K |
| | | | | residue in IL-6R dimer interface. F-Y | |
| | | | | mutant signals at 50% | |
| Y169 | | 30-70% | 0-15% | range of mutants - buried/structural | K |
| | | | | residue | |

Cluster3 D3 signalling cluster

| | | | | |
|---|---|---|---|---|
| N211 | 75% | N.T. | N-D mutant, <10% binding to gp130 | S |
| W214 | 70% | >150% | W-Q mutant - solvent exposed, | Y |
| | | | possibly part of site of gp130 binding | |
| | | | face | |
| V259 | 40% | 140% | exposed V-N mutant - maybe | Y |
| | | | involved in gp130 interaction | |
| I260 | 0% | N.T. | I-D mutation. buried hydrophobic | Y |
| | | | residue | |
| H261 | 115% | 10% | H-I mutation involved in gp130 | Y |
| | | | interaction | |
| D262 | 30% | <10% | D-G mutant. H-bonds to stabilise | Y |
| | | | binding loop and maybe interacts with | |
| | | | gp130 | |

N.T. = not tested;
R = Reference
K = Kalai et al., 1997, Blood 89: 1319-1333;
Y = Yawata et al., 1993, EMBO J. 12: 1705-1712; and
S = Salvati et al., 1995, J. Biol. Chem. 270: 12242-12249.

The Hexameric Signalling Complex

A hexameric complex (FIG. 6a) incorporating the IL-6R dimer can be constructed in a manner that is compatible with the available mutational and functional data present in the literature except for the mutations in Cluster 3, which have no cognate binding partner in this model. Additionally, the placement of the three C-terminal fibronectin type III domains (D₄, D₅ and D₆) from gp130 has not been considered. Several orientations of these domains are possible but in the absence of any structural information they are difficult to dock with sufficient surety. Using homology models of these three domains, one can orient in such a way so they contact Cluster 3 on D₃ of IL-6R and form a disulfide link proposed between D₅ of gp130 underneath the complex as shown schematically in FIG. 6b.

The recent structure of the Ig and CBD domain of gp130 complexed with viral IL-6 reveals a possible dimeric relationship between the human IL-6 and the binding domains of gp130. The dimeric relationship of the viral IL-6/gp130 complex is a crystallographic 2-fold relationship, as in the IL-6R dimer. Incorporating the same dimeric relationship between all the IL-6 binding receptor domains, it is possible to construct a model for the signalling complex (FIG. 6a). In this model, two IL-6 molecules bind to the IL-6R dimer via site I (FIG. 6) followed by two gp130 molecules each binding through sites II and III of different IL-6 molecules in a similar way to the viral IL-6/gp130 fragment complex. The signalling molecule gp130 would bind the IL-6/IL-6R complex with D₃ pointing away from the membrane. The remaining three FnIII domains of each gp130 would orientate towards the membrane, and the signalling activated by dimerisation of the gp130 membrane proximal FnIII domains (FIG. 6b) under the bridge of the IL-6R dimer likely through disulfide crosslinking. Although this model is substantially different from current proposed models, it is consistent with the available biological data. Other gp130 signalling complexes (e.g., IL-11) could act through a similar mechanism. A notable feature of this model is the binding orientation of IL-6 to IL-6R is rotated 180° compared to the analogous positions in growth hormone, prolactin and erythropoeitin receptor structures, and the IL-12 structure.

The crystal structure of IL-6R and the model of the hexameric complex will provide the basis for the design of mutations of the proteins involved in this complex. It also enables the design/selection of small molecular weight antagonists and agonists to IL-6 signalling that can be developed into therapeutics targeted to the diseases modulated by IL-6 signalling. The selection of a number of small molecular weight compounds and testing for activity as antagonists or agonists to IL-6 signalling is described below.

### Example 2

### Screening for Agonist/Antagonists of IL-6R

In Silico Screening

The sIL-6R structural information provided in Appendix I was used in an in silico screen for compounds having complementarity to the ligand binding surfaces of IL-6R defined by loops L1-L7. The screen was conducted on compounds in the NCI database using DOCK and in-house ranking and re-scoring protocols. Briefly, the orientation from the docking algorithm considered as the correct orientation was calculated in a normalised rank-by-number strategy (Wang and Wang, 2001, Journal of Chemical Information and Computer Sciences 41(5):1422-6) using scoring functions based on the DOCK energy function (Kuntz et al., 1982, J. Mol Biol 161: 269-288), SCORE (Wang et al, 1988, J Mol Model 4: 379-394), chemscore (Gohlke et al, 2000, J Mol Biol 295: 337-336), potential of mean force (Muegge et al, 1999, J Med. Chem. 4: 379-394), SMOG (DeWitte et al, 1996, J Am Chem Soc. 118: 11733-11744). The scores associated with each are then re-ranked in a rank-by-rank strategy (Wang and Wang, 2001). The top 100 compounds are then sorted by calculated octanol/water solubility (log P) (Wang, 1997, J. Chem. Inf. Comput. Sci. 37: 615), with compounds with a logP greater than 6 excluded from the hit-list. Representative examples of the top 100 compounds are listed in Table 3.

High Capacity Screening Assay for Detection of Agonists/Antagonists of the IL6/IL6Rα/gp130 Interaction.

Ten of the compounds listed in Table 3 were tested for their ability to modulate binding of IL-6 to IL-6R/gp130. The high throughput screening assay used for this purpose detects the binding of europium-labelled human IL6 (Eu-hIL6) to human soluble interleukin 6 receptor α-chain (hsIL6Rα) and hsgp130 immobilised on the surface of wells of a microtitre plate. The protocol followed was essentially as follows:

Europium Labelling of hIL-6

HIL-6 (2.13 mg, 100 nmol) was mixed with 1 mg (1,500 nmol) of Eu labelling reagent in 100 mM NaHCO₃ buffer; pH 9.3 and incubated at 4° C. for 48 h. The Eu-labelled IL-6 was then separated from unreacted labelling reagent and high molecular weight protein aggregates using a Pharmacia Superdex-200 column (HR 16/10) equilibrated in a buffer containing 50 mM Tris-HCl; pH 7.75, 0.9% NaCl, and 0.1% sodium azide. The column was run at a flow rate of 0.5 mL/min and fractions of 0.5 mL were collected.

The fractions were assayed for europium by diluting 10,000-fold in 200 μL of DELFIA enhancement solution and measuring europium fluorescence. The Eu-IL6 eluted (14-15 min) after high molecular weight protein aggregates (11-12 min) and before the unreacted labelling reagent (19 min). The concentration of Eu-IL6 was determined from the absorbance at 280 nm (0.5 AU/mg/mL) and the labelling stoichiometry based on a molecular mass of 21,300.

Screening Assay Protocol

Assays were performed in 384-well black microtitre plates. 50 μL of hsgp130 (2.5 μg/mL in PBS; pH 7.2) was added to each well in columns 1-22 of the assay plates. PBS was added only to wells in columns 23-24. The samples were then incubated at 4° C. overnight. Plates were washed 4 times with 100 μL of DELFIA® Wash Buffer using the EMBLA 384-well plate washer. DELFIA® Assay Buffer (25 μL) was added to all wells using the Multidrop 384-well dispenser.

Test compounds were reconstituted in methanol and transferred (3 μL samples) to the appropriate assay plate wells using the SAGIAN Multimek. Methanol was allowed to evaporate from assay plates by incubating at room temperature for 15 minutes.

A solution containing Eu-hIL6 (90 ng/mL) and hsIL6Rα (2 μg/mL) was prepared in DELFIA® Assay Buffer and added (25 μL samples) to all plate wells using the Multidrop 384-well dispenser. The plates were incubated at room temperature for 2 hours. Plate wells were then washed 8 times with 100 μL/well of DELFIA® Wash Buffer using the EMBLA 384-plate washer. DELFIA® Enhancement Solution (75 μL) was added to each well and the plates were then covered and incubated overnight at room temperature. Plates were then read on the Wallac 1420 Victor Multilabel Counter set at the Europium protocol for 384-well plates.

In order to examine specificity, the compounds were also assayed against the IGF receptor system in an identical manner.

Results of the compounds tested using this assay procedure are shown in FIG. 7. FIG. 7A shows the results obtained using the gp130/IL-6Rα/IL-6 system, and FIG. 7B shows the results obtained using the IGF-1R/IGF-1 system. These results show that compound 39914 acts specifically as an antagonist of IL-6 binding to IL-6R/gp130 and that compounds 17791 and 56681 act specifically as agonists of IL-6 binding to IL-6R/gp130.

Modelling studies have shown that compounds 39914 and 17791 are likely to interact with IL-6R via the groove on IL-6R defined by residues Phe229, Tyr230, Phe279, Glu278, Glu163, Cys192, Pro162 and Leu108 (see FIG. 8).

TABLE 3
Examples of compounds identified by in silico screening.

| NSC | Formula | CAS No. | Sample Name/structure |
|---|---|---|---|
| 23127 | C₄₂H₂₅N₃O₆ | 128-89-2 | N-(4-((5-(benzoylamino)-9,10- |
| | | | dioxo-9,10-dihydro-1-anthracenyl) |
| | | | amino)-9,10-dioxo-9,10-dihydro-1- |
| | | | anthracenyl)benzamide |
| 35855 | C₃₄H₁₆Cl₂N₂O₆ | 6962-69-2 | 1,2-bis((3-chloro-1,4-dioxo-1,4- |
| | | | dihydro-2-naphthalenyl)amino) |
| | | | anthra-9,10-quinone |
| 39911 | C₂₉H₁₄ClNO₅ | 6336-94-3 | 1-chloro-N-(9,10-dioxo-9,10- |
| | | | dihydro-2-anthracenyl)-9,10-dioxo- |
| | | | 9,10-dihydro-2- |
| | | | anthracenecarboxamide |
| 39918 | C₃₉H₂₁N₃O₆ | 6941-47-5 | N-(3-methyl-2,7-dioxo-2,7-dihydro- |
| | | | 3H-naphtho[1,2,3-de]quinolin-6-yl)- |
| | | | 5,8,14-trioxo-5,8,13,14- |
| | | | tetrahydronaphtho[2,3-c]acridine- |
| | | | 10-carboxamide |
| 51530 | C₂₈H₂₂N₂O₄ | 6636-38-0 | 1,2-dihydroxy-5,8-di(4-toluidino) |
| | | | anthra-9,10-quinone |
| 72123 | C₄₂H₄₅N₄O₅ | 36519-42-3 | Serpentinine |
| 95172 | C₂₇H₁₉NO | 31863-38-4 | 1-(4-benzo[a]anthracen-7- |
| | | | ylbenzoyl)aziridine |
| 102061 | C₂₄H₁₄O₆ | 27172-29-8 | 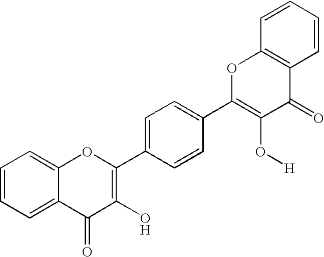 |
| 127622 | C₂₄H₁₆N₂O₁₀ | 25351-81-9 | dimethyl 5-((4,5-dihydroxy-8- |
| | | | (hydroxy(oxido)amino)-9,10-dioxo- |
| | | | 9,10-dihydro-1-anthracenyl) |
| | | | amino)isophthalate |
| 137564 | C₃₀H₂₄O₂S | 51347-04-7 | 2,5-bis(diphenylmethylene) |
| | | | tetrahydrothiophene1,1-dioxide |
| 219732 | C₃₀H₂₆N₄ | 58478-33-4 | N¹, N⁴-di(9-acridinyl)-1,4-butane |
| | | | diamine |
| 244432 | C₃₀H₁₈O₁₂ | 13191-64-5 | Aurofusarin |
| 252124 | C₂₈H₂₄N₁₀O₃ | 80266-34-8 | N-(4-(((4-((3-(hydroxy(oxido) |
| | | | amino)-9-acridinyl)amino)anilino) |
| | | | carbonyl)amino)phenyl)dicarbonimido/ |
| | | | ic diamide/imido |
| 299135 | C₄₄H₂₄N₄O₈ | 4430-70-0 | 1-amino-N-(4-(((1-amino-9,10- |
| | | | dioxo-9,10-dihydro-2-anthracenyl) |
| | | | carbonyl)amino)-9,10-dioxo-9,10- |
| | | | dihydro-1-anthracenyl)-9,10-dioxo- |
| | | | 9,10-dihydro-2-anthracene |
| | | | carboxamide |
| 304394 | C₃₁H₂₈Cl₂N₈ | 77476-26-7 | 2-(1-(7-(N-((4-chloroanilino) |
| | | | (imino)methyl)ethanehydrazonoyl)- |
| | | | 9H-fluoren-2-yl)ethylidene)-N- |
| | | | (4-chlorophenyl)hydrazine |
| | | | carboximidamide |
| 334710 | C₃₂H₁₈N₂O₅ | 70655-11-7 | 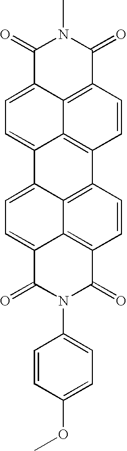 |
| 346568 | C₂₈H₁₈N₄O₂ | 75357-01 | 4-methyl-2-(4-(4-methyl[1,3] |
| | | | oxazolo[4,5-c] quinolin-2-yl) |
| | | | phenyl)[1,3]oxazolo[4,5-c] quinoline |
| 351369 | C₂₈H₁₆O₅ | 51870-19-0 | 6,9-dihydroxy-1,3-diphenylanthra |
| | | | [2,3-c]furan-5,10-dione |
| 112929 | C₄₂H₃₈O₂₀ | 81-27-6 | Sennoside A |
| 661748 | C₄₂H₄₂O₁₂ | 17912-85-5 | Hopeaphenol |
| 307240 | C₅₁H₄₃N₁₃O₁₂S₆ | 56377-79-8 | Nosiheptide |
| 8680 | C₃₄H₂₈N₆O₁₄S₄ | 314-13-6 | 4-amino-6-((4′-((8-amino-1- |
| | | | hydroxy-5,7-disulfo-2-naphthyl) |
| | | | diazenyl)-3,3′-dimethyl[1,1′- |
| | | | biphenyl]-4-yl)diazenyl)-5-hydroxy- |
| | | | 1,3-naphthalene disulfonic acid |
| 65849 | C₃₄H₂₈N₆O₁₄S₄ | 6968-33-8 | 4-amino-6-((4′-((8-amino-1- |
| | | | hydroxy-5,7-disulfo-2- |
| | | | naphthyl)diazenyl)-3,3′- |
| | | | dimethyl[1,1-biphenyl]-4-yl) |
| | | | diazenyl)-5-hydroxy-1,3- |
| | | | naphthalenedisulfonic acid |
| 210354 | C₂₅H₁₇FN₆O₄S | 42447-68-7 | 4-((3-(4-(6-amino-9H-purin-8-yl) |
| | | | benzoyl)anilino)carbonyl)benzene |
| | | | sulfonyl fluoride |
| 9618 | C₃₇H₃₁N₃O₉S₃ | 7401-32-3 | 4-(4-((4-(4-sulfoanilino)-2,5- |
| | | | cyclohexadien-1-ylidene)(4-(4- |
| | | | sulfoanilino)phenyl)methyl) |
| | | | anilino)benzenesulfonic acid |
| 367934 | C₄₀H₂₉NO₆ | 84166-05-2 | 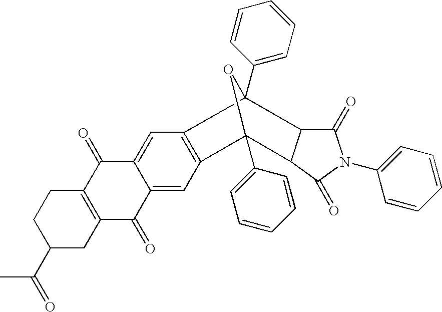 |
| 56681 | C₂₁H₁₂BrNO₄ | 6633-33-6 | 2-(((3-bromo-9-oxo-9H-fluoren- |
| | | | 2-yl)amino)carbonyl)benzoic acid |
| 252124 | C₂₈H₂₄N₁₀O₃ | 80266-34-8 | N-(4-(((4-((3-(hydroxy(oxido) |
| | | | amino)-9-acridinyl)amino)anilino) |
| | | | carbonyl)amino)phenyl)dicarbonimido/ |
| | | | ic diamide/imido |
| 367629 | C₂₃H₁₁N₃O₄ | 81092-84-4 | 12-(hydroxy(oxido)amino)naphtho |
| | | | [1′,2′,3′:4,5]quino[2,1-b] |
| | | | quinazoline-5,10-dione |
| 180974 | C₃₄H₃₂N₂O₅ | 51076-20-1 | Tricordatine |
| 17791 | C₂₃H₁₈NO | 72666-55-8 | 1-[1,1′-biphenyl]-4-yl-2-(2⁵- |
| | | | isoquinolin-2-yl)ethanone |
| 39914 | C₂₉H₁₆N₂O₅ | 6336-97-6 | 1-amino-N-(9,10-dioxo-9,10- |
| | | | dihydro-2-anthracenyl)-9,10-dioxo- |
| | | | 9,10-dihydro-2- |
| | | | anthracenecarboxamide |
| 72275 | C₄₉H₃₃N₇O₆S₇ | 8064-60-6 | Primuline |
| 377102 | C₃₈H₄₀N₄O | 85335-06-4 | 11-(3-ethylidene- |
| | | | 1,2,3,4,6,7,12,12b-octahydro |
| | | | indolo[2,3-a]quinolizin-2-yl)-10,11- |
| | | | didehydrostrychnidine |
| 39965 | C₂₉H₁₄N₂O₄S | 6937-84-4 | 2-(1-amino-9,10-dioxo-9,10- |
| | | | dihydro-2-anthracenyl)anthra[2, |
| | | | 1-d][1,3]thiazole-6,11-dione |
| 51306 | C₂₁H₁₀ BrNO₃ | 6344-57-6 | 2-(7-bromo-9-oxo-9H-fluoren-2-yl)- |
| | | | 1H-isoindole-1,3(2H)-dione |

The disclosure of all publications referred to in this application are incorporated herein by reference.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

APPENDIX I
Structural coordinates of sIL-6R

| [symbol] | [symbol] | [symbol] | [symbol] | [symbol] | [symbol] | [symbol] | [symbol] | [symbol] |
|---|---|---|---|---|---|---|---|---|
| 1 | CB | LEU | 1 | 0.449 | 42.372 | 88.777 | 1.00 | 101.99 |
| 2 | CG | LEU | 1 | −0.872 | 41.888 | 89.392 | 1.00 | 94.37 |
| 3 | CD1 | LEU | 1 | −1.205 | 40.494 | 88.893 | 1.00 | 92.50 |
| 4 | CD2 | LEU | 1 | −0.747 | 41.898 | 90.908 | 1.00 | 88.11 |
| 5 | C | LEU | 1 | −0.377 | 43.406 | 86.628 | 1.00 | 99.90 |
| 6 | O | LEU | 1 | −0.151 | 44.619 | 86.572 | 1.00 | 96.65 |
| 7 | N | LEU | 1 | 2.074 | 42.766 | 86.934 | 1.00 | 93.31 |
| 8 | CA | LEU | 1 | 0.644 | 42.431 | 87.249 | 1.00 | 100.21 |
| 9 | N | ALA | 2 | −1.494 | 42.849 | 86.154 | 1.00 | 99.76 |
| 10 | CA | ALA | 2 | −2.580 | 43.615 | 85.548 | 1.00 | 95.84 |
| 11 | CB | ALA | 2 | −2.914 | 43.038 | 84.183 | 1.00 | 85.11 |
| 12 | C | ALA | 2 | −3.782 | 43.547 | 86.523 | 1.00 | 100.62 |
| 13 | O | ALA | 2 | −3.616 | 43.865 | 87.695 | 1.00 | 96.47 |
| 14 | N | PRO | 3 | −4.990 | 43.128 | 86.082 | 1.00 | 104.63 |
| 15 | CD | PRO | 3 | −5.587 | 42.853 | 84.757 | 1.00 | 106.80 |
| 16 | CA | PRO | 3 | −6.048 | 43.111 | 87.096 | 1.00 | 103.58 |
| 17 | CB | PRO | 3 | −7.117 | 42.246 | 86.439 | 1.00 | 98.19 |
| 18 | CG | PRO | 3 | −7.099 | 42.783 | 85.066 | 1.00 | 97.53 |
| 19 | C | PRO | 3 | −5.752 | 42.702 | 88.542 | 1.00 | 103.09 |
| 20 | O | PRO | 3 | −4.669 | 42.222 | 88.881 | 1.00 | 100.54 |
| 21 | N | ARG | 4 | −6.770 | 42.924 | 89.370 | 1.00 | 103.31 |
| 22 | CA | ARG | 4 | −6.780 | 42.661 | 90.802 | 1.00 | 100.67 |
| 23 | CB | ARG | 4 | −8.221 | 42.387 | 91.252 | 1.00 | 104.27 |
| 24 | CG | ARG | 4 | −9.099 | 43.636 | 91.315 | 1.00 | 102.65 |
| 25 | CD | ARG | 4 | −8.751 | 44.506 | 92.515 | 1.00 | 103.47 |
| 26 | NE | ARG | 4 | −9.538 | 45.738 | 92.547 | 1.00 | 103.54 |
| 27 | CZ | ARG | 4 | −9.598 | 46.564 | 93.589 | 1.00 | 103.37 |
| 28 | NH1 | ARG | 4 | −8.918 | 46.294 | 94.697 | 1.00 | 99.61 |
| 29 | NH2 | ARG | 4 | −10.340 | 47.663 | 93.523 | 1.00 | 104.86 |
| 30 | C | ARG | 4 | −5.854 | 41.600 | 91.387 | 1.00 | 96.59 |
| 31 | O | ARG | 4 | −4.630 | 41.756 | 91.383 | 1.00 | 96.87 |
| 32 | N | ARG | 5 | −6.426 | 40.527 | 91.917 | 1.00 | 90.24 |
| 33 | CA | ARG | 5 | −5.583 | 39.531 | 92.530 | 1.00 | 84.74 |
| 34 | CB | ARG | 5 | −5.956 | 39.379 | 94.009 | 1.00 | 87.33 |
| 35 | CG | ARG | 5 | −7.438 | 39.238 | 94.289 | 1.00 | 86.83 |
| 36 | CD | ARG | 5 | −7.709 | 39.272 | 95.796 | 1.00 | 84.68 |
| 37 | NE | ARG | 5 | −7.035 | 38.187 | 96.504 | 0.00 | 86.06 |
| 38 | CZ | ARG | 5 | −7.106 | 37.995 | 97.818 | 0.00 | 86.05 |
| 39 | NH1 | ARG | 5 | −7.822 | 38.817 | 98.573 | 0.00 | 86.25 |
| 40 | NH2 | ARG | 5 | −6.460 | 36.981 | 98.378 | 0.00 | 86.25 |
| 41 | C | ARG | 5 | −5.425 | 38.163 | 91.877 | 1.00 | 81.24 |
| 42 | O | ARG | 5 | −6.174 | 37.746 | 90.983 | 1.00 | 67.00 |
| 43 | N | CYS | 6 | −4.387 | 37.492 | 92.361 | 1.00 | 78.26 |
| 44 | CA | CYS | 6 | −3.962 | 36.175 | 91.914 | 1.00 | 73.58 |
| 45 | C | CYS | 6 | −3.912 | 35.289 | 93.148 | 1.00 | 63.52 |
| 46 | O | CYS | 6 | −2.840 | 34.927 | 93.623 | 1.00 | 61.36 |
| 47 | CB | CYS | 6 | −2.571 | 36.314 | 91.285 | 1.00 | 61.71 |
| 48 | SG | CYS | 6 | −1.860 | 34.803 | 90.596 | 1.00 | 58.67 |
| 49 | N | PRO | 7 | −5.079 | 34.953 | 93.702 | 1.00 | 64.27 |
| 50 | CD | PRO | 7 | −6.440 | 35.188 | 93.180 | 1.00 | 58.88 |
| 51 | CA | PRO | 7 | −5.103 | 34.098 | 94.899 | 1.00 | 64.89 |
| 52 | CB | PRO | 7 | −6.513 | 34.320 | 95.441 | 1.00 | 62.19 |
| 53 | CG | PRO | 7 | −7.334 | 34.418 | 94.178 | 1.00 | 62.11 |
| 54 | C | PRO | 7 | −4.831 | 32.610 | 94.577 | 1.00 | 66.48 |
| 55 | O | PRO | 7 | −4.798 | 32.200 | 93.399 | 1.00 | 51.46 |
| 56 | N | ALA | 8 | −4.632 | 31.806 | 95.619 | 1.00 | 59.58 |
| 57 | CA | ALA | 8 | −4.399 | 30.391 | 95.422 | 1.00 | 64.43 |
| 58 | CB | ALA | 8 | −3.785 | 29.779 | 96.654 | 1.00 | 64.83 |
| 59 | C | ALA | 8 | −5.758 | 29.768 | 95.152 | 1.00 | 71.86 |
| 60 | O | ALA | 8 | −6.787 | 30.460 | 95.202 | 1.00 | 63.92 |
| 61 | N | GLN | 9 | −5.758 | 28.466 | 94.860 | 1.00 | 72.66 |
| 62 | CA | GLN | 9 | −6.991 | 27.736 | 94.601 | 1.00 | 72.67 |
| 63 | CB | GLN | 9 | −6.669 | 26.379 | 94.002 | 1.00 | 72.94 |
| 64 | CG | GLN | 9 | −7.340 | 26.147 | 92.671 | 1.00 | 79.11 |
| 65 | CD | GLN | 9 | −6.800 | 27.062 | 91.612 | 1.00 | 77.56 |
| 66 | OE1 | GLN | 9 | −7.552 | 27.637 | 90.827 | 1.00 | 61.51 |
| 67 | NE2 | GLN | 9 | −5.480 | 27.203 | 91.579 | 1.00 | 79.60 |
| 68 | C | GLN | 9 | −7.785 | 27.564 | 95.899 | 1.00 | 66.36 |
| 69 | O | GLN | 9 | −7.209 | 27.379 | 96.957 | 1.00 | 59.92 |
| 70 | N | GLU | 10 | −9.110 | 27.622 | 95.807 | 1.00 | 77.06 |
| 71 | CA | GLU | 10 | −9.981 | 27.524 | 96.983 | 1.00 | 79.66 |
| 72 | CB | GLU | 10 | −11.439 | 27.763 | 96.583 | 1.00 | 73.18 |
| 73 | CG | GLU | 10 | −11.689 | 29.046 | 95.811 | 1.00 | 71.73 |
| 74 | CD | GLU | 10 | −11.299 | 30.286 | 96.591 | 0.00 | 75.29 |
| 75 | OE1 | GLU | 10 | −11.727 | 30.414 | 97.758 | 0.00 | 75.50 |
| 76 | OE2 | GLU | 10 | −10.572 | 31.136 | 96.036 | 0.00 | 75.50 |
| 77 | C | GLU | 10 | −9.909 | 26.228 | 97.794 | 1.00 | 84.43 |
| 78 | O | GLU | 10 | −10.113 | 26.247 | 99.021 | 1.00 | 86.07 |
| 79 | N | VAL | 11 | −9.627 | 25.115 | 97.119 | 1.00 | 77.75 |
| 80 | CA | VAL | 11 | −9.558 | 23.799 | 97.777 | 1.00 | 80.23 |
| 81 | CB | VAL | 11 | −8.481 | 23.760 | 98.897 | 1.00 | 81.11 |
| 82 | CG1 | VAL | 11 | −8.281 | 22.339 | 99.356 | 1.00 | 83.81 |
| 83 | CG2 | VAL | 11 | −7.164 | 24.335 | 98.400 | 1.00 | 80.64 |
| 84 | C | VAL | 11 | −10.914 | 23.432 | 98.398 | 1.00 | 73.95 |
| 85 | O | VAL | 11 | −11.523 | 24.244 | 99.101 | 1.00 | 78.59 |
| 86 | N | ALA | 12 | −11.377 | 22.209 | 98.141 | 1.00 | 73.28 |
| 87 | CA | ALA | 12 | −12.673 | 21.735 | 98.646 | 1.00 | 75.11 |
| 88 | CB | ALA | 12 | −12.980 | 20.367 | 98.057 | 1.00 | 73.67 |
| 89 | C | ALA | 12 | −12.817 | 21.693 | 100.180 | 1.00 | 82.50 |
| 90 | O | ALA | 12 | −11.835 | 21.822 | 100.926 | 1.00 | 83.38 |
| 91 | N | ARG | 13 | −14.055 | 21.515 | 100.639 | 1.00 | 83.59 |
| 92 | CA | ARG | 13 | −14.374 | 21.466 | 102.070 | 1.00 | 81.64 |
| 93 | CB | ARG | 13 | −15.848 | 21.096 | 102.261 | 0.00 | 81.09 |
| 94 | CG | ARG | 13 | −16.824 | 22.105 | 101.672 | 0.00 | 81.08 |
| 95 | CD | ARG | 13 | −18.265 | 21.722 | 101.974 | 0.00 | 81.22 |
| 96 | NE | ARG | 13 | −18.642 | 20.453 | 101.356 | 0.00 | 81.49 |
| 97 | CZ | ARG | 13 | −18.750 | 20.262 | 100.045 | 0.00 | 81.62 |
| 98 | NH1 | ARG | 13 | −18.512 | 21.259 | 99.203 | 0.00 | 81.72 |
| 99 | NH2 | ARG | 13 | −19.098 | 19.072 | 99.574 | 0.00 | 81.72 |
| 100 | C | ARG | 13 | −13.501 | 20.515 | 102.899 | 1.00 | 80.15 |
| 101 | O | ARG | 13 | −13.347 | 19.335 | 102.568 | 1.00 | 77.92 |
| 102 | N | GLY | 14 | −12.932 | 21.050 | 103.976 | 1.00 | 74.94 |
| 103 | CA | GLY | 14 | −12.093 | 20.266 | 104.863 | 1.00 | 75.60 |
| 104 | C | GLY | 14 | −10.943 | 19.494 | 104.231 | 1.00 | 82.74 |
| 105 | O | GLY | 14 | −10.543 | 18.457 | 104.772 | 1.00 | 82.02 |
| 106 | N | VAL | 15 | −10.431 | 19.958 | 103.087 | 1.00 | 73.38 |
| 107 | CA | VAL | 15 | −9.294 | 19.304 | 102.453 | 1.00 | 57.66 |
| 108 | CB | VAL | 15 | −9.256 | 19.546 | 100.923 | 1.00 | 61.09 |
| 109 | CG1 | VAL | 15 | −7.886 | 19.156 | 100.345 | 1.00 | 48.64 |
| 110 | CG2 | VAL | 15 | −10.339 | 18.743 | 100.242 | 1.00 | 42.61 |
| 111 | C | VAL | 15 | −8.044 | 19.922 | 103.099 | 1.00 | 66.02 |
| 112 | O | VAL | 15 | −7.994 | 21.114 | 103.373 | 1.00 | 67.62 |
| 113 | N | LEU | 16 | −7.050 | 19.088 | 103.360 | 1.00 | 65.90 |
| 114 | CA | LEU | 16 | −5.799 | 19.499 | 103.975 | 1.00 | 54.43 |
| 115 | CB | LEU | 16 | −5.389 | 18.458 | 105.009 | 1.00 | 61.09 |
| 116 | CG | LEU | 16 | −4.329 | 18.691 | 106.074 | 1.00 | 54.68 |
| 117 | CD1 | LEU | 16 | −4.921 | 19.504 | 107.252 | 1.00 | 55.60 |
| 118 | CD2 | LEU | 16 | −3.877 | 17.332 | 106.577 | 1.00 | 53.84 |
| 119 | C | LEU | 16 | −4.787 | 19.476 | 102.845 | 1.00 | 62.00 |
| 120 | O | LEU | 16 | −4.784 | 18.539 | 102.034 | 1.00 | 56.46 |
| 121 | N | THR | 17 | −3.917 | 20.479 | 102.797 | 1.00 | 44.10 |
| 122 | CA | THR | 17 | −2.918 | 20.533 | 101.754 | 1.00 | 44.95 |
| 123 | CB | THR | 17 | −3.074 | 21.817 | 100.905 | 1.00 | 48.77 |
| 124 | OG1 | THR | 17 | −3.119 | 22.943 | 101.785 | 1.00 | 48.72 |
| 125 | CG2 | THR | 17 | −4.385 | 21.796 | 100.095 | 1.00 | 48.05 |
| 126 | C | THR | 17 | −1.577 | 20.573 | 102.427 | 1.00 | 49.81 |
| 127 | O | THR | 17 | −1.495 | 20.786 | 103.628 | 1.00 | 53.80 |
| 128 | N | SER | 18 | −0.531 | 20.358 | 101.647 | 1.00 | 37.06 |
| 129 | CA | SER | 18 | 0.811 | 20.431 | 102.154 | 1.00 | 36.34 |
| 130 | CB | SER | 18 | 1.132 | 19.315 | 103.169 | 1.00 | 45.34 |
| 131 | OG | SER | 18 | 0.597 | 18.069 | 102.775 | 1.00 | 63.40 |
| 132 | C | SER | 18 | 1.758 | 20.344 | 100.994 | 1.00 | 47.68 |
| 133 | O | SER | 18 | 1.408 | 19.882 | 99.908 | 1.00 | 48.83 |
| 134 | N | LEU | 19 | 2.962 | 20.820 | 101.248 | 1.00 | 47.97 |
| 135 | CA | LEU | 19 | 4.016 | 20.833 | 100.271 | 1.00 | 53.12 |
| 136 | CB | LEU | 19 | 4.963 | 21.986 | 100.579 | 1.00 | 51.40 |
| 137 | CG | LEU | 19 | 4.262 | 23.281 | 100.984 | 1.00 | 56.98 |
| 138 | CD1 | LEU | 19 | 5.317 | 24.279 | 101.366 | 1.00 | 58.94 |
| 139 | CD2 | LEU | 19 | 3.401 | 23.812 | 99.829 | 1.00 | 56.85 |
| 140 | C | LEU | 19 | 4.768 | 19.526 | 100.377 | 1.00 | 53.21 |
| 141 | O | LEU | 19 | 4.694 | 18.848 | 101.393 | 1.00 | 60.27 |
| 142 | N | PRO | 20 | 5.498 | 19.160 | 99.324 | 1.00 | 54.31 |
| 143 | CD | PRO | 20 | 5.511 | 19.781 | 97.982 | 1.00 | 53.88 |
| 144 | CA | PRO | 20 | 6.266 | 17.917 | 99.353 | 1.00 | 45.95 |
| 145 | CB | PRO | 20 | 6.796 | 17.789 | 97.926 | 1.00 | 47.82 |
| 146 | CG | PRO | 20 | 5.807 | 18.601 | 97.095 | 1.00 | 59.03 |
| 147 | C | PRO | 20 | 7.412 | 18.122 | 100.335 | 1.00 | 53.81 |
| 148 | O | PRO | 20 | 8.015 | 19.210 | 100.372 | 1.00 | 43.31 |
| 149 | N | GLY | 21 | 7.712 | 17.066 | 101.095 | 1.00 | 49.09 |
| 150 | CA | GLY | 21 | 8.785 | 17.081 | 102.078 | 1.00 | 43.34 |
| 151 | C | GLY | 21 | 8.300 | 17.368 | 103.488 | 1.00 | 35.95 |
| 152 | O | GLY | 21 | 9.019 | 17.193 | 104.457 | 1.00 | 39.88 |
| 153 | N | ASP | 22 | 7.063 | 17.823 | 103.612 | 1.00 | 43.97 |
| 154 | CA | ASP | 22 | 6.553 | 18.166 | 104.934 | 1.00 | 48.72 |
| 155 | CB | ASP | 22 | 5.387 | 19.170 | 104.842 | 1.00 | 40.75 |
| 156 | CG | ASP | 22 | 5.856 | 20.621 | 104.619 | 1.00 | 65.71 |
| 157 | OD1 | ASP | 22 | 7.091 | 20.902 | 104.720 | 1.00 | 65.01 |
| 158 | OD2 | ASP | 22 | 4.969 | 21.478 | 104.349 | 1.00 | 59.85 |
| 159 | C | ASP | 22 | 6.042 | 16.927 | 105.604 | 1.00 | 52.26 |
| 160 | O | ASP | 22 | 5.764 | 15.944 | 104.931 | 1.00 | 58.05 |
| 161 | N | SER | 23 | 5.961 | 16.972 | 106.931 | 1.00 | 44.50 |
| 162 | CA | SER | 23 | 5.376 | 15.889 | 107.685 | 1.00 | 37.87 |
| 163 | CB | SER | 23 | 6.013 | 15.753 | 109.049 | 1.00 | 32.56 |
| 164 | OG | SER | 23 | 7.254 | 15.069 | 108.913 | 1.00 | 59.71 |
| 165 | C | SER | 23 | 3.931 | 16.317 | 107.847 | 1.00 | 38.38 |
| 166 | O | SER | 23 | 3.654 | 17.504 | 107.908 | 1.00 | 51.39 |
| 167 | N | VAL | 24 | 3.013 | 15.366 | 107.883 | 1.00 | 41.93 |
| 168 | CA | VAL | 24 | 1.604 | 15.686 | 108.061 | 1.00 | 46.54 |
| 169 | CB | VAL | 24 | 0.804 | 15.494 | 106.759 | 1.00 | 40.44 |
| 170 | CG1 | VAL | 24 | −0.682 | 15.795 | 106.970 | 1.00 | 36.10 |
| 171 | CG2 | VAL | 24 | 1.383 | 16.388 | 105.678 | 1.00 | 35.61 |
| 172 | C | VAL | 24 | 1.047 | 14.765 | 109.136 | 1.00 | 52.56 |
| 173 | O | VAL | 24 | 1.372 | 13.588 | 109.187 | 1.00 | 52.11 |
| 174 | N | THR | 25 | 0.233 | 15.316 | 110.022 | 1.00 | 53.11 |
| 175 | CA | THR | 25 | −0.339 | 14.497 | 111.057 | 1.00 | 55.62 |
| 176 | CB | THR | 25 | −0.198 | 15.153 | 112.462 | 1.00 | 34.57 |
| 177 | OG1 | THR | 25 | 1.181 | 15.211 | 112.805 | 1.00 | 48.14 |
| 178 | CG2 | THR | 25 | −0.853 | 14.333 | 113.498 | 1.00 | 46.34 |
| 179 | C | THR | 25 | −1.797 | 14.214 | 110.728 | 1.00 | 64.24 |
| 180 | O | THR | 25 | −2.690 | 15.060 | 110.896 | 1.00 | 57.60 |
| 181 | N | LEU | 26 | −2.013 | 13.007 | 110.218 | 1.00 | 65.53 |
| 182 | CA | LEU | 26 | −3.347 | 12.556 | 109.914 | 1.00 | 62.85 |
| 183 | CB | LEU | 26 | −3.321 | 11.163 | 109.280 | 1.00 | 66.87 |
| 184 | CG | LEU | 26 | −2.607 | 10.950 | 107.937 | 1.00 | 66.83 |
| 185 | CD1 | LEU | 26 | −2.545 | 9.446 | 107.621 | 1.00 | 62.48 |
| 186 | CD2 | LEU | 26 | −3.312 | 11.695 | 106.855 | 1.00 | 51.71 |
| 187 | C | LEU | 26 | −3.941 | 12.474 | 111.304 | 1.00 | 61.75 |
| 188 | O | LEW | 26 | −3.359 | 11.878 | 112.209 | 1.00 | 62.35 |
| 189 | N | THR | 27 | −5.088 | 13.095 | 111.483 | 1.00 | 66.71 |
| 190 | CA | THR | 27 | −5.738 | 13.071 | 112.776 | 1.00 | 67.80 |
| 191 | CB | THR | 27 | −5.724 | 14.460 | 113.388 | 1.00 | 61.33 |
| 192 | OG1 | THR | 27 | −4.362 | 14.908 | 113.439 | 1.00 | 63.93 |
| 193 | CG2 | THR | 27 | −6.320 | 14.446 | 114.774 | 1.00 | 60.73 |
| 194 | C | THR | 27 | −7.151 | 12.578 | 112.591 | 1.00 | 63.45 |
| 195 | O | THR | 27 | −7.997 | 13.259 | 112.039 | 1.00 | 62.43 |
| 196 | N | CYS | 28 | −7.387 | 11.356 | 113.033 | 1.00 | 78.61 |
| 197 | CA | CYS | 28 | −8.702 | 10.763 | 112.919 | 1.00 | 79.00 |
| 198 | C | CYS | 28 | −9.701 | 11.657 | 113.640 | 1.00 | 76.66 |
| 199 | O | CYS | 28 | −9.552 | 11.934 | 114.839 | 1.00 | 70.85 |
| 200 | CB | CYS | 28 | −8.708 | 9.389 | 113.551 | 1.00 | 78.96 |
| 201 | SG | CYS | 28 | −10.222 | 8.474 | 113.170 | 1.00 | 91.84 |
| 202 | N | PRO | 29 | −10.720 | 12.136 | 112.911 | 1.00 | 75.64 |
| 203 | CD | PRO | 29 | −10.843 | 11.998 | 111.448 | 1.00 | 75.90 |
| 204 | CA | PRO | 29 | −11.771 | 13.010 | 113.449 | 1.00 | 78.49 |
| 205 | CB | PRO | 29 | −12.577 | 13.381 | 112.210 | 1.00 | 81.18 |
| 206 | CG | PRO | 29 | −11.542 | 13.263 | 111.079 | 1.00 | 83.97 |
| 207 | C | PRO | 29 | −12.646 | 12.327 | 114.504 | 1.00 | 81.15 |
| 208 | O | PRO | 29 | −12.916 | 12.886 | 115.574 | 1.00 | 78.68 |
| 209 | N | GLY | 30 | −13.061 | 11.103 | 114.198 | 1.00 | 78.44 |
| 210 | CA | GLY | 30 | −13.932 | 10.364 | 115.090 | 1.00 | 80.19 |
| 211 | C | GLY | 30 | −13.327 | 9.653 | 116.278 | 1.00 | 79.60 |
| 212 | O | GLY | 30 | −14.041 | 8.931 | 116.969 | 1.00 | 79.98 |
| 213 | N | VAL | 31 | −12.033 | 9.819 | 116.530 | 1.00 | 73.95 |
| 214 | CA | VAL | 31 | −11.453 | 9.146 | 117.684 | 1.00 | 72.63 |
| 215 | CB | VAL | 31 | −10.359 | 8.142 | 117.292 | 1.00 | 65.98 |
| 216 | CG1 | VAL | 31 | −9.847 | 7.464 | 118.530 | 1.00 | 65.37 |
| 217 | CG2 | VAL | 31 | −10.914 | 7.087 | 116.336 | 1.00 | 69.40 |
| 218 | C | VAL | 31 | −10.888 | 10.144 | 118.683 | 1.00 | 73.31 |
| 219 | O | VAL | 31 | −10.029 | 10.967 | 118.357 | 1.00 | 68.46 |
| 220 | N | GLU | 32 | −11.387 | 10.051 | 119.911 | 1.00 | 68.79 |
| 221 | CA | GLU | 32 | −10.989 | 10.941 | 120.995 | 1.00 | 75.74 |
| 222 | CB | GLU | 32 | −11.594 | 10.444 | 122.315 | 1.00 | 79.09 |
| 223 | CG | GLU | 32 | −13.117 | 10.366 | 122.307 | 0.00 | 78.48 |
| 224 | CD | GLU | 32 | −13.692 | 9.863 | 123.620 | 0.00 | 78.92 |
| 225 | OE1 | GLU | 32 | −14.933 | 9.766 | 123.728 | 0.00 | 79.02 |
| 226 | OE2 | GLU | 32 | −12.906 | 9.563 | 124.543 | 0.00 | 79.02 |
| 227 | C | GLU | 32 | −9.476 | 11.104 | 121.139 | 1.00 | 74.42 |
| 228 | O | GLU | 32 | −8.700 | 10.232 | 120.752 | 1.00 | 64.96 |
| 229 | N | PRO | 33 | −9.040 | 12.236 | 121.714 | 1.00 | 82.52 |
| 230 | CD | PRO | 33 | −9.812 | 13.381 | 122.231 | 1.00 | 81.58 |
| 231 | CA | PRO | 33 | −7.602 | 12.459 | 121.886 | 1.00 | 85.29 |
| 232 | CB | PRO | 33 | −7.541 | 13.870 | 122.488 | 1.00 | 79.15 |
| 233 | CG | PRO | 33 | −8.847 | 13.985 | 123.227 | 1.00 | 79.56 |
| 234 | C | PRO | 33 | −6.848 | 11.422 | 122.732 | 1.00 | 83.28 |
| 235 | O | PRO | 33 | −5.616 | 11.386 | 122.699 | 1.00 | 86.54 |
| 236 | N | GLU | 34 | −7.553 | 10.573 | 123.475 | 1.00 | 74.84 |
| 237 | CA | GLU | 34 | −6.833 | 9.609 | 124.303 | 1.00 | 85.61 |
| 238 | CB | GLU | 34 | −6.954 | 10.003 | 125.782 | 1.00 | 85.04 |
| 239 | CG | GLU | 34 | −8.357 | 9.891 | 126.355 | 1.00 | 91.95 |
| 240 | CD | GLU | 34 | −9.298 | 10.957 | 125.828 | 1.00 | 91.82 |
| 241 | OE1 | GLU | 34 | −9.139 | 12.136 | 126.221 | 1.00 | 97.09 |
| 242 | OE2 | GLU | 34 | −10.192 | 10.615 | 125.022 | 1.00 | 85.80 |
| 243 | C | GLU | 34 | −7.130 | 8.108 | 124.169 | 1.00 | 86.02 |
| 244 | O | GLU | 34 | −6.658 | 7.326 | 124.988 | 1.00 | 86.82 |
| 245 | N | ASP | 35 | −7.877 | 7.684 | 123.153 | 1.00 | 88.67 |
| 246 | CA | ASP | 35 | −8.167 | 6.258 | 123.026 | 1.00 | 89.92 |
| 247 | CB | ASP | 35 | −9.389 | 6.014 | 122.130 | 1.00 | 90.08 |
| 248 | CG | ASP | 35 | −10.125 | 4.710 | 122.481 | 1.00 | 94.14 |
| 249 | OD1 | ASP | 35 | −9.512 | 3.805 | 123.110 | 1.00 | 85.08 |
| 250 | OD2 | ASP | 35 | −11.321 | 4.596 | 122.117 | 1.00 | 90.82 |
| 251 | C | ASP | 35 | −6.972 | 5.480 | 122.472 | 1.00 | 89.41 |
| 252 | O | ASP | 35 | −6.711 | 5.483 | 121.263 | 1.00 | 94.62 |
| 253 | N | ASN | 36 | −6.262 | 4.803 | 123.369 | 1.00 | 86.90 |
| 254 | CA | ASN | 36 | −5.096 | 4.013 | 123.003 | 1.00 | 78.62 |
| 255 | CB | ASN | 36 | −4.437 | 3.448 | 124.268 | 1.00 | 83.78 |
| 256 | CG | ASN | 36 | −3.841 | 4.526 | 125.158 | 1.00 | 90.38 |
| 257 | OD1 | ASN | 36 | −2.795 | 5.111 | 124.843 | 1.00 | 90.31 |
| 258 | ND2 | ASN | 36 | −4.515 | 4.787 | 126.274 | 1.00 | 91.32 |
| 259 | C | ASN | 36 | −5.482 | 2.863 | 122.070 | 1.00 | 76.89 |
| 260 | O | ASN | 36 | −4.631 | 2.053 | 121.683 | 1.00 | 72.83 |
| 261 | N | ALA | 37 | −6.761 | 2.785 | 121.708 | 1.00 | 73.54 |
| 262 | CA | ALA | 37 | −7.224 | 1.707 | 120.832 | 1.00 | 75.00 |
| 263 | CB | ALA | 37 | −8.753 | 1.796 | 120.622 | 1.00 | 66.82 |
| 264 | C | ALA | 37 | −6.499 | 1.712 | 119.478 | 1.00 | 72.68 |
| 265 | O | ALA | 37 | −6.061 | 2.757 | 118.980 | 1.00 | 50.84 |
| 266 | N | THR | 38 | −6.352 | 0.534 | 118.889 | 1.00 | 70.07 |
| 267 | CA | THR | 38 | −5.704 | 0.481 | 117.602 | 1.00 | 74.82 |
| 268 | CB | THR | 38 | −5.417 | −0.956 | 117.146 | 1.00 | 71.82 |
| 269 | OG1 | THR | 38 | −4.575 | −1.593 | 118.109 | 1.00 | 82.67 |
| 270 | CG2 | THR | 38 | −4.709 | −0.957 | 115.776 | 1.00 | 71.84 |
| 271 | C | THR | 38 | −6.653 | 1.113 | 116.604 | 1.00 | 76.60 |
| 272 | O | THR | 38 | −7.871 | 0.895 | 116.661 | 1.00 | 74.96 |
| 273 | N | VAL | 39 | −6.100 | 1.941 | 115.725 | 1.00 | 71.86 |
| 274 | CA | VAL | 39 | −6.894 | 2.550 | 114.677 | 1.00 | 69.71 |
| 275 | CB | VAL | 39 | −6.802 | 4.101 | 114.669 | 1.00 | 62.14 |
| 276 | CG1 | VAL | 39 | −7.425 | 4.639 | 113.406 | 1.00 | 71.47 |
| 277 | CG2 | VAL | 39 | −7.561 | 4.684 | 115.840 | 1.00 | 64.03 |
| 278 | C | VAL | 39 | −6.314 | 1.981 | 113.377 | 1.00 | 67.00 |
| 279 | O | VAL | 39 | −5.117 | 1.700 | 113.277 | 1.00 | 63.20 |
| 280 | N | HIS | 40 | −7.188 | 1.758 | 112.408 | 1.00 | 64.17 |
| 281 | CA | HIS | 40 | −6.769 | 1.257 | 111.120 | 1.00 | 59.06 |
| 282 | CB | HIS | 40 | −7.513 | −0.014 | 110.784 | 1.00 | 55.54 |
| 283 | CG | HIS | 40 | −7.040 | −1.184 | 111.570 | 1.00 | 60.37 |
| 284 | CD2 | HIS | 40 | −7.433 | −1.670 | 112.771 | 1.00 | 48.89 |
| 285 | ND1 | HIS | 40 | −5.956 | −1.939 | 111.183 | 1.00 | 59.70 |
| 286 | CE1 | HIS | 40 | −5.697 | −2.838 | 112.114 | 1.00 | 55.97 |
| 287 | NE2 | HIS | 40 | −6.576 | −2.694 | 113.087 | 1.00 | 62.89 |
| 288 | C | HIS | 40 | −7.045 | 2.322 | 110.079 | 1.00 | 59.08 |
| 289 | O | HIS | 40 | −8.064 | 3.023 | 110.127 | 1.00 | 53.03 |
| 290 | N | TRP | 41 | −6.103 | 2.442 | 109.153 | 1.00 | 51.62 |
| 291 | CA | TRP | 41 | −6.197 | 3.401 | 108.080 | 1.00 | 49.90 |
| 292 | CB | TRP | 41 | −5.019 | 4.375 | 108.121 | 1.00 | 56.38 |
| 293 | CG | TRP | 41 | −4.947 | 5.165 | 109.352 | 1.00 | 51.16 |
| 294 | CD2 | TRP | 41 | −5.472 | 6.471 | 109.553 | 1.00 | 60.65 |
| 295 | CE2 | TRP | 41 | −5.160 | 6.846 | 110.880 | 1.00 | 59.64 |
| 296 | CE3 | TRP | 41 | −6.173 | 7.369 | 108.743 | 1.00 | 57.13 |
| 297 | CD1 | TRP | 41 | −4.359 | 4.798 | 110.533 | 1.00 | 59.46 |
| 298 | NE1 | TRP | 41 | −4.485 | 5.802 | 111.453 | 1.00 | 62.96 |
| 299 | CZ2 | TRP | 41 | −5.519 | 8.073 | 111.409 | 1.00 | 56.70 |
| 300 | CZ3 | TRP | 41 | −6.535 | 8.595 | 109.274 | 1.00 | 65.95 |
| 301 | CH2 | TRP | 41 | −6.205 | 8.937 | 110.596 | 1.00 | 62.56 |
| 302 | C | TRP | 41 | −6.195 | 2.713 | 106.731 | 1.00 | 57.85 |
| 303 | O | TRP | 41 | −5.486 | 1.712 | 106.487 | 1.00 | 50.96 |
| 304 | N | VAL | 42 | −6.997 | 3.263 | 105.841 | 1.00 | 52.35 |
| 305 | CA | VAL | 42 | −7.044 | 2.725 | 104.514 | 1.00 | 56.46 |
| 306 | CB | VAL | 42 | −8.424 | 2.103 | 104.194 | 1.00 | 57.82 |
| 307 | CG1 | VAL | 42 | −8.423 | 1.565 | 102.767 | 1.00 | 54.15 |
| 308 | CG2 | VAL | 42 | −8.724 | 0.963 | 105.171 | 1.00 | 50.16 |
| 309 | C | VAL | 42 | −6.793 | 3.944 | 103.680 | 1.00 | 52.80 |
| 310 | O | VAL | 42 | −7.498 | 4.931 | 103.823 | 1.00 | 54.05 |
| 311 | N | LEU | 43 | −5.753 | 3.888 | 102.853 | 1.00 | 55.97 |
| 312 | CA | LEU | 43 | −5.403 | 5.003 | 101.989 | 1.00 | 59.83 |
| 313 | CB | LEU | 43 | −3.893 | 5.250 | 102.001 | 1.00 | 55.98 |
| 314 | CG | LEU | 43 | −3.329 | 5.989 | 100.769 | 1.00 | 53.52 |
| 315 | CD1 | LEU | 43 | −3.876 | 7.424 | 100.683 | 1.00 | 41.04 |
| 316 | CD2 | LEU | 43 | −1.778 | 5.961 | 100.834 | 1.00 | 48.18 |
| 317 | C | LEU | 43 | −5.847 | 4.704 | 100.569 | 1.00 | 64.86 |
| 318 | O | LEU | 43 | −5.537 | 3.630 | 100.027 | 1.00 | 54.65 |
| 319 | N | ARG | 44 | −6.554 | 5.668 | 99.974 | 1.00 | 65.34 |
| 320 | CA | ARG | 44 | −7.051 | 5.547 | 98.597 | 1.00 | 71.53 |
| 321 | CB | ARG | 44 | −8.594 | 5.505 | 98.597 | 1.00 | 68.85 |
| 322 | CG | ARG | 44 | −9.156 | 4.294 | 99.368 | 1.00 | 77.94 |
| 323 | CD | ARG | 44 | −10.669 | 4.043 | 99.170 | 1.00 | 78.71 |
| 324 | NE | ARG | 44 | −11.075 | 2.763 | 99.769 | 1.00 | 74.34 |
| 325 | CZ | ARG | 44 | −11.496 | 2.595 | 101.028 | 1.00 | 79.10 |
| 326 | NH1 | ARG | 44 | −11.594 | 3.625 | 101.873 | 1.00 | 55.57 |
| 327 | NH2 | ARG | 44 | −11.791 | 1.371 | 101.459 | 1.00 | 74.96 |
| 328 | C | ARG | 44 | −6.543 | 6.678 | 97.697 | 1.00 | 62.72 |
| 329 | O | ARG | 44 | −7.168 | 7.728 | 97.598 | 1.00 | 65.18 |
| 330 | N | LYS | 45 | −5.402 | 6.453 | 97.056 | 1.00 | 58.94 |
| 331 | CA | LYS | 45 | −4.796 | 7.433 | 96.157 | 1.00 | 60.24 |
| 332 | CB | LYS | 45 | −3.455 | 6.902 | 95.653 | 1.00 | 53.69 |
| 333 | CG | LYS | 45 | −2.395 | 6.716 | 96.767 | 1.00 | 62.98 |
| 334 | CD | LYS | 45 | −1.051 | 6.226 | 96.202 | 1.00 | 52.29 |
| 335 | CE | LYS | 45 | −0.002 | 6.007 | 97.311 | 1.00 | 68.99 |
| 336 | NZ | LYS | 45 | 1.398 | 5.812 | 96.791 | 1.00 | 58.39 |
| 337 | C | LYS | 45 | −5.719 | 7.777 | 94.977 | 1.00 | 66.70 |
| 338 | O | LYS | 45 | −6.594 | 7.000 | 94.634 | 1.00 | 60.79 |
| 339 | N | PRO | 46 | −5.539 | 8.961 | 94.349 | 1.00 | 76.24 |
| 340 | CD | PRO | 46 | −4.867 | 10.144 | 94.910 | 1.00 | 72.65 |
| 341 | CA | PRO | 46 | −6.395 | 9.360 | 93.218 | 1.00 | 74.98 |
| 342 | CB | PRO | 46 | −6.285 | 10.883 | 93.210 | 1.00 | 71.65 |
| 343 | CG | PRO | 46 | −5.893 | 11.219 | 94.628 | 1.00 | 80.40 |
| 344 | C | PRO | 46 | −6.075 | 8.789 | 91.845 | 1.00 | 79.19 |
| 345 | O | PRO | 46 | −6.775 | 9.095 | 90.880 | 1.00 | 82.19 |
| 346 | N | ALA | 47 | −5.025 | 7.978 | 91.741 | 1.00 | 82.33 |
| 347 | CA | ALA | 47 | −4.656 | 7.398 | 90.450 | 1.00 | 84.51 |
| 348 | CB | ALA | 47 | −3.232 | 6.852 | 90.501 | 1.00 | 70.10 |
| 349 | C | ALA | 47 | −5.633 | 6.290 | 90.053 | 1.00 | 86.16 |
| 350 | O | ALA | 47 | −6.031 | 5.473 | 90.890 | 1.00 | 87.14 |
| 351 | N | ALA | 48 | −6.020 | 6.282 | 88.777 | 1.00 | 87.33 |
| 352 | CA | ALA | 48 | −6.941 | 5.278 | 88.243 | 1.00 | 87.92 |
| 353 | CB | ALA | 48 | −7.197 | 5.537 | 86.747 | 1.00 | 82.34 |
| 354 | C | ALA | 48 | −6.343 | 3.884 | 88.460 | 1.00 | 90.04 |
| 355 | O | ALA | 48 | −5.117 | 3.705 | 88.387 | 1.00 | 82.32 |
| 356 | N | GLY | 49 | −7.208 | 2.902 | 88.728 | 1.00 | 95.77 |
| 357 | CA | GLY | 49 | −6.731 | 1.551 | 88.989 | 1.00 | 93.69 |
| 358 | C | GLY | 49 | −5.650 | 1.647 | 90.050 | 1.00 | 97.85 |
| 359 | O | GLY | 49 | −4.495 | 1.289 | 89.807 | 1.00 | 93.57 |
| 360 | N | SER | 50 | −6.043 | 2.145 | 91.225 | 1.00 | 100.32 |
| 361 | CA | SER | 50 | −5.140 | 2.354 | 92.353 | 1.00 | 98.95 |
| 362 | CB | SER | 50 | −5.775 | 3.338 | 93.348 | 1.00 | 99.14 |
| 363 | OG | SER | 50 | −4.832 | 3.786 | 94.310 | 1.00 | 99.30 |
| 364 | C | SER | 50 | −4.692 | 1.085 | 93.082 | 1.00 | 99.10 |
| 365 | O | SER | 50 | −4.483 | 0.047 | 92.462 | 1.00 | 96.91 |
| 366 | N | HIS | 51 | −4.569 | 1.173 | 94.404 | 1.00 | 100.90 |
| 367 | CA | HIS | 51 | −4.078 | 0.056 | 95.210 | 1.00 | 102.38 |
| 368 | CB | HIS | 51 | −2.582 | −0.125 | 94.912 | 1.00 | 103.47 |
| 369 | CG | HIS | 51 | −1.807 | 1.164 | 94.918 | 1.00 | 103.87 |
| 370 | CD2 | HIS | 51 | −1.636 | 2.102 | 95.884 | 1.00 | 98.07 |
| 371 | ND1 | HIS | 51 | −1.104 | 1.616 | 93.820 | 1.00 | 103.86 |
| 372 | CE1 | HIS | 51 | −0.533 | 2.773 | 94.109 | 1.00 | 101.19 |
| 373 | NE2 | HIS | 51 | −0.841 | 3.090 | 95.355 | 1.00 | 97.91 |
| 374 | C | HIS | 51 | −4.257 | 0.274 | 96.722 | 1.00 | 96.96 |
| 375 | O | HIS | 51 | −3.266 | 0.315 | 97.455 | 1.00 | 95.98 |
| 376 | N | PRO | 52 | −5.511 | 0.380 | 97.206 | 1.00 | 91.94 |
| 377 | CD | PRO | 52 | −6.727 | −0.010 | 96.470 | 1.00 | 93.73 |
| 378 | CA | PRO | 52 | −5.822 | 0.597 | 98.629 | 1.00 | 87.96 |
| 379 | CB | PRO | 52 | −7.164 | −0.105 | 98.799 | 1.00 | 88.73 |
| 380 | CG | PRO | 52 | −7.833 | 0.189 | 97.518 | 1.00 | 90.59 |
| 381 | C | PRO | 52 | −4.775 | 0.101 | 99.625 | 1.00 | 79.22 |
| 382 | O | PRO | 52 | −4.414 | −1.071 | 99.627 | 1.00 | 76.14 |
| 383 | N | SER | 53 | −4.284 | 1.018 | 100.456 | 1.00 | 80.39 |
| 384 | CA | SER | 53 | −3.283 | 0.706 | 101.476 | 1.00 | 74.21 |
| 385 | CB | SER | 53 | −2.323 | 1.886 | 101.655 | 1.00 | 84.01 |
| 386 | OG | SER | 53 | −1.275 | 1.858 | 100.700 | 1.00 | 88.72 |
| 387 | C | SER | 53 | −3.963 | 0.404 | 102.807 | 1.00 | 68.27 |
| 388 | O | SER | 53 | −4.979 | 1.023 | 103.139 | 1.00 | 64.18 |
| 389 | N | ARG | 54 | −3.406 | −0.549 | 103.557 | 1.00 | 64.51 |
| 390 | CA | ARG | 54 | −3.965 | −0.931 | 104.858 | 1.00 | 67.89 |
| 391 | CB | ARG | 54 | −4.679 | −2.278 | 104.734 | 1.00 | 68.57 |
| 392 | CG | ARG | 54 | −5.920 | −2.182 | 103.866 | 1.00 | 63.14 |
| 393 | CD | ARG | 54 | −6.573 | −3.523 | 103.525 | 1.00 | 69.89 |
| 394 | NE | ARG | 54 | −7.644 | −3.310 | 102.551 | 1.00 | 77.10 |
| 395 | CZ | ARG | 54 | −8.744 | −2.604 | 102.805 | 1.00 | 76.59 |
| 396 | NH1 | ARG | 54 | −8.914 | −2.069 | 104.004 | 1.00 | 74.58 |
| 397 | NH2 | ARG | 54 | −9.650 | −2.394 | 101.856 | 1.00 | 70.56 |
| 398 | C | ARG | 54 | −2.924 | −0.943 | 105.992 | 1.00 | 60.53 |
| 399 | O | ARG | 54 | −2.051 | −1.792 | 106.050 | 1.00 | 53.27 |
| 400 | N | TRP | 55 | −3.039 | 0.021 | 106.903 | 1.00 | 59.18 |
| 401 | CA | TRP | 55 | −2.081 | 0.149 | 107.990 | 1.00 | 60.59 |
| 402 | CB | TRP | 55 | −1.279 | 1.462 | 107.824 | 1.00 | 50.36 |
| 403 | CG | TRP | 55 | −0.649 | 1.696 | 106.447 | 1.00 | 49.83 |
| 404 | CD2 | TRP | 55 | −0.354 | 2.965 | 105.839 | 1.00 | 42.60 |
| 405 | CE2 | TRP | 55 | 0.283 | 2.696 | 104.597 | 1.00 | 50.18 |
| 406 | CE3 | TRP | 55 | −0.562 | 4.294 | 106.221 | 1.00 | 34.80 |
| 407 | CD1 | TRP | 55 | −0.199 | 0.752 | 105.582 | 1.00 | 39.67 |
| 408 | NE1 | TRP | 55 | 0.360 | 1.335 | 104.471 | 1.00 | 48.41 |
| 409 | CZ2 | TRP | 55 | 0.724 | 3.716 | 103.730 | 1.00 | 35.19 |
| 410 | CZ3 | TRP | 55 | −0.122 | 5.315 | 105.368 | 1.00 | 40.98 |
| 411 | CH2 | TRP | 55 | 0.515 | 5.016 | 104.138 | 1.00 | 52.09 |
| 412 | C | TRP | 55 | −2.741 | 0.159 | 109.378 | 1.00 | 64.36 |
| 413 | O | TRP | 55 | −3.937 | 0.467 | 109.504 | 1.00 | 62.24 |
| 414 | N | ALA | 56 | −1.953 | −0.182 | 110.406 | 1.00 | 56.70 |
| 415 | CA | ALA | 56 | −2.417 | −0.132 | 111.797 | 1.00 | 56.96 |
| 416 | CB | ALA | 56 | −2.440 | −1.510 | 112.431 | 1.00 | 43.05 |
| 417 | C | ALA | 56 | −1.556 | 0.794 | 112.656 | 1.00 | 61.79 |
| 418 | O | ALA | 56 | −0.305 | 0.688 | 112.674 | 1.00 | 52.89 |
| 419 | N | GLY | 57 | −2.245 | 1.693 | 113.366 | 1.00 | 54.08 |
| 420 | CA | GLY | 57 | −1.576 | 2.606 | 114.278 | 1.00 | 61.77 |
| 421 | C | GLY | 57 | −2.262 | 2.639 | 115.647 | 1.00 | 66.52 |
| 422 | O | GLY | 57 | −3.452 | 2.320 | 115.765 | 1.00 | 66.82 |
| 423 | N | MET | 58 | −1.529 | 3.004 | 116.695 | 1.00 | 57.96 |
| 424 | CA | MET | 58 | −2.144 | 3.084 | 118.025 | 1.00 | 63.85 |
| 425 | CB | MET | 58 | −1.116 | 2.750 | 119.111 | 1.00 | 55.90 |
| 426 | CG | MET | 58 | −1.666 | 2.694 | 120.516 | 1.00 | 57.48 |
| 427 | SD | MET | 58 | −0.402 | 2.166 | 121.746 | 1.00 | 63.83 |
| 428 | CE | MET | 58 | −1.429 | 1.785 | 123.173 | 1.00 | 60.54 |
| 429 | C | MET | 58 | −2.703 | 4.503 | 118.234 | 1.00 | 67.72 |
| 430 | O | MET | 58 | −1.987 | 5.506 | 118.090 | 1.00 | 64.22 |
| 431 | N | GLY | 59 | −3.989 | 4.586 | 118.552 | 1.00 | 67.18 |
| 432 | CA | GLY | 59 | −4.594 | 5.880 | 118.757 | 1.00 | 64.80 |
| 433 | C | GLY | 59 | −5.040 | 6.544 | 117.462 | 1.00 | 71.90 |
| 434 | O | GLY | 59 | −5.006 | 5.932 | 116.389 | 1.00 | 62.06 |
| 435 | N | ARG | 60 | −5.425 | 7.818 | 117.570 | 1.00 | 68.35 |
| 436 | CA | ARG | 60 | −5.940 | 8.574 | 116.443 | 1.00 | 61.14 |
| 437 | CB | ARG | 60 | −6.952 | 9.606 | 116.951 | 1.00 | 62.34 |
| 438 | CG | ARG | 60 | −6.362 | 10.882 | 117.530 | 1.00 | 59.80 |
| 439 | CD | ARG | 60 | −7.324 | 11.487 | 118.561 | 1.00 | 71.88 |
| 440 | NE | ARG | 60 | −7.167 | 12.933 | 118.752 | 1.00 | 74.16 |
| 441 | CZ | ARG | 60 | −7.952 | 13.859 | 118.197 | 1.00 | 69.68 |
| 442 | NH1 | ARG | 60 | −8.976 | 13.511 | 117.407 | 1.00 | 46.24 |
| 443 | NH2 | ARG | 60 | −7.697 | 15.144 | 118.423 | 1.00 | 64.41 |
| 444 | C | ARG | 60 | −4.932 | 9.263 | 115.545 | 1.00 | 58.42 |
| 445 | O | ARG | 60 | −5.314 | 9.766 | 114.503 | 1.00 | 65.21 |
| 446 | N | ARG | 61 | −3.661 | 9.292 | 115.934 | 1.00 | 50.09 |
| 447 | CA | ARG | 61 | −2.642 | 9.954 | 115.132 | 1.00 | 47.76 |
| 448 | CB | ARG | 61 | −1.717 | 10.835 | 116.006 | 1.00 | 57.06 |
| 449 | CG | ARG | 61 | −2.353 | 12.125 | 116.538 | 1.00 | 57.54 |
| 450 | CD | ARG | 61 | −1.301 | 13.082 | 117.103 | 1.00 | 62.78 |
| 451 | NE | ARG | 61 | −1.842 | 14.418 | 117.436 | 1.00 | 76.54 |
| 452 | CZ | ARG | 61 | −2.635 | 15.164 | 116.653 | 1.00 | 72.22 |
| 453 | NH1 | ARG | 61 | −3.023 | 14.729 | 115.465 | 1.00 | 71.05 |
| 454 | NH2 | ARG | 61 | −3.028 | 16.371 | 117.045 | 1.00 | 72.76 |
| 455 | C | ARG | 61 | −1.779 | 9.006 | 114.322 | 1.00 | 50.59 |
| 456 | O | ARG | 61 | −1.532 | 7.875 | 114.701 | 1.00 | 43.89 |
| 457 | N | LEU | 62 | −1.305 | 9.499 | 113.192 | 1.00 | 47.93 |
| 458 | CA | LEU | 62 | −0.471 | 8.718 | 112.317 | 1.00 | 47.86 |
| 459 | CB | LEU | 62 | −1.309 | 7.859 | 111.366 | 1.00 | 51.27 |
| 460 | CG | LEU | 62 | −0.478 | 7.072 | 110.352 | 1.00 | 55.21 |
| 461 | CD1 | LEU | 62 | 0.343 | 6.018 | 111.079 | 1.00 | 49.34 |
| 462 | CD2 | LEU | 62 | −1.402 | 6.438 | 109.310 | 1.00 | 62.13 |
| 463 | C | LEU | 62 | 0.264 | 9.758 | 111.542 | 1.00 | 40.31 |
| 464 | O | LEU | 62 | −0.330 | 10.553 | 110.843 | 1.00 | 47.28 |
| 465 | N | LEU | 63 | 1.574 | 9.738 | 111.667 | 1.00 | 45.58 |
| 466 | CA | LEU | 63 | 2.410 | 10.697 | 111.010 | 1.00 | 48.22 |
| 467 | CB | LEU | 63 | 3.603 | 10.952 | 111.915 | 1.00 | 52.66 |
| 468 | CG | LEU | 63 | 4.480 | 12.194 | 111.808 | 1.00 | 63.27 |
| 469 | CD1 | LEU | 63 | 5.359 | 12.129 | 110.560 | 1.00 | 59.94 |
| 470 | CD2 | LEU | 63 | 3.574 | 13.406 | 111.829 | 1.00 | 59.24 |
| 471 | C | LEU | 63 | 2.864 | 10.227 | 109.622 | 1.00 | 57.85 |
| 472 | O | LEU | 63 | 3.147 | 9.039 | 109.404 | 1.00 | 52.19 |
| 473 | N | LEU | 64 | 2.911 | 11.160 | 108.677 | 1.00 | 49.48 |
| 474 | CA | LEU | 64 | 3.381 | 10.845 | 107.343 | 1.00 | 48.87 |
| 475 | CB | LEU | 64 | 2.375 | 11.278 | 106.274 | 1.00 | 45.23 |
| 476 | CG | LEU | 64 | 0.939 | 10.742 | 106.387 | 1.00 | 46.41 |
| 477 | CD1 | LEU | 64 | 0.099 | 11.323 | 105.303 | 1.00 | 52.43 |
| 478 | CD2 | LEU | 64 | 0.931 | 9.223 | 106.261 | 1.00 | 50.15 |
| 479 | C | LEU | 64 | 4.687 | 11.599 | 107.185 | 1.00 | 50.59 |
| 480 | O | LEU | 64 | 4.739 | 12.821 | 107.256 | 1.00 | 50.61 |
| 481 | N | ARG | 65 | 5.752 | 10.846 | 107.019 | 1.00 | 40.29 |
| 482 | CA | ARG | 65 | 7.052 | 11.416 | 106.830 | 1.00 | 54.71 |
| 483 | CB | ARG | 65 | 8.098 | 10.342 | 107.109 | 1.00 | 48.29 |
| 484 | CG | ARG | 65 | 9.540 | 10.716 | 106.867 | 1.00 | 65.38 |
| 485 | CD | ARG | 65 | 10.381 | 9.440 | 106.778 | 1.00 | 61.84 |
| 486 | NE | ARG | 65 | 11.770 | 9.692 | 106.395 | 1.00 | 76.35 |
| 487 | CZ | ARG | 65 | 12.619 | 8.744 | 105.989 | 1.00 | 84.78 |
| 488 | NH1 | ARG | 65 | 12.227 | 7.467 | 105.909 | 1.00 | 82.41 |
| 489 | NH2 | ARG | 65 | 13.863 | 9.068 | 105.651 | 1.00 | 78.69 |
| 490 | C | ARG | 65 | 7.184 | 11.926 | 105.386 | 1.00 | 52.15 |
| 491 | O | ARG | 65 | 6.572 | 11.397 | 104.464 | 1.00 | 51.21 |
| 492 | N | SER | 66 | 7.976 | 12.976 | 105.237 | 1.00 | 48.47 |
| 493 | CA | SER | 66 | 8.319 | 13.597 | 103.975 | 1.00 | 45.49 |
| 494 | CB | SER | 66 | 9.779 | 13.213 | 103.674 | 1.00 | 45.32 |
| 495 | OG | SER | 66 | 10.168 | 13.555 | 102.364 | 1.00 | 70.17 |
| 496 | C | SER | 66 | 7.377 | 13.291 | 102.795 | 1.00 | 42.85 |
| 497 | O | SER | 66 | 7.801 | 12.838 | 101.741 | 1.00 | 41.58 |
| 498 | N | VAL | 67 | 6.093 | 13.562 | 103.000 | 1.00 | 40.45 |
| 499 | CA | VAL | 67 | 5.057 | 13.369 | 102.006 | 1.00 | 39.39 |
| 500 | CB | VAL | 67 | 3.797 | 14.105 | 102.420 | 1.00 | 37.84 |
| 501 | CG1 | VAL | 67 | 2.734 | 13.899 | 101.422 | 1.00 | 61.68 |
| 502 | CG2 | VAL | 67 | 3.335 | 13.584 | 103.724 | 1.00 | 34.69 |
| 503 | C | VAL | 67 | 5.464 | 13.853 | 100.603 | 1.00 | 52.57 |
| 504 | O | VAL | 67 | 6.255 | 14.788 | 100.431 | 1.00 | 44.66 |
| 505 | N | GLN | 68 | 4.881 | 13.222 | 99.596 | 1.00 | 41.81 |
| 506 | CA | GLN | 68 | 5.207 | 13.537 | 98.218 | 1.00 | 45.96 |
| 507 | CB | GLN | 68 | 6.020 | 12.382 | 97.600 | 1.00 | 40.84 |
| 508 | CG | GLN | 68 | 7.423 | 12.190 | 98.256 | 1.00 | 41.84 |
| 509 | CD | GLN | 68 | 8.268 | 13.455 | 98.175 | 1.00 | 50.56 |
| 510 | OE1 | GLN | 68 | 8.262 | 14.131 | 97.150 | 1.00 | 50.20 |
| 511 | NE2 | GLN | 68 | 8.998 | 13.779 | 99.249 | 1.00 | 47.56 |
| 512 | C | GLN | 68 | 3.906 | 13.728 | 97.505 | 1.00 | 39.57 |
| 513 | O | GLN | 68 | 2.862 | 13.438 | 98.065 | 1.00 | 44.24 |
| 514 | N | LEU | 69 | 3.963 | 14.231 | 96.280 | 1.00 | 42.17 |
| 515 | CA | LEU | 69 | 2.756 | 14.468 | 95.500 | 1.00 | 47.79 |
| 516 | CB | LEU | 69 | 3.138 | 14.971 | 94.090 | 1.00 | 47.86 |
| 517 | CG | LEU | 69 | 3.843 | 16.352 | 94.023 | 1.00 | 46.84 |
| 518 | CD1 | LEU | 69 | 4.296 | 16.605 | 92.637 | 1.00 | 37.74 |
| 519 | CD2 | LEU | 69 | 2.929 | 17.487 | 94.478 | 1.00 | 39.16 |
| 520 | C | LEU | 69 | 1.907 | 13.198 | 95.452 | 1.00 | 50.47 |
| 521 | O | LEU | 69 | 0.691 | 13.237 | 95.672 | 1.00 | 44.43 |
| 522 | N | HIS | 70 | 2.568 | 12.064 | 95.227 | 1.00 | 48.13 |
| 523 | CA | HIS | 70 | 1.884 | 10.775 | 95.173 | 1.00 | 47.08 |
| 524 | CB | HIS | 70 | 2.855 | 9.700 | 94.660 | 1.00 | 51.12 |
| 525 | CG | HIS | 70 | 4.068 | 9.521 | 95.520 | 1.00 | 57.96 |
| 526 | CD2 | HIS | 70 | 4.198 | 9.318 | 96.858 | 1.00 | 50.12 |
| 527 | ND1 | HIS | 70 | 5.353 | 9.643 | 95.026 | 1.00 | 61.23 |
| 528 | CE1 | HIS | 70 | 6.220 | 9.539 | 96.020 | 1.00 | 51.08 |
| 529 | NE2 | HIS | 70 | 5.545 | 9.345 | 97.142 | 1.00 | 57.79 |
| 530 | C | HIS | 70 | 1.287 | 10.320 | 96.514 | 1.00 | 51.84 |
| 531 | O | HIS | 70 | 0.713 | 9.245 | 96.584 | 1.00 | 54.76 |
| 532 | N | ASP | 71 | 1.455 | 11.091 | 97.590 | 1.00 | 47.38 |
| 533 | CA | ASP | 71 | 0.866 | 10.688 | 98.863 | 1.00 | 47.33 |
| 534 | CB | ASP | 71 | 1.706 | 11.097 | 100.089 | 1.00 | 42.05 |
| 535 | CG | ASP | 71 | 2.992 | 10.269 | 100.228 | 1.00 | 49.63 |
| 536 | OD1 | ASP | 71 | 2.923 | 9.019 | 100.214 | 1.00 | 52.98 |
| 537 | OD2 | ASP | 71 | 4.082 | 10.866 | 100.353 | 1.00 | 51.23 |
| 538 | C | ASP | 71 | −0.476 | 11.351 | 98.928 | 1.00 | 50.80 |
| 539 | O | ASP | 71 | −1.211 | 11.188 | 99.904 | 1.00 | 49.43 |
| 540 | N | SER | 72 | −0.805 | 12.129 | 97.898 | 1.00 | 52.17 |
| 541 | CA | SER | 72 | −2.130 | 12.736 | 97.884 | 1.00 | 54.20 |
| 542 | CB | SER | 72 | −2.376 | 13.596 | 96.643 | 1.00 | 39.77 |
| 543 | OG | SER | 72 | −1.773 | 14.856 | 96.763 | 1.00 | 51.83 |
| 544 | C | SER | 72 | −3.055 | 11.556 | 97.836 | 1.00 | 52.86 |
| 545 | O | SER | 72 | −2.732 | 10.537 | 97.204 | 1.00 | 57.51 |
| 546 | N | GLY | 73 | −4.192 | 11.693 | 98.507 | 1.00 | 53.54 |
| 547 | CA | GLY | 73 | −5.167 | 10.632 | 98.524 | 1.00 | 55.41 |
| 548 | C | GLY | 73 | −6.175 | 10.789 | 99.638 | 1.00 | 56.10 |
| 549 | O | GLY | 73 | −6.269 | 11.823 | 100.289 | 1.00 | 57.39 |
| 550 | N | ASN | 74 | −6.954 | 9.745 | 99.854 | 1.00 | 62.16 |
| 551 | CA | ASN | 74 | −7.961 | 9.775 | 100.891 | 1.00 | 68.78 |
| 552 | CB | ASN | 74 | −9.330 | 9.477 | 100.270 | 1.00 | 73.41 |
| 553 | CG | ASN | 74 | −10.058 | 10.742 | 99.847 | 1.00 | 71.59 |
| 554 | OD1 | ASN | 74 | −10.677 | 11.408 | 100.668 | 1.00 | 77.51 |
| 555 | ND2 | ASN | 74 | −9.963 | 11.092 | 98.575 | 1.00 | 81.67 |
| 556 | C | ASN | 74 | −7.609 | 8.792 | 102.002 | 1.00 | 69.26 |
| 557 | O | ASN | 74 | −7.547 | 7.576 | 101.786 | 1.00 | 64.42 |
| 558 | N | TYR | 75 | −7.332 | 9.327 | 103.187 | 1.00 | 67.46 |
| 559 | CA | TYR | 75 | −6.985 | 8.475 | 104.311 | 1.00 | 60.02 |
| 560 | CB | TYR | 75 | −5.862 | 9.092 | 105.170 | 1.00 | 52.32 |
| 561 | CG | TYR | 75 | −4.571 | 9.204 | 104.410 | 1.00 | 41.71 |
| 562 | CD1 | TYR | 75 | −4.443 | 10.110 | 103.369 | 1.00 | 33.39 |
| 563 | CE1 | TYR | 75 | −3.299 | 10.160 | 102.600 | 1.00 | 38.06 |
| 564 | CD2 | TYR | 75 | −3.512 | 8.342 | 104.666 | 1.00 | 4 |
| 565 | CE2 | TYR | 75 | −2.350 | 8.378 | 103.904 | 1.00 | 43.61 |
| 566 | CZ | TYR | 75 | −2.246 | 9.301 | 102.870 | 1.00 | 44.97 |
| 567 | OH | TYR | 75 | −1.068 | 9.415 | 102.160 | 1.00 | 40.63 |
| 568 | C | TYR | 75 | −8.207 | 8.260 | 105.147 | 1.00 | 59.98 |
| 569 | O | TYR | 75 | −8.626 | 9.154 | 105.879 | 1.00 | 57.57 |
| 570 | N | SER | 76 | −8.778 | 7.063 | 105.012 | 1.00 | 69.14 |
| 571 | CA | SER | 76 | −9.952 | 6.661 | 105.778 | 1.00 | 74.53 |
| 572 | CB | SER | 76 | −10.820 | 5.689 | 104.985 | 1.00 | 68.66 |
| 573 | OG | SER | 76 | −11.604 | 6.389 | 104.039 | 1.00 | 75.07 |
| 574 | C | SER | 76 | −9.542 | 6.025 | 107.105 | 1.00 | 77.27 |
| 575 | O | SER | 76 | −8.766 | 5.053 | 107.181 | 1.00 | 68.93 |
| 576 | N | CYS | 77 | −10.083 | 6.606 | 108.159 | 1.00 | 78.72 |
| 577 | CA | CYS | 77 | −9.803 | 6.158 | 109.499 | 1.00 | 86.89 |
| 578 | C | CYS | 77 | −10.924 | 5.250 | 110.046 | 1.00 | 83.28 |
| 579 | O | CYS | 77 | −12.095 | 5.642 | 110.074 | 1.00 | 83.09 |
| 580 | CB | CYS | 77 | −9.604 | 7.394 | 110.385 | 1.00 | 85.54 |
| 581 | SG | CYS | 77 | −9.484 | 6.913 | 112.116 | 1.00 | 108.50 |
| 582 | N | TYR | 78 | −10.566 | 4.035 | 110.466 | 1.00 | 81.54 |
| 583 | CA | TYR | 78 | −11.551 | 3.095 | 111.012 | 1.00 | 78.92 |
| 584 | CB | TYR | 78 | −11.668 | 1.833 | 110.131 | 1.00 | 72.07 |
| 585 | CG | TYR | 78 | −11.967 | 2.094 | 108.670 | 1.00 | 70.82 |
| 586 | CD1 | TYR | 78 | −10.946 | 2.389 | 107.767 | 1.00 | 77.93 |
| 587 | CE1 | TYR | 78 | −11.220 | 2.626 | 106.419 | 1.00 | 76.94 |
| 588 | CD2 | TYR | 78 | −13.270 | 2.048 | 108.188 | 1.00 | 70.90 |
| 589 | CE2 | TYR | 78 | −13.560 | 2.290 | 106.848 | 1.00 | 65.48 |
| 590 | CZ | TYR | 78 | −12.532 | 2.575 | 105.965 | 1.00 | 80.82 |
| 591 | OH | TYR | 78 | −12.809 | 2.793 | 104.626 | 1.00 | 88.20 |
| 592 | C | TYR | 78 | −11.235 | 2.669 | 112.456 | 1.00 | 80.75 |
| 593 | O | TYR | 78 | −10.103 | 2.263 | 112.782 | 1.00 | 70.45 |
| 594 | N | ARG | 79 | −12.247 | 2.782 | 113.314 | 1.00 | 79.63 |
| 595 | CA | ARG | 79 | −12.143 | 2.398 | 114.726 | 1.00 | 85.33 |
| 596 | CB | ARG | 79 | −12.542 | 3.575 | 115.620 | 1.00 | 82.02 |
| 597 | CG | ARG | 79 | −12.664 | 3.218 | 117.097 | 1.00 | 84.64 |
| 598 | CD | ARG | 79 | −11.326 | 2.868 | 117.740 | 1.00 | 83.04 |
| 599 | NE | ARG | 79 | −11.510 | 2.235 | 119.051 | 1.00 | 88.00 |
| 600 | CZ | ARG | 79 | −12.199 | 2.763 | 120.064 | 1.00 | 82.53 |
| 601 | NH1 | ARG | 79 | −12.783 | 3.954 | 119.948 | 1.00 | 79.11 |
| 602 | NH2 | ARG | 79 | −12.329 | 2.080 | 121.188 | 1.00 | 77.31 |
| 603 | C | ARG | 79 | −13.064 | 1.193 | 114.995 | 1.00 | 85.02 |
| 604 | O | ARG | 79 | −14.211 | 1.351 | 115.428 | 1.00 | 78.72 |
| 605 | N | ALA | 80 | −12.545 | −0.006 | 114.726 | 1.00 | 87.23 |
| 606 | CA | ALA | 80 | −13.293 | −1.252 | 114.897 | 1.00 | 84.46 |
| 607 | CB | ALA | 80 | −13.868 | −1.338 | 116.318 | 1.00 | 79.94 |
| 608 | C | ALA | 80 | −14.416 | −1.346 | 113.858 | 1.00 | 85.99 |
| 609 | O | ALA | 80 | −14.426 | −2.249 | 113.018 | 1.00 | 81.86 |
| 610 | N | GLY | 81 | −15.341 | −0.389 | 113.906 | 1.00 | 89.93 |
| 611 | CA | GLY | 81 | −16.470 | −0.379 | 112.988 | 1.00 | 92.08 |
| 612 | C | GLY | 81 | −16.174 | −0.022 | 111.545 | 1.00 | 87.28 |
| 613 | O | GLY | 81 | −15.677 | −0.851 | 110.781 | 1.00 | 85.96 |
| 614 | N | ARG | 82 | −16.521 | 1.205 | 111.169 | 0.00 | 92.45 |
| 615 | CA | ARG | 82 | −16.291 | 1.710 | 109.820 | 0.00 | 95.52 |
| 616 | CB | ARG | 82 | −17.331 | 1.149 | 108.838 | 0.00 | 94.95 |
| 617 | CG | ARG | 82 | −17.003 | −0.248 | 108.313 | 0.00 | 94.58 |
| 618 | CD | ARG | 82 | −18.068 | −0.768 | 107.355 | 0.00 | 94.22 |
| 619 | NE | ARG | 82 | −17.744 | −2.101 | 106.847 | 0.00 | 93.88 |
| 620 | CZ | ARG | 82 | −18.537 | −2.814 | 106.053 | 0.00 | 93.71 |
| 621 | NH1 | ARG | 82 | −19.709 | −2.325 | 105.669 | 0.00 | 93.58 |
| 622 | NH2 | ARG | 82 | −18.160 | −4.017 | 105.641 | 0.00 | 93.58 |
| 623 | C | ARG | 82 | −16.310 | 3.239 | 109.800 | 0.00 | 98.67 |
| 624 | O | ARG | 82 | −15.377 | 3.867 | 109.302 | 0.00 | 98.14 |
| 625 | N | PRO | 83 | −17.373 | 3.864 | 110.337 | 1.00 | 103.39 |
| 626 | CD | PRO | 83 | −18.700 | 3.327 | 110.702 | 1.00 | 105.19 |
| 627 | CA | PRO | 83 | −17.400 | 5.331 | 110.329 | 1.00 | 104.87 |
| 628 | CB | PRO | 83 | −18.901 | 5.642 | 110.372 | 1.00 | 103.05 |
| 629 | CG | PRO | 83 | −19.428 | 4.559 | 111.245 | 1.00 | 101.61 |
| 630 | C | PRO | 83 | −16.620 | 6.010 | 111.473 | 1.00 | 99.40 |
| 631 | O | PRO | 83 | −17.082 | 6.064 | 112.614 | 1.00 | 97.91 |
| 632 | N | ALA | 84 | −15.435 | 6.525 | 111.146 | 1.00 | 97.87 |
| 633 | CA | ALA | 84 | −14.578 | 7.228 | 112.106 | 1.00 | 94.69 |
| 634 | CB | ALA | 84 | −13.433 | 6.315 | 112.574 | 1.00 | 91.69 |
| 635 | C | ALA | 84 | −14.021 | 8.485 | 111.420 | 1.00 | 91.93 |
| 636 | O | ALA | 84 | −13.427 | 9.356 | 112.062 | 1.00 | 80.79 |
| 637 | N | GLY | 85 | −14.217 | 8.552 | 110.102 | 1.00 | 89.61 |
| 638 | CA | GLY | 85 | −13.787 | 9.702 | 109.325 | 1.00 | 76.77 |
| 639 | C | GLY | 85 | −12.726 | 9.461 | 108.273 | 1.00 | 74.02 |
| 640 | O | GLY | 85 | −12.143 | 8.374 | 108.181 | 1.00 | 78.43 |
| 641 | N | THR | 86 | −12.506 | 10.482 | 107.449 | 1.00 | 66.80 |
| 642 | CA | THR | 86 | −11.469 | 10.450 | 106.429 | 1.00 | 61.26 |
| 643 | CB | THR | 86 | −12.021 | 10.241 | 105.010 | 1.00 | 62.31 |
| 644 | OG1 | THR | 86 | −12.532 | 8.914 | 104.882 | 1.00 | 67.30 |
| 645 | CG2 | THR | 86 | −10.911 | 10.467 | 103.969 | 1.00 | 54.98 |
| 646 | C | THR | 86 | −10.779 | 11.807 | 106.446 | 1.00 | 69.17 |
| 647 | O | THR | 86 | −11.400 | 12.828 | 106.742 | 1.00 | 69.62 |
| 648 | N | VAL | 87 | −9.486 | 11.807 | 106.146 | 1.00 | 72.13 |
| 649 | CA | VAL | 87 | −8.706 | 13.029 | 106.079 | 1.00 | 65.24 |
| 650 | CB | VAL | 87 | −7.405 | 12.918 | 106.932 | 1.00 | 66.87 |
| 651 | CG1 | VAL | 87 | −6.521 | 14.144 | 106.729 | 1.00 | 53.62 |
| 652 | CG2 | VAL | 87 | −7.752 | 12.758 | 108.385 | 1.00 | 69.44 |
| 653 | C | VAL | 87 | −8.314 | 13.156 | 104.610 | 1.00 | 62.08 |
| 654 | O | VAL | 87 | −7.520 | 12.356 | 104.123 | 1.00 | 68.19 |
| 655 | N | HIS | 88 | −8.860 | 14.141 | 103.903 | 1.00 | 55.26 |
| 656 | CA | HIS | 88 | −8.510 | 14.342 | 102.487 | 1.00 | 55.55 |
| 657 | CB | HIS | 88 | −9.629 | 15.079 | 101.750 | 1.00 | 45.86 |
| 658 | CG | HIS | 88 | −10.961 | 14.406 | 101.871 | 1.00 | 68.29 |
| 659 | CD2 | HIS | 88 | −11.786 | 13.883 | 100.933 | 1.00 | 70.79 |
| 660 | ND1 | HIS | 88 | −11.572 | 14.178 | 103.089 | 1.00 | 72.48 |
| 661 | CE1 | HIS | 88 | −12.715 | 13.543 | 102.894 | 1.00 | 74.65 |
| 662 | NE2 | HIS | 88 | −12.868 | 13.352 | 101.596 | 1.00 | 77.31 |
| 663 | C | HIS | 88 | −7.217 | 15.137 | 102.374 | 1.00 | 54.27 |
| 664 | O | HIS | 88 | −7.183 | 16.320 | 102.687 | 1.00 | 56.68 |
| 665 | N | LEU | 89 | −6.163 | 14.476 | 101.920 | 1.00 | 51.05 |
| 666 | CA | LEU | 89 | −4.848 | 15.089 | 101.768 | 1.00 | 55.76 |
| 667 | CB | LEU | 89 | −3.782 | 14.210 | 102.400 | 1.00 | 44.10 |
| 668 | CG | LEU | 89 | −2.339 | 14.683 | 102.233 | 1.00 | 63.17 |
| 669 | CD1 | LEU | 89 | −2.136 | 15.929 | 103.069 | 1.00 | 52.92 |
| 670 | CD2 | LEU | 89 | −1.354 | 13.579 | 102.657 | 1.00 | 49.65 |
| 671 | C | LEU | 89 | −4.419 | 15.381 | 100.335 | 1.00 | 60.30 |
| 672 | O | LEU | 89 | −4.197 | 14.481 | 99.522 | 1.00 | 63.48 |
| 673 | N | LEU | 90 | −4.262 | 16.658 | 100.038 | 1.00 | 62.02 |
| 674 | CA | LEU | 90 | −3.829 | 17.056 | 98.721 | 1.00 | 53.90 |
| 675 | CB | LEU | 90 | −4.868 | 18.023 | 98.135 | 1.00 | 57.62 |
| 676 | CG | LEU | 90 | −4.464 | 18.887 | 96.947 | 1.00 | 65.94 |
| 677 | CD1 | LEU | 90 | −4.011 | 17.974 | 95.846 | 1.00 | 49.14 |
| 678 | CD2 | LEU | 90 | −5.616 | 19.820 | 96.536 | 1.00 | 51.17 |
| 679 | C | LEU | 90 | −2.434 | 17.688 | 98.822 | 1.00 | 51.80 |
| 680 | O | LEU | 90 | −2.309 | 18.823 | 99.242 | 1.00 | 54.93 |
| 681 | N | VAL | 91 | −1.391 | 16.928 | 98.484 | 1.00 | 44.81 |
| 682 | CA | VAL | 91 | −0.026 | 17.442 | 98.518 | 1.00 | 43.52 |
| 683 | CB | VAL | 91 | 6 | 16.303 | 98.684 | 1.00 | 37.98 |
| 684 | CG1 | VAL | 91 | 2.410 | 16.851 | 98.859 | 1.00 | 34.55 |
| 685 | CG2 | VAL | 91 | 0.674 | 15.491 | 99.902 | 1.00 | 41.03 |
| 686 | C | VAL | 91 | 0.157 | 18.167 | 97.188 | 1.00 | 50.08 |
| 687 | O | VAL | 91 | −0.077 | 17.594 | 96.124 | 1.00 | 54.83 |
| 688 | N | ASP | 92 | 0.561 | 19.433 | 97.235 | 1.00 | 50.26 |
| 689 | CA | ASP | 92 | 0.661 | 20.200 | 95.992 | 1.00 | 49.93 |
| 690 | CB | ASP | 92 | −0.608 | 21.068 | 95.833 | 1.00 | 44.53 |
| 691 | CG | ASP | 92 | −0.878 | 21.491 | 94.382 | 1.00 | 50.72 |
| 692 | OD1 | ASP | 92 | −0.001 | 21.315 | 93.509 | 1.00 | 60.21 |
| 693 | OD2 | ASP | 92 | −1.980 | 22.014 | 94.118 | 1.00 | 49.19 |
| 694 | C | ASP | 92 | 1.914 | 21.046 | 95.904 | 1.00 | 43.21 |
| 695 | O | ASP | 92 | 2.558 | 21.328 | 96.903 | 1.00 | 50.41 |
| 696 | N | VAL | 93 | 2.254 | 21.447 | 94.693 | 1.00 | 48.54 |
| 697 | CA | VAL | 93 | 3.457 | 22.235 | 94.422 | 1.00 | 56.64 |
| 698 | CB | VAL | 93 | 4.117 | 21.763 | 93.062 | 1.00 | 65.00 |
| 699 | CG1 | VAL | 93 | 5.482 | 22.367 | 92.892 | 1.00 | 57.14 |
| 700 | CG2 | VAL | 93 | 4.166 | 20.247 | 92.972 | 1.00 | 57.19 |
| 701 | C | VAL | 93 | 3.094 | 23.707 | 94.224 | 1.00 | 54.91 |
| 702 | O | VAL | 93 | 1.960 | 24.007 | 93.848 | 1.00 | 48.69 |
| 703 | N | PRO | 94 | 4.027 | 24.637 | 94.545 | 1.00 | 57.90 |
| 704 | CD | PRO | 94 | 4.926 | 24.399 | 95.690 | 1.00 | 61.07 |
| 705 | CA | PRO | 94 | 3.848 | 26.093 | 94.376 | 1.00 | 51.73 |
| 706 | CB | PRO | 94 | 4.912 | 26.693 | 95.278 | 1.00 | 46.67 |
| 707 | CG | PRO | 94 | 4.923 | 25.779 | 96.407 | 1.00 | 48.86 |
| 708 | C | PRO | 94 | 4.171 | 26.338 | 92.910 | 1.00 | 46.65 |
| 709 | O | PRO | 94 | 5.210 | 25.883 | 92.415 | 1.00 | 42.39 |
| 710 | N | PRO | 95 | 3.304 | 27.076 | 92.191 | 1.00 | 40.25 |
| 711 | CD | PRO | 95 | 2.088 | 27.787 | 92.592 | 1.00 | 30.77 |
| 712 | CA | PRO | 95 | 3.617 | 27.290 | 90.772 | 1.00 | 39.13 |
| 713 | CB | PRO | 95 | 2.431 | 28.095 | 90.249 | 1.00 | 29.26 |
| 714 | CG | PRO | 95 | 1.361 | 27.904 | 91.295 | 1.00 | 41.20 |
| 715 | C | PRO | 95 | 4.923 | 27.967 | 90.462 | 1.00 | 43.75 |
| 716 | O | PRO | 95 | 5.509 | 28.663 | 91.290 | 1.00 | 50.53 |
| 717 | N | GLU | 96 | 5.406 | 27.718 | 89.252 | 1.00 | 54.99 |
| 718 | CA | GLU | 96 | 6.610 | 28.390 | 88.788 | 1.00 | 47.41 |
| 719 | CB | GLU | 96 | 7.141 | 27.703 | 87.544 | 1.00 | 57.60 |
| 720 | CG | GLU | 96 | 7.311 | 26.220 | 87.691 | 1.00 | 48.45 |
| 721 | CD | GLU | 96 | 7.901 | 25.595 | 86.435 | 1.00 | 68.71 |
| 722 | OE1 | GLU | 96 | 8.410 | 26.357 | 85.566 | 1.00 | 62.79 |
| 723 | OE2 | GLU | 96 | 7.860 | 24.342 | 86.327 | 1.00 | 70.89 |
| 724 | C | GLU | 96 | 6.049 | 29.771 | 88.410 | 1.00 | 47.41 |
| 725 | O | GLU | 97 | 4.827 | 29.892 | 88.171 | 1.00 | 39.12 |
| 726 | N | GLU | 97 | 6.892 | 30.808 | 88.369 | 1.00 | 42.01 |
| 727 | CA | GLU | 97 | 6.361 | 32.129 | 88.005 | 1.00 | 44.48 |
| 728 | CB | GLU | 97 | 7.296 | 33.276 | 88.384 | 1.00 | 35.88 |
| 729 | CG | GLU | 97 | 6.643 | 34.632 | 88.048 | 1.00 | 52.12 |
| 730 | CD | GLU | 97 | 7.160 | 35.806 | 88.863 | 1.00 | 56.72 |
| 731 | OE1 | GLU | 97 | 7.257 | 35.699 | 90.109 | 1.00 | 62.38 |
| 732 | OE2 | GLU | 97 | 7.443 | 36.855 | 88.247 | 1.00 | 60.92 |
| 733 | C | GLU | 97 | 6.137 | 32.118 | 86.508 | 1.00 | 33.61 |
| 734 | O | GLU | 97 | 7.029 | 31.839 | 85.763 | 1.00 | 30.35 |
| 735 | N | PRO | 98 | 4.932 | 32.437 | 86.045 | 1.00 | 39.63 |
| 736 | CD | PRO | 98 | 3.619 | 32.750 | 86.603 | 1.00 | 35.46 |
| 737 | CA | PRO | 98 | 4.886 | 32.354 | 84.583 | 1.00 | 39.23 |
| 738 | CB | PRO | 98 | 3.404 | 32.545 | 84.271 | 1.00 | 37.32 |
| 739 | CG | PRO | 98 | 2.699 | 32.173 | 85.527 | 1.00 | 42.18 |
| 740 | C | PRO | 98 | 5.803 | 33.338 | 83.832 | 1.00 | 43.37 |
| 741 | O | PRO | 98 | 6.213 | 34.372 | 84.375 | 1.00 | 34.56 |
| 742 | N | GLN | 99 | 6.173 | 32.955 | 82.613 | 1.00 | 37.43 |
| 743 | CA | GLN | 99 | 7.000 | 33.792 | 81.726 | 1.00 | 46.36 |
| 744 | CB | GLN | 99 | 8.282 | 33.080 | 81.248 | 1.00 | 49.33 |
| 745 | CG | GLN | 99 | 9.371 | 32.848 | 82.276 | 1.00 | 42.67 |
| 746 | CD | GLN | 99 | 9.529 | 33.999 | 83.198 | 1.00 | 56.22 |
| 747 | OE1 | GLN | 99 | 9.015 | 33.966 | 84.321 | 1.00 | 55.11 |
| 748 | NE2 | GLN | 99 | 10.223 | 35.050 | 82.743 | 1.00 | 50.83 |
| 749 | C | GLN | 99 | 6.057 | 33.906 | 80.540 | 1.00 | 48.35 |
| 750 | O | GLN | 99 | 5.966 | 33.004 | 79.730 | 1.00 | 48.46 |
| 751 | N | LEU | 100 | 5.369 | 35.021 | 80.432 | 1.00 | 48.85 |
| 752 | CA | LEU | 100 | 4.372 | 35.168 | 79.396 | 1.00 | 46.80 |
| 753 | CB | LEU | 100 | 3.259 | 36.125 | 79.897 | 1.00 | 33.22 |
| 754 | CG | LEU | 100 | 1.993 | 36.407 | 79.078 | 1.00 | 46.78 |
| 755 | CD1 | LEU | 100 | 1.174 | 35.167 | 78.984 | 1.00 | 40.64 |
| 756 | CD2 | LEU | 100 | 1.175 | 37.525 | 79.732 | 1.00 | 36.60 |
| 757 | C | LEU | 100 | 4.893 | 35.605 | 78.054 | 1.00 | 40.31 |
| 758 | O | LEU | 100 | 5.724 | 36.492 | 77.949 | 1.00 | 46.96 |
| 759 | N | SER | 101 | 4.380 | 34.961 | 77.022 | 1.00 | 39.64 |
| 760 | CA | SER | 101 | 4.740 | 35.292 | 75.652 | 1.00 | 41.21 |
| 761 | CB | SER | 101 | 5.499 | 34.143 | 74.972 | 1.00 | 43.96 |
| 762 | OG | SER | 101 | 5.895 | 34.520 | 73.659 | 1.00 | 59.52 |
| 763 | C | SER | 101 | 3.408 | 35.490 | 74.974 | 1.00 | 34.29 |
| 764 | O | SER | 101 | 2.591 | 34.543 | 74.923 | 1.00 | 41.42 |
| 765 | N | CYS | 102 | 3.150 | 36.720 | 74.514 | 1.00 | 37.29 |
| 766 | CA | CYS | 102 | 1.887 | 37.006 | 73.803 | 1.00 | 46.92 |
| 767 | C | CYS | 102 | 2.209 | 37.307 | 72.328 | 1.00 | 49.40 |
| 768 | O | CYS | 102 | 3.242 | 37.906 | 72.041 | 1.00 | 41.30 |
| 769 | CB | CYS | 102 | 1.157 | 38.201 | 74.397 | 1.00 | 45.80 |
| 770 | SG | CYS | 102 | 0.362 | 38.045 | 76.021 | 1.00 | 47.25 |
| 771 | N | PHE | 103 | 1.340 | 36.895 | 71.401 | 1.00 | 39.80 |
| 772 | CA | PHE | 103 | 1.613 | 37.133 | 69.983 | 1.00 | 37.94 |
| 773 | CB | PHE | 103 | 2.638 | 36.114 | 69.477 | 1.00 | 31.69 |
| 774 | CG | PHE | 103 | 2.147 | 34.659 | 69.546 | 1.00 | 36.43 |
| 775 | CD1 | PHE | 103 | 1.410 | 34.109 | 68.511 | 1.00 | 31.53 |
| 776 | CD2 | PHE | 103 | 2.389 | 33.881 | 70.657 | 1.00 | 35.41 |
| 777 | CE1 | PHE | 103 | 0.921 | 32.809 | 68.586 | 1.00 | 43.33 |
| 778 | CE2 | PHE | 103 | 1.904 | 32.580 | 70.741 | 1.00 | 46.34 |
| 779 | CZ | PHE | 103 | 1.163 | 32.039 | 69.700 | 1.00 | 40.86 |
| 780 | C | PHE | 103 | 0.369 | 37.033 | 69.129 | 1.00 | 42.42 |
| 781 | O | PHE | 103 | −0.670 | 36.556 | 69.585 | 1.00 | 45.54 |
| 782 | N | ARG | 104 | 0.481 | 37.489 | 67.883 | 1.00 | 36.44 |
| 783 | CA | ARG | 104 | −0.610 | 37.423 | 66.937 | 1.00 | 30.87 |
| 784 | CB | ARG | 104 | −1.499 | 38.665 | 67.108 | 1.00 | 37.76 |
| 785 | CG | ARG | 104 | −2.450 | 38.937 | 65.972 | 1.00 | 35.03 |
| 786 | CD | ARG | 104 | −3.837 | 39.231 | 66.399 | 1.00 | 34.41 |
| 787 | NE | ARG | 104 | −4.168 | 40.647 | 66.414 | 1.00 | 38.04 |
| 788 | CZ | ARG | 104 | −5.418 | 41.095 | 66.509 | 1.00 | 47.99 |
| 789 | NH1 | ARG | 104 | −6.415 | 40.235 | 66.574 | 1.00 | 41.92 |
| 790 | NH2 | ARG | 104 | −5.677 | 42.391 | 66.605 | 1.00 | 49.53 |
| 791 | C | ARG | 104 | −0.104 | 37.244 | 65.476 | 1.00 | 36.29 |
| 792 | O | ARG | 104 | 0.473 | 38.140 | 64.871 | 1.00 | 34.66 |
| 793 | N | LYS | 105 | −0.323 | 36.050 | 64.943 | 1.00 | 38.88 |
| 794 | CA | LYS | 105 | 0.067 | 35.706 | 63.574 | 1.00 | 48.56 |
| 795 | CB | LYS | 105 | −0.084 | 34.197 | 63.389 | 1.00 | 38.91 |
| 796 | CG | LYS | 105 | 0.771 | 33.367 | 64.349 | 1.00 | 37.33 |
| 797 | CD | LYS | 105 | 2.241 | 33.760 | 64.233 | 1.00 | 54.88 |
| 798 | CE | LYS | 105 | 3.103 | 33.179 | 65.359 | 1.00 | 65.25 |
| 799 | NZ | LYS | 105 | 3.400 | 31.742 | 65.201 | 1.00 | 49.35 |
| 800 | C | LYS | 105 | −0.634 | 36.482 | 62.411 | 1.00 | 49.69 |
| 801 | O | LYS | 105 | 0.033 | 36.915 | 61.495 | 1.00 | 53.72 |
| 802 | N | SER | 106 | −1.954 | 36.662 | 62.434 | 1.00 | 46.97 |
| 803 | CA | SER | 106 | −2.618 | 37.427 | 61.375 | 1.00 | 46.49 |
| 804 | CB | SER | 106 | −3.401 | 36.515 | 60.446 | 1.00 | 48.20 |
| 805 | OG | SER | 106 | −4.006 | 35.537 | 61.209 | 1.00 | 47.79 |
| 806 | C | SER | 106 | −3.533 | 38.471 | 61.980 | 1.00 | 48.92 |
| 807 | O | SER | 106 | −3.914 | 38.352 | 63.137 | 1.00 | 49.81 |
| 808 | N | PRO | 107 | −3.909 | 39.504 | 61.202 | 1.00 | 42.09 |
| 809 | CD | PRO | 107 | −3.722 | 39.610 | 59.744 | 1.00 | 31.34 |
| 810 | CA | PRO | 107 | −4.777 | 40.585 | 61.691 | 1.00 | 41.24 |
| 811 | CB | PRO | 107 | −4.971 | 41.484 | 60.459 | 1.00 | 44.71 |
| 812 | CG | PRO | 107 | −3.855 | 41.098 | 59.537 | 1.00 | 46.51 |
| 813 | C | PRO | 107 | −6.124 | 40.200 | 62.302 | 1.00 | 51.12 |
| 814 | O | PRO | 107 | −6.663 | 40.937 | 63.165 | 1.00 | 46.09 |
| 815 | N | LEU | 108 | −6.654 | 39.058 | 61.856 | 1.00 | 48.55 |
| 816 | CA | LEU | 108 | −7.958 | 38.589 | 62.308 | 1.00 | 57.21 |
| 817 | CB | LEU | 108 | −8.827 | 38.178 | 61.106 | 1.00 | 46.02 |
| 818 | CG | LEU | 108 | −9.973 | 39.090 | 60.604 | 1.00 | 54.59 |
| 819 | CD1 | LEU | 108 | −9.837 | 40.553 | 61.070 | 1.00 | 39.70 |
| 820 | CD2 | LEU | 108 | −10.008 | 38.987 | 59.082 | 1.00 | 41.31 |
| 821 | C | LEU | 108 | −7.958 | 37.468 | 63.324 | 1.00 | 58.78 |
| 822 | O | LEU | 108 | −9.012 | 37.173 | 63.884 | 1.00 | 62.46 |
| 823 | N | SER | 109 | −6.813 | 36.830 | 63.564 | 1.00 | 50.34 |
| 824 | CA | SER | 109 | −6.778 | 35.767 | 64.568 | 1.00 | 40.58 |
| 825 | CB | SER | 109 | −5.640 | 34.796 | 64.320 | 1.00 | 43.38 |
| 826 | OG | SER | 109 | −4.405 | 35.475 | 64.272 | 1.00 | 70.48 |
| 827 | C | SER | 109 | −6.623 | 36.419 | 65.928 | 1.00 | 41.40 |
| 828 | O | SER | 109 | −6.268 | 37.603 | 66.039 | 1.00 | 36.98 |
| 829 | N | ASN | 110 | −6.919 | 35.660 | 66.971 | 1.00 | 39.51 |
| 830 | CA | ASN | 110 | −6.836 | 36.213 | 68.323 | 1.00 | 42.42 |
| 831 | CB | ASN | 110 | −7.557 | 35.327 | 69.319 | 1.00 | 51.49 |
| 832 | CG | ASN | 110 | −9.035 | 35.418 | 69.200 | 1.00 | 52.17 |
| 833 | OD1 | ASN | 110 | −9.593 | 36.489 | 68.950 | 1.00 | 52.02 |
| 834 | ND2 | ASN | 110 | −9.686 | 34.307 | 69.399 | 1.00 | 45.26 |
| 835 | C | ASN | 110 | −5.450 | 36.370 | 68.840 | 1.00 | 40.70 |
| 836 | O | ASN | 110 | −4.542 | 35.692 | 68.389 | 1.00 | 34.47 |
| 837 | N | VAL | 111 | −5.284 | 37.273 | 69.804 | 1.00 | 44.99 |
| 838 | CA | VAL | 111 | −3.979 | 37.410 | 70.432 | 1.00 | 30.99 |
| 839 | CB | VAL | 111 | −3.885 | 38.627 | 71.317 | 1.00 | 31.57 |
| 840 | CG1 | VAL | 111 | −2.627 | 38.521 | 72.191 | 1.00 | 43.27 |
| 841 | CG2 | VAL | 111 | −3.805 | 39.900 | 70.436 | 1.00 | 31.41 |
| 842 | C | VAL | 111 | −3.855 | 36.162 | 71.281 | 1.00 | 31.76 |
| 843 | O | VAL | 111 | −4.804 | 35.763 | 71.942 | 1.00 | 35.82 |
| 844 | N | VAL | 112 | −2.722 | 35.489 | 71.184 | 1.00 | 41.08 |
| 845 | CA | VAL | 112 | −2.492 | 34.314 | 72.000 | 1.00 | 35.85 |
| 846 | CB | VAL | 112 | −2.014 | 33.109 | 71.196 | 1.00 | 43.27 |
| 847 | CG1 | VAL | 112 | −1.963 | 31.912 | 72.098 | 1.00 | 36.63 |
| 848 | CG2 | VAL | 112 | −2.953 | 32.824 | 70.022 | 1.00 | 48.26 |
| 849 | C | VAL | 112 | −1.432 | 34.660 | 73.031 | 1.00 | 38.53 |
| 850 | O | VAL | 112 | −0.430 | 35.325 | 72.736 | 1.00 | 43.19 |
| 851 | N | CYS | 113 | −1.653 | 34.215 | 74.261 | 1.00 | 46.31 |
| 852 | CA | CYS | 113 | −0.691 | 34.468 | 75.317 | 1.00 | 41.85 |
| 853 | C | CYS | 113 | −0.424 | 33.136 | 75.966 | 1.00 | 42.26 |
| 854 | O | CYS | 113 | −1.349 | 32.467 | 76.412 | 1.00 | 43.58 |
| 855 | CB | CYS | 113 | −1.258 | 35.448 | 76.315 | 1.00 | 40.17 |
| 856 | SG | CYS | 113 | −1.472 | 37.152 | 75.750 | 1.00 | 47.14 |
| 857 | N | GLU | 114 | 0.851 | 32.756 | 75.997 | 1.00 | 40.00 |
| 858 | CA | GLU | 114 | 1.255 | 31.467 | 76.536 | 1.00 | 47.49 |
| 859 | CB | GLU | 114 | 1.461 | 30.469 | 75.379 | 1.00 | 38.32 |
| 860 | CG | GLU | 114 | 2.398 | 31.017 | 74.338 | 1.00 | 47.09 |
| 861 | CD | GLU | 114 | 2.874 | 29.995 | 73.310 | 1.00 | 62.17 |
| 862 | OE1 | GLU | 114 | 2.030 | 29.229 | 72.772 | 1.00 | 54.04 |
| 863 | OE2 | GLU | 114 | 4.107 | 29.985 | 73.031 | 1.00 | 62.75 |
| 864 | C | GLU | 114 | 2.523 | 31.488 | 77.408 | 1.00 | 46.47 |
| 865 | O | GLU | 114 | 3.252 | 32.479 | 77.479 | 1.00 | 43.21 |
| 866 | N | TRP | 115 | 2.757 | 30.345 | 78.042 | 1.00 | 39.36 |
| 867 | CA | TRP | 115 | 3.867 | 30.092 | 78.925 | 1.00 | 38.92 |
| 868 | CB | TRP | 115 | 3.585 | 30.583 | 80.357 | 1.00 | 32.46 |
| 869 | CG | TRP | 115 | 4.558 | 29.976 | 81.373 | 1.00 | 33.00 |
| 870 | CD2 | TRP | 115 | 4.201 | 29.393 | 82.628 | 1.00 | 19.31 |
| 871 | CE2 | TRP | 115 | 5.398 | 29.136 | 83.338 | 1.00 | 25.04 |
| 872 | CE3 | TRP | 115 | 2.989 | 29.088 | 83.228 | 1.00 | 36.87 |
| 873 | CD1 | TRP | 115 | 5.956 | 30.018 | 81.349 | 1.00 | 22.44 |
| 874 | NE1 | TRP | 115 | 6.450 | 29.516 | 82.542 | 1.00 | 31.17 |
| 875 | CZ2 | TRP | 115 | 5.401 | 28.596 | 84.603 | 1.00 | 46.54 |
| 876 | CZ3 | TRP | 115 | 2.995 | 28.539 | 84.503 | 1.00 | 51.37 |
| 877 | CH2 | TRP | 115 | 4.188 | 28.304 | 85.176 | 1.00 | 36.41 |
| 878 | C | TRP | 115 | 3.954 | 28.588 | 79.001 | 1.00 | 43.57 |
| 879 | O | TRP | 115 | 2.946 | 27.913 | 79.265 | 1.00 | 54.50 |
| 880 | N | GLY | 116 | 5.147 | 28.057 | 78.793 | 1.00 | 48.25 |
| 881 | CA | GLY | 116 | 5.319 | 26.625 | 78.906 | 1.00 | 34.75 |
| 882 | C | GLY | 116 | 6.136 | 26.410 | 80.170 | 1.00 | 45.74 |
| 883 | O | GLY | 116 | 7.302 | 26.815 | 80.206 | 1.00 | 40.47 |
| 884 | N | PRO | 117 | 5.552 | 25.833 | 81.239 | 1.00 | 34.02 |
| 885 | CD | PRO | 117 | 4.113 | 25.583 | 81.425 | 1.00 | 32.14 |
| 886 | CA | PRO | 117 | 6.296 | 25.589 | 82.480 | 1.00 | 38.56 |
| 887 | CB | PRO | 117 | 5.277 | 24.861 | 83.348 | 1.00 | 44.21 |
| 888 | CG | PRO | 117 | 3.966 | 25.485 | 82.926 | 1.00 | 38.49 |
| 889 | C | PRO | 117 | 7.519 | 24.725 | 82.205 | 1.00 | 42.91 |
| 890 | O | PRO | 117 | 7.550 | 24.020 | 81.211 | 1.00 | 52.27 |
| 891 | N | ARG | 118 | 8.517 | 24.795 | 83.080 | 1.00 | 52.30 |
| 892 | CA | ARG | 118 | 9.738 | 23.999 | 82.962 | 1.00 | 60.14 |
| 893 | CB | ARG | 118 | 10.835 | 24.513 | 83.914 | 1.00 | 53.02 |
| 894 | CG | ARG | 118 | 11.454 | 25.823 | 83.511 | 1.00 | 67.97 |
| 895 | CD | ARG | 118 | 12.620 | 26.219 | 84.408 | 1.00 | 76.97 |
| 896 | NE | ARG | 118 | 12.268 | 26.431 | 85.818 | 1.00 | 80.60 |
| 897 | CZ | ARG | 118 | 11.326 | 27.269 | 86.252 | 1.00 | 86.10 |
| 898 | NH1 | ARG | 118 | 10.608 | 27.988 | 85.388 | 1.00 | 81.53 |
| 899 | NH2 | ARG | 118 | 11.116 | 27.407 | 87.556 | 1.00 | 84.72 |
| 900 | C | ARG | 118 | 9.439 | 22.543 | 83.322 | 1.00 | 59.67 |
| 901 | O | ARG | 118 | 10.216 | 21.655 | 82.992 | 1.00 | 65.23 |
| 902 | N | SER | 119 | 8.322 | 22.320 | 84.009 | 1.00 | 51.40 |
| 903 | CA | SER | 119 | 7.871 | 20.985 | 84.442 | 1.00 | 60.61 |
| 904 | CB | SER | 119 | 8.235 | 20.706 | 85.922 | 1.00 | 61.14 |
| 905 | OG | SER | 119 | 9.636 | 20.614 | 86.156 | 1.00 | 61.55 |
| 906 | C | SER | 119 | 6.346 | 20.904 | 84.314 | 1.00 | 57.51 |
| 907 | O | SER | 119 | 5.653 | 21.926 | 84.330 | 1.00 | 56.20 |
| 908 | N | THR | 120 | 5.829 | 19.687 | 84.208 | 1.00 | 53.08 |
| 909 | CA | THR | 120 | 4.393 | 19.465 | 84.079 | 1.00 | 55.91 |
| 910 | CB | THR | 120 | 4.136 | 18.027 | 83.657 | 1.00 | 43.60 |
| 911 | OG1 | THR | 120 | 4.653 | 17.896 | 82.337 | 1.00 | 60.80 |
| 912 | CG2 | THR | 120 | 2.673 | 17.674 | 83.641 | 1.00 | 31.27 |
| 913 | C | THR | 120 | 3.644 | 19.839 | 85.350 | 1.00 | 57.63 |
| 914 | O | THR | 120 | 3.784 | 19.218 | 86.411 | 1.00 | 49.94 |
| 915 | N | PRO | 121 | 2.837 | 20.892 | 85.253 | 1.00 | 62.50 |
| 916 | CD | PRO | 121 | 2.479 | 21.656 | 84.047 | 1.00 | 64.06 |
| 917 | CA | PRO | 121 | 2.073 | 21.359 | 86.398 | 1.00 | 63.14 |
| 918 | CB | PRO | 121 | 1.442 | 22.645 | 85.874 | 1.00 | 52.64 |
| 919 | CG | PRO | 121 | 1.174 | 22.309 | 84.477 | 1.00 | 69.13 |
| 920 | C | PRO | 121 | 1.059 | 20.346 | 86.851 | 1.00 | 49.47 |
| 921 | O | PRO | 121 | 0.516 | 19.610 | 86.041 | 1.00 | 58.72 |
| 922 | N | SER | 122 | 0.809 | 20.350 | 88.159 | 1.00 | 55.49 |
| 923 | CA | SER | 122 | −0.147 | 19.464 | 88.820 | 1.00 | 48.82 |
| 924 | CB | SER | 122 | −0.187 | 19.778 | 90.331 | 1.00 | 55.95 |
| 925 | OG | SER | 122 | −1.141 | 20.792 | 90.664 | 1.00 | 54.54 |
| 926 | C | SER | 122 | −1.532 | 19.640 | 88.211 | 1.00 | 44.78 |
| 927 | O | SER | 122 | −1.737 | 20.547 | 87.442 | 1.00 | 59.65 |
| 928 | N | LEU | 123 | −2.492 | 18.794 | 88.567 | 1.00 | 54.52 |
| 929 | CA | LEU | 123 | −3.831 | 18.913 | 88.000 | 1.00 | 57.07 |
| 930 | CB | LEU | 123 | −4.563 | 17.557 | 88.054 | 1.00 | 55.73 |
| 931 | CG | LEU | 123 | −3.896 | 16.420 | 87.247 | 1.00 | 67.94 |
| 932 | CD1 | LEU | 123 | −4.740 | 15.152 | 87.363 | 1.00 | 56.21 |
| 933 | CD2 | LEU | 123 | −3.717 | 16.826 | 85.764 | 1.00 | 38.88 |
| 934 | C | LEU | 123 | −4.651 | 19.995 | 88.691 | 1.00 | 56.28 |
| 935 | O | LEU | 123 | −5.738 | 20.361 | 88.228 | 1.00 | 59.85 |
| 936 | N | THR | 124 | −4.145 | 20.503 | 89.806 | 1.00 | 52.98 |
| 937 | CA | THR | 124 | −4.849 | 21.567 | 90.514 | 1.00 | 53.57 |
| 938 | CB | THR | 124 | −4.828 | 21.373 | 92.061 | 1.00 | 56.34 |
| 939 | OG1 | THR | 124 | −3.482 | 21.128 | 92.490 | 1.00 | 53.00 |
| 940 | CG2 | THR | 124 | −5.744 | 20.255 | 92.493 | 1.00 | 44.62 |
| 941 | C | THR | 124 | −4.210 | 22.939 | 90.223 | 1.00 | 56.80 |
| 942 | O | THR | 124 | −4.422 | 23.891 | 90.989 | 1.00 | 57.07 |
| 943 | N | THR | 125 | −3.440 | 23.038 | 89.134 | 1.00 | 43.79 |
| 944 | CA | THR | 125 | −2.765 | 24.289 | 88.763 | 1.00 | 46.89 |
| 945 | CB | THR | 125 | −1.307 | 24.038 | 88.312 | 1.00 | 47.29 |
| 946 | OG1 | THR | 125 | −0.562 | 23.426 | 89.370 | 1.00 | 43.72 |
| 947 | CG2 | THR | 125 | −0.656 | 25.319 | 87.926 | 1.00 | 41.17 |
| 948 | C | THR | 125 | −3.463 | 24.989 | 87.607 | 1.00 | 48.99 |
| 949 | O | THR | 125 | −3.290 | 24.582 | 86.476 | 1.00 | 64.28 |
| 950 | N | LYS | 126 | −4.251 | 26.025 | 87.880 | 1.00 | 38.84 |
| 951 | CA | LYS | 126 | −4.936 | 26.753 | 86.820 | 1.00 | 45.34 |
| 952 | CB | LYS | 126 | −6.405 | 27.014 | 87.184 | 1.00 | 50.48 |
| 953 | CG | LYS | 126 | −7.163 | 25.773 | 87.617 | 1.00 | 58.30 |
| 954 | CD | LYS | 126 | −6.992 | 24.658 | 86.597 | 1.00 | 61.76 |
| 955 | CE | LYS | 126 | −7.619 | 23.349 | 87.057 | 1.00 | 64.05 |
| 956 | NZ | LYS | 126 | −7.663 | 22.402 | 85.903 | 1.00 | 68.15 |
| 957 | C | LYS | 126 | −4.268 | 28.085 | 86.505 | 1.00 | 44.50 |
| 958 | O | LYS | 126 | −3.585 | 28.681 | 87.347 | 1.00 | 43.84 |
| 959 | N | ALA | 127 | −4.481 | 28.564 | 85.287 | 1.00 | 45.08 |
| 960 | CA | ALA | 127 | −3.902 | 29.837 | 84.860 | 1.00 | 37.24 |
| 961 | CB | ALA | 127 | −2.821 | 29.600 | 83.813 | 1.00 | 42.13 |
| 962 | C | ALA | 127 | −4.999 | 30.679 | 84.276 | 1.00 | 36.63 |
| 963 | O | ALA | 127 | −5.969 | 30.160 | 83.766 | 1.00 | 54.88 |
| 964 | N | VAL | 128 | −4.873 | 31.988 | 84.367 | 1.00 | 42.57 |
| 965 | CA | VAL | 128 | −5.877 | 32.840 | 83.771 | 1.00 | 38.06 |
| 966 | CB | VAL | 128 | −7.009 | 33.266 | 84.770 | 1.00 | 46.10 |
| 967 | CG1 | VAL | 128 | −7.771 | 32.038 | 85.313 | 1.00 | 36.01 |
| 968 | CG2 | VAL | 128 | −6.410 | 34.066 | 85.887 | 1.00 | 39.87 |
| 969 | C | VAL | 128 | −5.121 | 34.077 | 83.340 | 1.00 | 45.97 |
| 970 | O | VAL | 128 | −4.073 | 34.415 | 83.888 | 1.00 | 37.87 |
| 971 | N | LEU | 129 | −5.666 | 34.757 | 82.352 | 1.00 | 39.37 |
| 972 | CA | LEU | 129 | −5.048 | 35.943 | 81.855 | 1.00 | 35.49 |
| 973 | CB | LEU | 129 | −5.136 | 35.963 | 80.319 | 1.00 | 28.09 |
| 974 | CG | LEU | 129 | −4.174 | 36.966 | 79.670 | 1.00 | 48.04 |
| 975 | CD1 | LEU | 129 | −2.726 | 36.395 | 79.701 | 1.00 | 31.77 |
| 976 | CD2 | LEU | 129 | −4.638 | 37.275 | 78.234 | 1.00 | 42.39 |
| 977 | C | LEU | 129 | −5.787 | 37.134 | 82.469 | 1.00 | 38.11 |
| 978 | O | LEU | 129 | −7.008 | 37.243 | 82.340 | 1.00 | 48.40 |
| 979 | N | LEU | 130 | −5.050 | 37.994 | 83.175 | 1.00 | 31.69 |
| 980 | CA | LEU | 130 | −5.615 | 39.194 | 83.757 | 1.00 | 35.11 |
| 981 | CB | LEU | 130 | −4.866 | 39.598 | 85.032 | 1.00 | 33.65 |
| 982 | CG | LEU | 130 | −4.847 | 38.612 | 86.216 | 1.00 | 46.79 |
| 983 | CD1 | LEU | 130 | −4.508 | 39.390 | 87.424 | 1.00 | 40.22 |
| 984 | CD2 | LEU | 130 | −6.195 | 37.942 | 86.447 | 1.00 | 52.37 |
| 985 | C | LEU | 130 | −5.367 | 40.223 | 82.665 | 1.00 | 39.15 |
| 986 | O | LEU | 130 | −4.231 | 40.347 | 82.184 | 1.00 | 47.42 |
| 987 | N | VAL | 131 | −6.427 | 40.915 | 82.254 | 1.00 | 37.89 |
| 988 | CA | VAL | 131 | −6.370 | 41.930 | 81.213 | 1.00 | 33.95 |
| 989 | CB | VAL | 131 | −7.237 | 41.501 | 79.984 | 1.00 | 33.95 |
| 990 | CG1 | VAL | 131 | −7.350 | 42.661 | 78.893 | 1.00 | 27.58 |
| 991 | CG2 | VAL | 131 | −6.631 | 40.238 | 79.373 | 1.00 | 31.78 |
| 992 | C | VAL | 131 | −6.845 | 43.309 | 81.688 | 1.00 | 43.64 |
| 993 | O | VAL | 131 | −7.890 | 43.434 | 82.322 | 1.00 | 49.13 |
| 994 | N | ARG | 132 | −6.060 | 44.333 | 81.385 | 1.00 | 40.64 |
| 995 | CA | ARG | 132 | −6.410 | 45.709 | 81.706 | 1.00 | 43.19 |
| 996 | CB | ARG | 132 | −5.438 | 46.341 | 82.683 | 1.00 | 36.47 |
| 997 | CG | ARG | 132 | −5.967 | 47.623 | 83.306 | 1.00 | 63.76 |
| 998 | CD | ARG | 132 | −4.950 | 48.322 | 84.236 | 1.00 | 68.20 |
| 999 | NE | ARG | 132 | −4.678 | 47.611 | 85.487 | 1.00 | 75.94 |
| 1000 | CZ | ARG | 132 | −5.584 | 47.379 | 86.436 | 1.00 | 79.15 |
| 1001 | NH1 | ARG | 132 | −6.839 | 47.798 | 86.280 | 1.00 | 76.47 |
| 1002 | NH2 | ARG | 132 | −5.234 | 46.743 | 87.550 | 1.00 | 69.95 |
| 1003 | C | ARG | 132 | −6.351 | 46.450 | 80.369 | 1.00 | 47.07 |
| 1004 | O | ARG | 132 | −5.303 | 46.601 | 79.756 | 1.00 | 39.17 |
| 1005 | N | LYS | 133 | −7.522 | 46.876 | 79.928 | 1.00 | 48.96 |
| 1006 | CA | LYS | 133 | −7.724 | 47.574 | 78.683 | 1.00 | 44.69 |
| 1007 | CB | LYS | 133 | −8.948 | 46.955 | 78.011 | 1.00 | 44.54 |
| 1008 | CG | LYS | 133 | −9.426 | 47.649 | 76.751 | 1.00 | 62.07 |
| 1009 | CD | LYS | 133 | −10.552 | 46.839 | 76.128 | 1.00 | 62.69 |
| 1010 | CE | LYS | 133 | −11.031 | 47.434 | 74.833 | 1.00 | 61.89 |
| 1011 | NZ | LYS | 133 | −12.068 | 46.534 | 74.226 | 1.00 | 71.97 |
| 1012 | C | LYS | 133 | −7.922 | 49.079 | 78.921 | 1.00 | 45.45 |
| 1013 | O | LYS | 133 | −8.675 | 49.487 | 79.788 | 1.00 | 49.88 |
| 1014 | N | PHE | 134 | −7.212 | 49.883 | 78.151 | 1.00 | 41.56 |
| 1015 | CA | PHE | 134 | −7.275 | 51.331 | 78.223 | 1.00 | 43.83 |
| 1016 | CB | PHE | 134 | −5.875 | 51.935 | 78.421 | 1.00 | 39.49 |
| 1017 | CG | PHE | 134 | −5.176 | 51.501 | 79.691 | 1.00 | 43.93 |
| 1018 | CD1 | PHE | 134 | −4.819 | 50.148 | 79.901 | 1.00 | 39.01 |
| 1019 | CD2 | PHE | 134 | −4.902 | 52.434 | 80.697 | 1.00 | 39.93 |
| 1020 | CE1 | PHE | 134 | −4.217 | 49.741 | 81.099 | 1.00 | 37.66 |
| 1021 | CE2 | PHE | 134 | −4.283 | 52.037 | 81.924 | 1.00 | 37.66 |
| 1022 | CZ | PHE | 134 | −3.951 | 50.700 | 82.117 | 1.00 | 44.21 |
| 1023 | C | PHE | 134 | −7.824 | 51.775 | 76.849 | 1.00 | 61.24 |
| 1024 | O | PHE | 134 | −7.065 | 51.910 | 75.883 | 1.00 | 57.59 |
| 1025 | N | GLN | 135 | −9.140 | 51.975 | 76.750 | 1.00 | 68.86 |
| 1026 | CA | GLN | 135 | −9.730 | 52.396 | 75.486 | 1.00 | 64.84 |
| 1027 | CB | GLN | 135 | −10.956 | 51.548 | 75.155 | 1.00 | 65.15 |
| 1028 | CG | GLN | 135 | −11.246 | 51.477 | 73.652 | 1.00 | 64.67 |
| 1029 | CD | GLN | 135 | −12.469 | 50.643 | 73.311 | 1.00 | 63.72 |
| 1030 | OE1 | GLN | 135 | −12.694 | 50.303 | 72.148 | 1.00 | 68.97 |
| 1031 | NE2 | GLN | 135 | −13.271 | 50.314 | 74.324 | 1.00 | 73.32 |
| 1032 | C | GLN | 135 | −10.105 | 53.865 | 75.549 | 1.00 | 62.68 |
| 1033 | O | GLN | 135 | −9.313 | 54.689 | 76.010 | 1.00 | 72.04 |
| 1034 | N | ASN | 136 | −11.303 | 54.200 | 75.079 | 0.00 | 68.19 |
| 1035 | CA | ASN | 136 | −11.775 | 55.581 | 75.103 | 0.00 | 68.93 |
| 1036 | CB | ASN | 136 | −12.631 | 55.877 | 73.870 | 0.00 | 69.87 |
| 1037 | CG | ASN | 136 | −11.866 | 55.696 | 72.573 | 0.00 | 70.23 |
| 1038 | OD1 | ASN | 136 | −10.857 | 56.360 | 72.335 | 0.00 | 70.48 |
| 1039 | ND2 | ASN | 136 | −12.346 | 54.794 | 71.725 | 0.00 | 70.48 |
| 1040 | C | ASN | 136 | −12.597 | 55.787 | 76.370 | 0.00 | 69.66 |
| 1041 | O | ASN | 136 | −13.076 | 56.886 | 76.650 | 0.00 | 69.81 |
| 1042 | N | SER | 137 | −12.752 | 54.705 | 77.125 | 0.00 | 69.87 |
| 1043 | CA | SER | 137 | −13.495 | 54.713 | 78.379 | 0.00 | 69.97 |
| 1044 | CB | SER | 137 | −14.644 | 53.705 | 78.311 | 0.00 | 70.20 |
| 1045 | OG | SER | 137 | −14.158 | 52.400 | 78.044 | 0.00 | 70.46 |
| 1046 | C | SER | 137 | −12.521 | 54.320 | 79.485 | 0.00 | 69.72 |
| 1047 | O | SER | 137 | −11.395 | 53.909 | 79.204 | 0.00 | 69.90 |
| 1048 | N | PRO | 138 | −12.934 | 54.438 | 80.756 | 0.00 | 69.29 |
| 1049 | CD | PRO | 138 | −14.238 | 54.851 | 81.300 | 0.00 | 69.40 |
| 1050 | CA | PRO | 138 | −12.013 | 54.065 | 81.834 | 0.00 | 68.42 |
| 1051 | CB | PRO | 138 | −12.832 | 54.329 | 83.098 | 0.00 | 69.04 |
| 1052 | CG | PRO | 138 | −14.250 | 54.157 | 82.637 | 0.00 | 69.33 |
| 1053 | C | PRO | 138 | −11.510 | 52.623 | 81.735 | 0.00 | 67.00 |
| 1054 | O | PRO | 138 | −12.240 | 51.723 | 81.318 | 0.00 | 67.59 |
| 1055 | N | ALA | 139 | −10.252 | 52.427 | 82.118 | 1.00 | 63.13 |
| 1056 | CA | ALA | 139 | −9.592 | 51.120 | 82.085 | 1.00 | 59.19 |
| 1057 | CB | ALA | 139 | −8.335 | 51.173 | 82.931 | 1.00 | 63.39 |
| 1058 | C | ALA | 139 | −10.454 | 49.943 | 82.539 | 1.00 | 62.09 |
| 1059 | O | ALA | 139 | −10.708 | 49.775 | 83.729 | 1.00 | 67.13 |
| 1060 | N | GLU | 140 | −10.894 | 49.111 | 81.608 | 1.00 | 60.46 |
| 1061 | CA | GLU | 140 | −11.717 | 47.976 | 81.996 | 1.00 | 64.65 |
| 1062 | CB | GLU | 140 | −12.777 | 47.688 | 80.928 | 1.00 | 71.73 |
| 1063 | CG | GLU | 140 | −13.723 | 46.521 | 81.281 | 1.00 | 82.73 |
| 1064 | CD | GLU | 140 | −14.586 | 46.070 | 80.092 | 1.00 | 92.27 |
| 1065 | OE1 | GLU | 140 | −14.009 | 45.757 | 79.018 | 1.00 | 88.04 |
| 1066 | OE2 | GLU | 140 | −15.834 | 46.022 | 80.232 | 1.00 | 82.56 |
| 1067 | C | GLU | 140 | −10.861 | 46.727 | 82.230 | 1.00 | 65.14 |
| 1068 | O | GLU | 140 | −10.036 | 46.360 | 81.394 | 1.00 | 66.10 |
| 1069 | N | ASP | 141 | −11.058 | 46.099 | 83.384 | 1.00 | 50.75 |
| 1070 | CA | ASP | 141 | −10.355 | 44.882 | 83.771 | 1.00 | 55.14 |
| 1071 | CB | ASP | 141 | −10.067 | 44.891 | 85.265 | 1.00 | 50.52 |
| 1072 | CG | ASP | 141 | −8.791 | 45.611 | 85.610 | 1.00 | 62.66 |
| 1073 | OD1 | ASP | 141 | −8.187 | 46.242 | 84.723 | 1.00 | 71.61 |
| 1074 | OD2 | ASP | 141 | −8.384 | 45.543 | 86.781 | 1.00 | 74.87 |
| 1075 | C | ASP | 141 | −11.190 | 43.646 | 83.475 | 1.00 | 53.62 |
| 1076 | O | ASP | 141 | −12.409 | 43.698 | 83.508 | 1.00 | 69.30 |
| 1077 | N | PHE | 142 | −10.543 | 42.535 | 83.159 | 1.00 | 52.10 |
| 1078 | CA | PHE | 142 | −11.271 | 41.299 | 82.948 | 1.00 | 41.64 |
| 1079 | CB | PHE | 142 | −12.312 | 41.436 | 81.805 | 1.00 | 46.98 |
| 1080 | CG | PHE | 142 | −11.736 | 41.515 | 80.419 | 1.00 | 47.60 |
| 1081 | CD1 | PHE | 142 | −11.598 | 40.371 | 79.645 | 1.00 | 51.87 |
| 1082 | CD2 | PHE | 142 | −11.375 | 42.736 | 79.873 | 1.00 | 48.50 |
| 1083 | CE1 | PHE | 142 | −11.103 | 40.446 | 78.328 | 1.00 | 58.30 |
| 1084 | CE2 | PHE | 142 | −10.885 | 42.820 | 78.569 | 1.00 | 48.21 |
| 1085 | CZ | PHE | 142 | −10.751 | 41.672 | 77.797 | 1.00 | 48.26 |
| 1086 | C | PHE | 142 | −10.341 | 40.103 | 82.766 | 1.00 | 46.75 |
| 1087 | O | PHE | 142 | −9.119 | 40.259 | 82.704 | 1.00 | 49.75 |
| 1088 | N | GLN | 143 | −10.914 | 38.910 | 82.701 | 1.00 | 41.28 |
| 1089 | CA | GLN | 143 | −10.118 | 37.711 | 82.577 | 1.00 | 42.26 |
| 1090 | CB | GLN | 143 | −10.265 | 36.836 | 83.803 | 1.00 | 38.91 |
| 1091 | CG | GLN | 143 | −9.970 | 37.523 | 85.115 | 1.00 | 57.41 |
| 1092 | CD | GLN | 143 | −10.011 | 36.544 | 86.257 | 1.00 | 59.52 |
| 1093 | OE1 | GLN | 143 | −10.380 | 35.382 | 86.077 | 1.00 | 49.80 |
| 1094 | NE2 | GLN | 143 | −9.633 | 37.000 | 87.433 | 1.00 | 54.07 |
| 1095 | C | GLN | 143 | −10.466 | 36.873 | 81.399 | 1.00 | 46.34 |
| 1096 | O | GLN | 143 | −11.574 | 36.906 | 80.907 | 1.00 | 63.36 |
| 1097 | N | GLU | 144 | −9.492 | 36.103 | 80.958 | 1.00 | 46.31 |
| 1098 | CA | GLU | 144 | −9.665 | 35.203 | 79.843 | 1.00 | 49.21 |
| 1099 | CB | GLU | 144 | −8.801 | 35.670 | 78.658 | 1.00 | 53.60 |
| 1100 | CG | GLU | 144 | −9.256 | 36.942 | 77.965 | 1.00 | 41.81 |
| 1101 | CD | GLU | 144 | −10.606 | 36.782 | 77.314 | 1.00 | 50.94 |
| 1102 | OE1 | GLU | 144 | −10.979 | 35.631 | 76.993 | 1.00 | 50.03 |
| 1103 | OE2 | GLU | 144 | −11.298 | 37.806 | 77.112 | 1.00 | 54.72 |
| 1104 | C | GLU | 144 | −9.129 | 33.885 | 80.380 | 1.00 | 50.35 |
| 1105 | O | GLU | 144 | −8.056 | 33.849 | 80.972 | 1.00 | 47.59 |
| 1106 | N | PRO | 145 | −9.853 | 32.782 | 80.186 | 1.00 | 51.51 |
| 1107 | CD | PRO | 145 | −11.164 | 32.547 | 79.555 | 1.00 | 53.00 |
| 1108 | CA | PRO | 145 | −9.282 | 31.544 | 80.728 | 1.00 | 51.96 |
| 1109 | CB | PRO | 145 | −10.450 | 30.558 | 80.630 | 1.00 | 58.73 |
| 1110 | CG | PRO | 145 | −11.190 | 31.046 | 79.407 | 1.00 | 51.68 |
| 1111 | C | PRO | 145 | −8.049 | 31.084 | 79.948 | 1.00 | 53.19 |
| 1112 | O | PRO | 145 | −7.731 | 31.633 | 78.887 | 1.00 | 55.41 |
| 1113 | N | CYS | 146 | −7.341 | 30.097 | 80.486 | 1.00 | 47.54 |
| 1114 | CA | CYS | 146 | −6.160 | 29.561 | 79.813 | 1.00 | 50.29 |
| 1115 | C | CYS | 146 | −6.248 | 28.070 | 79.929 | 1.00 | 35.56 |
| 1116 | O | CYS | 146 | −6.528 | 27.564 | 80.970 | 1.00 | 44.36 |
| 1117 | CB | CYS | 146 | −4.855 | 30.016 | 80.470 | 1.00 | 42.18 |
| 1118 | SG | CYS | 146 | −4.705 | 31.802 | 80.581 | 1.00 | 55.51 |
| 1119 | N | GLN | 147 | −5.952 | 27.365 | 78.859 | 1.00 | 39.89 |
| 1120 | CA | GLN | 147 | −6.033 | 25.939 | 78.886 | 1.00 | 42.60 |
| 1121 | CB | GLN | 147 | −6.885 | 25.466 | 77.702 | 1.00 | 47.04 |
| 1122 | CG | GLN | 147 | −8.226 | 26.235 | 77.563 | 1.00 | 56.19 |
| 1123 | CD | GLN | 147 | −8.085 | 27.631 | 76.903 | 1.00 | 60.33 |
| 1124 | OE1 | GLN | 147 | −8.648 | 28.614 | 77.382 | 1.00 | 66.53 |
| 1125 | NE2 | GLN | 147 | −7.349 | 27.704 | 75.794 | 1.00 | 59.18 |
| 1126 | C | GLN | 147 | −4.659 | 25.332 | 78.842 | 1.00 | 44.77 |
| 1127 | O | GLN | 147 | −3.749 | 25.863 | 78.194 | 1.00 | 50.02 |
| 1128 | N | TYR | 148 | −4.481 | 24.237 | 79.570 | 1.00 | 52.73 |
| 1129 | CA | TYR | 148 | −3.201 | 23.578 | 79.532 | 1.00 | 45.08 |
| 1130 | CB | TYR | 148 | −2.898 | 22.767 | 80.768 | 1.00 | 36.59 |
| 1131 | CG | TYR | 148 | −1.477 | 22.297 | 80.721 | 1.00 | 43.75 |
| 1132 | CD1 | TYR | 148 | −0.425 | 23.211 | 80.736 | 1.00 | 39.07 |
| 1133 | CE1 | TYR | 148 | 0.892 | 22.791 | 80.628 | 1.00 | 55.87 |
| 1134 | CD2 | TYR | 148 | −1.170 | 20.944 | 80.602 | 1.00 | 48.15 |
| 1135 | CE2 | TYR | 148 | 0.147 | 20.511 | 80.503 | 1.00 | 32.87 |
| 1136 | CZ | TYR | 148 | 1.168 | 21.434 | 80.508 | 1.00 | 51.39 |
| 1137 | OH | TYR | 148 | 2.457 | 21.017 | 80.324 | 1.00 | 50.55 |
| 1138 | C | TYR | 148 | −3.297 | 22.654 | 78.369 | 1.00 | 52.84 |
| 1139 | O | TYR | 148 | −4.294 | 21.978 | 78.185 | 1.00 | 63.18 |
| 1140 | N | SER | 149 | −2.259 | 22.649 | 77.557 | 1.00 | 64.15 |
| 1141 | CA | SER | 149 | −2.240 | 21.815 | 76.388 | 1.00 | 57.56 |
| 1142 | CB | SER | 149 | −1.826 | 22.635 | 75.174 | 1.00 | 50.84 |
| 1143 | OG | SER | 149 | −1.218 | 21.798 | 74.208 | 1.00 | 70.89 |
| 1144 | C | SER | 149 | −1.254 | 20.699 | 76.639 | 1.00 | 68.42 |
| 1145 | O | SER | 149 | −0.030 | 20.927 | 76.751 | 1.00 | 56.21 |
| 1146 | N | GLN | 150 | −1.797 | 19.489 | 76.763 | 1.00 | 70.67 |
| 1147 | CA | GLN | 150 | −0.970 | 18.316 | 76.977 | 1.00 | 70.90 |
| 1148 | CB | GLN | 150 | −1.844 | 17.077 | 77.109 | 1.00 | 68.03 |
| 1149 | CG | GLN | 150 | −2.503 | 16.982 | 78.473 | 1.00 | 76.85 |
| 1150 | CD | GLN | 150 | −1.916 | 15.871 | 79.304 | 1.00 | 75.14 |
| 1151 | OE1 | GLN | 150 | −2.272 | 14.714 | 79.121 | 1.00 | 86.99 |
| 1152 | NE2 | GLN | 150 | −0.999 | 16.208 | 80.204 | 1.00 | 68.25 |
| 1153 | C | GLN | 150 | 0.004 | 18.181 | 75.819 | 1.00 | 66.79 |
| 1154 | O | GLN | 150 | 1.174 | 17.881 | 76.043 | 1.00 | 68.07 |
| 1155 | N | GLU | 151 | −0.469 | 18.431 | 74.593 | 1.00 | 65.24 |
| 1156 | CA | GLU | 151 | 0.401 | 18.350 | 73.415 | 1.00 | 66.37 |
| 1157 | CB | GLU | 151 | −0.339 | 18.750 | 72.100 | 1.00 | 71.26 |
| 1158 | CG | GLU | 151 | −1.182 | 17.635 | 71.421 | 1.00 | 72.87 |
| 1159 | CD | GLU | 151 | −1.203 | 17.699 | 69.866 | 1.00 | 83.21 |
| 1160 | OE1 | GLU | 151 | −0.180 | 18.104 | 69.257 | 1.00 | 85.87 |
| 1161 | OE2 | GLU | 151 | −2.228 | 17.309 | 69.244 | 1.00 | 66.61 |
| 1162 | C | GLU | 151 | 1.596 | 19.276 | 73.639 | 1.00 | 69.20 |
| 1163 | O | GLU | 151 | 2.685 | 18.802 | 73.982 | 1.00 | 61.03 |
| 1164 | N | SER | 152 | 1.379 | 20.586 | 73.464 | 1.00 | 59.32 |
| 1165 | CA | SER | 152 | 2.430 | 21.591 | 73.625 | 1.00 | 61.34 |
| 1166 | CB | SER | 152 | 1.923 | 22.973 | 73.231 | 1.00 | 64.13 |
| 1167 | OG | SER | 152 | 0.965 | 23.447 | 74.163 | 1.00 | 63.70 |
| 1168 | C | SER | 152 | 3.042 | 21.718 | 75.016 | 1.00 | 55.29 |
| 1169 | O | SER | 152 | 4.142 | 22.244 | 75.145 | 1.00 | 55.79 |
| 1170 | N | GLN | 153 | 2.366 | 21.234 | 76.055 | 1.00 | 49.35 |
| 1171 | CA | GLN | 153 | 2.929 | 21.369 | 77.410 | 1.00 | 59.77 |
| 1172 | CB | GLN | 153 | 4.349 | 20.746 | 77.481 | 1.00 | 58.60 |
| 1173 | CG | GLN | 153 | 4.417 | 19.258 | 77.866 | 1.00 | 62.84 |
| 1174 | CD | GLN | 153 | 4.113 | 18.922 | 79.363 | 1.00 | 68.70 |
| 1175 | OE1 | GLN | 153 | 4.907 | 19.341 | 80.240 | 1.00 | 78.04 |
| 1176 | NE2 | GLN | 153 | 2.966 | 18.369 | 79.647 | 1.00 | 66.89 |
| 1177 | C | GLN | 153 | 3.024 | 22.868 | 77.779 | 1.00 | 51.81 |
| 1178 | O | GLN | 153 | 4.016 | 23.291 | 78.365 | 1.00 | 54.37 |
| 1179 | N | LYS | 154 | 2.004 | 23.647 | 77.404 | 1.00 | 46.16 |
| 1180 | CA | LYS | 154 | 1.927 | 25.096 | 77.653 | 1.00 | 50.57 |
| 1181 | CB | LYS | 154 | 2.285 | 25.885 | 76.412 | 1.00 | 38.45 |
| 1182 | CG | LYS | 154 | 3.742 | 25.904 | 76.020 | 1.00 | 54.11 |
| 1183 | CD | LYS | 154 | 3.830 | 26.551 | 74.641 | 1.00 | 48.62 |
| 1184 | CE | LYS | 154 | 5.224 | 26.547 | 74.082 | 1.00 | 41.06 |
| 1185 | NZ | LYS | 154 | 5.217 | 26.886 | 72.613 | 1.00 | 57.60 |
| 1186 | C | LYS | 154 | 0.541 | 25.596 | 78.074 | 1.00 | 54.92 |
| 1187 | O | LYS | 154 | −0.498 | 25.024 | 77.709 | 1.00 | 43.66 |
| 1188 | N | PHE | 155 | 0.531 | 26.664 | 78.867 | 1.00 | 48.03 |
| 1189 | CA | PHE | 155 | −0.732 | 27.261 | 79.242 | 1.00 | 39.54 |
| 1190 | CB | PHE | 155 | −0.627 | 28.007 | 80.573 | 1.00 | 41.40 |
| 1191 | CG | PHE | 155 | −0.731 | 27.117 | 81.784 | 1.00 | 36.68 |
| 1192 | CD1 | PHE | 155 | 0.413 | 26.753 | 82.510 | 1.00 | 41.11 |
| 1193 | CD2 | PHE | 155 | −1.965 | 26.652 | 82.209 | 1.00 | 33.06 |
| 1194 | CE1 | PHE | 155 | 0.319 | 25.935 | 83.644 | 1.00 | 39.67 |
| 1195 | CE2 | PHE | 155 | −2.071 | 25.827 | 83.358 | 1.00 | 36.90 |
| 1196 | CZ | PHE | 155 | −0.931 | 25.476 | 84.063 | 1.00 | 44.92 |
| 1197 | C | PHE | 155 | −0.966 | 28.230 | 78.078 | 1.00 | 39.92 |
| 1198 | O | PHE | 155 | −0.031 | 28.860 | 77.617 | 1.00 | 37.12 |
| 1199 | N | SER | 156 | −2.201 | 28.327 | 77.592 | 1.00 | 36.69 |
| 1200 | CA | SER | 156 | −2.485 | 29.176 | 76.449 | 1.00 | 37.61 |
| 1201 | CB | SER | 156 | −2.478 | 28.316 | 75.181 | 1.00 | 38.42 |
| 1202 | OG | SER | 156 | −2.566 | 29.095 | 74.009 | 1.00 | 46.67 |
| 1203 | C | SER | 156 | −3.833 | 29.875 | 76.596 | 1.00 | 44.98 |
| 1204 | O | SER | 156 | −4.796 | 29.274 | 76.987 | 1.00 | 39.16 |
| 1205 | N | CYS | 157 | −3.883 | 31.157 | 76.264 | 1.00 | 43.71 |
| 1206 | CA | CYS | 157 | −5.101 | 31.926 | 76.397 | 1.00 | 41.17 |
| 1207 | C | CYS | 157 | −5.282 | 32.799 | 75.179 | 1.00 | 39.87 |
| 1208 | O | CYS | 157 | −4.339 | 33.126 | 74.495 | 1.00 | 42.77 |
| 1209 | CB | CYS | 157 | −5.054 | 32.833 | 77.651 | 1.00 | 38.54 |
| 1210 | SG | CYS | 157 | −3.759 | 32.411 | 78.866 | 1.00 | 54.87 |
| 1211 | N | GLN | 158 | −6.517 | 33.186 | 74.917 | 1.00 | 43.33 |
| 1212 | CA | GLN | 158 | −6.763 | 34.041 | 73.798 | 1.00 | 42.10 |
| 1213 | CB | GLN | 158 | −7.571 | 33.309 | 72.749 | 1.00 | 47.26 |
| 1214 | CG | GLN | 158 | −6.812 | 32.192 | 72.145 | 1.00 | 42.30 |
| 1215 | CD | GLN | 158 | −7.489 | 31.668 | 70.929 | 1.00 | 56.10 |
| 1216 | OE1 | GLN | 158 | −8.161 | 32.416 | 70.193 | 1.00 | 48.87 |
| 1217 | NE2 | GLN | 158 | −7.311 | 30.377 | 70.683 | 1.00 | 51.80 |
| 1218 | C | GLN | 158 | −7.469 | 35.285 | 74.199 | 1.00 | 38.01 |
| 1219 | O | GLN | 158 | −8.276 | 35.305 | 75.114 | 1.00 | 48.28 |
| 1220 | N | LEU | 159 | −7.156 | 36.345 | 73.495 | 1.00 | 42.09 |
| 1221 | CA | LEU | 159 | −7.798 | 37.611 | 73.754 | 1.00 | 42.11 |
| 1222 | CB | LEU | 159 | −6.796 | 38.545 | 74.392 | 1.00 | 31.90 |
| 1223 | CG | LEU | 159 | −7.072 | 40.048 | 74.459 | 1.00 | 30.38 |
| 1224 | CD1 | LEU | 159 | −8.292 | 40.319 | 75.321 | 1.00 | 35.27 |
| 1225 | CD2 | LEU | 159 | −5.838 | 40.723 | 75.049 | 1.00 | 27.23 |
| 1226 | C | LEU | 159 | −8.224 | 38.084 | 72.359 | 1.00 | 46.03 |
| 1227 | O | LEU | 159 | −7.427 | 38.089 | 71.416 | 1.00 | 41.40 |
| 1228 | N | ALA | 160 | −9.501 | 38.413 | 72.227 | 1.00 | 40.78 |
| 1229 | CA | ALA | 160 | −10.059 | 38.881 | 70.960 | 1.00 | 49.80 |
| 1230 | CB | ALA | 160 | −11.578 | 38.612 | 70.910 | 1.00 | 32.43 |
| 1231 | C | ALA | 160 | −9.808 | 40.373 | 70.849 | 1.00 | 46.69 |
| 1232 | O | ALA | 160 | −10.298 | 41.132 | 71.658 | 1.00 | 50.91 |
| 1233 | N | VAL | 161 | −9.012 | 40.795 | 69.875 | 1.00 | 53.33 |
| 1234 | CA | VAL | 161 | −8.778 | 42.222 | 69.699 | 1.00 | 49.12 |
| 1235 | CB | VAL | 161 | −7.277 | 42.541 | 69.673 | 1.00 | 49.30 |
| 1236 | CG1 | VAL | 161 | −7.061 | 44.008 | 69.398 | 1.00 | 48.16 |
| 1237 | CG2 | VAL | 161 | −6.640 | 42.156 | 71 | 1.00 | 50.62 |
| 1238 | C | VAL | 161 | −9.434 | 42.586 | 68.367 | 1.00 | 54.33 |
| 1239 | O | VAL | 161 | −9.015 | 42.102 | 67.330 | 1.00 | 63.24 |
| 1240 | N | PRO | 162 | −10.509 | 43.400 | 68.389 | 1.00 | 59.46 |
| 1241 | CD | PRO | 162 | −11.089 | 44.145 | 69.521 | 1.00 | 48.50 |
| 1242 | CA | PRO | 162 | −11.173 | 43.777 | 67.137 | 1.00 | 55.33 |
| 1243 | CB | PRO | 162 | −12.288 | 44.713 | 67.606 | 1.00 | 54.21 |
| 1244 | CG | PRO | 162 | −11.710 | 45.327 | 68.836 | 1.00 | 50.74 |
| 1245 | C | PRO | 162 | −10.177 | 44.479 | 66.246 | 1.00 | 52.41 |
| 1246 | O | PRO | 162 | −9.202 | 45.017 | 66.753 | 1.00 | 50.05 |
| 1247 | N | GLU | 163 | −10.406 | 44.459 | 64.931 | 1.00 | 54.02 |
| 1248 | CA | GLU | 163 | −9.487 | 45.119 | 64.023 | 1.00 | 51.65 |
| 1249 | CB | GLU | 163 | −9.740 | 44.683 | 62.586 | 1.00 | 61.56 |
| 1250 | CG | GLU | 163 | −8.554 | 43.905 | 61.944 | 1.00 | 77.44 |
| 1251 | CD | GLU | 163 | −7.148 | 44.459 | 62.304 | 1.00 | 77.96 |
| 1252 | OE1 | GLU | 163 | −6.616 | 44.107 | 63.387 | 1.00 | 79.06 |
| 1253 | OE2 | GLU | 163 | −6.575 | 45.237 | 61.504 | 1.00 | 72.23 |
| 1254 | C | GLU | 163 | −9.549 | 46.656 | 64.138 | 1.00 | 57.77 |
| 1255 | O | GLU | 163 | −10.599 | 47.234 | 64.424 | 1.00 | 43.25 |
| 1256 | N | GLY | 164 | −8.399 | 47.299 | 63.935 | 1.00 | 49.64 |
| 1257 | CA | GLY | 164 | −8.318 | 48.739 | 64.050 | 1.00 | 59.59 |
| 1258 | C | GLY | 164 | −8.357 | 49.208 | 65.501 | 1.00 | 64.82 |
| 1259 | O | GLY | 164 | −8.383 | 50.417 | 65.766 | 1.00 | 63.60 |
| 1260 | N | ASP | 165 | −8.360 | 48.262 | 66.442 | 1.00 | 52.82 |
| 1261 | CA | ASP | 165 | −8.414 | 48.592 | 67.869 | 1.00 | 49.58 |
| 1262 | CB | ASP | 165 | −8.510 | 47.288 | 68.654 | 1.00 | 56.63 |
| 1263 | CG | ASP | 165 | −8.918 | 47.486 | 70.094 | 1.00 | 66.56 |
| 1264 | OD1 | ASP | 165 | −8.697 | 48.589 | 70.640 | 1.00 | 71.82 |
| 1265 | OD2 | ASP | 165 | −9.451 | 46.519 | 70.684 | 1.00 | 53.01 |
| 1266 | C | ASP | 165 | −7.171 | 49.405 | 68.303 | 1.00 | 52.81 |
| 1267 | O | ASP | 165 | −6.039 | 48.937 | 68.187 | 1.00 | 56.02 |
| 1268 | N | SER | 166 | −7.374 | 50.629 | 68.774 | 1.00 | 52.02 |
| 1269 | CA | SER | 166 | −6.258 | 51.457 | 69.223 | 1.00 | 51.68 |
| 1270 | CB | SER | 166 | −6.481 | 52.908 | 68.839 | 1.00 | 54.84 |
| 1271 | OG | SER | 166 | −6.563 | 53.028 | 67.434 | 1.00 | 82.35 |
| 1272 | C | SER | 166 | −6.091 | 51.392 | 70.735 | 1.00 | 51.79 |
| 1273 | O | SER | 166 | −5.357 | 52.188 | 71.320 | 1.00 | 44.25 |
| 1274 | N | SER | 167 | −6.798 | 50.480 | 71.385 | 1.00 | 37.58 |
| 1275 | CA | SER | 167 | −6.656 | 50.376 | 72.831 | 1.00 | 52.11 |
| 1276 | CB | SER | 167 | −7.723 | 49.466 | 73.438 | 1.00 | 39.52 |
| 1277 | OG | SER | 167 | −8.979 | 49.629 | 72.827 | 1.00 | 52.25 |
| 1278 | C | SER | 167 | −5.290 | 49.780 | 73.171 | 1.00 | 48.80 |
| 1279 | O | SER | 167 | −4.653 | 49.129 | 72.354 | 1.00 | 47.94 |
| 1280 | N | PHE | 168 | −4.834 | 50.051 | 74.376 | 1.00 | 43.43 |
| 1281 | CA | PHE | 168 | −3.611 | 49.469 | 74.837 | 1.00 | 44.42 |
| 1282 | CB | PHE | 168 | −2.726 | 50.478 | 75.539 | 1.00 | 26.37 |
| 1283 | CG | PHE | 168 | −1.993 | 51.335 | 74.585 | 1.00 | 39.66 |
| 1284 | CD1 | PHE | 168 | −2.609 | 52.449 | 74.013 | 1.00 | 33.60 |
| 1285 | CD2 | PHE | 168 | −0.710 | 50.993 | 74.181 | 1.00 | 29.87 |
| 1286 | CE1 | PHE | 168 | −1.952 | 53.193 | 73.058 | 1.00 | 40.75 |
| 1287 | CE2 | PHE | 168 | −0.662 | 51.728 | 73.227 | 1.00 | 41.69 |
| 1288 | CZ | PHE | 168 | −0.048 | 52.834 | 72.656 | 1.00 | 35.65 |
| 1289 | C | PHE | 168 | −4.085 | 48.407 | 75.764 | 1.00 | 43.72 |
| 1290 | O | PHE | 168 | −5.201 | 48.466 | 76.299 | 1.00 | 44.37 |
| 1291 | N | TYR | 169 | −3.257 | 47.392 | 75.901 | 1.00 | 49.15 |
| 1292 | CA | TYR | 169 | −3.600 | 46.297 | 76.760 | 1.00 | 35.79 |
| 1293 | CB | TYR | 169 | −3.977 | 45.071 | 75.962 | 1.00 | 40.55 |
| 1294 | CG | TYR | 169 | −5.251 | 45.231 | 75.195 | 1.00 | 45.83 |
| 1295 | CD1 | TYR | 169 | −5.270 | 45.855 | 73.940 | 1.00 | 48.25 |
| 1296 | CE1 | TYR | 169 | −6.449 | 45.939 | 73.198 | 1.00 | 44.96 |
| 1297 | CD2 | TYR | 169 | −6.437 | 44.717 | 75.683 | 1.00 | 50.32 |
| 1298 | CE2 | TYR | 169 | −7.617 | 44.811 | 74.948 | 1.00 | 49.48 |
| 1299 | CZ | TYR | 169 | −7.610 | 45.416 | 73.714 | 1.00 | 44.53 |
| 1300 | OH | TYR | 169 | −8.772 | 45.485 | 73.009 | 1.00 | 47.89 |
| 1301 | C | TYR | 169 | −2.425 | 45.991 | 77.615 | 1.00 | 34.14 |
| 1302 | O | TYR | 169 | −1.277 | 46.140 | 77.181 | 1.00 | 36.21 |
| 1303 | N | ILE | 170 | −2.722 | 45.664 | 78.868 | 1.00 | 33.46 |
| 1304 | CA | ILE | 170 | −1.700 | 45.239 | 79.798 | 1.00 | 38.42 |
| 1305 | CB | ILE | 170 | −1.575 | 46.166 | 83 | 1.00 | 39.43 |
| 1306 | CG2 | ILE | 170 | −0.486 | 45.607 | 81.944 | 1.00 | 27.43 |
| 1307 | CG1 | ILE | 170 | −1.223 | 47.585 | 80.520 | 1.00 | 38.20 |
| 1308 | CD1 | ILE | 170 | −0.933 | 48.572 | 81.630 | 1.00 | 36.44 |
| 1309 | C | ILE | 170 | −2.138 | 43.830 | 80.238 | 1.00 | 41.74 |
| 1310 | O | ILE | 170 | −3.236 | 43.636 | 80.743 | 1.00 | 42.03 |
| 1311 | N | VAL | 171 | −1.301 | 42.837 | 79.984 | 1.00 | 42.79 |
| 1312 | CA | VAL | 171 | −1.640 | 41.478 | 80.370 | 1.00 | 33.58 |
| 1313 | CB | VAL | 171 | −1.806 | 40.518 | 79.129 | 1.00 | 32.57 |
| 1314 | CG1 | VAL | 171 | −2.967 | 40 | 78.246 | 1.00 | 43.01 |
| 1315 | CG2 | VAL | 171 | −0.546 | 40.458 | 78.313 | 1.00 | 33.95 |
| 1316 | C | VAL | 171 | −0.614 | 40.870 | 81.299 | 1.00 | 39.22 |
| 1317 | O | VAL | 171 | 0.564 | 41.213 | 81.275 | 1.00 | 45.36 |
| 1318 | N | SER | 172 | −1.088 | 39.944 | 82.107 | 1.00 | 37.48 |
| 1319 | CA | SER | 172 | −0.268 | 39.228 | 83.046 | 1.00 | 37.92 |
| 1320 | CB | SER | 172 | −0.201 | 39.996 | 84.371 | 1.00 | 37.35 |
| 1321 | OG | SER | 172 | 0.809 | 39.443 | 85.188 | 1.00 | 51.10 |
| 1322 | C | SER | 172 | −0.943 | 37.870 | 83.279 | 1.00 | 30.61 |
| 1323 | O | SER | 172 | −2.160 | 37.768 | 83.340 | 1.00 | 40.90 |
| 1324 | N | MET | 173 | −0.171 | 36.826 | 83.431 | 1.00 | 35.71 |
| 1325 | CA | MET | 173 | −0.813 | 35.538 | 83.664 | 1.00 | 37.99 |
| 1326 | CB | MET | 173 | −0.225 | 34.460 | 82.761 | 1.00 | 34.90 |
| 1327 | CG | MET | 173 | −0.813 | 33.070 | 82.966 | 1.00 | 39.85 |
| 1328 | SD | MET | 173 | 0.141 | 31.885 | 81.981 | 1.00 | 46.54 |
| 1329 | CE | MET | 173 | −0.497 | 32.252 | 80.338 | 1.00 | 27.07 |
| 1330 | C | MET | 173 | −0.625 | 35.155 | 85.119 | 1.00 | 40.81 |
| 1331 | O | MET | 173 | 0.469 | 35.239 | 85.689 | 1.00 | 41.35 |
| 1332 | N | CYS | 174 | −1.731 | 34.779 | 85.721 | 1.00 | 33.01 |
| 1333 | CA | CYS | 174 | −1.739 | 34.337 | 87.073 | 1.00 | 36.17 |
| 1334 | C | CYS | 174 | −1.865 | 32.815 | 87.036 | 1.00 | 44.26 |
| 1335 | O | CYS | 174 | −2.745 | 32.290 | 86.353 | 1.00 | 55.09 |
| 1336 | CB | CYS | 174 | −2.938 | 34.930 | 87.784 | 1.00 | 35.89 |
| 1337 | SG | CYS | 174 | −3.295 | 34.104 | 89.345 | 1.00 | 62.53 |
| 1338 | N | VAL | 175 | −0.966 | 32.112 | 87.730 | 1.00 | 45.18 |
| 1339 | CA | VAL | 175 | −1.048 | 30.665 | 87.835 | 1.00 | 43.88 |
| 1340 | CB | VAL | 175 | 0.141 | 29.950 | 87.245 | 1.00 | 51.65 |
| 1341 | CG1 | VAL | 175 | 0.026 | 28.484 | 87.545 | 1.00 | 45.86 |
| 1342 | CG2 | VAL | 175 | 0.149 | 30.131 | 85.738 | 1.00 | 69.66 |
| 1343 | C | VAL | 175 | −1.126 | 30.344 | 89.309 | 1.00 | 53.01 |
| 1344 | O | VAL | 175 | −0.357 | 30.888 | 90.103 | 1.00 | 50.61 |
| 1345 | N | ALA | 176 | −2.071 | 29.474 | 89.670 | 1.00 | 49.03 |
| 1346 | CA | ALA | 176 | −2.274 | 29.109 | 91.052 | 1.00 | 36.27 |
| 1347 | CB | ALA | 176 | −3.389 | 29.900 | 91.635 | 1.00 | 49.89 |
| 1348 | C | ALA | 176 | −2.547 | 27.651 | 91.252 | 1.00 | 46.90 |
| 1349 | O | ALA | 176 | −3.153 | 27.000 | 90.414 | 1.00 | 44.96 |
| 1350 | N | SER | 177 | −2.076 | 27.141 | 92.384 | 1.00 | 39.29 |
| 1351 | CA | SER | 177 | −2.287 | 25.754 | 92.749 | 1.00 | 46.65 |
| 1352 | CB | SER | 177 | −0.944 | 25.023 | 92.833 | 1.00 | 45.22 |
| 1353 | OG | SER | 177 | −0.080 | 25.607 | 93.798 | 1.00 | 50.99 |
| 1354 | C | SER | 177 | −2.944 | 25.926 | 94.109 | 1.00 | 44.29 |
| 1355 | O | SER | 177 | −3.227 | 27.059 | 94.494 | 1.00 | 47.84 |
| 1356 | N | SER | 178 | −3.204 | 24.845 | 94.843 | 1.00 | 49.38 |
| 1357 | CA | SER | 178 | −3.848 | 24.995 | 96.157 | 1.00 | 34.08 |
| 1358 | CB | SER | 178 | −4.442 | 23.672 | 96.613 | 1.00 | 46.73 |
| 1359 | OG | SER | 178 | −3.499 | 22.624 | 96.456 | 1.00 | 61.09 |
| 1360 | C | SER | 178 | −2.912 | 25.507 | 97.218 | 1.00 | 39.70 |
| 1361 | O | SER | 178 | −3.363 | 25.840 | 98.315 | 1.00 | 60.42 |
| 1362 | N | VAL | 179 | −1.612 | 25.580 | 96.906 | 1.00 | 43.01 |
| 1363 | CA | VAL | 179 | −0.619 | 26.058 | 97.871 | 1.00 | 42.31 |
| 1364 | CB | VAL | 179 | 0.462 | 24.951 | 98.212 | 1.00 | 52.49 |
| 1365 | CG1 | VAL | 179 | −0.181 | 23.794 | 98.949 | 1.00 | 44.01 |
| 1366 | CG2 | VAL | 179 | 1.199 | 24.498 | 96.926 | 1.00 | 29.17 |
| 1367 | C | VAL | 179 | 0.164 | 27.323 | 97.465 | 1.00 | 52.42 |
| 1368 | O | VAL | 179 | 1.211 | 27.612 | 98.045 | 1.00 | 48.69 |
| 1369 | N | GLY | 180 | −0.336 | 28.068 | 96.483 | 1.00 | 56.29 |
| 1370 | CA | GLY | 180 | 0.362 | 29.264 | 96.045 | 1.00 | 47.34 |
| 1371 | C | GLY | 180 | −0.076 | 29.770 | 94.675 | 1.00 | 47.02 |
| 1372 | O | GLY | 180 | −0.829 | 29.147 | 93.953 | 1.00 | 45.51 |
| 1373 | N | SER | 181 | 0.392 | 30.945 | 94.315 | 1.00 | 48.02 |
| 1374 | CA | SER | 181 | 0.025 | 31.501 | 93.044 | 1.00 | 40.20 |
| 1375 | CB | SER | 181 | −1.288 | 32.260 | 93.158 | 1.00 | 50.45 |
| 1376 | OG | SER | 181 | −1.191 | 33.233 | 94.170 | 1.00 | 46.34 |
| 1377 | C | SER | 181 | 1.137 | 32.428 | 92.650 | 1.00 | 37.49 |
| 1378 | O | SER | 181 | 1.888 | 32.861 | 93.491 | 1.00 | 39.35 |
| 1379 | N | LYS | 182 | 1.282 | 32.665 | 91.357 | 1.00 | 30.89 |
| 1380 | CA | LYS | 182 | 2.277 | 33.589 | 90.879 | 1.00 | 36.58 |
| 1381 | CB | LYS | 182 | 3.560 | 32.875 | 90.442 | 1.00 | 39.17 |
| 1382 | CG | LYS | 182 | 4.349 | 32.187 | 91.527 | 1.00 | 27.02 |
| 1383 | CD | LYS | 182 | 4.792 | 33.111 | 92.613 | 1.00 | 38.02 |
| 1384 | CE | LYS | 182 | 6.160 | 32.643 | 93.148 | 1.00 | 56.92 |
| 1385 | NZ | LYS | 182 | 6.890 | 33.625 | 94.008 | 1.00 | 44.06 |
| 1386 | C | LYS | 182 | 1.703 | 34.364 | 89.693 | 1.00 | 39.37 |
| 1387 | O | LYS | 182 | 0.736 | 33.959 | 89.068 | 1.00 | 40.66 |
| 1388 | N | PHE | 183 | 2.295 | 35.498 | 89.383 | 1.00 | 40.26 |
| 1389 | CA | PHE | 183 | 1.817 | 36.230 | 88.249 | 1.00 | 31.06 |
| 1390 | CB | PHE | 183 | 0.968 | 37.436 | 88.660 | 1.00 | 37.20 |
| 1391 | CG | PHE | 183 | 1.624 | 38.358 | 89.641 | 1.00 | 52.97 |
| 1392 | CD1 | PHE | 183 | 2.659 | 39.246 | 89.229 | 1.00 | 52.23 |
| 1393 | CD2 | PHE | 183 | 1.200 | 38.376 | 90.980 | 1.00 | 44.37 |
| 1394 | CE1 | PHE | 183 | 3.271 | 40.146 | 90.146 | 1.00 | 49.03 |
| 1395 | CE2 | PHE | 183 | 1.806 | 39.284 | 91.917 | 1.00 | 49.53 |
| 1396 | CZ | PHE | 183 | 2.835 | 40.156 | 91.489 | 1.00 | 55.76 |
| 1397 | C | PHE | 183 | 3.049 | 36.611 | 87.543 | 1.00 | 32.62 |
| 1398 | O | PHE | 183 | 4.101 | 36.769 | 88.168 | 1.00 | 37.47 |
| 1399 | N | SER | 184 | 2.942 | 36.701 | 86.229 | 1.00 | 26.17 |
| 1400 | CA | SER | 184 | 4.073 | 37.060 | 85.434 | 1.00 | 23.09 |
| 1401 | CB | SER | 184 | 3.871 | 36.597 | 84.017 | 1.00 | 28.43 |
| 1402 | OG | SER | 184 | 2.610 | 37.018 | 83.528 | 1.00 | 33.08 |
| 1403 | C | SER | 184 | 4.224 | 38.543 | 85.370 | 1.00 | 32.48 |
| 1404 | O | SER | 184 | 3.338 | 39.286 | 85.771 | 1.00 | 30.51 |
| 1405 | N | LYS | 185 | 5.370 | 38.937 | 84.827 | 1.00 | 31.37 |
| 1406 | CA | LYS | 185 | 5.657 | 40.311 | 84.502 | 1.00 | 37.36 |
| 1407 | CB | LYS | 185 | 7.026 | 40.443 | 83.854 | 1.00 | 30.26 |
| 1408 | CG | LYS | 185 | 8.167 | 40.252 | 84.835 | 1.00 | 41.57 |
| 1409 | CD | LYS | 185 | 9.511 | 40.599 | 84.201 | 1.00 | 39.51 |
| 1410 | CE | LYS | 185 | 10.637 | 40.269 | 85.175 | 1.00 | 52.19 |
| 1411 | NZ | LYS | 185 | 11.990 | 40.715 | 84.679 | 1.00 | 66.48 |
| 1412 | C | LYS | 185 | 4.609 | 40.622 | 83.448 | 1.00 | 31.96 |
| 1413 | O | LYS | 185 | 4.127 | 39.750 | 82.717 | 1.00 | 40.83 |
| 1414 | N | THR | 186 | 4.269 | 41.883 | 83.370 | 1.00 | 48.13 |
| 1415 | CA | THR | 186 | 3.274 | 42.397 | 82.444 | 1.00 | 34.03 |
| 1416 | CB | THR | 186 | 2.989 | 43.837 | 82.866 | 1.00 | 31.33 |
| 1417 | OG1 | THR | 186 | 1.953 | 43.837 | 83.875 | 1.00 | 45.68 |
| 1418 | CG2 | THR | 186 | 2.636 | 44.661 | 81.736 | 1.00 | 44.07 |
| 1419 | C | THR | 186 | 3.746 | 42.344 | 80.983 | 1.00 | 38.66 |
| 1420 | O | THR | 186 | 4.939 | 42.358 | 80.683 | 1.00 | 28.83 |
| 1421 | N | GLN | 187 | 2.785 | 42.262 | 80.087 | 1.00 | 33.99 |
| 1422 | CA | GLN | 187 | 3.045 | 42.301 | 78.665 | 1.00 | 41.07 |
| 1423 | CB | GLN | 187 | 2.809 | 40.907 | 78.085 | 1.00 | 33.31 |
| 1424 | CG | GLN | 187 | 3.059 | 40.793 | 76.626 | 1.00 | 58.84 |
| 1425 | CD | GLN | 187 | 4.452 | 40.323 | 76.306 | 1.00 | 59.15 |
| 1426 | OE1 | GLN | 187 | 4.723 | 39.904 | 75.161 | 1.00 | 66.55 |
| 1427 | NE2 | GLN | 187 | 5.354 | 40.379 | 77.299 | 1.00 | 43.86 |
| 1428 | C | GLN | 187 | 2.050 | 43.377 | 78.143 | 1.00 | 33.77 |
| 1429 | O | GLN | 187 | 0.844 | 43.227 | 78.283 | 1.00 | 38.55 |
| 1430 | N | THR | 188 | 2.589 | 44.495 | 77.640 | 1.00 | 42.84 |
| 1431 | CA | THR | 188 | 1.830 | 45.653 | 77.120 | 1.00 | 29.85 |
| 1432 | CB | THR | 188 | 2.329 | 47.011 | 77.634 | 1.00 | 31.08 |
| 1433 | OG1 | THR | 188 | 2.608 | 46.953 | 79.030 | 1.00 | 40.81 |
| 1434 | CG2 | THR | 188 | 1.264 | 48.086 | 77.362 | 1.00 | 27.81 |
| 1435 | C | THR | 188 | 1.890 | 45.835 | 75.621 | 1.00 | 30.99 |
| 1436 | O | THR | 188 | 2.927 | 45.686 | 75.014 | 1.00 | 52.50 |
| 1437 | N | PHE | 189 | 0.785 | 46.225 | 75.034 | 1.00 | 32.54 |
| 1438 | CA | PHE | 189 | 0.725 | 46.477 | 73.603 | 1.00 | 31.88 |
| 1439 | CB | PHE | 189 | 0.906 | 45.165 | 72.852 | 1.00 | 31.77 |
| 1440 | CG | PHE | 189 | −0.094 | 44.168 | 73.196 | 1.00 | 43.97 |
| 1441 | CD1 | PHE | 189 | −1.348 | 44.207 | 72.606 | 1.00 | 46.62 |
| 1442 | CD2 | PHE | 189 | 0.148 | 43.251 | 74.214 | 1.00 | 44.39 |
| 1443 | CE1 | PHE | 189 | −2.349 | 43.361 | 73.028 | 1.00 | 37.55 |
| 1444 | CE2 | PHE | 189 | −0.871 | 42.399 | 74.639 | 1.00 | 35.33 |
| 1445 | CZ | PHE | 189 | −2.105 | 42.460 | 74.051 | 1.00 | 34.37 |
| 1446 | C | PHE | 189 | −0.570 | 47.161 | 73.114 | 1.00 | 37.61 |
| 1447 | O | PHE | 189 | −1.638 | 47.111 | 73.730 | 1.00 | 30.14 |
| 1448 | N | GLN | 190 | −0.446 | 47.826 | 71.989 | 1.00 | 37.99 |
| 1449 | CA | GLN | 190 | −1.594 | 48.448 | 71.374 | 1.00 | 40.93 |
| 1450 | CB | GLN | 190 | −1.166 | 49.533 | 70.428 | 1.00 | 46.59 |
| 1451 | CG | GLN | 190 | −2.311 | 50.255 | 69.887 | 1.00 | 45.55 |
| 1452 | CD | GLN | 190 | −1.906 | 51.602 | 69.461 | 1.00 | 52.98 |
| 1453 | OE1 | GLN | 190 | −0.892 | 51.760 | 68.767 | 1.00 | 53.30 |
| 1454 | NE2 | GLN | 190 | −2.685 | 52.604 | 69.857 | 1.00 | 50.89 |
| 1455 | C | GLN | 190 | −2.224 | 47.349 | 70.577 | 1.00 | 42.26 |
| 1456 | O | GLN | 190 | −1.511 | 46.560 | 69.990 | 1.00 | 43.02 |
| 1457 | N | GLY | 191 | −3.555 | 47.290 | 70.566 | 1.00 | 56.59 |
| 1458 | CA | GLY | 191 | −4.257 | 46.239 | 69.853 | 1.00 | 45.60 |
| 1459 | C | GLY | 191 | −3.694 | 45.989 | 68.471 | 1.00 | 63.13 |
| 1460 | O | GLY | 191 | −3.495 | 44.845 | 68.071 | 1.00 | 52.46 |
| 1461 | N | CYS | 192 | −3.423 | 47.072 | 67.746 | 1.00 | 66.67 |
| 1462 | CA | CYS | 192 | −2.895 | 46.991 | 66.379 | 1.00 | 76.68 |
| 1463 | C | CYS | 192 | −1.402 | 47.218 | 66.259 | 1.00 | 72.10 |
| 1464 | O | CYS | 192 | −0.973 | 48.059 | 65.469 | 1.00 | 79.18 |
| 1465 | CB | CYS | 192 | −3.607 | 48.009 | 65.494 | 1.00 | 80.63 |
| 1466 | SG | CYS | 192 | −4.015 | 49.532 | 66.393 | 1.00 | 88.30 |
| 1467 | N | GLY | 193 | −0.619 | 46.466 | 67.033 | 1.00 | 72.36 |
| 1468 | CA | GLY | 193 | 0.826 | 46.600 | 67.006 | 1.00 | 54.45 |
| 1469 | C | GLY | 193 | 1.541 | 45.308 | 67.351 | 1.00 | 58.63 |
| 1470 | O | GLY | 193 | 2.739 | 45.159 | 67.084 | 1.00 | 53.13 |
| 1471 | N | ILE | 194 | 0.815 | 44.358 | 67.930 | 1.00 | 52.04 |
| 1472 | CA | ILE | 194 | 1.443 | 43.118 | 68.334 | 1.00 | 44.38 |
| 1473 | CB | ILE | 194 | 0.660 | 42.447 | 69.514 | 1.00 | 45.56 |
| 1474 | CG2 | ILE | 194 | −0.668 | 41.928 | 69.057 | 1.00 | 36.30 |
| 1475 | CG1 | ILE | 194 | 1.491 | 41.309 | 70.131 | 1.00 | 41.08 |
| 1476 | CD1 | ILE | 194 | 0.873 | 40.741 | 71.341 | 1.00 | 39.86 |
| 1477 | C | ILE | 194 | 1.639 | 42.144 | 67.187 | 1.00 | 46.45 |
| 1478 | O | ILE | 194 | 2.532 | 41.295 | 67.248 | 1.00 | 43.39 |
| 1479 | N | LEU | 195 | 0.841 | 42.292 | 66.131 | 1.00 | 41.08 |
| 1480 | CA | LEU | 195 | 0.931 | 41.424 | 64.956 | 1.00 | 30.61 |
| 1481 | CB | LEU | 195 | 0.126 | 42.025 | 63.773 | 1.00 | 26.45 |
| 1482 | CG | LEU | 195 | 0.171 | 41.279 | 62.420 | 1.00 | 23.92 |
| 1483 | CD1 | LEU | 195 | −0.518 | 39.951 | 62.532 | 1.00 | 27.31 |
| 1484 | CD2 | LEU | 195 | −0.439 | 42.055 | 61.359 | 1.00 | 23.89 |
| 1485 | C | LEU | 195 | 2.342 | 41.118 | 64.471 | 1.00 | 34.62 |
| 1486 | O | LEU | 195 | 3.142 | 42.012 | 64.232 | 1.00 | 35.89 |
| 1487 | N | GLN | 196 | 2.641 | 39.835 | 64.350 | 1.00 | 28.59 |
| 1488 | CA | GLN | 196 | 3.906 | 39.372 | 63.785 | 1.00 | 32.44 |
| 1489 | CB | GLN | 196 | 4.984 | 39.080 | 64.837 | 1.00 | 33.23 |
| 1490 | CG | GLN | 196 | 6.380 | 39.055 | 64.163 | 1.00 | 43.56 |
| 1491 | CD | GLN | 196 | 7.492 | 38.359 | 64.960 | 1.00 | 38.91 |
| 1492 | OE1 | GLN | 196 | 8.683 | 38.477 | 64.630 | 1.00 | 39.55 |
| 1493 | NE2 | GLN | 196 | 7.111 | 37.628 | 65.984 | 1.00 | 35.47 |
| 1494 | C | GLN | 196 | 3.658 | 38.105 | 62.934 | 1.00 | 32.49 |
| 1495 | O | GLN | 196 | 3.368 | 37.028 | 63.449 | 1.00 | 34.28 |
| 1496 | N | PRO | 197 | 3.753 | 38.229 | 61.610 | 1.00 | 28.02 |
| 1497 | CD | PRO | 197 | 4.130 | 39.402 | 60.806 | 1.00 | 34.21 |
| 1498 | CA | PRO | 197 | 3.520 | 37.048 | 60.772 | 1.00 | 27.67 |
| 1499 | CB | PRO | 197 | 3.506 | 37.611 | 59.347 | 1.00 | 27.89 |
| 1500 | CG | PRO | 197 | 3.479 | 39.110 | 59.484 | 1.00 | 24.68 |
| 1501 | C | PRO | 197 | 4.636 | 36.024 | 60.936 | 1.00 | 33.52 |
| 1502 | O | PRO | 197 | 5.714 | 36.383 | 61.349 | 1.00 | 30.59 |
| 1503 | N | ASP | 198 | 4.361 | 34.749 | 60.648 | 1.00 | 34.23 |
| 1504 | CA | ASP | 198 | 5.428 | 33.742 | 60.672 | 1.00 | 26.24 |
| 1505 | CB | ASP | 198 | 4.901 | 32.320 | 60.479 | 1.00 | 37.69 |
| 1506 | CG | ASP | 198 | 4.397 | 31.706 | 61.769 | 1.00 | 45.16 |
| 1507 | OD1 | ASP | 198 | 5.129 | 31.767 | 62.785 | 1.00 | 45.51 |
| 1508 | OD2 | ASP | 198 | 3.273 | 31.158 | 61.758 | 1.00 | 48.53 |
| 1509 | C | ASP | 198 | 6.306 | 34.056 | 59.464 | 1.00 | 37.68 |
| 1510 | O | ASP | 198 | 5.867 | 34.839 | 58.546 | 1.00 | 33.16 |
| 1511 | N | PRO | 199 | 7.550 | 33.467 | 59.422 | 1.00 | 38.92 |
| 1512 | CD | PRO | 199 | 8.095 | 32.414 | 60.293 | 1.00 | 28.03 |
| 1513 | CA | PRO | 199 | 8.495 | 33.684 | 58.318 | 1.00 | 29.63 |
| 1514 | CB | PRO | 199 | 9.707 | 32.845 | 58.720 | 1.00 | 33.41 |
| 1515 | CG | PRO | 199 | 9.560 | 32.688 | 60.204 | 1.00 | 34.88 |
| 1516 | C | PRO | 199 | 7.927 | 33.222 | 56.982 | 1.00 | 34.71 |
| 1517 | O | PRO | 199 | 7.012 | 32.361 | 56.909 | 1.00 | 36.65 |
| 1518 | N | PRO | 200 | 8.390 | 33.849 | 55.914 | 1.00 | 32.63 |
| 1519 | CD | PRO | 200 | 8.776 | 35.264 | 55.871 | 1.00 | 34.81 |
| 1520 | CA | PRO | 200 | 7.880 | 33.421 | 54.624 | 1.00 | 30.35 |
| 1521 | CB | PRO | 200 | 8.668 | 34.278 | 53.655 | 1.00 | 29.67 |
| 1522 | CG | PRO | 200 | 8.665 | 35.599 | 54.351 | 1.00 | 34.13 |
| 1523 | C | PRO | 200 | 8.119 | 31.927 | 54.512 | 1.00 | 28.47 |
| 1524 | O | PRO | 200 | 8.986 | 31.404 | 55.155 | 1.00 | 34.08 |
| 1525 | N | ALA | 201 | 7.314 | 31.242 | 53.715 | 1.00 | 41.16 |
| 1526 | CA | ALA | 201 | 7.433 | 29.807 | 53.592 | 1.00 | 42.80 |
| 1527 | CB | ALA | 201 | 6.098 | 29.185 | 53.927 | 1.00 | 40.91 |
| 1528 | C | ALA | 201 | 7.913 | 29.308 | 52.224 | 1.00 | 47.51 |
| 1529 | O | ALA | 201 | 7.742 | 29.980 | 51.202 | 1.00 | 42.15 |
| 1530 | N | ASN | 202 | 8.480 | 28.103 | 52.250 | 1.00 | 42.07 |
| 1531 | CA | ASN | 202 | 9.036 | 27.379 | 51.104 | 1.00 | 46.41 |
| 1532 | CB | ASN | 202 | 7.947 | 26.604 | 50.389 | 1.00 | 53.30 |
| 1533 | CG | ASN | 202 | 7.276 | 25.590 | 51.295 | 1.00 | 68.86 |
| 1534 | OD1 | ASN | 202 | 7.928 | 24.941 | 52.123 | 1.00 | 65.65 |
| 1535 | ND2 | ASN | 202 | 5.968 | 25.450 | 51.136 | 1.00 | 67.11 |
| 1536 | C | ASN | 202 | 9.819 | 28.216 | 50.118 | 1.00 | 44.72 |
| 1537 | O | ASN | 202 | 9.415 | 28.390 | 48.964 | 1.00 | 49.52 |
| 1538 | N | ILE | 203 | 10.951 | 28.717 | 50.599 | 1.00 | 39.53 |
| 1539 | CA | ILE | 203 | 11.852 | 29.558 | 49.830 | 1.00 | 39.99 |
| 1540 | CB | ILE | 203 | 12.959 | 30.178 | 50.703 | 1.00 | 41.37 |
| 1541 | CG2 | ILE | 203 | 13.777 | 31.140 | 49.852 | 1.00 | 40.07 |
| 1542 | CG1 | ILE | 203 | 12.398 | 30.772 | 52.009 | 1.00 | 46.01 |
| 1543 | CD1 | ILE | 203 | 11.450 | 31.894 | 51.864 | 1.00 | 57.61 |
| 1544 | C | ILE | 203 | 12.620 | 28.713 | 48.843 | 1.00 | 47.66 |
| 1545 | O | ILE | 203 | 13.333 | 27.811 | 49.265 | 1.00 | 48.78 |
| 1546 | N | THR | 204 | 12.504 | 28.985 | 47.546 | 1.00 | 37.38 |
| 1547 | CA | THR | 204 | 13.319 | 28.224 | 46.610 | 1.00 | 39.50 |
| 1548 | CB | THR | 204 | 12.543 | 27.530 | 45.456 | 1.00 | 49.71 |
| 1549 | OG1 | THR | 204 | 11.879 | 28.528 | 44.680 | 1.00 | 54.83 |
| 1550 | CG2 | THR | 204 | 11.585 | 26.500 | 45.969 | 1.00 | 38.24 |
| 1551 | C | THR | 204 | 14.225 | 29.207 | 45.939 | 1.00 | 40.01 |
| 1552 | O | THR | 204 | 13.813 | 30.326 | 45.637 | 1.00 | 41.74 |
| 1553 | N | VAL | 205 | 15.467 | 28.791 | 45.716 | 1.00 | 36.98 |
| 1554 | CA | VAL | 205 | 16.436 | 29.638 | 45.042 | 1.00 | 41.75 |
| 1555 | CB | VAL | 205 | 17.700 | 29.866 | 45.875 | 1.00 | 39.67 |
| 1556 | CG1 | VAL | 205 | 18.618 | 30.869 | 45.160 | 1.00 | 37.36 |
| 1557 | CG2 | VAL | 205 | 17.325 | 30.360 | 47.241 | 1.00 | 30.87 |
| 1558 | C | VAL | 205 | 16.765 | 28.789 | 43.844 | 1.00 | 48.46 |
| 1559 | O | VAL | 205 | 17.029 | 27.584 | 43.977 | 1.00 | 52.22 |
| 1560 | N | THR | 206 | 16.758 | 29.403 | 42.672 | 1.00 | 40.56 |
| 1561 | CA | THR | 206 | 16.970 | 28.627 | 41.466 | 1.00 | 48.05 |
| 1562 | CB | THR | 206 | 15.601 | 28.237 | 40.876 | 1.00 | 39.58 |
| 1563 | OG1 | THR | 206 | 14.936 | 27.414 | 41.826 | 1.00 | 48.82 |
| 1564 | CG2 | THR | 206 | 15.725 | 27.491 | 39.592 | 1.00 | 53.89 |
| 1565 | C | THR | 206 | 17.782 | 29.344 | 40.440 | 1.00 | 45.98 |
| 1566 | O | THR | 206 | 17.539 | 30.515 | 40.164 | 1.00 | 48.97 |
| 1567 | N | ALA | 207 | 18.745 | 28.622 | 39.873 | 1.00 | 43.24 |
| 1568 | CA | ALA | 207 | 19.593 | 29.188 | 38.845 | 1.00 | 45.85 |
| 1569 | CB | ALA | 207 | 20.833 | 28.294 | 38.614 | 1.00 | 42.68 |
| 1570 | C | ALA | 207 | 18.813 | 29.373 | 37.533 | 1.00 | 47.75 |
| 1571 | O | ALA | 207 | 17.964 | 28.562 | 37.164 | 1.00 | 41.36 |
| 1572 | N | VAL | 208 | 19.094 | 30.476 | 36.859 | 1.00 | 39.02 |
| 1573 | CA | VAL | 208 | 18.482 | 30.764 | 35.591 | 1.00 | 41.83 |
| 1574 | CB | VAL | 208 | 18.011 | 32.197 | 35.527 | 1.00 | 35.72 |
| 1575 | CG1 | VAL | 208 | 17.245 | 32.390 | 34.252 | 1.00 | 26.28 |
| 1576 | CG2 | VAL | 208 | 17.201 | 32.526 | 36.741 | 1.00 | 43.47 |
| 1577 | C | VAL | 208 | 19.519 | 30.543 | 34.469 | 1.00 | 41.28 |
| 1578 | O | VAL | 208 | 20.438 | 31.329 | 34.296 | 1.00 | 43.82 |
| 1579 | N | ALA | 209 | 19.345 | 29.453 | 33.732 | 1.00 | 47.12 |
| 1580 | CA | ALA | 209 | 20.182 | 29.061 | 32.599 | 1.00 | 33.47 |
| 1581 | CB | ALA | 209 | 19.393 | 28.125 | 31.699 | 1.00 | 26.18 |
| 1582 | C | ALA | 209 | 20.708 | 30.209 | 31.752 | 1.00 | 45.92 |
| 1583 | O | ALA | 209 | 19.943 | 31.106 | 31.333 | 1.00 | 33.82 |
| 1584 | N | ARG | 210 | 22.017 | 30.167 | 31.495 | 1.00 | 37.98 |
| 1585 | CA | ARG | 210 | 22.675 | 31.162 | 30.643 | 1.00 | 39.92 |
| 1586 | CB | ARG | 210 | 21.961 | 31.224 | 29.271 | 1.00 | 42.71 |
| 1587 | CG | ARG | 210 | 21.982 | 29.885 | 28.443 | 1.00 | 35.37 |
| 1588 | CD | ARG | 210 | 20.870 | 29.842 | 27.286 | 1.00 | 50.76 |
| 1589 | NE | ARG | 210 | 22 | 30.890 | 26.258 | 1.00 | 53.73 |
| 1590 | CZ | ARG | 210 | 19.986 | 31.423 | 25.561 | 1.00 | 62.22 |
| 1591 | NH1 | ARG | 210 | 18.748 | 31.026 | 25.779 | 1.00 | 56.79 |
| 1592 | NH2 | ARG | 210 | 20.200 | 32.342 | 24.609 | 1.00 | 59.59 |
| 1593 | C | ARG | 210 | 22.832 | 32.573 | 31.209 | 1.00 | 40.24 |
| 1594 | O | ARG | 210 | 23.208 | 33.493 | 30.488 | 1.00 | 49.75 |
| 1595 | N | ASN | 211 | 22.553 | 32.747 | 32.492 | 1.00 | 45.65 |
| 1596 | CA | ASN | 211 | 22.669 | 34.057 | 33.155 | 1.00 | 45.30 |
| 1597 | CB | ASN | 211 | 21.274 | 34.584 | 33.482 | 1.00 | 43.53 |
| 1598 | CG | ASN | 211 | 20.573 | 35.114 | 32.272 | 1.00 | 33.16 |
| 1599 | OD1 | ASN | 211 | 20.691 | 36.298 | 31.933 | 1.00 | 50.54 |
| 1600 | ND2 | ASN | 211 | 19.856 | 34.253 | 31.596 | 1.00 | 38.92 |
| 1601 | C | ASN | 211 | 23.503 | 33.845 | 34.425 | 1.00 | 46.66 |
| 1602 | O | ASN | 211 | 22.980 | 33.628 | 35.513 | 1.00 | 33.69 |
| 1603 | N | PRO | 212 | 24.829 | 33.902 | 34.271 | 1.00 | 45.45 |
| 1604 | CD | PRO | 212 | 25.541 | 34.441 | 33.095 | 1.00 | 44.03 |
| 1605 | CA | PRO | 212 | 25.754 | 33.697 | 35.376 | 1.00 | 48.12 |
| 1606 | CB | PRO | 212 | 27.119 | 34.034 | 34.756 | 1.00 | 42.31 |
| 1607 | CG | PRO | 212 | 26.809 | 34.992 | 33.712 | 1.00 | 33.99 |
| 1608 | C | PRO | 212 | 25.503 | 34.434 | 36.670 | 1.00 | 53.40 |
| 1609 | O | PRO | 212 | 25.941 | 33.962 | 37.738 | 1.00 | 60.10 |
| 1610 | N | ARG | 213 | 24.774 | 35.547 | 36.614 | 1.00 | 36.45 |
| 1611 | CA | ARG | 213 | 24.574 | 36.304 | 37.841 | 1.00 | 44.53 |
| 1612 | CB | ARG | 213 | 25.090 | 37.717 | 37.590 | 1.00 | 47.17 |
| 1613 | CG | ARG | 213 | 26.587 | 37.730 | 37.230 | 1.00 | 43.48 |
| 1614 | CD | ARG | 213 | 27.361 | 38.347 | 38.353 | 1.00 | 52.40 |
| 1615 | NE | ARG | 213 | 27.296 | 39.803 | 38.293 | 1.00 | 55.09 |
| 1616 | CZ | ARG | 213 | 27.627 | 40.621 | 39.293 | 1.00 | 54.96 |
| 1617 | NH1 | ARG | 213 | 28.047 | 40.143 | 40.456 | 1.00 | 51.54 |
| 1618 | NH2 | ARG | 213 | 27.548 | 41.930 | 39.118 | 1.00 | 54.51 |
| 1619 | C | ARG | 213 | 23.155 | 36.312 | 38.433 | 1.00 | 51.87 |
| 1620 | O | ARG | 213 | 22.833 | 37.086 | 39.339 | 1.00 | 58.98 |
| 1621 | N | TRP | 214 | 22.322 | 35.405 | 37.951 | 1.00 | 43.12 |
| 1622 | CA | TRP | 214 | 20.951 | 35.340 | 38.381 | 1.00 | 37.74 |
| 1623 | CB | TRP | 214 | 20.049 | 35.270 | 37.156 | 1.00 | 38.75 |
| 1624 | CG | TRP | 214 | 20.039 | 36.503 | 36.368 | 1.00 | 29.79 |
| 1625 | CD2 | TRP | 214 | 19.055 | 36.894 | 35.414 | 1.00 | 37.85 |
| 1626 | CE2 | TRP | 214 | 19.519 | 38.076 | 34.802 | 1.00 | 34.15 |
| 1627 | CE3 | TRP | 214 | 17.822 | 36.359 | 35.013 | 1.00 | 49.41 |
| 1628 | CD1 | TRP | 214 | 21.017 | 37.432 | 36.316 | 1.00 | 30.36 |
| 1629 | NE1 | TRP | 214 | 20.723 | 38.379 | 35.377 | 1.00 | 44.13 |
| 1630 | CZ2 | TRP | 214 | 18.796 | 38.747 | 33.798 | 1.00 | 46.25 |
| 1631 | CZ3 | TRP | 214 | 17.099 | 37.020 | 34.017 | 1.00 | 53.33 |
| 1632 | CH2 | TRP | 214 | 17.595 | 38.205 | 33.419 | 1.00 | 50.73 |
| 1633 | C | TRP | 214 | 20.578 | 34.220 | 39.313 | 1.00 | 37.36 |
| 1634 | O | TRP | 214 | 21.148 | 33.132 | 39.249 | 1.00 | 51.38 |
| 1635 | N | LEU | 215 | 19.629 | 34.511 | 40.205 | 1.00 | 34.14 |
| 1636 | CA | LEU | 215 | 19.069 | 33.514 | 41.121 | 1.00 | 27.35 |
| 1637 | CB | LEU | 215 | 19.672 | 33.673 | 42.493 | 1.00 | 34.73 |
| 1638 | CG | LEU | 215 | 21.083 | 33.104 | 42.662 | 1.00 | 38.60 |
| 1639 | CD1 | LEU | 215 | 21.593 | 33.430 | 44.065 | 1.00 | 43.95 |
| 1640 | CD2 | LEU | 215 | 21.032 | 31.607 | 42.479 | 1.00 | 34.00 |
| 1641 | C | LEU | 215 | 17.544 | 33.791 | 41.137 | 1.00 | 36.60 |
| 1642 | O | LEU | 215 | 17.114 | 34.923 | 41.389 | 1.00 | 38.55 |
| 1643 | N | SER | 216 | 16.726 | 32.795 | 40.797 | 1.00 | 40.81 |
| 1644 | CA | SER | 216 | 15.277 | 33.017 | 40.793 | 1.00 | 42.27 |
| 1645 | CB | SER | 216 | 14.574 | 32.232 | 39.695 | 1.00 | 36.64 |
| 1646 | OG | SER | 216 | 13.180 | 32.147 | 39.918 | 1.00 | 41.26 |
| 1647 | C | SER | 216 | 14.750 | 32.577 | 42.134 | 1.00 | 43.67 |
| 1648 | O | SER | 216 | 14.786 | 31.396 | 42.449 | 1.00 | 35.43 |
| 1649 | N | VAL | 217 | 14.228 | 33.527 | 42.911 | 1.00 | 31.29 |
| 1650 | CA | VAL | 217 | 13.733 | 33.180 | 44.247 | 1.00 | 33.24 |
| 1651 | CB | VAL | 217 | 14.420 | 34.068 | 45.355 | 1.00 | 30.91 |
| 1652 | CG1 | VAL | 217 | 14.175 | 33.504 | 46.753 | 1.00 | 40.72 |
| 1653 | CG2 | VAL | 217 | 15.901 | 34.116 | 45.103 | 1.00 | 40.98 |
| 1654 | C | VAL | 217 | 12.229 | 33.323 | 44.326 | 1.00 | 36.87 |
| 1655 | O | VAL | 217 | 11.634 | 34.343 | 43.905 | 1.00 | 37.09 |
| 1656 | N | THR | 218 | 11.621 | 32.279 | 44.861 | 1.00 | 30.79 |
| 1657 | CA | THR | 218 | 10.179 | 32.249 | 45.038 | 1.00 | 36.09 |
| 1658 | CB | THR | 218 | 9.485 | 31.173 | 44.099 | 1.00 | 39.51 |
| 1659 | OG1 | THR | 218 | 9.928 | 29.853 | 44.446 | 1.00 | 36.70 |
| 1660 | CG2 | THR | 218 | 9.818 | 31.444 | 42.594 | 1.00 | 18.81 |
| 1661 | C | THR | 218 | 9.983 | 31.915 | 46.507 | 1.00 | 39.46 |
| 1662 | O | THR | 218 | 10.965 | 31.670 | 47.221 | 1.00 | 39.20 |
| 1663 | N | TRP | 219 | 8.729 | 31.929 | 46.951 | 1.00 | 38.62 |
| 1664 | CA | TRP | 219 | 8.356 | 31.623 | 48.330 | 1.00 | 33.93 |
| 1665 | CB | TRP | 219 | 9.063 | 32.575 | 49.292 | 1.00 | 26.62 |
| 1666 | CG | TRP | 219 | 8.690 | 34.045 | 49.129 | 1.00 | 26.06 |
| 1667 | CD2 | TRP | 219 | 9.364 | 35.030 | 48.316 | 1.00 | 23.67 |
| 1668 | CE2 | TRP | 219 | 8.724 | 36.271 | 48.544 | 1.00 | 32.01 |
| 1669 | CE3 | TRP | 219 | 10.441 | 34.983 | 47.427 | 1.00 | 25.03 |
| 1670 | CD1 | TRP | 219 | 7.697 | 34.711 | 49.785 | 1.00 | 24.37 |
| 1671 | NE1 | TRP | 219 | 7.717 | 36.055 | 49.448 | 1.00 | 34.14 |
| 1672 | CZ2 | TRP | 219 | 9.121 | 37.442 | 47.915 | 1.00 | 23.85 |
| 1673 | CZ3 | TRP | 219 | 10.846 | 36.139 | 46.812 | 1.00 | 23.84 |
| 1674 | CH2 | TRP | 219 | 10.181 | 37.374 | 47.058 | 1.00 | 26.35 |
| 1675 | C | TRP | 219 | 6.850 | 31.837 | 48.457 | 1.00 | 43.62 |
| 1676 | O | TRP | 219 | 6.245 | 32.474 | 47.598 | 1.00 | 44.36 |
| 1677 | N | GLN | 220 | 6.242 | 31.327 | 49.520 | 1.00 | 39.65 |
| 1678 | CA | GLN | 220 | 4.809 | 31.577 | 49.713 | 1.00 | 51.57 |
| 1679 | CB | GLN | 220 | 4.015 | 30.325 | 49.412 | 1.00 | 50.40 |
| 1680 | CG | GLN | 220 | 4.448 | 29.180 | 50.228 | 1.00 | 59.69 |
| 1681 | CD | GLN | 220 | 4.167 | 27.912 | 49.509 | 1.00 | 58.33 |
| 1682 | OE1 | GLN | 220 | 4.044 | 27.915 | 48.291 | 1.00 | 51.98 |
| 1683 | NE2 | GLN | 220 | 4.070 | 26.812 | 50.245 | 1.00 | 52.32 |
| 1684 | C | GLN | 220 | 4.414 | 32.084 | 51.093 | 1.00 | 47.17 |
| 1685 | O | GLN | 220 | 5.190 | 31.978 | 52.049 | 1.00 | 50.98 |
| 1686 | N | ASP | 221 | 3.203 | 32.649 | 51.185 | 1.00 | 57.45 |
| 1687 | CA | ASP | 221 | 2.653 | 33.169 | 52.456 | 1.00 | 42.11 |
| 1688 | CB | ASP | 221 | 1.183 | 33.601 | 52.292 | 1.00 | 44.43 |
| 1689 | CG | ASP | 221 | 1.020 | 35.000 | 51.677 | 1.00 | 46.93 |
| 1690 | OD1 | ASP | 221 | −0.142 | 35.420 | 51.423 | 1.00 | 44.50 |
| 1691 | OD2 | ASP | 221 | 2.036 | 35.691 | 51.453 | 1.00 | 40.84 |
| 1692 | C | ASP | 221 | 2.713 | 32.045 | 53.485 | 1.00 | 36.84 |
| 1693 | O | ASP | 221 | 2.631 | 30.850 | 53.150 | 1.00 | 36.07 |
| 1694 | N | PRO | 222 | 2.913 | 32.399 | 54.760 | 1.00 | 40.08 |
| 1695 | CD | PRO | 222 | 3.421 | 33.621 | 55.394 | 1.00 | 34.72 |
| 1696 | CA | PRO | 222 | 2.951 | 31.293 | 55.707 | 1.00 | 33.91 |
| 1697 | CB | PRO | 222 | 3.539 | 31.930 | 56.950 | 1.00 | 26.83 |
| 1698 | CG | PRO | 222 | 3.168 | 33.296 | 56.829 | 1.00 | 36.91 |
| 1699 | C | PRO | 222 | 1.523 | 30.850 | 55.916 | 1.00 | 43.03 |
| 1700 | O | PRO | 222 | 0.612 | 31.634 | 55.716 | 1.00 | 35.51 |
| 1701 | N | HIS | 223 | 1.363 | 29.586 | 56.284 | 1.00 | 37.13 |
| 1702 | CA | HIS | 223 | 0.086 | 28.977 | 56.581 | 1.00 | 47.60 |
| 1703 | CB | HIS | 223 | 0.315 | 27.592 | 57.202 | 1.00 | 57.92 |
| 1704 | CG | HIS | 223 | −0.023 | 26.465 | 56.284 | 1.00 | 71.35 |
| 1705 | CD2 | HIS | 223 | −0.973 | 25.503 | 56.369 | 1.00 | 75.37 |
| 1706 | ND1 | HIS | 223 | 0.625 | 26.263 | 55.080 | 1.00 | 64.60 |
| 1707 | CE1 | HIS | 223 | 0.092 | 25.224 | 54.465 | 1.00 | 71.90 |
| 1708 | NE2 | HIS | 223 | −0.882 | 24.745 | 55.224 | 1.00 | 88.57 |
| 1709 | C | HIS | 223 | −0.788 | 29.808 | 57.523 | 1.00 | 39.58 |
| 1710 | O | HIS | 223 | −1.939 | 30.078 | 57.211 | 1.00 | 52.25 |
| 1711 | N | SER | 224 | −0.229 | 30.206 | 58.663 | 1.00 | 40.65 |
| 1712 | CA | SER | 224 | −0.947 | 30.983 | 59.661 | 1.00 | 35.59 |
| 1713 | CB | SER | 224 | −0.059 | 31.276 | 60.876 | 1.00 | 33.27 |
| 1714 | OG | SER | 224 | 1.119 | 31.967 | 60.482 | 1.00 | 42.92 |
| 1715 | C | SER | 224 | −1.494 | 32.291 | 59.107 | 1.00 | 38.41 |
| 1716 | O | SER | 224 | −2.399 | 32.884 | 59.692 | 1.00 | 46.86 |
| 1717 | N | TRP | 225 | −0.967 | 32.762 | 57.986 | 1.00 | 39.37 |
| 1718 | CA | TRP | 225 | −1.492 | 34.011 | 57.445 | 1.00 | 26.77 |
| 1719 | CB | TRP | 225 | −0.397 | 34.796 | 56.724 | 1.00 | 28.30 |
| 1720 | CG | TRP | 225 | −0.797 | 36.171 | 56.269 | 1.00 | 33.60 |
| 1721 | CD2 | TRP | 225 | −0.736 | 37.408 | 57.031 | 1.00 | 28.41 |
| 1722 | CE2 | TRP | 225 | −1.119 | 38.449 | 56.164 | 1.00 | 22.11 |
| 1723 | CE3 | TRP | 225 | −0.383 | 37.728 | 58.345 | 1.00 | 27.00 |
| 1724 | CD1 | TRP | 225 | −1.218 | 36.517 | 55.028 | 1.00 | 32.53 |
| 1725 | NE1 | TRP | 225 | −1.410 | 37.882 | 54.952 | 1.00 | 34.65 |
| 1726 | CZ2 | TRP | 225 | −1.149 | 39.784 | 56.559 | 1.00 | 20.87 |
| 1727 | CZ3 | TRP | 225 | −0.408 | 39.063 | 58.735 | 1.00 | 37.63 |
| 1728 | CH2 | TRP | 225 | −0.787 | 40.082 | 57.834 | 1.00 | 23.37 |
| 1729 | C | TRP | 225 | −2.607 | 33.630 | 56.511 | 1.00 | 36.75 |
| 1730 | O | TRP | 225 | −2.393 | 33.490 | 55.311 | 1.00 | 41.44 |
| 1731 | N | ASN | 226 | −3.784 | 33.370 | 57.099 | 1.00 | 50.65 |
| 1732 | CA | ASN | 226 | −4.979 | 33.011 | 56.331 | 1.00 | 45.84 |
| 1733 | CB | ASN | 226 | −5.951 | 32.088 | 57.091 | 1.00 | 43.86 |
| 1734 | CG | ASN | 226 | −5.964 | 32.312 | 58.634 | 1.00 | 44.68 |
| 1735 | OD1 | ASN | 226 | −5.689 | 33.393 | 59.136 | 1.00 | 48.37 |
| 1736 | ND2 | ASN | 226 | −6.323 | 31.276 | 59.377 | 1.00 | 42.39 |
| 1737 | C | ASN | 226 | −5.634 | 34.326 | 55.992 | 1.00 | 53.43 |
| 1738 | O | ASN | 226 | −5.008 | 35.388 | 56.094 | 1.00 | 68.67 |
| 1739 | N | SER | 227 | −6.889 | 34.284 | 55.614 | 1.00 | 54.23 |
| 1740 | CA | SER | 227 | −7.579 | 35.499 | 55.182 | 1.00 | 68.04 |
| 1741 | CB | SER | 227 | −7.634 | 36.569 | 56.269 | 1.00 | 60.18 |
| 1742 | OG | SER | 227 | −8.604 | 37.546 | 55.880 | 1.00 | 64.30 |
| 1743 | C | SER | 227 | −6.880 | 36.088 | 53.953 | 1.00 | 61.23 |
| 1744 | O | SER | 227 | −5.655 | 36.120 | 53.874 | 1.00 | 77.30 |
| 1745 | N | SER | 228 | −7.677 | 36.550 | 52.999 | 1.00 | 57.48 |
| 1746 | CA | SER | 228 | −7.173 | 37.116 | 51.770 | 1.00 | 49.04 |
| 1747 | CB | SER | 228 | −7.985 | 36.598 | 50.602 | 1.00 | 59.59 |
| 1748 | OG | SER | 228 | −9.311 | 37.091 | 50.696 | 1.00 | 75.43 |
| 1749 | C | SER | 228 | −7.342 | 38.607 | 51.862 | 1.00 | 44.40 |
| 1750 | O | SER | 228 | −6.871 | 39.369 | 51.019 | 1.00 | 54.22 |
| 1751 | N | PHE | 229 | −7.994 | 39.035 | 52.917 | 1.00 | 45.52 |
| 1752 | CA | PHE | 229 | −8.221 | 40.458 | 53.102 | 1.00 | 51.10 |
| 1753 | CB | PHE | 229 | −9.374 | 40.642 | 54.097 | 1.00 | 55.23 |
| 1754 | CG | PHE | 229 | −10.653 | 39.974 | 53.647 | 1.00 | 52.41 |
| 1755 | CD1 | PHE | 229 | −11.576 | 39.494 | 54.573 | 1.00 | 64.42 |
| 1756 | CD2 | PHE | 229 | −10.911 | 39.786 | 52.279 | 1.00 | 59.12 |
| 1757 | CE1 | PHE | 229 | −12.755 | 38.821 | 54.151 | 1.00 | 62.79 |
| 1758 | CE2 | PHE | 229 | −12.073 | 39.122 | 51.846 | 1.00 | 71.29 |
| 1759 | CZ | PHE | 229 | −12.998 | 38.637 | 52.789 | 1.00 | 61.72 |
| 1760 | C | PHE | 229 | −6.962 | 41.210 | 53.531 | 1.00 | 51.96 |
| 1761 | O | PHE | 229 | −6.924 | 42.448 | 53.502 | 1.00 | 48.19 |
| 1762 | N | TYR | 230 | −5.924 | 40.472 | 53.926 | 1.00 | 48.37 |
| 1763 | CA | TYR | 230 | −4.679 | 41.132 | 54.340 | 1.00 | 50.08 |
| 1764 | CB | TYR | 230 | −4.451 | 40.989 | 55.860 | 1.00 | 46.47 |
| 1765 | CG | TYR | 230 | −5.475 | 41.729 | 56.691 | 1.00 | 45.87 |
| 1766 | CD1 | TYR | 230 | −6.661 | 41.126 | 57.090 | 1.00 | 36.39 |
| 1767 | CE1 | TYR | 230 | −7.613 | 41.852 | 57.856 | 1.00 | 44.55 |
| 1768 | CD2 | TYR | 230 | −5.259 | 43.054 | 57.052 | 1.00 | 48.88 |
| 1769 | CE2 | TYR | 230 | −6.193 | 43.783 | 57.785 | 1.00 | 31.64 |
| 1770 | CZ | TYR | 230 | −7.357 | 43.186 | 58.201 | 1.00 | 50.47 |
| 1771 | OH | TYR | 230 | −8.196 | 43.914 | 59.042 | 1.00 | 56.40 |
| 1772 | C | TYR | 230 | −3.491 | 40.586 | 53.564 | 1.00 | 36.44 |
| 1773 | O | TYR | 230 | −3.270 | 39.396 | 53.516 | 1.00 | 38.09 |
| 1774 | N | ARG | 231 | −2.745 | 41.466 | 52.926 | 1.00 | 36.84 |
| 1775 | CA | ARG | 231 | −1.593 | 45 | 52.176 | 1.00 | 42.22 |
| 1776 | CB | ARG | 231 | −1.502 | 41.733 | 50.837 | 1.00 | 44.23 |
| 1777 | CG | ARG | 231 | −2.725 | 41.636 | 49.983 | 1.00 | 53.59 |
| 1778 | CD | ARG | 231 | −2.658 | 40.423 | 49.087 | 1.00 | 58.62 |
| 1779 | NE | ARG | 231 | −1.658 | 40.618 | 48.040 | 1.00 | 64.22 |
| 1780 | CZ | ARG | 231 | −1.442 | 39.773 | 47.043 | 1.00 | 57.63 |
| 1781 | NH1 | ARG | 231 | −2.168 | 38.668 | 46.955 | 1.00 | 58.85 |
| 1782 | NH2 | ARG | 231 | −0.492 | 40.025 | 46.145 | 1.00 | 63.77 |
| 1783 | C | ARG | 231 | −0.298 | 41.224 | 52.963 | 1.00 | 36.69 |
| 1784 | O | ARG | 231 | −0.194 | 42.075 | 53.869 | 1.00 | 33.85 |
| 1785 | N | LEU | 232 | 0.693 | 40.450 | 52.576 | 1.00 | 34.25 |
| 1786 | CA | LEU | 232 | 2.026 | 40.521 | 53.152 | 1.00 | 26.86 |
| 1787 | CB | LEU | 232 | 2.521 | 39.114 | 53.394 | 1.00 | 29.55 |
| 1788 | CG | LEU | 232 | 2.770 | 38.605 | 54.810 | 1.00 | 38.27 |
| 1789 | CD1 | LEU | 232 | 1.997 | 39.422 | 55.842 | 1.00 | 26.31 |
| 1790 | CD2 | LEU | 232 | 2.502 | 37.088 | 54.827 | 1.00 | 25.14 |
| 1791 | C | LEU | 232 | 2.950 | 41.230 | 52.165 | 1.00 | 30.03 |
| 1792 | O | LEU | 232 | 2.818 | 41.096 | 50.961 | 1.00 | 48.11 |
| 1793 | N | ARG | 233 | 3.862 | 42.029 | 52.685 | 1.00 | 40.21 |
| 1794 | CA | ARG | 233 | 4.867 | 42.714 | 51.882 | 1.00 | 34.96 |
| 1795 | CB | ARG | 233 | 4.956 | 44.141 | 52.385 | 1.00 | 43.37 |
| 1796 | CG | ARG | 233 | 6.096 | 44.932 | 51.844 | 1.00 | 46.20 |
| 1797 | CD | ARG | 233 | 6.032 | 46.348 | 52.379 | 1.00 | 35.26 |
| 1798 | NE | ARG | 233 | 4.777 | 47.032 | 52.083 | 1.00 | 36.72 |
| 1799 | CZ | ARG | 233 | 4.382 | 47.455 | 50.884 | 1.00 | 40.21 |
| 1800 | NH1 | ARG | 233 | 5.132 | 47.254 | 49.816 | 1.00 | 43.90 |
| 1801 | NH2 | ARG | 233 | 3.263 | 48.172 | 50.769 | 1.00 | 36.59 |
| 1802 | C | ARG | 233 | 6.185 | 41.948 | 52.190 | 1.00 | 38.11 |
| 1803 | O | ARG | 233 | 6.366 | 41.509 | 53.319 | 1.00 | 27.19 |
| 1804 | N | PHE | 234 | 7.107 | 41.762 | 51.244 | 1.00 | 33.42 |
| 1805 | CA | PHE | 234 | 8.325 | 41.037 | 51.645 | 1.00 | 37.96 |
| 1806 | CB | PHE | 234 | 8.435 | 39.685 | 50.946 | 1.00 | 33.16 |
| 1807 | CG | PHE | 234 | 7.262 | 38.797 | 51.149 | 1.00 | 24.95 |
| 1808 | CD1 | PHE | 234 | 6.170 | 38.858 | 50.274 | 1.00 | 37.71 |
| 1809 | CD2 | PHE | 234 | 7.236 | 37.883 | 52.195 | 1.00 | 33.93 |
| 1810 | CE1 | PHE | 234 | 5.089 | 38.012 | 50.437 | 1.00 | 24.30 |
| 1811 | CE2 | PHE | 234 | 6.145 | 37.021 | 52.375 | 1.00 | 33.01 |
| 1812 | CZ | PHE | 234 | 5.067 | 37.081 | 51.489 | 1.00 | 22.44 |
| 1813 | C | PHE | 234 | 9.710 | 41.706 | 51.548 | 1.00 | 44.48 |
| 1814 | O | PHE | 234 | 9.951 | 42.654 | 50.807 | 1.00 | 39.31 |
| 1815 | N | GLU | 235 | 10.637 | 41.191 | 52.330 | 1.00 | 38.10 |
| 1816 | CA | GLU | 235 | 11.974 | 41.733 | 52.277 | 1.00 | 32.97 |
| 1817 | CB | GLU | 235 | 12.295 | 42.525 | 53.542 | 1.00 | 28.08 |
| 1818 | CG | GLU | 235 | 13.612 | 43.325 | 53.437 | 1.00 | 31.10 |
| 1819 | CD | GLU | 235 | 13.829 | 44.207 | 54.669 | 1.00 | 44.53 |
| 1820 | OE1 | GLU | 235 | 13.767 | 43.656 | 55.782 | 1.00 | 37.00 |
| 1821 | OE2 | GLU | 235 | 14.067 | 45.437 | 54.536 | 1.00 | 43.38 |
| 1822 | C | GLU | 235 | 12.929 | 40.560 | 52.086 | 1.00 | 26.98 |
| 1823 | O | GLU | 235 | 12.803 | 39.520 | 52.735 | 1.00 | 27.63 |
| 1824 | N | LEU | 236 | 13.841 | 40.706 | 51.132 | 1.00 | 35.28 |
| 1825 | CA | LEU | 236 | 14.802 | 39.649 | 50.870 | 1.00 | 38.83 |
| 1826 | CB | LEU | 236 | 14.577 | 39.144 | 49.454 | 1.00 | 42.59 |
| 1827 | CG | LEU | 236 | 15.577 | 38.252 | 48.723 | 1.00 | 43.05 |
| 1828 | CD1 | LEU | 236 | 14.848 | 37.473 | 47.654 | 1.00 | 52.72 |
| 1829 | CD2 | LEU | 236 | 16.633 | 39.107 | 48.077 | 1.00 | 49.10 |
| 1830 | C | LEU | 236 | 16.284 | 40.017 | 51.111 | 1.00 | 37.17 |
| 1831 | O | LEU | 236 | 16.734 | 41.151 | 50.963 | 1.00 | 31.44 |
| 1832 | N | ARG | 237 | 17.051 | 39.039 | 51.517 | 1.00 | 34.35 |
| 1833 | CA | ARG | 237 | 18.451 | 39.316 | 51.730 | 1.00 | 39.43 |
| 1834 | CB | ARG | 237 | 18.730 | 39.665 | 53.212 | 1.00 | 29.18 |
| 1835 | CG | ARG | 237 | 18.677 | 38.489 | 54.209 | 1.00 | 30.42 |
| 1836 | CD | ARG | 237 | 19.078 | 39.099 | 55.570 | 1.00 | 36.95 |
| 1837 | NE | ARG | 237 | 19.220 | 38.175 | 56.676 | 1.00 | 31.85 |
| 1838 | CZ | ARG | 237 | 19.408 | 38.592 | 57.931 | 1.00 | 34.62 |
| 1839 | NH1 | ARG | 237 | 19.491 | 39.888 | 58.226 | 1.00 | 32.34 |
| 1840 | NH2 | ARG | 237 | 19.457 | 37.737 | 58.895 | 1.00 | 23.82 |
| 1841 | C | ARG | 237 | 19.250 | 38.107 | 51.285 | 1.00 | 40.07 |
| 1842 | O | ARG | 237 | 18.803 | 36.948 | 51.429 | 1.00 | 36.37 |
| 1843 | N | TYR | 238 | 20.405 | 38.390 | 50.697 | 1.00 | 35.86 |
| 1844 | CA | TYR | 238 | 21.278 | 37.332 | 50.215 | 1.00 | 43.63 |
| 1845 | CB | TYR | 238 | 20.950 | 37.002 | 48.750 | 1.00 | 35.84 |
| 1846 | CG | TYR | 238 | 21.127 | 38.172 | 47.808 | 1.00 | 31.36 |
| 1847 | CD1 | TYR | 238 | 22.297 | 38.335 | 47.089 | 1.00 | 35.38 |
| 1848 | CE1 | TYR | 238 | 22.479 | 39.434 | 46.237 | 1.00 | 37.61 |
| 1849 | CD2 | TYR | 238 | 20.124 | 39.129 | 47.652 | 1.00 | 39.53 |
| 1850 | CE2 | TYR | 238 | 20.283 | 40.226 | 46.795 | 1.00 | 33.69 |
| 1851 | CZ | TYR | 238 | 21.474 | 40.364 | 46.096 | 1.00 | 44.16 |
| 1852 | OH | TYR | 238 | 21.670 | 41.421 | 45.263 | 1.00 | 47.16 |
| 1853 | C | TYR | 238 | 22.746 | 37.705 | 50.319 | 1.00 | 46.65 |
| 1854 | O | TYR | 238 | 23.122 | 38.879 | 50.336 | 1.00 | 43.79 |
| 1855 | N | ARG | 239 | 23.577 | 36.685 | 50.370 | 1.00 | 45.60 |
| 1856 | CA | ARG | 239 | 25.005 | 36.907 | 50.423 | 1.00 | 50.19 |
| 1857 | CB | ARG | 239 | 25.469 | 37.208 | 51.865 | 1.00 | 40.99 |
| 1858 | CG | ARG | 239 | 25.467 | 36.026 | 52.787 | 1.00 | 33.67 |
| 1859 | CD | ARG | 239 | 25.684 | 36.494 | 54.242 | 1.00 | 36.88 |
| 1860 | NE | ARG | 239 | 25.322 | 35.446 | 55.210 | 1.00 | 33.50 |
| 1861 | CZ | ARG | 239 | 25.923 | 34.262 | 55.296 | 1.00 | 41.08 |
| 1862 | NH1 | ARG | 239 | 26.925 | 33.981 | 54.476 | 1.00 | 38.34 |
| 1863 | NH2 | ARG | 239 | 25.509 | 33.352 | 56.184 | 1.00 | 37.14 |
| 1864 | C | ARG | 239 | 25.700 | 35.669 | 49.900 | 1.00 | 49.27 |
| 1865 | O | ARG | 239 | 25.119 | 34.570 | 49.913 | 1.00 | 42.58 |
| 1866 | N | ALA | 240 | 26.934 | 35.858 | 49.426 | 1.00 | 52.31 |
| 1867 | CA | ALA | 240 | 27.757 | 34.748 | 48.955 | 1.00 | 38.15 |
| 1868 | CB | ALA | 240 | 29.043 | 35.274 | 48.391 | 1.00 | 43.96 |
| 1869 | C | ALA | 240 | 27.987 | 33.991 | 50.254 | 1.00 | 38.23 |
| 1870 | O | ALA | 240 | 28.128 | 34.588 | 51.313 | 1.00 | 47.79 |
| 1871 | N | GLU | 241 | 27.964 | 32.678 | 50.200 | 1.00 | 39.86 |
| 1872 | CA | GLU | 241 | 28.122 | 31.897 | 51.407 | 1.00 | 50.52 |
| 1873 | CB | GLU | 241 | 28.158 | 30.426 | 51.017 | 1.00 | 60.70 |
| 1874 | CG | GLU | 241 | 28.293 | 29.452 | 52.140 | 1.00 | 58.15 |
| 1875 | CD | GLU | 241 | 28.087 | 28.041 | 51.642 | 1.00 | 72.57 |
| 1876 | OE1 | GLU | 241 | 26.937 | 27.561 | 51.708 | 1.00 | 79.46 |
| 1877 | OE2 | GLU | 241 | 29.062 | 27.420 | 51.157 | 1.00 | 67.86 |
| 1878 | C | GLU | 241 | 29.380 | 32.299 | 52.170 | 1.00 | 55.56 |
| 1879 | O | GLU | 241 | 29.411 | 32.266 | 53.402 | 1.00 | 47.41 |
| 1880 | N | ARG | 242 | 30.404 | 32.705 | 51.420 | 1.00 | 62.76 |
| 1881 | CA | ARG | 242 | 31.695 | 33.122 | 51.968 | 1.00 | 63.11 |
| 1882 | CB | ARG | 242 | 32.747 | 33.023 | 50.890 | 1.00 | 60.32 |
| 1883 | CG | ARG | 242 | 32.667 | 34.130 | 49.865 | 1.00 | 69.88 |
| 1884 | CD | ARG | 242 | 33.680 | 33.865 | 48.764 | 1.00 | 71.90 |
| 1885 | NE | ARG | 242 | 33.874 | 35.016 | 47.901 | 1.00 | 75.36 |
| 1886 | CZ | ARG | 242 | 34.519 | 34.957 | 46.742 | 1.00 | 85.19 |
| 1887 | NH1 | ARG | 242 | 35.022 | 33.792 | 46.333 | 1.00 | 81.84 |
| 1888 | NH2 | ARG | 242 | 34.651 | 36.049 | 45.987 | 1.00 | 74.21 |
| 1889 | C | ARG | 242 | 31.751 | 34.540 | 52.556 | 1.00 | 71.41 |
| 1890 | O | ARG | 242 | 32.575 | 34.816 | 53.428 | 1.00 | 69.71 |
| 1891 | N | SER | 243 | 30.891 | 35.440 | 52.085 | 1.00 | 69.39 |
| 1892 | CA | SER | 243 | 30.896 | 36.807 | 52.600 | 1.00 | 71.04 |
| 1893 | CB | SER | 243 | 30.338 | 37.791 | 51.557 | 1.00 | 79.60 |
| 1894 | OG | SER | 243 | 31 | 39.055 | 51.624 | 1.00 | 78.06 |
| 1895 | C | SER | 243 | 30.079 | 36.865 | 53.879 | 1.00 | 69.07 |
| 1896 | O | SER | 243 | 29.294 | 35.950 | 54.158 | 1.00 | 67.88 |
| 1897 | N | LYS | 244 | 30.285 | 37.927 | 54.661 | 1.00 | 58.86 |
| 1898 | CA | LYS | 244 | 29.570 | 38.096 | 55.930 | 1.00 | 60.14 |
| 1899 | CB | LYS | 244 | 30.503 | 38.565 | 57.052 | 1.00 | 60.04 |
| 1900 | CG | LYS | 244 | 31.255 | 37.462 | 57.792 | 1.00 | 58.68 |
| 1901 | CD | LYS | 244 | 32.118 | 38.032 | 58.908 | 0.00 | 62.15 |
| 1902 | CE | LYS | 244 | 32.848 | 36.930 | 59.659 | 0.00 | 62.43 |
| 1903 | NZ | LYS | 244 | 33.691 | 37.472 | 60.762 | 0.00 | 62.97 |
| 1904 | C | LYS | 244 | 28.445 | 39.095 | 55.834 | 1.00 | 61.79 |
| 1905 | O | LYS | 244 | 27.550 | 39.072 | 56.668 | 1.00 | 62.89 |
| 1906 | N | THR | 245 | 28.465 | 39.963 | 54.823 | 1.00 | 55.14 |
| 1907 | CA | THR | 245 | 27.411 | 40.952 | 54.724 | 1.00 | 46.49 |
| 1908 | CB | THR | 245 | 27.996 | 42.390 | 54.614 | 1.00 | 49.94 |
| 1909 | OG1 | THR | 245 | 28.685 | 42.545 | 53.370 | 1.00 | 55.00 |
| 1910 | CG2 | THR | 245 | 28.959 | 42.649 | 55.782 | 1.00 | 60.75 |
| 1911 | C | THR | 245 | 26.345 | 40.787 | 53.649 | 1.00 | 53.39 |
| 1912 | O | THR | 245 | 26.628 | 40.613 | 52.450 | 1.00 | 49.67 |
| 1913 | N | PHE | 246 | 25.099 | 40.884 | 54.104 | 1.00 | 50.84 |
| 1914 | CA | PHE | 246 | 23.946 | 40.775 | 53.235 | 1.00 | 42.46 |
| 1915 | CB | PHE | 246 | 22.703 | 40.488 | 54.083 | 1.00 | 40.90 |
| 1916 | CG | PHE | 246 | 22.613 | 39.093 | 54.594 | 1.00 | 42.14 |
| 1917 | CD1 | PHE | 246 | 22.887 | 38.808 | 55.914 | 1.00 | 40.17 |
| 1918 | CD2 | PHE | 246 | 22.229 | 38.054 | 53.756 | 1.00 | 33.93 |
| 1919 | CE1 | PHE | 246 | 22.784 | 37.502 | 56.396 | 1.00 | 40.18 |
| 1920 | CE2 | PHE | 246 | 22.125 | 36.745 | 54.233 | 1.00 | 35.22 |
| 1921 | CZ | PHE | 246 | 22.400 | 36.464 | 55.546 | 1.00 | 41.58 |
| 1922 | C | PHE | 246 | 23.652 | 42.030 | 52.386 | 1.00 | 39.02 |
| 1923 | O | PHE | 246 | 23.954 | 43.163 | 52.757 | 1.00 | 40.27 |
| 1924 | N | THR | 247 | 23.050 | 41.786 | 51.237 | 1.00 | 36.74 |
| 1925 | CA | THR | 247 | 22.539 | 42.821 | 50.337 | 1.00 | 35.16 |
| 1926 | CB | THR | 247 | 22.930 | 42.526 | 48.878 | 1.00 | 51.27 |
| 1927 | OG1 | THR | 247 | 24.328 | 42.844 | 48.718 | 1.00 | 56.62 |
| 1928 | CG2 | THR | 247 | 22.088 | 43.339 | 47.881 | 1.00 | 41.69 |
| 1929 | C | THR | 247 | 21.039 | 42.585 | 50.662 | 1.00 | 38.05 |
| 1930 | O | THR | 247 | 20.577 | 41.441 | 50.725 | 1.00 | 37.69 |
| 1931 | N | THR | 248 | 20.321 | 43.656 | 50.988 | 1.00 | 46.24 |
| 1932 | CA | THR | 248 | 18.918 | 43.535 | 51.395 | 1.00 | 46.62 |
| 1933 | CB | THR | 248 | 18.705 | 44.000 | 52.865 | 1.00 | 40.53 |
| 1934 | OG1 | THR | 248 | 19.702 | 43.373 | 53.693 | 1.00 | 42.40 |
| 1935 | CG2 | THR | 248 | 17.287 | 43.524 | 53.414 | 1.00 | 32.11 |
| 1936 | C | THR | 248 | 18.054 | 44.314 | 50.462 | 1.00 | 48.65 |
| 1937 | O | THR | 248 | 18.385 | 45.439 | 50.090 | 1.00 | 44.44 |
| 1938 | N | TRP | 249 | 16.942 | 43.700 | 50.078 | 1.00 | 40.58 |
| 1939 | CA | TRP | 249 | 16.028 | 44.318 | 49.120 | 1.00 | 50.64 |
| 1940 | CB | TRP | 249 | 16.149 | 43.619 | 47.745 | 1.00 | 55.42 |
| 1941 | CG | TRP | 249 | 17.446 | 43.758 | 46.964 | 1.00 | 61.34 |
| 1942 | CD2 | TRP | 249 | 17.801 | 43.025 | 45.771 | 1.00 | 71.58 |
| 1943 | CE2 | TRP | 249 | 18.977 | 43.630 | 45.238 | 1.00 | 65.13 |
| 1944 | CE3 | TRP | 249 | 17.233 | 41.919 | 45.095 | 1.00 | 66.83 |
| 1945 | CD1 | TRP | 249 | 18.401 | 44.725 | 47.106 | 1.00 | 63.11 |
| 1946 | NE1 | TRP | 249 | 19.315 | 44.660 | 46.072 | 1.00 | 65.20 |
| 1947 | CZ2 | TRP | 249 | 19.598 | 43.174 | 44.053 | 1.00 | 74.50 |
| 1948 | CZ3 | TRP | 249 | 17.851 | 41.457 | 43.905 | 1.00 | 77.25 |
| 1949 | CH2 | TRP | 249 | 19.028 | 42.092 | 43.399 | 1.00 | 78.89 |
| 1950 | C | TRP | 249 | 14.564 | 44.189 | 49.537 | 1.00 | 44.23 |
| 1951 | O | TRP | 249 | 14.169 | 43.181 | 50.127 | 1.00 | 44.26 |
| 1952 | N | MET | 250 | 13.762 | 45.208 | 49.259 | 1.00 | 35.13 |
| 1953 | CA | MET | 250 | 12.321 | 45.077 | 49.499 | 1.00 | 41.64 |
| 1954 | CB | MET | 250 | 11.640 | 46.432 | 49.719 | 1.00 | 33.96 |
| 1955 | CG | MET | 250 | 11.966 | 47.136 | 58 | 1.00 | 41.53 |
| 1956 | SD | MET | 250 | 11.146 | 46.319 | 52.447 | 1.00 | 54.10 |
| 1957 | CE | MET | 250 | 9.492 | 45.726 | 51.556 | 1.00 | 34.59 |
| 1958 | C | MET | 250 | 11.757 | 44.492 | 48.178 | 1.00 | 45.74 |
| 1959 | O | MET | 250 | 11.996 | 45.055 | 47.120 | 1.00 | 32.67 |
| 1960 | N | VAL | 251 | 11.069 | 43.352 | 48.231 | 1.00 | 42.22 |
| 1961 | CA | VAL | 251 | 10.423 | 42.794 | 47.049 | 1.00 | 38.75 |
| 1962 | CB | VAL | 251 | 9.856 | 41.447 | 47.413 | 1.00 | 49.52 |
| 1963 | CG1 | VAL | 251 | 9.207 | 40.787 | 46.200 | 1.00 | 37.23 |
| 1964 | CG2 | VAL | 251 | 10.991 | 40.594 | 47.983 | 1.00 | 44.18 |
| 1965 | C | VAL | 251 | 9.292 | 43.761 | 46.628 | 1.00 | 43.35 |
| 1966 | O | VAL | 251 | 8.419 | 44.081 | 47.416 | 1.00 | 50.69 |
| 1967 | N | LYS | 252 | 9.283 | 44.263 | 45.408 | 1.00 | 41.06 |
| 1968 | CA | LYS | 252 | 8.213 | 45.218 | 45.094 | 1.00 | 42.19 |
| 1969 | CB | LYS | 252 | 8.672 | 46.176 | 43.985 | 1.00 | 37.24 |
| 1970 | CG | LYS | 252 | 8.567 | 45.593 | 42.547 | 1.00 | 47.21 |
| 1971 | CD | LYS | 252 | 9.375 | 46.410 | 41.547 | 0.00 | 46.21 |
| 1972 | CE | LYS | 252 | 10.871 | 46.291 | 41.808 | 0.00 | 47.33 |
| 1973 | NZ | LYS | 252 | 11.352 | 44.886 | 41.675 | 0.00 | 47.25 |
| 1974 | C | LYS | 252 | 6.835 | 44.637 | 44.730 | 1.00 | 26.17 |
| 1975 | O | LYS | 252 | 6.687 | 43.438 | 44.454 | 1.00 | 47.89 |
| 1976 | N | ASP | 253 | 5.826 | 45.488 | 44.791 | 1.00 | 42.16 |
| 1977 | CA | ASP | 253 | 4.467 | 45.125 | 44.384 | 1.00 | 37.78 |
| 1978 | CB | ASP | 253 | 4.459 | 44.980 | 42.848 | 1.00 | 39.37 |
| 1979 | CG | ASP | 253 | 4.638 | 46.319 | 42.129 | 1.00 | 47.98 |
| 1980 | OD1 | ASP | 253 | 4.800 | 46.281 | 40.881 | 1.00 | 61.76 |
| 1981 | OD2 | ASP | 253 | 4.602 | 47.396 | 42.798 | 1.00 | 45.42 |
| 1982 | C | ASP | 253 | 3.813 | 43.903 | 45.018 | 1.00 | 33.71 |
| 1983 | O | ASP | 253 | 2.979 | 43.241 | 44.402 | 1.00 | 35.54 |
| 1984 | N | LEU | 254 | 4.208 | 43.602 | 46.238 | 1.00 | 32.03 |
| 1985 | CA | LEU | 254 | 3.656 | 42.494 | 46.973 | 1.00 | 30.30 |
| 1986 | CB | LEU | 254 | 2.151 | 42.737 | 47.169 | 1.00 | 40.57 |
| 1987 | CG | LEU | 254 | 1.847 | 44.031 | 47.923 | 1.00 | 45.55 |
| 1988 | CD1 | LEU | 254 | 0.290 | 44.246 | 48.135 | 1.00 | 33.19 |
| 1989 | CD2 | LEU | 254 | 2.605 | 43.921 | 49.260 | 1.00 | 43.39 |
| 1990 | C | LEU | 254 | 3.914 | 41.116 | 46.349 | 1.00 | 38.42 |
| 1991 | O | LEU | 254 | 3.287 | 40.103 | 46.723 | 1.00 | 39.22 |
| 1992 | N | GLN | 255 | 4.875 | 41.054 | 45.440 | 1.00 | 40.77 |
| 1993 | CA | GLN | 255 | 5.176 | 39.800 | 44.750 | 1.00 | 34.97 |
| 1994 | CB | GLN | 255 | 6.111 | 40.077 | 43.566 | 1.00 | 37.38 |
| 1995 | CG | GLN | 255 | 5.484 | 41.044 | 42.597 | 1.00 | 61.25 |
| 1996 | CD | GLN | 255 | 6.363 | 41.399 | 41.436 | 1.00 | 50.25 |
| 1997 | OE1 | GLN | 255 | 7.439 | 41.959 | 41.602 | 1.00 | 59.72 |
| 1998 | NE2 | GLN | 255 | 5.893 | 41.095 | 40.244 | 1.00 | 55.69 |
| 1999 | C | GLN | 255 | 5.756 | 38.743 | 45.648 | 1.00 | 35.43 |
| 2000 | O | GLN | 255 | 6.300 | 39.070 | 46.699 | 1.00 | 36.43 |
| 2001 | N | HIS | 256 | 5.640 | 37.486 | 45.226 | 1.00 | 24.94 |
| 2002 | CA | HIS | 256 | 6.177 | 36.356 | 45.983 | 1.00 | 31.81 |
| 2003 | CB | HIS | 256 | 5.117 | 35.271 | 46.173 | 1.00 | 30.35 |
| 2004 | CG | HIS | 256 | 4.034 | 35.649 | 47.127 | 1.00 | 33.39 |
| 2005 | CD2 | HIS | 256 | 3.826 | 35.305 | 48.414 | 1.00 | 38.37 |
| 2006 | ND1 | HIS | 256 | 2.999 | 36.484 | 46.783 | 1.00 | 32.74 |
| 2007 | CE1 | HIS | 256 | 2.201 | 36.638 | 47.817 | 1.00 | 26.75 |
| 2008 | NE2 | HIS | 256 | 2.677 | 35.936 | 48.817 | 1.00 | 33.83 |
| 2009 | C | HIS | 256 | 7.391 | 35.718 | 45.310 | 1.00 | 38.16 |
| 2010 | O | HIS | 256 | 7.651 | 34.533 | 45.457 | 1.00 | 42.32 |
| 2011 | N | HIS | 257 | 8.128 | 36.504 | 44.554 | 1.00 | 30.63 |
| 2012 | CA | HIS | 257 | 9.264 | 35.957 | 43.885 | 1.00 | 37.12 |
| 2013 | CB | HIS | 257 | 8.854 | 35.207 | 42.606 | 1.00 | 41.87 |
| 2014 | CG | HIS | 257 | 8.490 | 36.125 | 41.482 | 1.00 | 39.91 |
| 2015 | CD2 | HIS | 257 | 9.251 | 36.675 | 40.503 | 1.00 | 33.87 |
| 2016 | ND1 | HIS | 257 | 7.267 | 36.758 | 41.420 | 1.00 | 47.18 |
| 2017 | CE1 | HIS | 257 | 7.301 | 37.673 | 40.467 | 1.00 | 49.26 |
| 2018 | NE2 | HIS | 257 | 8.494 | 37.643 | 39.899 | 1.00 | 52.84 |
| 2019 | C | HIS | 257 | 10.023 | 37.154 | 43.465 | 1.00 | 43.28 |
| 2020 | O | HIS | 257 | 9.472 | 38.256 | 43.421 | 1.00 | 30.96 |
| 2021 | N | CYS | 258 | 11.290 | 36.925 | 43.152 | 1.00 | 39.41 |
| 2022 | CA | CYS | 258 | 12.116 | 37.981 | 42.621 | 1.00 | 42.81 |
| 2023 | CB | CYS | 258 | 12.307 | 39.099 | 43.669 | 1.00 | 53.73 |
| 2024 | SG | CYS | 258 | 13.843 | 39.174 | 44.589 | 1.00 | 55.13 |
| 2025 | C | CYS | 258 | 13.421 | 37.370 | 42.110 | 1.00 | 37.34 |
| 2026 | O | CYS | 258 | 13.829 | 36.276 | 42.502 | 1.00 | 43.29 |
| 2027 | N | VAL | 259 | 14.060 | 38.057 | 41.192 | 1.00 | 39.86 |
| 2028 | CA | VAL | 259 | 15.281 | 37.526 | 40.618 | 1.00 | 41.36 |
| 2029 | CB | VAL | 259 | 15.188 | 37.459 | 39.071 | 1.00 | 38.54 |
| 2030 | CG1 | VAL | 259 | 16.531 | 37.000 | 38.448 | 1.00 | 45.29 |
| 2031 | CG2 | VAL | 259 | 14.133 | 36.501 | 38.697 | 1.00 | 29.53 |
| 2032 | C | VAL | 259 | 16.474 | 38.341 | 45 | 1.00 | 36.03 |
| 2033 | O | VAL | 259 | 16.541 | 39.522 | 40.724 | 1.00 | 39.67 |
| 2034 | N | ILE | 260 | 17.398 | 37.697 | 41.690 | 1.00 | 44.08 |
| 2035 | CA | ILE | 260 | 18.648 | 38.344 | 42.083 | 1.00 | 38.84 |
| 2036 | CB | ILE | 260 | 19.355 | 37.449 | 43.095 | 1.00 | 41.64 |
| 2037 | CG2 | ILE | 260 | 20.823 | 37.964 | 43.364 | 1.00 | 50.74 |
| 2038 | CG1 | ILE | 260 | 18.490 | 37.385 | 44.370 | 1.00 | 28.55 |
| 2039 | CD1 | ILE | 260 | 18.968 | 36.455 | 45.459 | 1.00 | 23.68 |
| 2040 | C | ILE | 260 | 19.432 | 38.483 | 40.754 | 1.00 | 35.50 |
| 2041 | O | ILE | 260 | 19.628 | 37.490 | 40.031 | 1.00 | 33.20 |
| 2042 | N | HIS | 261 | 19.807 | 39.710 | 40.395 | 1.00 | 26.09 |
| 2043 | CA | HIS | 261 | 20.534 | 39.948 | 39.133 | 1.00 | 43.38 |
| 2044 | CB | HIS | 261 | 19.938 | 41.124 | 38.362 | 1.00 | 36.99 |
| 2045 | CG | HIS | 261 | 18.589 | 40.865 | 37.776 | 1.00 | 61.38 |
| 2046 | CD2 | HIS | 261 | 17.462 | 41.614 | 37.782 | 1.00 | 57.02 |
| 2047 | ND1 | HIS | 261 | 18.319 | 39.779 | 36.973 | 1.00 | 66.71 |
| 2048 | CE1 | HIS | 261 | 17.089 | 39.875 | 36.500 | 1.00 | 62.22 |
| 2049 | NE2 | HIS | 261 | 16.548 | 40.982 | 36.975 | 1.00 | 60.94 |
| 2050 | C | HIS | 261 | 22.047 | 40.207 | 39.237 | 1.00 | 36.64 |
| 2051 | O | HIS | 261 | 22.707 | 40.448 | 38.215 | 1.00 | 42.86 |
| 2052 | N | ASP | 262 | 22.600 | 40.141 | 40.438 | 1.00 | 36.00 |
| 2053 | CA | ASP | 262 | 24.023 | 40.431 | 40.606 | 1.00 | 49.39 |
| 2054 | CB | ASP | 262 | 24.217 | 41.888 | 41.060 | 1.00 | 43.72 |
| 2055 | CG | ASP | 262 | 23.524 | 42.177 | 42.375 | 1.00 | 61.27 |
| 2056 | OD1 | ASP | 262 | 23.483 | 43.365 | 42.782 | 1.00 | 55.33 |
| 2057 | OD2 | ASP | 262 | 23.022 | 41.200 | 42.998 | 1.00 | 58.69 |
| 2058 | C | ASP | 262 | 24.698 | 39.511 | 41.579 | 1.00 | 38.22 |
| 2059 | O | ASP | 262 | 25.588 | 39.926 | 42.307 | 1.00 | 54.56 |
| 2060 | N | ALA | 263 | 24.252 | 38.259 | 41.602 | 1.00 | 42.47 |
| 2061 | CA | ALA | 263 | 24.825 | 37.239 | 42.456 | 1.00 | 39.03 |
| 2062 | CB | ALA | 263 | 23.986 | 36.027 | 42.358 | 1.00 | 30.33 |
| 2063 | C | ALA | 263 | 26.263 | 36.934 | 41.967 | 1.00 | 47.65 |
| 2064 | O | ALA | 263 | 26.567 | 37.066 | 40.773 | 1.00 | 58.15 |
| 2065 | N | TRP | 264 | 27.146 | 36.515 | 42.862 | 1.00 | 56.14 |
| 2066 | CA | TRP | 264 | 28.539 | 36.209 | 42.468 | 1.00 | 59.61 |
| 2067 | CB | TRP | 264 | 29.426 | 36.114 | 43.708 | 1.00 | 67.31 |
| 2068 | CG | TRP | 264 | 29.871 | 37.409 | 44.305 | 1.00 | 76.50 |
| 2069 | CD2 | TRP | 264 | 31.220 | 37.911 | 44.357 | 1.00 | 87.22 |
| 2070 | CE2 | TRP | 264 | 31.192 | 39.110 | 45.111 | 1.00 | 91.59 |
| 2071 | CE3 | TRP | 264 | 32.452 | 37.461 | 43.842 | 1.00 | 89.25 |
| 2072 | CD1 | TRP | 264 | 29.108 | 38.301 | 44.995 | 1.00 | 81.58 |
| 2073 | NE1 | TRP | 264 | 29.892 | 39.323 | 45.488 | 1.00 | 82.49 |
| 2074 | CZ2 | TRP | 264 | 32.350 | 39.870 | 45.364 | 1.00 | 95.10 |
| 2075 | CZ3 | TRP | 264 | 33.610 | 38.217 | 44.096 | 1.00 | 87.89 |
| 2076 | CH2 | TRP | 264 | 33.545 | 39.406 | 44.851 | 1.00 | 94.39 |
| 2077 | C | TRP | 264 | 28.688 | 34.901 | 41.670 | 1.00 | 48.82 |
| 2078 | O | TRP | 264 | 28.446 | 33.825 | 42.207 | 1.00 | 52.91 |
| 2079 | N | SER | 265 | 29.135 | 34.993 | 40.415 | 1.00 | 51.22 |
| 2080 | CA | SER | 265 | 29.280 | 33.821 | 39.538 | 1.00 | 49.09 |
| 2081 | CB | SER | 265 | 30.036 | 34.183 | 38.252 | 1.00 | 65.78 |
| 2082 | OG | SER | 265 | 29.776 | 33.244 | 37.189 | 1.00 | 69.91 |
| 2083 | C | SER | 265 | 29.899 | 32.572 | 40.150 | 1.00 | 49.64 |
| 2084 | O | SER | 265 | 30.927 | 32.622 | 40.793 | 1.00 | 50.72 |
| 2085 | N | GLY | 266 | 29.222 | 31.450 | 39.947 | 1.00 | 46.94 |
| 2086 | CA | GLY | 266 | 29.662 | 30.166 | 40.461 | 1.00 | 46.53 |
| 2087 | C | GLY | 266 | 29.641 | 29.951 | 41.967 | 1.00 | 53.65 |
| 2088 | O | GLY | 266 | 29.908 | 28.850 | 42.433 | 1.00 | 53.60 |
| 2089 | N | LEU | 267 | 29.327 | 30.974 | 42.747 | 1.00 | 54.99 |
| 2090 | CA | LEU | 267 | 29.325 | 30.788 | 44.194 | 1.00 | 39.64 |
| 2091 | CB | LEU | 267 | 29.813 | 32.051 | 44.883 | 1.00 | 47.50 |
| 2092 | CG | LEU | 267 | 31.214 | 32.554 | 44.527 | 1.00 | 50.96 |
| 2093 | CD1 | LEU | 267 | 31.589 | 33.521 | 45.617 | 1.00 | 52.06 |
| 2094 | CD2 | LEU | 267 | 32.239 | 31.423 | 44.446 | 1.00 | 34.64 |
| 2095 | C | LEU | 267 | 27.971 | 30.426 | 44.765 | 1.00 | 51.63 |
| 2096 | O | LEU | 267 | 26.929 | 30.905 | 44.273 | 1.00 | 47.09 |
| 2097 | N | ARG | 268 | 27.997 | 29.574 | 45.794 | 1.00 | 45.44 |
| 2098 | CA | ARG | 268 | 26.796 | 29.148 | 46.502 | 1.00 | 46.75 |
| 2099 | CB | ARG | 268 | 27.101 | 28.031 | 47.501 | 1.00 | 41.61 |
| 2100 | CG | ARG | 268 | 27.609 | 26.722 | 46.917 | 1.00 | 56.54 |
| 2101 | CD | ARG | 268 | 28.505 | 25.981 | 47.936 | 1.00 | 73.31 |
| 2102 | NE | ARG | 268 | 27.776 | 25.267 | 48.989 | 1.00 | 77.49 |
| 2103 | CZ | ARG | 268 | 27.057 | 24.162 | 48.782 | 1.00 | 81.37 |
| 2104 | NH1 | ARG | 268 | 26.950 | 23.641 | 47.560 | 1.00 | 77.10 |
| 2105 | NH2 | ARG | 268 | 26.486 | 23.546 | 49.805 | 1.00 | 72.41 |
| 2106 | C | ARG | 268 | 26.429 | 30.388 | 47.283 | 1.00 | 48.78 |
| 2107 | O | ARG | 268 | 27.311 | 31.045 | 47.793 | 1.00 | 46.05 |
| 2108 | N | HIS | 269 | 25.145 | 30.719 | 47.371 | 1.00 | 38.53 |
| 2109 | CA | HIS | 269 | 24.728 | 31.912 | 48.098 | 1.00 | 43.12 |
| 2110 | CB | HIS | 269 | 24.104 | 32.951 | 47.170 | 1.00 | 41.97 |
| 2111 | CG | HIS | 269 | 25.064 | 33.617 | 46.246 | 1.00 | 31.33 |
| 2112 | CD2 | HIS | 269 | 25.675 | 33.180 | 45.121 | 1.00 | 36.37 |
| 2113 | ND1 | HIS | 269 | 25.346 | 34.961 | 46.329 | 1.00 | 41.38 |
| 2114 | CE1 | HIS | 269 | 26.071 | 35.327 | 45.288 | 1.00 | 35.07 |
| 2115 | NE2 | HIS | 269 | 26.282 | 34.265 | 44.535 | 1.00 | 38.25 |
| 2116 | C | HIS | 269 | 23.678 | 31.540 | 49.086 | 1.00 | 45.22 |
| 2117 | O | HIS | 269 | 23.040 | 30.494 | 48.948 | 1.00 | 42.50 |
| 2118 | N | VAL | 270 | 23.498 | 32.404 | 50.085 | 1.00 | 45.32 |
| 2119 | CA | VAL | 270 | 22.455 | 32.193 | 51.087 | 1.00 | 43.16 |
| 2120 | CB | VAL | 270 | 23.002 | 32.208 | 52.548 | 1.00 | 41.99 |
| 2121 | CG1 | VAL | 270 | 21.841 | 32.030 | 53.533 | 1.00 | 52.03 |
| 2122 | CG2 | VAL | 270 | 23.965 | 31.086 | 52.744 | 1.00 | 39.14 |
| 2123 | C | VAL | 270 | 21.409 | 33.306 | 50.896 | 1.00 | 42.49 |
| 2124 | O | VAL | 270 | 21.734 | 34.467 | 50.617 | 1.00 | 37.04 |
| 2125 | N | VAL | 271 | 20.156 | 32.910 | 58 | 1.00 | 34.60 |
| 2126 | CA | VAL | 271 | 19.023 | 33.799 | 50.842 | 1.00 | 34.97 |
| 2127 | CB | VAL | 271 | 18.286 | 33.475 | 49.545 | 1.00 | 32.81 |
| 2128 | CG1 | VAL | 271 | 17.138 | 34.428 | 49.319 | 1.00 | 38.75 |
| 2129 | CG2 | VAL | 271 | 19.281 | 33.507 | 48.387 | 1.00 | 40.59 |
| 2130 | C | VAL | 271 | 18.045 | 33.674 | 52.003 | 1.00 | 35.77 |
| 2131 | O | VAL | 271 | 17.808 | 32.600 | 52.536 | 1.00 | 36.30 |
| 2132 | N | GLN | 272 | 17.489 | 34.796 | 52.400 | 1.00 | 35.17 |
| 2133 | CA | GLN | 272 | 16.524 | 34.775 | 53.485 | 1.00 | 48.95 |
| 2134 | CB | GLN | 272 | 17.221 | 35.139 | 54.818 | 1.00 | 40.50 |
| 2135 | CG | GLN | 272 | 18.055 | 34.000 | 55.424 | 1.00 | 34.14 |
| 2136 | CD | GLN | 272 | 18.580 | 34.371 | 56.816 | 1.00 | 34.66 |
| 2137 | OE1 | GLN | 272 | 19.231 | 35.406 | 56.988 | 1.00 | 33.14 |
| 2138 | NE2 | GLN | 272 | 18.277 | 33.540 | 57.809 | 1.00 | 32.17 |
| 2139 | C | GLN | 272 | 15.376 | 35.744 | 53.186 | 1.00 | 33.43 |
| 2140 | O | GLN | 272 | 15.579 | 36.727 | 52.458 | 1.00 | 31.69 |
| 2141 | N | LEU | 273 | 14.201 | 35.462 | 53.761 | 1.00 | 34.83 |
| 2142 | CA | LEU | 273 | 13.001 | 36.293 | 53.575 | 1.00 | 41.03 |
| 2143 | CB | LEU | 273 | 12.023 | 35.656 | 52.578 | 1.00 | 34.77 |
| 2144 | CG | LEU | 273 | 12.009 | 35.742 | 51.048 | 1.00 | 35.06 |
| 2145 | CD1 | LEU | 273 | 11.751 | 37.166 | 50.546 | 1.00 | 36.25 |
| 2146 | CD2 | LEU | 273 | 13.269 | 35.141 | 50.524 | 1.00 | 28.96 |
| 2147 | C | LEU | 273 | 12.213 | 36.532 | 54.868 | 1.00 | 41.75 |
| 2148 | O | LEU | 273 | 12.112 | 35.654 | 55.730 | 1.00 | 30.43 |
| 2149 | N | ARG | 274 | 11.642 | 37.728 | 54.978 | 1.00 | 28.00 |
| 2150 | CA | ARG | 274 | 10.834 | 38.064 | 56.123 | 1.00 | 37.09 |
| 2151 | CB | ARG | 274 | 11.643 | 38.861 | 57.150 | 1.00 | 37.16 |
| 2152 | CG | ARG | 274 | 12.067 | 40.283 | 56.756 | 1.00 | 23.22 |
| 2153 | CD | ARG | 274 | 12.673 | 40.922 | 57.986 | 1.00 | 28.07 |
| 2154 | NE | ARG | 274 | 13.178 | 42.274 | 57.746 | 1.00 | 29.61 |
| 2155 | CZ | ARG | 274 | 13.712 | 43.056 | 58.678 | 1.00 | 29.94 |
| 2156 | NH1 | ARG | 274 | 13.804 | 42.636 | 59.931 | 1.00 | 28.24 |
| 2157 | NH2 | ARG | 274 | 14.160 | 44.252 | 58.353 | 1.00 | 29.72 |
| 2158 | C | ARG | 274 | 9.616 | 38.858 | 55.620 | 1.00 | 49.84 |
| 2159 | O | ARG | 274 | 9.666 | 39.534 | 54.577 | 1.00 | 43.65 |
| 2160 | N | ALA | 275 | 8.523 | 38.764 | 56.365 | 1.00 | 44.60 |
| 2161 | CA | ALA | 275 | 7.290 | 39.420 | 55.971 | 1.00 | 39.29 |
| 2162 | CB | ALA | 275 | 6.270 | 38.340 | 55.620 | 1.00 | 33.60 |
| 2163 | C | ALA | 275 | 6.665 | 40.422 | 56.955 | 1.00 | 43.50 |
| 2164 | O | ALA | 275 | 6.786 | 40.313 | 58.181 | 1.00 | 43.88 |
| 2165 | N | GLN | 276 | 5.995 | 41.411 | 56.397 | 1.00 | 32.08 |
| 2166 | CA | GLN | 276 | 5.302 | 42.387 | 57.199 | 1.00 | 33.85 |
| 2167 | CB | GLN | 276 | 6.131 | 43.649 | 57.339 | 1.00 | 33.94 |
| 2168 | CG | GLN | 276 | 5.480 | 44.625 | 58.288 | 1.00 | 41.15 |
| 2169 | CD | GLN | 276 | 6.200 | 45.947 | 58.327 | 1.00 | 48.00 |
| 2170 | OE1 | GLN | 276 | 6.414 | 46.554 | 57.281 | 1.00 | 35.34 |
| 2171 | NE2 | GLN | 276 | 6.575 | 46.413 | 59.543 | 1.00 | 43.26 |
| 2172 | C | GLN | 276 | 3.944 | 42.733 | 56.523 | 1.00 | 38.59 |
| 2173 | O | GLN | 276 | 3.859 | 42.884 | 55.301 | 1.00 | 36.70 |
| 2174 | N | GLU | 277 | 2.887 | 42.824 | 57.313 | 1.00 | 32.28 |
| 2175 | CA | GLU | 277 | 1.583 | 43.193 | 56.789 | 1.00 | 33.47 |
| 2176 | CB | GLU | 277 | 0.621 | 43.451 | 57.952 | 1.00 | 32.73 |
| 2177 | CG | GLU | 277 | −0.789 | 43.765 | 57.555 | 1.00 | 30.25 |
| 2178 | CD | GLU | 277 | −0.939 | 45.228 | 57.218 | 1.00 | 31.54 |
| 2179 | OE1 | GLU | 277 | −0.275 | 46.064 | 57.869 | 1.00 | 56.80 |
| 2180 | OE2 | GLU | 277 | −1.713 | 45.564 | 56.300 | 1.00 | 64.09 |
| 2181 | C | GLU | 277 | 1.760 | 44.444 | 55.898 | 1.00 | 35.01 |
| 2182 | O | GLU | 277 | 2.372 | 45.437 | 56.283 | 1.00 | 34.51 |
| 2183 | N | GLU | 278 | 1.183 | 44.368 | 54.710 | 1.00 | 34.29 |
| 2184 | CA | GLU | 278 | 1.344 | 45.363 | 53.679 | 1.00 | 30.45 |
| 2185 | CB | GLU | 278 | 0.367 | 45.051 | 52.519 | 1.00 | 38.90 |
| 2186 | CG | GLU | 278 | −1.110 | 45.297 | 52.911 | 1.00 | 57.32 |
| 2187 | CD | GLU | 278 | −2.115 | 45.005 | 51.799 | 1.00 | 59.36 |
| 2188 | OE1 | GLU | 278 | −2.857 | 44.002 | 51.933 | 1.00 | 68.35 |
| 2189 | OE2 | GLU | 278 | −2.170 | 45.772 | 50.807 | 1.00 | 57.17 |
| 2190 | C | GLU | 278 | 1.274 | 46.825 | 54.043 | 1.00 | 33.01 |
| 2191 | O | GLU | 278 | 1.834 | 47.640 | 53.344 | 1.00 | 46.38 |
| 2192 | N | PHE | 279 | 0.622 | 47.177 | 55.130 | 1.00 | 41.52 |
| 2193 | CA | PHE | 279 | 0.501 | 48.586 | 55.481 | 1.00 | 43.18 |
| 2194 | CB | PHE | 279 | −0.954 | 48.911 | 55.844 | 1.00 | 42.01 |
| 2195 | CG | PHE | 279 | −1.913 | 48.705 | 54.700 | 1.00 | 42.33 |
| 2196 | CD1 | PHE | 279 | −2.828 | 47.663 | 54.715 | 1.00 | 39.07 |
| 2197 | CD2 | PHE | 279 | −1.856 | 49.521 | 53.588 | 1.00 | 47.16 |
| 2198 | CE1 | PHE | 279 | −3.673 | 47.424 | 53.643 | 1.00 | 48.80 |
| 2199 | CE2 | PHE | 279 | −2.703 | 49.294 | 52.493 | 1.00 | 55.38 |
| 2200 | CZ | PHE | 279 | −3.613 | 48.237 | 52.527 | 1.00 | 43.20 |
| 2201 | C | PHE | 279 | 1.410 | 49.074 | 56.587 | 1.00 | 51.23 |
| 2202 | O | PHE | 279 | 1.237 | 50.198 | 57.091 | 1.00 | 49.78 |
| 2203 | N | GLY | 280 | 2.366 | 48.231 | 56.970 | 1.00 | 39.06 |
| 2204 | CA | GLY | 280 | 3.300 | 48.584 | 58.026 | 1.00 | 48.46 |
| 2205 | C | GLY | 280 | 2.740 | 48.489 | 59.430 | 1.00 | 36.31 |
| 2206 | O | GLY | 280 | 3.103 | 49.265 | 60.298 | 1.00 | 59.08 |
| 2207 | N | GLN | 281 | 1.865 | 47.519 | 59.637 | 1.00 | 45.26 |
| 2208 | CA | GLN | 281 | 1.213 | 47.254 | 60.907 | 1.00 | 31.45 |
| 2209 | CB | GLN | 281 | −0.287 | 46.991 | 60.704 | 1.00 | 32.46 |
| 2210 | CG | GLN | 281 | −0.916 | 46.201 | 61.872 | 1.00 | 55.24 |
| 2211 | CD | GLN | 281 | −2.411 | 45.859 | 61.693 | 1.00 | 68.87 |
| 2212 | OE1 | GLN | 281 | −2.955 | 45.018 | 62.425 | 1.00 | 68.40 |
| 2213 | NE2 | GLN | 281 | −3.071 | 46.504 | 60.731 | 1.00 | 61.43 |
| 2214 | C | GLN | 281 | 1.867 | 46.009 | 61.482 | 1.00 | 38.87 |
| 2215 | O | GLN | 281 | 2.094 | 45.026 | 60.780 | 1.00 | 38.48 |
| 2216 | N | GLY | 282 | 2.171 | 46.046 | 62.766 | 1.00 | 45.52 |
| 2217 | CA | GLY | 282 | 2.797 | 44.902 | 63.376 | 1.00 | 35.12 |
| 2218 | C | GLY | 282 | 4.281 | 44.940 | 63.110 | 1.00 | 42.16 |
| 2219 | O | GLY | 282 | 4.832 | 45.968 | 62.736 | 1.00 | 48.53 |
| 2220 | N | GLU | 283 | 4.922 | 43.803 | 63.264 | 1.00 | 29.98 |
| 2221 | CA | GLU | 283 | 6.343 | 43.750 | 63.095 | 1.00 | 33.07 |
| 2222 | CB | GLU | 283 | 7.007 | 43.194 | 64.376 | 1.00 | 43.38 |
| 2223 | CG | GLU | 283 | 6.682 | 43.922 | 65.716 | 1.00 | 43.11 |
| 2224 | CD | GLU | 283 | 7.493 | 45.223 | 65.954 | 1.00 | 38.62 |
| 2225 | OE1 | GLU | 283 | 7.343 | 45.832 | 67.029 | 1.00 | 45.26 |
| 2226 | OE2 | GLU | 283 | 8.276 | 45.650 | 65.082 | 1.00 | 53.21 |
| 2227 | C | GLU | 283 | 6.677 | 42.823 | 61.952 | 1.00 | 44.10 |
| 2228 | O | GLU | 283 | 5.831 | 42.046 | 61.482 | 1.00 | 33.28 |
| 2229 | N | TRP | 284 | 7.936 | 42.894 | 61.527 | 1.00 | 38.66 |
| 2230 | CA | TRP | 284 | 8.426 | 42.020 | 60.502 | 1.00 | 29.41 |
| 2231 | CB | TRP | 284 | 9.808 | 42.418 | 60.039 | 1.00 | 35.71 |
| 2232 | CG | TRP | 284 | 9.804 | 43.538 | 59.145 | 1.00 | 33.19 |
| 2233 | CD2 | TRP | 284 | 9.559 | 43.481 | 57.732 | 1.00 | 21.81 |
| 2234 | CE2 | TRP | 284 | 9.637 | 44.814 | 57.240 | 1.00 | 31.28 |
| 2235 | CE3 | TRP | 284 | 9.300 | 42.441 | 56.837 | 1.00 | 28.55 |
| 2236 | CD1 | TRP | 284 | 10.010 | 44.845 | 59.462 | 1.00 | 30.06 |
| 2237 | NE1 | TRP | 284 | 9.913 | 45.632 | 58.309 | 1.00 | 35.13 |
| 2238 | CZ2 | TRP | 284 | 9.448 | 45.125 | 55.892 | 1.00 | 14.92 |
| 2239 | CZ3 | TRP | 284 | 9.121 | 42.754 | 55.492 | 1.00 | 16.12 |
| 2240 | CH2 | TRP | 284 | 9.198 | 44.082 | 55.035 | 1.00 | 23.05 |
| 2241 | C | TRP | 284 | 8.551 | 40.667 | 61.091 | 1.00 | 30.55 |
| 2242 | O | TRP | 284 | 8.836 | 40.530 | 62.276 | 1.00 | 43.89 |
| 2243 | N | SER | 285 | 8.320 | 39.650 | 60.257 | 1.00 | 39.67 |
| 2244 | CA | SER | 285 | 8.526 | 38.279 | 60.673 | 1.00 | 31.64 |
| 2245 | CB | SER | 285 | 8.116 | 37.269 | 59.569 | 1.00 | 43.69 |
| 2246 | OG | SER | 285 | 9.009 | 37.223 | 58.451 | 1.00 | 36.01 |
| 2247 | C | SER | 285 | 10.027 | 38.058 | 60.909 | 1.00 | 32.81 |
| 2248 | O | SER | 285 | 10.904 | 38.827 | 60.493 | 1.00 | 25.83 |
| 2249 | N | GLU | 286 | 10.297 | 36.970 | 61.589 | 1.00 | 38.11 |
| 2250 | CA | GLU | 286 | 11.641 | 36.564 | 61.769 | 1.00 | 32.54 |
| 2251 | CB | GLU | 286 | 11.649 | 35.374 | 62.699 | 1.00 | 41.34 |
| 2252 | CG | GLU | 286 | 11.264 | 35.698 | 64.119 | 1.00 | 47.36 |
| 2253 | CD | GLU | 286 | 12.123 | 36.813 | 64.706 | 1.00 | 59.93 |
| 2254 | OE1 | GLU | 286 | 13.367 | 36.744 | 64.553 | 1.00 | 53.75 |
| 2255 | OE2 | GLU | 286 | 11.547 | 37.750 | 65.320 | 1.00 | 56.44 |
| 2256 | C | GLU | 286 | 12.068 | 36.137 | 60.338 | 1.00 | 4 |
| 2257 | O | GLU | 286 | 11.231 | 35.749 | 59.524 | 1.00 | 38.34 |
| 2258 | N | TRP | 287 | 13.361 | 36.219 | 60.029 | 1.00 | 31.32 |
| 2259 | CA | TRP | 287 | 13.873 | 35.792 | 58.727 | 1.00 | 21.68 |
| 2260 | CB | TRP | 287 | 15.397 | 36.035 | 58.617 | 1.00 | 27.33 |
| 2261 | CG | TRP | 287 | 15.742 | 37.440 | 58.576 | 1.00 | 27.17 |
| 2262 | CD2 | TRP | 287 | 15.658 | 38.304 | 57.435 | 1.00 | 24.89 |
| 2263 | CE2 | TRP | 287 | 16.086 | 39.600 | 57.864 | 1.00 | 29.66 |
| 2264 | CE3 | TRP | 287 | 15.269 | 38.116 | 56.092 | 1.00 | 31.75 |
| 2265 | CD1 | TRP | 287 | 16.183 | 38.198 | 59.607 | 1.00 | 27.82 |
| 2266 | NE1 | TRP | 287 | 16.399 | 39.502 | 59.190 | 1.00 | 28.12 |
| 2267 | CZ2 | TRP | 287 | 16.142 | 40.697 | 57.014 | 1.00 | 22.70 |
| 2268 | CZ3 | TRP | 287 | 15.330 | 39.212 | 55.223 | 1.00 | 28.87 |
| 2269 | CH2 | TRP | 287 | 15.767 | 40.486 | 55.687 | 1.00 | 31.96 |
| 2270 | C | TRP | 287 | 13.636 | 34.304 | 58.561 | 1.00 | 26.43 |
| 2271 | O | TRP | 287 | 13.674 | 33.535 | 59.516 | 1.00 | 31.24 |
| 2272 | N | SER | 288 | 13.391 | 33.878 | 57.344 | 1.00 | 25.58 |
| 2273 | CA | SER | 288 | 13.214 | 32.446 | 57.146 | 1.00 | 38.12 |
| 2274 | CB | SER | 288 | 12.699 | 32.201 | 55.747 | 1.00 | 28.04 |
| 2275 | OG | SER | 288 | 13.545 | 32.885 | 54.853 | 1.00 | 26.76 |
| 2276 | C | SER | 288 | 14.570 | 31.745 | 57.291 | 1.00 | 36.42 |
| 2277 | O | SER | 288 | 15.612 | 32.392 | 57.221 | 1.00 | 33.69 |
| 2278 | N | PRO | 289 | 14.555 | 30.408 | 57.488 | 1.00 | 40.56 |
| 2279 | CD | PRO | 289 | 13.324 | 29.598 | 57.618 | 1.00 | 51.45 |
| 2280 | CA | PRO | 289 | 15.737 | 29.558 | 57.635 | 1.00 | 26.02 |
| 2281 | CB | PRO | 289 | 15.138 | 28.155 | 57.667 | 1.00 | 37.89 |
| 2282 | CG | PRO | 289 | 13.842 | 28.350 | 58.292 | 1.00 | 44.21 |
| 2283 | C | PRO | 289 | 16.509 | 29.800 | 56.331 | 1.00 | 38.96 |
| 2284 | O | PRO | 289 | 15.896 | 29.988 | 55.275 | 1.00 | 37.40 |
| 2285 | N | GLU | 290 | 17.840 | 29.776 | 56.402 | 1.00 | 45.45 |
| 2286 | CA | GLU | 290 | 18.684 | 30.025 | 55.246 | 1.00 | 39.83 |
| 2287 | CB | GLU | 290 | 20.164 | 29.926 | 55.617 | 1.00 | 56.29 |
| 2288 | CG | GLU | 290 | 20.549 | 30.709 | 56.869 | 1.00 | 71.73 |
| 2289 | CD | GLU | 290 | 22.060 | 30.807 | 57.057 | 1.00 | 75.52 |
| 2290 | OE1 | GLU | 290 | 22.711 | 31.524 | 56.264 | 1.00 | 66.31 |
| 2291 | OE2 | GLU | 290 | 22.591 | 30.168 | 58.000 | 1.00 | 81.24 |
| 2292 | C | GLU | 290 | 18.419 | 29.116 | 54.094 | 1.00 | 44.84 |
| 2293 | O | GLU | 290 | 18.368 | 27.891 | 54.248 | 1.00 | 48.20 |
| 2294 | N | ALA | 291 | 18.248 | 29.718 | 52.923 | 1.00 | 42.67 |
| 2295 | CA | ALA | 291 | 17.997 | 28.934 | 51.721 | 1.00 | 49.00 |
| 2296 | CB | ALA | 291 | 16.706 | 29.374 | 51.054 | 1.00 | 39.23 |
| 2297 | C | ALA | 291 | 19.170 | 29.226 | 50.821 | 1.00 | 53.16 |
| 2298 | O | ALA | 291 | 19.586 | 30.386 | 50.714 | 1.00 | 57.91 |
| 2299 | N | MET | 292 | 19.742 | 28.200 | 50.198 | 1.00 | 49.16 |
| 2300 | CA | MET | 292 | 20.857 | 28.492 | 49.306 | 1.00 | 51.93 |
| 2301 | CB | MET | 292 | 22.187 | 28.009 | 49.876 | 1.00 | 60.83 |
| 2302 | CG | MET | 292 | 22.333 | 26.542 | 50.216 | 1.00 | 63.53 |
| 2303 | SD | MET | 292 | 23.948 | 26.462 | 51.098 | 1.00 | 80.71 |
| 2304 | CE | MET | 292 | 25.153 | 26.380 | 49.696 | 1.00 | 56.94 |
| 2305 | C | MET | 292 | 20.712 | 28.010 | 47.897 | 1.00 | 50.87 |
| 2306 | O | MET | 292 | 19.865 | 27.162 | 47.599 | 1.00 | 41.34 |
| 2307 | N | GLY | 293 | 21.530 | 28.586 | 47.017 | 1.00 | 37.83 |
| 2308 | CA | GLY | 293 | 21.473 | 28.180 | 45.639 | 1.00 | 33.63 |
| 2309 | C | GLY | 293 | 22.672 | 28.740 | 44.958 | 1.00 | 42.01 |
| 2310 | O | GLY | 293 | 23.316 | 29.604 | 45.533 | 1.00 | 36.89 |
| 2311 | N | THR | 294 | 22.997 | 28.249 | 43.762 | 1.00 | 42.18 |
| 2312 | CA | THR | 294 | 24.145 | 28.798 | 43.056 | 1.00 | 45.71 |
| 2313 | CB | THR | 294 | 25.405 | 27.850 | 43.121 | 1.00 | 49.05 |
| 2314 | OG1 | THR | 294 | 25.253 | 26.760 | 42.229 | 1.00 | 63.33 |
| 2315 | CG2 | THR | 294 | 25.545 | 27.266 | 44.469 | 1.00 | 47.91 |
| 2316 | C | THR | 294 | 23.763 | 29.104 | 41.620 | 1.00 | 40.85 |
| 2317 | O | THR | 294 | 23.027 | 28.355 | 40.968 | 1.00 | 42.82 |
| 2318 | N | PRO | 295 | 24.233 | 30.238 | 41.113 | 1.00 | 28.04 |
| 2319 | CD | PRO | 295 | 24.987 | 31.275 | 41.822 | 1.00 | 28.46 |
| 2320 | CA | PRO | 295 | 23.910 | 30.616 | 39.734 | 1.00 | 29.71 |
| 2321 | CB | PRO | 295 | 24.586 | 31.980 | 39.542 | 1.00 | 19.02 |
| 2322 | CG | PRO | 295 | 24.790 | 32.474 | 40.952 | 1.00 | 28.89 |
| 2323 | C | PRO | 295 | 24.420 | 29.593 | 38.705 | 1.00 | 40.54 |
| 2324 | O | PRO | 295 | 25.258 | 28.755 | 39.021 | 1.00 | 38.79 |
| 2325 | N | TRP | 296 | 23.881 | 29.688 | 37.487 | 1.00 | 36.89 |
| 2326 | CA | TRP | 296 | 24.268 | 28.869 | 36.354 | 1.00 | 42.81 |
| 2327 | CB | TRP | 296 | 23.401 | 29.245 | 35.156 | 1.00 | 35.15 |
| 2328 | CG | TRP | 296 | 23.899 | 28.713 | 33.831 | 1.00 | 50.03 |
| 2329 | CD2 | TRP | 296 | 24.761 | 29.390 | 32.896 | 1.00 | 43.50 |
| 2330 | CE2 | TRP | 296 | 24.963 | 28.509 | 31.811 | 1.00 | 54.35 |
| 2331 | CE3 | TRP | 296 | 25.377 | 30.656 | 32.867 | 1.00 | 48.95 |
| 2332 | CD1 | TRP | 296 | 23.631 | 27.490 | 33.289 | 1.00 | 46.07 |
| 2333 | NE1 | TRP | 296 | 24.260 | 27.362 | 32.083 | 1.00 | 52.78 |
| 2334 | CZ2 | TRP | 296 | 25.758 | 28.853 | 30.692 | 1.00 | 39.39 |
| 2335 | CZ3 | TRP | 296 | 26.166 | 31 | 31.753 | 1.00 | 48.67 |
| 2336 | CH2 | TRP | 296 | 26.347 | 30.102 | 30.689 | 1.00 | 38.07 |
| 2337 | C | TRP | 296 | 25.757 | 29.084 | 35.970 | 1.00 | 37.10 |
| 2338 | O | TRP | 296 | 26.302 | 30.162 | 36.154 | 1.00 | 43.36 |
| 2339 | N | THR | 297 | 26.383 | 28.023 | 35.470 | 1.00 | 37.79 |
| 2340 | CA | THR | 297 | 27.749 | 28.027 | 34.975 | 1.00 | 46.98 |
| 2341 | CB | THR | 297 | 28.805 | 27.604 | 36.038 | 1.00 | 48.29 |
| 2342 | OG1 | THR | 297 | 28.544 | 26.272 | 36.491 | 1.00 | 59.02 |
| 2343 | CG2 | THR | 297 | 28.794 | 28.548 | 37.218 | 1.00 | 42.06 |
| 2344 | C | THR | 297 | 27.674 | 26.974 | 33.894 | 1.00 | 50.45 |
| 2345 | O | THR | 297 | 26.667 | 26.242 | 33.809 | 1.00 | 40.71 |
| 2346 | N | GLU | 298 | 28.687 | 26.925 | 33.031 | 1.00 | 45.38 |
| 2347 | CA | GLU | 298 | 28.704 | 25.895 | 31.998 | 1.00 | 51.45 |
| 2348 | CB | GLU | 298 | 29.483 | 26.350 | 30.769 | 1.00 | 48.68 |
| 2349 | CG | GLU | 298 | 28.966 | 27.615 | 30.166 | 1.00 | 54.52 |
| 2350 | CD | GLU | 298 | 29.881 | 28.166 | 29.089 | 1.00 | 71.17 |
| 2351 | OE1 | GLU | 298 | 31.060 | 28.518 | 29.397 | 1.00 | 73.91 |
| 2352 | OE2 | GLU | 298 | 29.408 | 28.248 | 27.935 | 1.00 | 58.35 |
| 2353 | C | GLU | 298 | 29.420 | 24.745 | 32.673 | 1.00 | 53.07 |
| 2354 | O | GLU | 298 | 30.317 | 24.975 | 33.498 | 1.00 | 52.23 |
| 2355 | N | SER | 299 | 29.019 | 23.525 | 32.317 | 1.00 | 54.52 |
| 2356 | CA | SER | 299 | 29.549 | 22.273 | 32.868 | 1.00 | 46.07 |
| 2357 | CB | SER | 299 | 28.828 | 21.116 | 32.186 | 1.00 | 50.73 |
| 2358 | OG | SER | 299 | 27.456 | 21.091 | 32.535 | 0.00 | 50.54 |
| 2359 | C | SER | 299 | 31.066 | 22.063 | 32.772 | 1.00 | 54.72 |
| 2360 | O | SER | 299 | 31.492 | 21.144 | 32.039 | 0.00 | 52.29 |
| 2361 | OXT | SER | 299 | 31.808 | 22.824 | 33.428 | 0.00 | 52.32 |
| 2362 | CB | CYS | 692 | −2.308 | 51.643 | 65.001 | 1.00 | 91.48 |
| 2363 | SG | CYS | 692 | −2.267 | 50.558 | 66.485 | 1.00 | 88.36 |
| 2364 | C | CYS | 692 | −4.614 | 52.731 | 65.000 | 1.00 | 95.54 |
| 2365 | O | CYS | 692 | −5.171 | 51.927 | 65.781 | 1.00 | 90.96 |
| 2366 | OXT | CYS | 692 | −5.252 | 53.355 | 64.114 | 1.00 | 95.28 |
| 2367 | N | CYS | 692 | −2.673 | 53.708 | 66.354 | 1.00 | 78.08 |
| 2368 | CA | CYS | 692 | −3.086 | 52.977 | 65.117 | 1.00 | 91.28 |
| 2369 | OH2 | TIP | 710 | 1.446 | 8.260 | 101.829 | 1.00 | 48.77 |
| 2370 | OH2 | TIP | 711 | −3.102 | 36.787 | 52.191 | 1.00 | 56.74 |
| 2371 | OH2 | TIP | 712 | 3.900 | 28.654 | 57.033 | 1.00 | 60.01 |
| 2372 | OH2 | TIP | 713 | −7.629 | 25.597 | 101.244 | 1.00 | 67.18 |
| 2373 | OH2 | TIP | 714 | 9.048 | 6.640 | 103.720 | 1.00 | 43.72 |
| 2374 | OH2 | TIP | 715 | −12.462 | 42.004 | 87.260 | 1.00 | 52.02 |
| 2375 | OH2 | TIP | 716 | −4.818 | −9.266 | 106.981 | 1.00 | 68.44 |
| 2376 | OH2 | TIP | 717 | 6.933 | 32.505 | 77.460 | 1.00 | 44.82 |
| 2377 | OH2 | TIP | 718 | 8.359 | 31.388 | 63.629 | 1.00 | 57.05 |
| 2378 | OH2 | TIP | 719 | 12.046 | 28.037 | 53.281 | 1.00 | 47.27 |
| 2379 | OH2 | TIP | 720 | 21.986 | 43.471 | 56.597 | 1.00 | 47.40 |
| 2380 | OH2 | TIP | 721 | −10.211 | 15.823 | 105.447 | 1.00 | 85.87 |
| 2381 | OH2 | TIP | 722 | 2.513 | 19.804 | 105.814 | 1.00 | 85.68 |
| 2382 | OH2 | TIP | 723 | 17.346 | 37 | 30.005 | 1.00 | 42.46 |
| 2383 | OH2 | TIP | 724 | 1.539 | 34.482 | 59.834 | 1.00 | 39.64 |
| 2384 | OH2 | TIP | 725 | 1.495 | 32.564 | 48.899 | 1.00 | 49.09 |
| 2385 | OH2 | TIP | 726 | 19.154 | 25.953 | 40.506 | 1.00 | 38.01 |
| 2386 | OH2 | TIP | 727 | 3.014 | 38.706 | 67.455 | 1.00 | 37.92 |
| 2387 | OH2 | TIP | 728 | 21.604 | 32.043 | 37.250 | 1.00 | 32.04 |
| 2388 | OH2 | TIP | 729 | 10.504 | 29.190 | 55.461 | 1.00 | 42.14 |
| 2389 | OH2 | TIP | 730 | 5.406 | 34.319 | 42.912 | 1.00 | 60.51 |
| 2390 | OH2 | TIP | 731 | 6.579 | 29.585 | 57.877 | 1.00 | 42.58 |
| 2391 | OH2 | TIP | 732 | 6.549 | 42.069 | 48.390 | 1.00 | 33.93 |
| 2392 | OH2 | TIP | 733 | 14.868 | 36.504 | 62.509 | 1.00 | 37.94 |
| 2393 | OH2 | TIP | 734 | −11.912 | 15.524 | 108.297 | 1.00 | 85.95 |
| 2394 | OH2 | TIP | 736 | 8.681 | 28.054 | 57.615 | 1.00 | 63.73 |
| 2395 | OH2 | TIP | 738 | 14.634 | 46.656 | 60.620 | 1.00 | 42.35 |
| 2396 | OH2 | TIP | 739 | −1.490 | 31.359 | 53.488 | 1.00 | 58.01 |
| 2397 | OH2 | TIP | 740 | −0.629 | 48.639 | 49.180 | 1.00 | 42.04 |
| 2398 | OH2 | TIP | 741 | 3.357 | 29.653 | 98.183 | 1.00 | 49.62 |
| 2399 | OH2 | TIP | 742 | −2.410 | 34.702 | 66.379 | 1.00 | 45.29 |
| 2400 | OH2 | TIP | 743 | 0.504 | 5.606 | 118.912 | 1.00 | 48.91 |
| 2401 | OH2 | TIP | 744 | −8.947 | 32.692 | 76.363 | 1.00 | 46.46 |
| 2402 | OH2 | TIP | 745 | 10.010 | 44.375 | 63.052 | 1.00 | 43.80 |
| 2403 | OH2 | TIP | 746 | −6.076 | 44.904 | 65.960 | 1.00 | 74.81 |
| 2404 | OH2 | TIP | 747 | 27.154 | 25.041 | 44.331 | 1.00 | 72.57 |
| 2405 | OH2 | TIP | 748 | −2.104 | 33.500 | 52.404 | 1.00 | 54.46 |
| 2406 | OH2 | TIP | 749 | 3.280 | 6.751 | 98.972 | 1.00 | 56.63 |
| 2407 | OH2 | TIP | 750 | 6.404 | 38.165 | 74.126 | 1.00 | 55.28 |
| 2408 | OH2 | TIP | 751 | −3.203 | 47.585 | 57.946 | 1.00 | 53.76 |
| 2409 | OH2 | TIP | 752 | 6.102 | 9.128 | 99.854 | 1.00 | 42.59 |
| 2410 | OH2 | TIP | 753 | −6.382 | 31.302 | 88.650 | 1.00 | 44.76 |
| 2411 | OH2 | TIP | 754 | −6.111 | 36.467 | 59.870 | 1.00 | 65.59 |
| 2412 | OH2 | TIP | 755 | −10.400 | 44.282 | 59.986 | 1.00 | 53.42 |
| 2413 | OH2 | TIP | 756 | −9.045 | −3.631 | 99.653 | 1.00 | 53.10 |
| 2414 | OH2 | TIP | 757 | 9.967 | 46.114 | 83.351 | 1.00 | 58.78 |
| 2415 | OH2 | TIP | 758 | 7.338 | 47.613 | 39.685 | 1.00 | 58.89 |
| 2416 | OH2 | TIP | 759 | 3.002 | 31.624 | 96.365 | 1.00 | 65.65 |
| 2417 | OH2 | TIP | 760 | 3.260 | 10.814 | 91.701 | 1.00 | 65.07 |
| 2418 | OH2 | TIP | 761 | 16.897 | 29.234 | 27.781 | 1.00 | 43.86 |
| 2419 | OH2 | TIP | 762 | 7.572 | 37.085 | 84.612 | 1.00 | 45.30 |
| 2420 | OH2 | TIP | 763 | 3.217 | 42.808 | 60.266 | 1.00 | 39.35 |
| 2421 | OH2 | TIP | 764 | 14.810 | 24.838 | 43.343 | 1.00 | 49.55 |
| 2422 | OH2 | TIP | 765 | −3.308 | 43.008 | 66.534 | 1.00 | 47.94 |
| 2423 | OH2 | TIP | 766 | 26.217 | 31.372 | 58.114 | 1.00 | 75.33 |
| 2424 | OH2 | TIP | 767 | 34.957 | 33.162 | 55.727 | 1.00 | 57.92 |
| 2425 | OH2 | TIP | 768 | −12.434 | 33.606 | 69.661 | 1.00 | 97.00 |
| 2426 | OH2 | TIP | 769 | −3.810 | 43.920 | 54.167 | 1.00 | 63.21 |
| 2427 | OH2 | TIP | 770 | 11.649 | 16.345 | 99.859 | 1.00 | 54.89 |
| 2428 | OH2 | TIP | 771 | 8.100 | 35.402 | 62.318 | 1.00 | 40.93 |
| 2429 | OH2 | TIP | 772 | 6.776 | 29.808 | 77.703 | 1.00 | 62.93 |
| 2430 | OH2 | TIP | 773 | 27.843 | 38.310 | 48.844 | 1.00 | 45.03 |
| 2431 | OH2 | TIP | 774 | 19.522 | 42.489 | 56.330 | 1.00 | 55.14 |
| 2432 | OH2 | TIP | 775 | 17.086 | 22.837 | 42.374 | 1.00 | 50.94 |
| 2433 | OH2 | TIP | 776 | 21.933 | 45.274 | 54.046 | 1.00 | 54.29 |
| 2434 | OH2 | TIP | 777 | 12.848 | 40.311 | 61.570 | 1.00 | 42.74 |
| 2435 | OH2 | TIP | 778 | 2.857 | 29.230 | 69.697 | 1.00 | 53.35 |
| 2436 | OH2 | TIP | 779 | 9.577 | 40.491 | 42.521 | 1.00 | 42.52 |
| 2437 | OH2 | TIP | 780 | 0.121 | 38.085 | 51.111 | 1.00 | 33.56 |
| 2438 | OH2 | TIP | 781 | 4.722 | 45.413 | 79.968 | 1.00 | 34.36 |
| 2439 | OH2 | TIP | 782 | 6.149 | 47.543 | 81.011 | 1.00 | 39.31 |
| 2440 | OH2 | TIP | 783 | 0.328 | 6.268 | 121.230 | 1.00 | 45.10 |
| 2441 | OH2 | TIP | 784 | −11.699 | 38.118 | 73.991 | 1.00 | 46.37 |
| 2442 | OH2 | TIP | 806 | −4.762 | 55.892 | 75.848 | 1.00 | 70.20 |
| 2443 | OH2 | TIP | 807 | −3.005 | 21.561 | 84.512 | 1.00 | 46.81 |
| 2444 | OH2 | TIP | 810 | 9.011 | 33.982 | 91.831 | 1.00 | 55.39 |
| 2445 | OH2 | TIP | 816 | 13.256 | 29.331 | 42.998 | 1.00 | 43.84 |
| 2446 | OH2 | TIP | 825 | 24.114 | 37.014 | 34.599 | 1.00 | 42.27 |
| 2447 | C1 | NAG | 631 | −6.967 | 31.492 | 60.665 | 1.00 | 60.68 |
| 2448 | C2 | NAG | 631 | −8.408 | 30.954 | 60.660 | 1.00 | 67.89 |
| 2449 | N2 | NAG | 631 | −9.208 | 31.634 | 59.660 | 1.00 | 67.82 |
| 2450 | C7 | NAG | 631 | −9.243 | 31.188 | 58.407 | 1.00 | 70.87 |
| 2451 | O7 | NAG | 631 | −8.908 | 30.044 | 58.088 | 1.00 | 66.23 |
| 2452 | C8 | NAG | 631 | −9.716 | 32.173 | 57.344 | 1.00 | 68.04 |
| 2453 | C3 | NAG | 631 | −9.063 | 31.108 | 62.044 | 1.00 | 74.12 |
| 2454 | O3 | NAG | 631 | −10.283 | 30.377 | 62.077 | 1.00 | 73.27 |
| 2455 | C4 | NAG | 631 | −8.151 | 30.592 | 63.166 | 1.00 | 77.89 |
| 2456 | O4 | NAG | 631 | −8.675 | 31 | 64.451 | 1.00 | 82.64 |
| 2457 | C5 | NAG | 631 | −6.754 | 31.166 | 63.008 | 1.00 | 74.10 |
| 2458 | O5 | NAG | 631 | −6.234 | 30.849 | 61.704 | 1.00 | 67.49 |
| 2459 | C6 | NAG | 631 | −5.820 | 30.574 | 64.033 | 1.00 | 62.90 |
| 2460 | O6 | NAG | 631 | −6.159 | 29.222 | 64.272 | 1.00 | 71.83 |
| 2461 | C1 | NAG | 632 | −8.955 | 30.030 | 65.409 | 1.00 | 78.67 |
| 2462 | C2 | NAG | 632 | −8.990 | 30.720 | 66.790 | 1.00 | 80.33 |
| 2463 | N2 | NAG | 632 | −7.629 | 31.045 | 67.194 | 1.00 | 61.31 |
| 2464 | C7 | NAG | 632 | −7.168 | 32.301 | 67.157 | 1.00 | 59.62 |
| 2465 | O7 | NAG | 632 | −7.846 | 33.287 | 66.820 | 1.00 | 36.83 |
| 2466 | C8 | NAG | 632 | −5.714 | 32.489 | 67.586 | 1.00 | 44.94 |
| 2467 | C3 | NAG | 632 | −9.655 | 29.856 | 67.885 | 1.00 | 83.61 |
| 2468 | O3 | NAG | 632 | −9.944 | 30.681 | 69.009 | 1.00 | 84.18 |
| 2469 | C4 | NAG | 632 | −10.959 | 29.180 | 67.408 | 1.00 | 87.97 |
| 2470 | O4 | NAG | 632 | −11.383 | 28.201 | 68.398 | 1.00 | 87.59 |
| 2471 | C5 | NAG | 632 | −10.716 | 28.503 | 66.038 | 1.00 | 92.69 |
| 2472 | O5 | NAG | 632 | −10.233 | 29.471 | 65.076 | 1.00 | 84.66 |
| 2473 | C6 | NAG | 632 | −11.985 | 27.929 | 65.437 | 1.00 | 89.36 |
| 2474 | O6 | NAG | 632 | −12.665 | 28.926 | 64.688 | 1.00 | 83.97 |
| 2475 | C1 | MAN | 633 | −12.686 | 27.687 | 68.372 | 1.00 | 95.32 |
| 2476 | C2 | MAN | 633 | −12.801 | 26.479 | 69.353 | 1.00 | 101.03 |
| 2477 | O2 | MAN | 633 | −12.368 | 26.860 | 70.662 | 1.00 | 91.92 |
| 2478 | C3 | MAN | 633 | −14.258 | 25.990 | 69.412 | 1.00 | 98.59 |
| 2479 | O3 | MAN | 633 | −14.391 | 24.881 | 70.297 | 1.00 | 98.76 |
| 2480 | C4 | MAN | 633 | −15.118 | 27.171 | 69.868 | 1.00 | 95.70 |
| 2481 | O4 | MAN | 633 | −16.471 | 26.774 | 70.047 | 1.00 | 90.82 |
| 2482 | C5 | MAN | 633 | −14.992 | 28.237 | 68.779 | 1.00 | 93.08 |
| 2483 | O5 | MAN | 633 | −13.626 | 28.731 | 68.730 | 1.00 | 96.41 |
| 2484 | C6 | MAN | 633 | −15.965 | 29.404 | 68.883 | 1.00 | 98.62 |
| 2485 | O6 | MAN | 633 | −15.403 | 30.528 | 69.592 | 1.00 | 99.10 |
| 2486 | C1 | MAN | 635 | −16.406 | 31.489 | 69.827 | 1.00 | 107.04 |
| 2487 | C2 | MAN | 635 | −15.819 | 32.869 | 69.953 | 1.00 | 101.17 |
| 2488 | O2 | MAN | 635 | −16.878 | 33.773 | 70.234 | 1.00 | 99.30 |
| 2489 | C3 | MAN | 635 | −14.824 | 32.889 | 71.108 | 1.00 | 108.13 |
| 2490 | O3 | MAN | 635 | −14.378 | 34.225 | 71.310 | 1.00 | 110.39 |
| 2491 | C4 | MAN | 635 | −15.426 | 32.338 | 72.434 | 1.00 | 109.37 |
| 2492 | O4 | MAN | 635 | −14.346 | 31.880 | 73.301 | 1.00 | 110.97 |
| 2493 | C5 | MAN | 635 | −16.393 | 31.136 | 72.221 | 1.00 | 111.45 |
| 2494 | O5 | MAN | 635 | −17.167 | 31.235 | 70.999 | 1.00 | 112.10 |
| 2495 | C6 | MAN | 635 | −17.403 | 32 | 73.333 | 1.00 | 105.71 |
| 2496 | O6 | MAN | 635 | −18.637 | 30.526 | 72.820 | 1.00 | 95.50 |
| 2497 | C1 | NAG | 636 | −13.383 | 32.812 | 73.749 | 1.00 | 108.37 |
| 2498 | C2 | NAG | 636 | −12.075 | 32.105 | 74.102 | 1.00 | 106.85 |
| 2499 | N2 | NAG | 636 | −11.507 | 31.453 | 72.929 | 1.00 | 108.14 |
| 2500 | C7 | NAG | 636 | −10.792 | 30.332 | 73.040 | 1.00 | 106.54 |
| 2501 | O7 | NAG | 636 | −9.778 | 30.245 | 73.737 | 1.00 | 104.97 |
| 2502 | C8 | NAG | 636 | −11.275 | 29.119 | 72.258 | 1.00 | 104.70 |
| 2503 | C3 | NAG | 636 | −12.311 | 31.095 | 75.236 | 1.00 | 107.63 |
| 2504 | O3 | NAG | 636 | −11.056 | 30.706 | 75.788 | 1.00 | 104.94 |
| 2505 | C4 | NAG | 636 | −13.195 | 31.628 | 76.378 | 1.00 | 104.08 |
| 2506 | O4 | NAG | 636 | −13.871 | 30.527 | 76.969 | 1.00 | 99.70 |
| 2507 | C5 | NAG | 636 | −14.253 | 32.684 | 75.987 | 1.00 | 102.18 |
| 2508 | O5 | NAG | 636 | −13.817 | 33.519 | 74.890 | 1.00 | 107.22 |
| 2509 | C6 | NAG | 636 | −14.498 | 33.632 | 77.143 | 1.00 | 96.31 |
| 2510 | O6 | NAG | 636 | −13.882 | 34.890 | 76.891 | 1.00 | 92.06 |
| 2511 | C1 | NAG | 621 | 5.295 | 24.278 | 51.654 | 1.00 | 8 |
| 2512 | C2 | NAG | 621 | 3.821 | 24.628 | 51.924 | 1.00 | 83.54 |
| 2513 | N2 | NAG | 621 | 3.754 | 25.613 | 52.996 | 1.00 | 76.26 |
| 2514 | C7 | NAG | 621 | 2.947 | 26.672 | 52.931 | 1.00 | 69.41 |
| 2515 | O7 | NAG | 621 | 3.110 | 27.660 | 53.647 | 1.00 | 66.32 |
| 2516 | C8 | NAG | 621 | 1.785 | 26.650 | 51.948 | 1.00 | 70.85 |
| 2517 | C3 | NAG | 621 | 3.016 | 23.397 | 52.341 | 1.00 | 86.42 |
| 2518 | O3 | NAG | 621 | 1.631 | 23.713 | 52.319 | 1.00 | 89.94 |
| 2519 | C4 | NAG | 621 | 3.270 | 22.196 | 51.435 | 1.00 | 87.26 |
| 2520 | O4 | NAG | 621 | 2.695 | 21.041 | 52.043 | 1.00 | 94.40 |
| 2521 | C5 | NAG | 621 | 4.781 | 21.975 | 51.221 | 1.00 | 87.92 |
| 2522 | O5 | NAG | 621 | 5.406 | 23.185 | 50.729 | 1.00 | 81.54 |
| 2523 | C6 | NAG | 621 | 5.081 | 20.862 | 50.217 | 1.00 | 86.52 |
| 2524 | O6 | NAG | 621 | 5.172 | 21.358 | 48.885 | 1.00 | 84.86 |
| 2525 | C1 | NAG | 611 | −11.148 | 11.437 | 97.806 | 1.00 | 91.26 |
| 2526 | C2 | NAG | 611 | −11.428 | 10.366 | 96.770 | 1.00 | 92.62 |
| 2527 | N2 | NAG | 611 | −10.273 | 10.291 | 95.893 | 1.00 | 86.94 |
| 2528 | C7 | NAG | 611 | −9.995 | 9.178 | 95.229 | 1.00 | 91.34 |
| 2529 | O7 | NAG | 611 | −9.630 | 8.142 | 95.789 | 1.00 | 85.62 |
| 2530 | C8 | NAG | 611 | −10.151 | 9.224 | 93.705 | 1.00 | 87.48 |
| 2531 | C3 | NAG | 611 | −12.698 | 10.687 | 95.951 | 1.00 | 94.52 |
| 2532 | O3 | NAG | 611 | −13.191 | 9.483 | 95.379 | 1.00 | 97.37 |
| 2533 | C4 | NAG | 611 | −13.852 | 11.361 | 96.742 | 1.00 | 95.70 |
| 2534 | O4 | NAG | 611 | −14.631 | 12.186 | 95.834 | 1.00 | 101.81 |
| 2535 | C5 | NAG | 611 | −13.361 | 12.255 | 97.899 | 1.00 | 95.74 |
| 2536 | O5 | NAG | 611 | −12.296 | 11.618 | 98.625 | 1.00 | 95.10 |
| 2537 | C6 | NAG | 611 | −14.448 | 12.546 | 98.908 | 1.00 | 94.16 |
| 2538 | O6 | NAG | 611 | −14.545 | 11.493 | 99.857 | 1.00 | 96.64 |
| 2539 | C1 | NAG | 612 | −15.716 | 11.624 | 95.153 | 1.00 | 107.99 |
| 2540 | C2 | NAG | 612 | −16.529 | 12.734 | 94.474 | 1.00 | 106.19 |
| 2541 | N2 | NAG | 612 | −17.084 | 13.648 | 95.452 | 1.00 | 103.35 |
| 2542 | C7 | NAG | 612 | −17.842 | 13.192 | 96.444 | 1.00 | 102.04 |
| 2543 | O7 | NAG | 612 | −17.377 | 12.921 | 97.545 | 1.00 | 100.56 |
| 2544 | C8 | NAG | 612 | −19.339 | 13.014 | 96.193 | 1.00 | 92.28 |
| 2545 | C3 | NAG | 612 | −17.654 | 12.138 | 93.632 | 1.00 | 107.09 |
| 2546 | O3 | NAG | 612 | −18.281 | 13.178 | 92.896 | 1.00 | 115.89 |
| 2547 | C4 | NAG | 612 | −17.123 | 11.074 | 92.665 | 1.00 | 107.17 |
| 2548 | O4 | NAG | 612 | −18.212 | 10.414 | 92.028 | 1.00 | 107.76 |
| 2549 | C5 | NAG | 612 | −16.279 | 10.050 | 93.423 | 1.00 | 109.10 |
| 2550 | O5 | NAG | 612 | −15.214 | 10.720 | 94.148 | 1.00 | 114.00 |
| 2551 | C6 | NAG | 612 | −15.646 | 9.014 | 92.499 | 1.00 | 103.51 |
| 2552 | O6 | NAG | 612 | −14.225 | 9.029 | 92.581 | 1.00 | 101.85 |
| 2553 | C1 | NAG | 641 | −4.167 | 5.922 | 127.113 | 1.00 | 99.54 |
| 2554 | C2 | NAG | 641 | −5.049 | 5.892 | 128.371 | 1.00 | 99.25 |
| 2555 | N2 | NAG | 641 | −6.430 | 6.155 | 128.009 | 1.00 | 96.88 |
| 2556 | C7 | NAG | 641 | −7.267 | 5.139 | 127.821 | 1.00 | 103.11 |
| 2557 | O7 | NAG | 641 | −7.715 | 4.451 | 128.751 | 1.00 | 100.91 |
| 2558 | C8 | NAG | 641 | −7.655 | 4.824 | 126.379 | 1.00 | 99.46 |
| 2559 | C3 | NAG | 641 | −4.617 | 6.895 | 129.443 | 1.00 | 97.28 |
| 2560 | O3 | NAG | 641 | −5.240 | 6.539 | 130.671 | 1.00 | 102.54 |
| 2561 | C4 | NAG | 641 | −3.099 | 6.926 | 129.649 | 1.00 | 95.74 |
| 2562 | O4 | NAG | 641 | −2.753 | 8.057 | 130.437 | 1.00 | 89.41 |
| 2563 | C5 | NAG | 641 | −2.369 | 6.995 | 128.302 | 1.00 | 102.02 |
| 2564 | O5 | NAG | 641 | −2.774 | 5.879 | 127.466 | 1.00 | 106.08 |
| 2565 | C6 | NAG | 641 | −0.848 | 6.924 | 128.461 | 1.00 | 95.69 |
| 2566 | O6 | NAG | 641 | −0.175 | 7.145 | 127.226 | 1.00 | 83.83 |
| 2567 | S | SO4 | 701 | 3.456 | 37.105 | 42.513 | 1.00 | 74.45 |
| 2568 | O1 | SO4 | 701 | 3.048 | 38.022 | 41.415 | 1.00 | 76.75 |
| 2569 | O2 | SO4 | 701 | 4.912 | 37.289 | 42.714 | 1.00 | 74.31 |
| 2570 | O3 | SO4 | 701 | 2.673 | 37.414 | 43.732 | 1.00 | 71.58 |
| 2571 | O4 | SO4 | 701 | 3.235 | 35.686 | 42.183 | 1.00 | 68.84 |
| 2572 | S | SO4 | 702 | 10.169 | 30.764 | 90.112 | 1.00 | 91.70 |
| 2573 | O1 | SO4 | 702 | 10.560 | 32.173 | 89.905 | 1.00 | 86.64 |
| 2574 | O2 | SO4 | 702 | 11.294 | 30.046 | 90.744 | 1.00 | 97.68 |
| 2575 | O3 | SO4 | 702 | 8.976 | 30.731 | 90.989 | 1.00 | 90.28 |
| 2576 | O4 | SO4 | 702 | 9.871 | 30.097 | 88.822 | 1.00 | 90.49 |
| 2577 | OH2 | TIP | 826 | 5.503 | 45.240 | 77.177 | 1.00 | 40.35 |
| 2578 | OH2 | TIP | 827 | 5.131 | 35.042 | 95.552 | 1.00 | 43.30 |
| 2579 | OH2 | TIP | 828 | 6.981 | 23.682 | 78.498 | 1.00 | 46.28 |
| 2580 | OH2 | TIP | 829 | 12.402 | 45.258 | 62.673 | 1.00 | 41.43 |
| 2581 | OH2 | TIP | 830 | 12.995 | 31.423 | 61.131 | 1.00 | 44.50 |

```
REMARK coordinates from minimization and B-factor refinement
REMARK refinement resolution: 6-2.4 A
REMARK starting r= 0.2240 free_r= 0.2963
REMARK final  r= 0.2202 free_r= 0.3008
REMARK rmsd bonds= 0.014632 rmsd angles= 1.96283
REMARK B rmsd for bonded mainchain atoms= 6.394 target= 3.5
REMARK B rmsd for bonded sidechain atoms= 7.922 target= 4
REMARK B rmsd for angle mainchain atoms= 7.619 target= 4
REMARK B rmsd for angle sidechain atoms= 9.105 target= 4.5
REMARK target= mlf final wa= 10
REMARK final rweight= 0.0800 (with wa= 10)
REMARK md-method= cartesian annealing schedule= slowcool
REMARK starting temperature= 2000 total md steps= 20 * 50
REMARK cycles= 2 coordinate steps= 20 B-factor steps= 10
REMARK sg= P4(3)2(1)2 a= 51.13 b= 51.13 c= 303.388 alpha= 90
      beta= 90 gamma= 90
REMARK topology file 1 : ../protein.top
REMARK topology file 2 : ../carbohydrate.top
REMARK topology file 3 : CNS_TOPPAR:water.top
REMARK topology file 4 : CNS_TOPPAR:ion.top
REMARK parameter file 1 : ../protein_rep.param
REMARK parameter file 2 : ../carbohydrate.param
REMARK parameter file 3 : CNS_TOPPAR:water_rep.param
REMARK parameter file 4 : CNS_TOPPAR:ion.param
REMARK molecular structure file: water_cyc9.5x.mtf
REMARK input coordinates: posi_cyc9.6x.pdb
REMARK reflection file= ggnew_hl.cv
REMARK ncs= none
REMARK B-correction resolution: 6-2.4
REMARK initial B-factor correction applied to fobs:
REMARK  B11= −6.369 B22= −6.369 B33= 12.737
REMARK  B12= 0.000 B13= 0.000 B23= 0.000
REMARK B-factor correction applied to coordinate array B: 1.790
REMARK bulk solvent: density level= 0.662903 e/A\hat{ }3,
              B-factor= 87.8363 A\hat{ }2
REMARK reflections with |Fobs|/sigma_F < 0.0 rejected
REMARK reflections with |Fobs| > 10000 * rms(Fobs) rejected
REMARK theoretical total number of refl. in resol. range: 15634
(100%)
REMARK number of unobserved reflections (no entry or |F|=0): 336
(2.1%)
```

| | |
|---|---|
| REMARK number of reflections rejected: | 0 ( 0.0%) |
| REMARK total number of reflections used: | 15298 ( 97.9%) |
| REMARK number of reflections in working set: | 14567 ( 93.2%) |
| REMARK number of reflections in test set: | 731 ( 4.7%) |

CRYST1 51.130 51.130 303.388 90.00 90.00 90.00 P 43 21 2
REMARK FILENAME=“refine_cyc9.7x.pdb”
REMARK DATE:16-Oct-00 11:00:40 created by user: jose
REMARK VERSION:1.0

Deduction of Dimer Coordinates
For a dimer comprising a first monomer unit and a second monomer unit, the structures coordinates (in Å) of the first monomer unit are defined by X1, Y1 and Z1 as set forth above, and the structure coordinates of the second monomer unit (in Å) are defined by X2, Y2 and Z2, which can be deduced from the following equations:
X2=51.13−Y1 (1)
Y2=51.13−X1 (2)
Z2=151.7−Z1 (3)

LENGTHY TABLE REFERENCED HERE
US20070032640A1-20070208-T00001
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070032640A1) An electronic copy of the table will also be available from the USPTO upon request and payment of the fee set forth in 37 CFR 1.19(b)(3).

1. A method of selecting or designing a compound that interacts with an IL-6 receptor and modulates an activity mediated by the receptor, the method comprising:
(a) assessing the stereochemical complementarity between a compound and a topographic region of the receptor, wherein the receptor comprises,
(i) amino acids 1-299 of the IL-6 receptor positioned at atomic coordinates as shown in Appendix I, or structural coordinates having a root mean square deviation from the backbone atoms of said amino acids of not more than 1.5 Å; or
(ii) one or more subsets of said amino acids related to the coordinates shown in Appendix I by whole body translations and/or rotations;
(b) obtaining a compound which possesses stereochemical complementarity to a topographic region of the receptor; and
(c) testing the compound for its ability to modulate an activity associated with the receptor.

2. A method as claimed in claim 1 wherein the topographic region of the IL-6 receptor is a ligand binding surface defined by residues 106-110, 133-138, 160-168, 190-193, 227-233, 250-256 or 276-281 and combinations thereof.

3. A method as claimed in claim 1 wherein the topographic region of the IL-6 receptor is a region on the homodimer interface defined by resides 1-5, 19-23, 65-69, 93-99, 118, 119, 132-141, 166-172, 179-196, 241-250, 261, 262, 272-276 or 282-290 and combinations thereof.

4. A method as claimed in claim 1 wherein the topographic region of the IL-6 receptor is defined by residues 11, 45, 46, 55, 62-66, 69-72, 75, 81, 88, 90-93, 122-124 and 178.

5. A method as claimed in claim 1 wherein the topographic region of the IL-6 receptor is defined by residues 233-239, 244-248 and 270-290.

6. A method for identifying a potential modulator compound for an IL-6 receptor which method comprises:
(a) providing a three-dimensional structure of amino acids 1-299 of an IL-6 receptor as defined by the atomic coordinates shown in Appendix I, or atomic coordinates having a root mean square deviation from the backbone atoms of said amino acids of not more than 1.5 Å, or one or more subsets of said amino acids, or one or more subsets of said amino acids related to the coordinates shown in Appendix I by whole body translations and/or rotations;
(b) providing the three-dimensional structure of a candidate compound; and
(c) assessing the stereochemical complementarity between the three-dimensional structure of step (b) and a topographical region of the three-dimensional structure of step (a).

7. A method as claimed in claim 6 which further comprises:
(d) synthesising or obtaining a candidate compound assessed in step (c) as possessing stereochemical complementarity with a topographical region of the three-dimensional structure of step (a);
(e) determining the ability of the candidate compound to interact with and/or modulate the activity of the IL-6 receptor.

8. A method as claimed in claim 6 wherein the topographic region of the IL-6 receptor is a ligand binding surface defined by residues 106-110, 133-138, 160-168, 190-193, 227-233, 250-256 or 276-281 and combinations thereof.

9. A method as claimed in claim 6 wherein the topographic region of the IL-6 receptor is a region on the homodimer interface defined by resides 1-5, 19-23, 65-69, 93-99, 118, 119, 132-141, 166-172, 179-196, 241-250, 261, 262, 272-276 or 282-290 and combinations thereof.

10. A method as claimed in claim 6, wherein the topographic region of the IL-6 receptor is defined by residues 11, 45, 46, 55, 62-66, 69-72, 75, 81, 88, 90-93, 122-124 and 178.

11. A method as claimed in claim 6 wherein the topographic region of the IL-6 receptor is defined by residues 233-239, 244-248 and 270-290.

12. A computer-assisted method for identifying potential compounds able to interact with an IL-6 receptor and thereby modulate an activity mediated by the receptor, using a programmed computer comprising a processor, an input device, and an output device, comprising the steps of:
(a) inputting into the programmed computer, through the input device, data comprising the atomic coordinates of amino acids 1-299 of the IL-6 receptor as shown in Appendix I, or structural coordinates having a root mean square deviation from the backbone atoms of said amino acids of not more than 1.5 Å, or one or more subsets of said amino acids, or one or more subsets of said amino acids related to the coordinates shown in Appendix I by whole body translations and/or rotations;
(b) generating, using computer methods, a set of atomic coordinates of a structure that possesses stereochemical complementarity to the atomic coordinates of amino acids 1-299 of the IL-6 receptor as shown in Appendix I, or structural coordinates having a root mean square deviation from the backbone atoms of said amino acids of not more than 1.5 Å, or one or more subsets of said amino acids, or one or more subsets of said amino acids related to the coordinates shown in Appendix I by whole body translations and/or rotations, thereby generating a criteria data set;
(c) comparing, using the processor, the criteria data set to a computer database of chemical structures;
(d) selecting from the database, using computer methods, chemical structures which are similar to a portion of said criteria data set; and
(e) outputting, to the output device, the selected chemical structures which are complementary to or similar to a portion of the criteria data set.

13. A method for evaluating the ability of a chemical entity to interact with an IL-6 receptor, said method comprising the steps of:
(a) creating a computer model of at least one region of the IL-6 receptor using structure coordinates wherein the root mean square deviation between said structure coordinates and the structure coordinates of amino acids 1-299 of IL-6 receptor as set forth in Appendix I is not more than about 1.5 Å;
(b) employing computational means to perform a fitting operation between the chemical entity and said computer model of the binding surface; and
(c) analysing the results of said fitting operation to quantify the association between the chemical entity and the binding surface model.

14. A computer for producing a three-dimensional representation of a molecule or molecular complex, wherein the computer comprises:
(a) a machine-readable data storage medium comprising a data storage material encoded with machine-readable data, wherein the machine readable data comprise the atomic coordinates of amino acids 1-299 of the IL-6 receptor as shown in Appendix I, or structural coordinates having a root mean square deviation from the backbone atoms of said amino acids of not more than 1.5 Å, or one or more subsets of said amino acids, or one or more subsets of said amino acids related to the coordinates shown in Appendix I by whole body translations and/or rotations;
(b) a working memory for storing instructions for processing the machine-readable data;
(c) a central-processing unit coupled to the working memory and to the machine-readable data storage medium, for processing the machine-readable data into the three dimensional representation; and
(d) an output hardware coupled to the central processing unit, for receiving the three-dimensional representation.

15. A method of selecting or designing a compound that interferes with the formation of an IL-6, IL-6 receptor, gp130 hexameric complex, the method comprising
(a) assessing the stereochemical complementarity between the compound and a topographic region of the complex, wherein the complex is characterised by
(i) the amino acids of IL-6, IL-6 receptor and gp130 positioned at atomic coordinates as shown in Appendix II, or structural coordinates having a root mean square deviation from the backbone atoms of said amino acids of not more than 1.5 Å; or
(ii) one or more subsets of said amino acids related to the coordinates shown in Appendix II by whole body translations and/or rotations;
(b) obtaining a compound which possesses stereochemical complementarity to a topographic region of the receptor; and
(c) testing the compound for its ability to interfere with the formation of the IL-6, IL-6 receptor, gp130 hexameric complex.

16. A method as claimed in claim 15 wherein the topographic region of the complex is selected from the group consisting of;
(i) amino acids 20, 24, 25, 27, 29, 31, 32, 35, 36, 39, 40-42, 45, 51, 52, 56, 60, 62-64, 69-71, 75-79, 93, 94, 110-115, 117-119, 121-123, 125, 126, 128-147, 151, 152, 155, 159, 161-169, 172, 173, 176, 177, 179, 180, 183 and 184 of IL-6 and combinations thereof; and
(ii) amino acids 1-5, 8-15, 49, 75-78, 114, 116, 132-137, 140-154, 163-172, 177-184, 193-196, 226, 227, 229, 231, 232, 281-283 and 285 of gp130 and combinations thereof; and
(iii) amino acids 1, 6, 107, 108, 135-139, 161-169, 190, 193, 226-231 and 277-281 of IL-6R and combinations thereof.

17. A computer-assisted method for identifying compounds that interfere with the formation of an IL-6, IL-6 receptor, gp130 hexameric complex, using a programmed computer comprising a processor, an input device, and an output device, comprising the steps of:
(a) inputting into the programmed computer, through the input device, data comprising the atomic coordinates of amino acids IL-6, IL-6 receptor and gp130 as shown in Appendix II, or structural coordinates having a root mean square deviation from the backbone atoms of said amino acids of not more than 1.5 Å, or one or more subsets of said amino acids, or one or more subsets of said amino acids related to the coordinates shown in Appendix II by whole body translations and/or rotations;
(b) generating, using computer methods, a set of atomic coordinates of a structure that possesses stereochemical complementarity to the atomic coordinates the IL-6, IL-6 receptor, gp130 hexameric complex as shown in Appendix II, or structural coordinates having a root mean square deviation from the backbone atoms of said amino acids of not more than 1.5 Å, or one or more subsets of said amino acids, or one or more subsets of said amino acids related to the coordinates shown in Appendix II by whole body translations and/or rotations, thereby generating a criteria data set;
(c) comparing, using the processor, the criteria data set to a computer database of chemical structures;
(d) selecting from the database, using computer methods, chemical structures which are similar to a portion of said criteria data set; and
(e) outputting, to the output device, the selected chemical structures which are complementary to or similar to a portion of the criteria data set.

18. A method for evaluating the ability of a chemical entity to interact with an IL-6, IL-6 receptor, gp130 hexameric complex, said method comprising the steps of:
(a) creating a computer model of at least one region of the IL-6, IL-6 receptor, gp130 hexameric complex using structure coordinates wherein the root mean square deviation between said structure coordinates and the structure coordinates set forth in Appendix I is not more than about 1.5 Å;
(b) employing computational means to perform a fitting operation between the chemical entity and said computer model; and
(c) analysing the results of said fitting operation to quantify the association between the chemical entity and the model.

19. A computer for producing a three-dimensional representation of a molecule or molecular complex, wherein the computer comprises:
(a) a machine-readable data storage medium comprising a data storage material encoded with machine-readable data, wherein the machine readable data comprise the atomic coordinates of the IL-6, IL-6 receptor, gp130 hexameric complex as shown in Appendix II, or structural coordinates having a root mean square deviation from the backbone atoms of said amino acids of not more than 1.5 Å, or one or more subsets of said amino acids, or one or more subsets of the coordinates shown in Appendix II by whole body translations and/or rotations;
(b) a working memory for storing instructions for processing the machine-readable data;
(c) a central-processing unit coupled to the working memory and to the machine-readable data storage medium, for processing the machine-readable data into the three dimensional representation; and
(d) an output hardware coupled to the central processing unit, for receiving the three-dimensional representation.

20. A crystalline composition comprising a crystal of an IL-6 receptor.

21. A composition according to claim 20 wherein the crystal has the structure defined by the atomic coordinates as shown in Appendix I.

22. A method of assessing the interaction between a compound and an IL-6 receptor, the method comprising contacting a crystalline composition according to claim 20 with the compound and measuring the level of binding of the compound to the crystal of the IL-6 receptor.

23. A method of using molecular replacement to obtain structural information about a molecule or a molecular complex of unknown structure, comprising the steps of:
(i) crystallising said molecule or molecular complex;
(ii) generating an X-ray diffraction pattern from said crystallized molecule or molecular complex;
(iii) applying at least a portion of the structure coordinates set forth in Appendix I to the X-ray diffraction pattern to generate a three-dimensional electron density map of at least a portion of the molecule or molecular complex whose structure is unknown.

24. A method according to claim 23 wherein the molecule of unknown structure is an IL-6 receptor or variant.

25. A method according to claim 23 wherein the molecular complex of unknown structure is a complex of an IL-6 receptor, or variant thereof, and a ligand.

26. A method for preventing or treating a disease associated with signaling by the IL-6 receptor which method comprises administering to a subject in need thereof a compound identified by a method comprising the step of assessing the stereochemical complementarity between the compound and a topographic region of the receptor, wherein the receptor comprises:
(i) amino acids 1-299 of the IL-6 receptor positioned at atomic coordinates as shown in Appendix I, or structural coordinates having a root mean square deviation from the backbone atoms of said amino acids of not more than 1.5 Å; or
(ii) one or more subsets of said amino acids related to the coordinates shown in Appendix I by whole body translations and/or rotations.

27. A method as claimed in claim 26 wherein the topographic region of the IL-6 receptor is a ligand binding surface defined by residues 106-110, 133-138, 160-168, 190-193, 227-233, 250-256 or 276-281 and combinations thereof.

28. A method as claimed in claim 26 wherein the topographic region of the IL-6 receptor is a region on the homodimer interface defined by resides 1-5, 19-23, 65-69, 93-99, 118, 119, 132-141, 166-172, 179-196, 241-250, 261, 262, 272-276 or 282-290 and combinations thereof.

29. A method as claimed in claim 26 wherein the topographic region of the IL-6 receptor is defined by residues 11, 45, 46, 55, 62-66, 69-72, 75, 81, 88, 90-93, 122-124 and 178.

30. A method as claimed in claim 26 wherein the topographic region of the IL-6 receptor is defined by residues 233-239, 244-248 and 270-290.

31. A method for preventing or treating a disease associated with signaling by the IL-6 receptor which method comprises administering to a subject in need thereof a compound identified by a method comprising the step of assessing the stereochemical complementarity between the compound and a topographic region of an IL-6, IL-6R, gp130 hexameric complex, wherein the hexameric complex comprises:
(i) the amino acids of IL-6, IL-6 receptor and gp130 positioned at atomic coordinates as shown in Appendix II, or structural coordinates having a root mean square deviation from the backbone atoms of said amino acids of not more than 1.5 Å; or
(ii) one or more subsets of said amino acids related to the coordinates shown in Appendix II by whole body translations and/or rotations.

32. A method as claimed in claim 31 wherein the topographic region of the complex is selected from the group consisting of;
(i) amino acids 20, 24, 25, 27, 29, 31, 32, 35, 36, 39, 40-42, 45, 51, 52, 56, 60, 62-64, 69-71, 75-79, 93, 94, 110-115, 117-119, 121-123, 125, 126, 128-147, 151, 152, 155, 159, 161-169, 172, 173, 176, 177, 179, 180, 183 and 184 of IL-6 and combinations thereof; and
(ii) amino acids 1-5, 8-15, 49, 75-78, 114, 116, 132-137, 140-154, 163-172, 177-184, 193-196, 226, 227, 229, 231, 232, 281-283 and 285 of gp130 and combinations thereof; and
(iii) amino acids 1, 6, 107, 108, 135-139, 161-169, 190, 193, 226-231 and 277-281 of IL-6R and combinations thereof.

33. A method as claimed in claim 26 wherein the disease is selected from multiple myeloma, lymphoma, inflammation, rheumatoid arthritis, prostate cancer, Castleman's disease, AIDS, mesangial proliferative glomerulonephritis, Kaposi's sarcoma, sepsis, osteoporosis and psoriasis.

34. A compound comprising an extracellular portion of IL-6R, wherein the extracellular portion is modified at one or more amino acids of IL-6R selected from the group consisting of:
(i) amino acids 106-110, 133-138, 160-168, 190-193, 227-233, 250-256 and 276-281; or
(ii) amino acids 1-5, 19-23, 65-69, 93-99, 118, 119, 132-141, 166-172, 179-196, 241-250, 261, 262, 272-276 and 282-290; or
(iii) amino acids 11, 45, 46, 55, 62-66, 69-72, 75, 81, 88, 90-93, 122-124 and 178; or
(iv) amino acids 233-239, 244-248 and 270-290; or
(v) amino acids 1, 6, 107, 108, 135-139, 161-169, 190, 193, 226-231 and 277-281.

35. A pharmaceutical composition comprising a compound as claimed in claim 34.

36. A method of preventing or treating a disease associated with signalling by the IL-6 receptor which method comprises administering to a subject in need thereof a composition according to claim 35.

37. A method of modulating the activity of an IL-6 receptor which method comprises contacting the IL-6 receptor with a compound of formula A-B-C, wherein
A consists of three fused 5-, 6- or 7-membered, saturated, unsaturated or aryl rings, optionally containing one or more heteroatoms and optionally substituted; or two non-fused 5- or 6-membered saturated, unsaturated or aryl rings, optionally containing one or more heteroatoms and optionally substituted;
C consists of three fused 5-, 6- or 7-membered, saturated, unsaturated or aryl rings, optionally containing one or more heteroatoms and optionally substituted; or two non-fused 5-, 6- or 7-membered saturated, unsaturated or aryl rings, optionally containing one or more heteroatoms and optionally substituted; and
B is an aliphatic linker having a length substantially equivalent to an ethylene moiety; wherein said compound has stereocomplementarity to a ligand binding topographic region of:
(i) amino acids 1-299 of the IL-6 receptor positioned at atomic coordinates as shown in Appendix I, or structural coordinates having a root mean square deviation from the backbone atoms of said amino acids of not more than 1.5 Å; or
(ii) one or more subsets of said amino acids related to the coordinates shown in Appendix I by whole body translations and/or rotations; said ligand binding topographic region being defined by residues 106-110, 133-138, 160-168, 190-193, 227-233, 250-256 and 276-281 of said IL-6 receptor, or combinations thereof.

38. A method according to claim 37 wherein:
A has the following formula 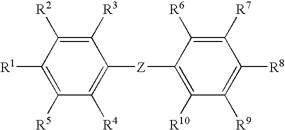
wherein,
(i) Z is a bond; or Z, R⁴ and R¹⁰ taken together form an optionally substituted, saturated, unsaturated or aryl ring having 5, 6 or 7 members, optionally containing one or more heteroatoms selected from O, N and S; or Z, R³ and R⁶ taken together form an aryl ring or a heteroaryl, cycloalkyl, cycloalkenyl or heterocyclyl ring having 5, 6 or 7 members, wherein said aryl ring or said heteroaryl, cycloalkyl, cycloalkenyl, heterocyclyl ring are optionally substituted;
(ii) R⁷, R⁸ or R⁹ are bonded to linker B;
(iii) R¹, R² and R⁵ are each independently, hydrogen, C₁-C₄ alkyl, halogen, OR²⁴ or NR²⁵R²⁶, where R²⁴, R²⁵ and R²⁶ are each independently hydrogen or C₁-C₄ alkyl;
(iv) R³ and R⁶ unless bonded together with Z are each independently, hydrogen, C₁-C₄ alkyl, halogen, O, OR²⁴ or NR²⁵R²⁶, where R²⁴, R²⁵ and R²⁶ are each independently hydrogen or C₁-C₄ alkyl;
(v) R⁴ and R¹⁰ unless bonded together with Z, are each independently, hydrogen, C₁-C₄ alkyl, halogen, O, OR²⁴ or NR²⁵R²⁶, where R²⁴, R²⁵ and R²⁶ are each independently hydrogen or C₁-C₄ alkyl;
(vi) R⁷, R⁸ and R⁹ unless bonded to linker B, are each independently, hydrogen, C₁-C₄ alkyl, halogen, O, OR²⁴ or NR²⁵R²⁶, where R²⁴, R²⁵ and R²⁶ are each independently hydrogen or C₁-C₄ alkyl;
B has the following formula: 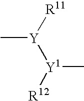
wherein,
(i) Y and Y¹ are each independently C, O, S or N, provided that Y and Y¹ are not both O, N or S;
(ii) R¹¹ and R¹² are each independently hydrogen, C₁-C₄ alkyl, halogen, O, OR²⁴ or NR²⁵R²⁶, where R²⁴, R²⁵ and R²⁶ are each independently hydrogen or C₁-C₄ alkyl;
C has the following formula: 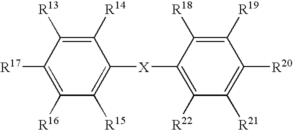
wherein,
(i) X is a bond; or X, R¹⁴ and R¹⁸ taken together form an optionally substituted, saturated, unsaturated or aryl ring having 5, 6 or 7 members, optionally containing one or more heteroatoms selected from O, N and S; or X, R¹⁵ and R²² taken together form an aryl ring or a heteroaryl, cycloalkyl, cycloalkenyl or heterocyclyl ring having 5, 6 or 7 members, wherein said aryl ring or said heteroaryl, cycloalkyl, cycloalkenyl, heterocyclyl ring are optionally substituted;
(ii) R¹³, R¹⁶ or R¹⁷ are bonded to linker B;
(iii) R¹⁹, R²⁰ and R²¹ are each independently, hydrogen, C₁-C₄ alkyl, halogen, O, OR²⁷ or NR²⁸R²⁹, where R²⁷, R²⁸ and R²⁹ are each independently hydrogen or C₁-C₄ alkyl;
(iv) R¹⁴ and R¹⁸ unless bonded together with Z are each independently, hydrogen, C₁-C₄ alkyl, halogen, O, OR²⁷ or NR²⁸R²⁹, where R²⁷, R²⁸ and R²⁹ are each independently hydrogen or C₁-C₄ alkyl;
(v) R¹⁵ and R²² unless bonded together with Z, are each independently, hydrogen, C₁-C₄ alkyl, halogen, O, OR²⁷ or NR²⁸R²⁹, where R²⁷, R²⁸ and R²⁹ are each independently hydrogen or C₁-C₄ alkyl;
(vi) R¹³, R¹⁶ and R¹⁷ unless bonded to linker B, are each independently, hydrogen, C₁-C₄ alkyl, halogen, O, OR²⁷ or NR²⁸R²⁹, where R²⁷, R²⁸ and R²⁹ are each independently hydrogen or C₁-C₄ alkyl.

39. A method of modulating the activity of an IL-6 receptor which method comprises contacting the IL-6 receptor with a compound of formula A-B-D, wherein
A consists of three fused 5-, 6- or 7-membered, saturated, unsaturated or aryl rings, optionally containing one or more heteroatoms and optionally substituted at any position; or two non-fused 5-, 6- or 7-membered saturated, unsaturated or aryl rings, optionally containing one or more heteroatoms and optionally substituted;
D consists of one, or two fused, 5-, 6- or 7-membered, saturated, unsaturated or aryl rings, optionally containing one or more heteroatoms and optionally substituted at any position; or two non-fused 5-, 6- or 7-membered saturated, unsaturated or aryl rings, optionally containing one or more heteroatoms and optionally substituted; and
B is an aliphatic linker having a length substantially equivalent to an ethylene moiety; wherein said compound has stereocomplementarity to a ligand binding topographic region of:
(i) amino acids 1-299 of the IL-6 receptor positioned at atomic coordinates as shown in Appendix I, or structural coordinates having a root mean square deviation from the backbone atoms of said amino acids of not more than 1.5 Å; or
(ii) one or more subsets of said amino acids related to the coordinates shown in Appendix I by whole body translations and/or rotations;
said ligand binding topographic region being defined by residues 106-110, 133-138, 160-168, 190-193, 227-233, 250-256 and 276-281 of said IL-6 receptor, or combinations thereof.

40. A method according to claim 39 wherein:
A has the following formula: 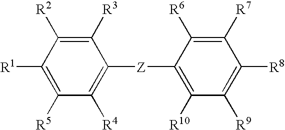
wherein,
(i) Z is a bond; or Z, R⁴ and R¹⁰ taken together form an optionally substituted, saturated, unsaturated or aryl ring having 5, 6 or 7 members, optionally containing one or more heteroatoms selected from O, N and S; or Z, R³ and R⁶ taken together form an aryl ring or a heteroaryl, cycloalkyl, cycloalkenyl or heterocyclyl ring having 5 or 6 members, wherein said aryl ring or said heteroaryl, cycloalkyl, cycloalkenyl, heterocyclyl ring are optionally substituted;
(ii) R⁷, R⁸ or R⁹ are bonded to linker B;
(iii) R¹, R² and R⁵ are each independently, hydrogen, C₁-C₄ alkyl, halogen, OR²⁴ or NR²⁵R²⁶, where R²⁴, R²⁵ and R²⁶ are each independently hydrogen or C₁-C₄ alkyl;
(iv) R³ and R⁶ unless bonded together with Z are each independently, hydrogen, C₁-C₄ alkyl, halogen, O, OR²⁴ or NR²⁵R²⁶, where R²⁴, R²⁵ and R²⁶ are each independently hydrogen or C₁-C₄ alkyl;
(v) R⁴ and R¹⁰ unless bonded together with Z, are each independently, hydrogen, C₁-C₄ alkyl, halogen, O, OR²⁴ or NR²⁵R²⁶, where R²⁴, R²⁵ and R²⁶ are each independently hydrogen or C₁-C₄ alkyl;
(vi) R⁷, R⁸ and R⁹ unless bonded to linker B, are each independently, hydrogen, C₁-C₄ alkyl, halogen, O, OR²⁴ or NR²⁵R²⁶, where R²⁴, R²⁵ and R²⁶ are each independently hydrogen or C₁-C₄ alkyl;
B has the following formula: 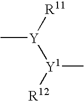
wherein,
Y and Y¹ are each independently C, O, S or N, provided that Y and Y¹ are not both O, N or S;
D has the following formula: 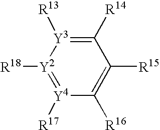
wherein,
(i) Y², Y³ or Y⁴ are each independently C, O, N or S, provided that at least two of Y², Y³ and Y⁴ are C;
(ii) R¹³, R¹⁷ or R¹⁸ are bonded to linker B;
(iii) R¹⁴, R¹⁵ and R¹⁶ are each independently, hydrogen, C₁-C₄ alkyl, halogen, O, OR²⁷ or NR²⁸R²⁹, where R²⁷, R²⁸ and R²⁹ are each independently hydrogen or C₁-C₄ alkyl; or
(iv) R¹⁶ is hydrogen, C₁-C₄ alkyl, halogen, O, OR²⁷ or NR²⁸R²⁹, where R²⁷, R²⁸ and R²⁹ are each independently hydrogen or C₁-C₄ alkyl, and R¹⁴ and R¹⁵ together form an optionally substituted, saturated, unsaturated or aryl ring having 5, 6 or 7 members, optionally containing one or more heteroatoms selected from O, N and S; or
(v) R¹⁴ is hydrogen, C₁-C₄ alkyl, halogen, O, OR²⁷ or NR²⁸R²⁹, where R²⁷, R²⁸ and R²⁹ are each independently hydrogen or C₁-C₄ alkyl R¹⁵ and R¹⁶ together form an optionally substituted, saturated, unsaturated or aryl ring having 5, 6 or 7 members, optionally containing one or more heteroatoms selected from O, N and S;
(vi) R¹³, R¹⁷ and R¹⁸ unless bonded to linker B, are each independently, hydrogen, C₁-C₄ alkyl, halogen, O, OR²⁷ or NR²⁸R²⁹, where R²⁷, R²⁸ and R²⁹ are each independently hydrogen or C₁-C₄ alkyl.

41. A method for preventing or treating a disease associated with signaling by the IL-6 receptor which method comprises administering to a subject in need thereof a compound as defined in claim 37.

42. A method as claimed in claim 7 wherein the topographic region of the IL-6 receptor is a ligand binding surface defined by residues 106-110, 133-138, 160-168, 190-193, 227-233, 250-256 or 276-281 and combinations thereof.

43. A method as claimed in claim 7 wherein the topographic region of the IL-6 receptor is a region on the homodimer interface defined by resides 1-5, 19-23, 65-69, 93-99, 118, 119, 132-141, 166-172, 179-196, 241-250, 261, 262, 272-276 or 282-290 and combinations thereof.

44. A method as claimed in claim 7 wherein the topographic region of the IL-6 receptor is defined by residues 11, 45, 46, 55, 62-66, 69-72, 75, 81, 88, 90-93, 122-124 and 178.

45. A method as claimed in claim 7 wherein the topographic region of the IL-6 receptor is defined by residues 233-239, 244-248 and 270-290.

46. A method for preventing or treating a disease associated with signaling by the IL-6 receptor which method comprises administering to a subject in need thereof a compound as defined in claim 38.

47. A method for preventing or treating a disease associated with signaling by the IL-6 receptor which method comprises administering to a subject in need thereof a compound as defined in claim 39.

48. A method for preventing or treating a disease associated with signaling by the IL-6 receptor which method comprises administering to a subject in need thereof a compound as defined in claim 40.


SEQUENCE LISTING (2 sequences)

SEQ ID NO: 1 - protein, 325 aa, Homo sapiens
LAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNIL

SEQ ID NO: 2 - protein, 5 aa, Artificial Sequence
  Description of Artificial Sequence Illustrative conserved peptide motif
  MOD_RES (3): Variable amino acid
WSXWS